(19) United States
(12) Patent Application Publication
(10) Pub. No.: US 2010/0317697 A1
(43) Pub. Date: Dec. 16, 2010

(54) Renin Inhibitors

(76) Inventors: John J. Baldwin, Gwynedd Valley, PA (US); Salvacion Cacatian, Blue Bell, PA (US); David Claremon, Maple Glen, PA (US); Lawrence W. Dillard, Yardley, PA (US); Patrick T. Flaherty, Pittsburgh, PA (US); Bahman Ghavimi-Alagha, Wilmington, DE (US); Damiano Ghirlanda, Legnago (IT); Alexey V. Ishchenko, Somerville, MA (US); Lara S. Kallander, King of Prussia, PA (US); Brian Lawhorn, King of Prussia, PA (US); Qing Lu, King of Prussia, PA (US); Gerard McGeehan, Garnet Valley, PA (US); Beth A. Knapp-Reid, King of Prussia, PA (US); Simon Semus, Collegeville, PA (US); Robert D. Simpson, Wilmington, DE (US); Suresh B. Singh, Kendall Park, NJ (US); Lamont R. Terrell, King of Prussia, PA (US); Colin Tice, Ambler, PA (US); Tritin Tran, King of Prussia, PA (US); Zherong Xu, Horsham, PA (US); Jing Yuan, Lansdale, PA (US); Wei Zhao, Eagleville, PA (US); Yongdong Y. Zhao, King of Prussia, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/665,217

(22) PCT Filed: Jun. 20, 2008
(86) PCT No.: PCT/US08/67650

§ 371 Date: Jun. 23, 2010

Related U.S. Application Data
(60) Provisional application No. 60/945,157, filed on Jun. 20, 2007.

(51) Int. Cl. A61K 31/445; C07D 211/06

(52) U.S. Cl. 514/330; 546/226

(57) ABSTRACT

Disclosed are compounds having the formula (I): wherein the R¹, R², R³, X, Y, A, L, and G are defined herein. These compounds bind to aspartic proteases to inhibit their activity and are useful in the treatment or amelioration of diseases associated with aspartic protease activity. Also disclosed are methods of use of the compounds of Formula I for ameliorating or treating aspartic protease related disorders in a subject in need thereof.

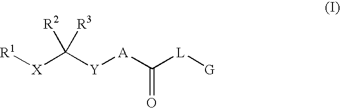

### BACKGROUND OF THE INVENTION

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of AT1 receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Il Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

### SUMMARY OF THE INVENTION

Compounds described herein have been found which are orally active and bind to renin to inhibit its activity. They are useful in the treatment or amelioration of diseases associated with renin activity.

In one embodiment the present invention is directed to a compound represented by Formula I:

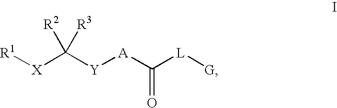

or a pharmaceutically acceptable salt thereof.

In the compounds of Formula I, R¹ is:

a) (C₁-C₁₂)alkyl, (C₃-C₇)cycloalkyl, (C₄-C₁₂)cycloalkyl(C₁-C₆)alkyl, halo(C₁-C₁₂)alkyl, halo(C₃-C₇)-cycloalkyl, halo(C₄-C₁₂)cycloalkyl(C₁-C₆)alkyl, or saturated heterocyclyl, each optionally substituted with 1 to 5 groups independently selected from the group consisting of halogen, (C₁-C₆)alkyl, halo(C₁-C₆)alkyl, and oxo; or

b) phenyl, naphthyl, heteroaryl, or bicyclic heteroaryl, each optionally substituted with 1 to 5 groups independently selected from the groups consisting of:

1) fluorine, chlorine, bromine, iodine, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkyl(C₁-C₆)alkyl, (C₂-C₆)alkenyl, (C₅-C₈)cycloalkenyl, (C₅-C₈)cycloalkyl(C₂-C₆)alkenyl, (C₂-C₈)alkynyl, (C₃-C₈)cycloalkyl(C₂-C₄)alkynyl, halo(C₁-C₈)alkyl, halo(C₃-C₆)cycloalkyl, halo(C₄-C₈)cycloalkyl(C₁-C₆)alkyl, (C₁-C₃)alkyl(C₄-C₈)cycloalkyl(C₁-C₆)alkyl, di(C₁-C₃)alkyl(C₄-C₈)cycloalkyl(C₁-C₆)alkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkyl(C₂-C₆)alkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)-cycloalkylalkoxy, (C₁-C₈)alkylthio, (C₃-C₈)cycloalkythio, (C₄-C₈)cycloalkyl(C₁-C₆)alkylthio, (C₁-C₃)alkyl(C₃-C₈)cycloalkythio, (C₁-C₃)alkyl(C₄-C₈)cycloalkyl(C₁-C₆)alkylthio, di(C₁-C₃)alkyl(C₃-C₈)-cycloalkythio, di(C₁-C₃)alkyl(C₄-C₈)cycloalkyl(C₁-C₆)alkylthio, halo(C₁-C₈)alkylthio, halo(C₃-C₈)-cycloalkythio, halo(C₄-C₈)-cycloalkyl(C₁-C₆)alkylthio, (C₁-C₈)alkylsulfinyl, (C₃-C₈)-cycloalkyl-sulfinyl, (C₄-C₈)cycloalkyl-alkylsulfinyl, (C₁-C₃)alkyl(C₃-C₈)cycloalkyl-sulfinyl, (C₁-C₃)alkyl(C₄-C₈)cycloalkyl-alkylsulfinyl, di(C₁-C₃)alkyl(C₃-C₈)cycloalkyl-sulfinyl, di(C₁-C₃)alkyl(C₄-C₈)cycloalkyl-alkylsulfinyl, halo(C₁-C₈)alkylsulfinyl, halo(C₃-C₈)cycloalkylsulfinyl, halo(C₄-C₈)cycloalkyl(C₁-C₆)alkylsulfinyl, (C₁-C₈)alkyl-sulfonyl, (C₃-C₈)cycloalkylsulfonyl, (C₄-C₈)cycloalkyl(C₁-C₆)alkylsulfonyl, (C₁-C₃)alkyl(C₃-C₈)cycloalkylsulfonyl, (C₁-C₃)alkyl(C₄-C₈)-cycloalkyl-alkylsulfonyl, di(C₁-C₃)alkyl(C₃-C₈)cycloalkylsulfonyl, di(C₁-C₃)alkyl(C₄-C₈)-cycloalkyl-alkylsulfonyl, halo(C₁-C₈)alkylsulfonyl, halo(C₃-C₈)cycloalkylsulfonyl, halo(C₄-C₈)cycloalkyl(C₁-C₆)alkyl-sulfonyl, (C₁-C₈)alkylamino, di(C₁-C₈)alkylamino, (C₁-C₆)alkoxy-(C₁-C₆)-alkoxy, halo(C₁-C₆)alkoxy(C₁-C₆)alkoxy, (C₁-C₈)alkoxycarbonyl, aminocarbonyl, (C₁-C₈)alkylamino-carbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)-cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio-(C₁-C₆)alkyl, (C₃-C₈)cycloalkythio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio-(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)-cycloalkyl-sulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈)-cycloalkyl(C₁-C₆)alkyl-sulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino-(C₁-C₆)alkyl, (C₁-C₈)alkoxycarbonyl(C₁-C₆)alkyl, (C₁-C₈)acyloxy(C₁-C₆)alkyl, aminocarbonyl-(C₁-C₆)alkyl, (C₁-C₈)alkylamino-carbonyl(C₁-C₆)alkyl, di(C₁-C₈)alkylamino-carbonyl(C₁-C₆)alkyl, (C₁-C₈)acylamino(C₁-C₆)alkyl, piperidino(C₁-C₆)alkyl, pyrrolidino(C₁-C₆)alkyl, (C₁-C₈)alkoxy-carbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl, and di(C₁-C₈)alkylaminocarboxy(C₁-C₆)alkyl; and

2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, and bicyclic heteroaryl(C₁-C₃)alkoxy, each optionally substituted with 1 to 5 groups independently selected from the group consisting of fluorine, chlorine, cyano, (C₁-C₆)alkyl, halo(C₁-C₆)alkyl, (C₁-C₆)alkoxy, halo(C₁-C₆)alkoxy, (C₁-C₆)alkylsulfonyl, (C₁-C₆)alkoxycarbonyl, and aminocarbonyl.

In the compounds of Formula I, X and Y are each independently CH₂ or a single bond.

In the compounds of Formula I, R² is a substituted or unsubstituted (C₁-C₁₂)alkyl, (C₂-C₁₂)alkenyl, (C₂-C₁₂)alkynyl, (C₁-C₁₂)alkoxy, (C₂-C₁₂)alkenyloxy, (C₁-C₁₂)alkylthio, (C₁-C₆)alkoxy(C₁-C₆)alkyl, (C₁-C₆)alkylthio-(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonyl-amino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonylamino(C₁-C₆)alkylthio, (C₃-C₄)cycloalkylcarbonyl-amino(C₁-C₆)alkyl, (C₃-C₄)-cycloalkylcarbonylamino(C₁-C₆)alkoxy, (C₃-C₄)cycloalkylcarbonyl-amino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkylsulfonylamino(C₁-C₆)alkyl, (C₁-C₆)alkyl-sulfonylamino(C₁-C₆)-alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)-alkylthio, aminocarbonyl(C₁-C₆)alkyl, aminocarbonyl(C₁-C₆)alkoxy, aminocarbonyl(C₁-C₆)-alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)-alkoxycarbonylamino, (C₁-C₁₂)alkylaminocarbonylamino, or (C₁-C₁₂)alkylcarbonylamino,

wherein the substituted (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino-(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonyl-amino(C₁-C₆)alkylthio, (C₃-C₄)cycloalkylcarbonylamino(C₁-C₆)alkyl, (C₃-C₄)cycloalkylcarbonylamino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonyl-amino(C₁-C₁₂)alkylthio, C₁-C₆)alkylsulfonylamino(C₁-C₆)alkyl, (C₁-C₆)alkylsulfonyl-amino(C₁-C₆)alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxy-carbonylamino(C₁-C₆)alkyl, (C₁-C₆)alkoxycarbonyl-amino(C₁-C₆)alkoxy, (C₁-C₆)-alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylamino-carbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino represented by R² is substituted by at least one of:

a) 1 to 6 halogen atoms; or

b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl, and halo(C₃-C₆)cycloalkoxy; and

wherein the thio-moiety of said unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonyl-amino(C₁-C₆)alkylthio, aminosulfonylamino(C₁-C₁₂)alkylthio, (C₁-C₆)alkyl-sulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio is optionally replaced by —S(O)— or —S(O)₂—; and

wherein the carbonyl moiety of said unsubstituted or substituted aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonyl-amino(C₁-C₆)alkoxy, (C₁-C₆)alkylcarbonylamino(C₁-C₆)alkylthio, (C₃-C₄)-cycloalkylcarbonyl-amino(C₁-C₆)alkyl, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonyl-amino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonyl-amino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino is optionally replaced by a thiocarbonyl moiety.

In the compounds of Formula I, R³ is: a) —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkylcarbonylamino, (C₁-C₆)-alkoxycarbonylamino, (C₁-C₆)alkylaminocarbonylamino, di(C₁-C₆)alkylaminocarbonylamino, (C₁-C₆)alkylsulfonylamino, (C₁-C₆)alkylaminosulfonylamino, or di(C₁-C₆)alkylaminosulfonyl-amino; or

b) phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkyl(C₁-C₆)alkylthio, halo(C₁-C₆)alkylthio, halo(C₃-C₆)cycloalkythio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkyl(C₁-C₆)alkylthio-(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈) cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl;

provided that when R³ is hydroxyl, halogen, or optionally substituted phenylamino or heteroarylamino, then R² is not an optionally substituted alkoxy, alkylthio or amino group as follows:

1) when R³ is hydroxyl, halogen, or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonyl-amino(C₁-C₁₂)alkoxy, (C₁-C₆)alkylcarbonyl-amino(C₁-C₆)alkoxy, (C₃-C₄)cycloalkylcarbonylamino-(C₁-C₆)alkoxy, aminosulfonylamino(C₁-C₁₂)alkoxy, (C₁-C₆)alkyl-sulfonylamino(C₁-C₆)alkoxy, formylamino(C₁-C₆)alkoxy, (C₁-C₆)alkoxycarbonyl-amino(C₁-C₆)alkoxy, (C₁-C₆)alkylamino-carbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl-(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkylcarbonylamino;

2) when R³ is hydroxyl, halogen, or optionally substituted phenylamino or heteroarylamino, then R² is not an unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkylsulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio, wherein the thio moiety is replaced by —S(O)— or —S(O)₂—; and

3) when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkylcarbonyl-amino(C₁-C₆)alkoxy, (C₁-C₆)alkylcarbonylamino(C₁-C₆)alkylthio, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkoxy, formylamino(C₁-C₆)alkylthio, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkoxy, aminocarboxy(C₁-C₆)alkylthio, (C₁-C₆)alkylamino-carboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino, wherein the carbonyl moiety is replaced by a thiocarbonyl moiety.

In the compounds of Formula I, A is a saturated or unsaturated 4-, 5-, 6-, or 7-membered ring which is optionally bridged by (CH₂)ₘ via bonds to two members of said ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from the group consisting of halogen, (C₁-C₆)alkyl, halo(C₁-C₆)alkyl, and oxo; where m is 1 to 3; and the carbonyl moiety and Y are attached to carbon or nitrogen atoms in ring A in a 1,2 or 1,3 or 1,4 relationship.

In the compounds of Formula I, L is a linear (C₁-C₈)alkyl chain which is optionally substituted by 1-4 groups independently selected from R⁴, R⁵, R⁶, and R⁷; wherein each R⁴, R⁵, R⁶, and R⁷ is independently selected from hydroxyl, carboxy (hydroxycarbonyl), amino, amido (aminocarbonyl-), and from the following optionally substituted groups:

1) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₂-C₃)alkenyl, (C₂-C₁₂)alkynyl, (C₃-C₈)cycloalkyl(C₂-C₃)alkynyl, (C₁-C₆)alkoxy, (C₃-C₆)cycloalkoxy, halo(C₁-C₆)alkoxy, halo(C₃-C₆)cycloalkoxy, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, (C₁-C₁₂)alkoxycarbonyl, (C₃-C₈)cycloalkyl-oxy-carbonyl, (C₁-C₆)alkylaminocarbonyl, di[(C₁-C₆)alkyl]aminocarbonyl, saturated heterocyclyl, and saturated heterocyclyl(C₁-C₃)alkyl; wherein each is optionally and independently substituted by a group selected from: halogen, cyano, hydroxyl, carboxy, amino, amido, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and

2) phenyl, naphthyl, heteroaryl, phenoxycarbonyl, naphthyloxycarbonyl, heteroaryloxycarbonyl, phenylaminocarbonyl, naphthylaminocarbonyl, heteroarylaminocarbonyl, (phenyl)((C₁-C₆)alkyl)aminocarbonyl, (naphthyl)((C₁-C₆)alkyl)aminocarbonyl, (hetero aryl)((C₁-C₆)alkyl)aminocarbonyl, phenyl(C₁-C₃)alkyl, naphthyl(C₁-C₃)alkyl, heteroaryl(C₁-C₃)alkyl, phenyl(C₁-C₃)alkoxycarbonyl, naphthyl(C₁-C₃)alkoxycarbonyl, heteroaryl(C₁-C₃)alkoxycarbonyl, phenyl(C₁-C₃)alkylaminocarbonyl, naphthyl(C₁-C₃)alkylaminocarbonyl, heteroaryl(C₁-C₃)alkylaminocarbonyl, (phenyl(C₁-C₃)alkyl)((C₁-C₆)alkyl)aminocarbonyl, (naphthyl(C₁-C₃)alkyl)((C₁-C₆)alkyl)aminocarbonyl, and (heteroaryl(C₁-C₃)alkyl))((C₁-C₆)alkyl)aminocarbonyl; each optionally substituted with 1 to 3 groups independently selected from fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkylsulfonyl, and (C₁-C₃)alkoxycarbonyl.

or L is —NH(C₂-C₆)alkyl optionally substituted by hydroxyl, carboxy (hydroxycarbonyl), amino, amido (aminocarbonyl-), or (C₁-C₆)alkoxycarbonyl, when R² is aminocarbonylamino(C₁-C₁₂)alkyl, aminocarbonylamino(C₁-C₁₂)alkoxy, aminocarbonyl-amino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonylamino(C₁-C₆)alkylthio, (C₃-C₄)cycloalkylcarbonyl-amino(C₁-C₆)alkyl, (C₃-C₄)-cycloalkylcarbonylamino(C₁-C₆)alkoxy, (C₃-C₄)cycloalkylcarbonyl-amino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkylsulfonylamino(C₁-C₆)alkyl, (C₁-C₆)alkyl-sulfonylamino(C₁-C₆)-alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)-alkylthio, aminocarbonyl(C₁-C₆)alkyl, aminocarbonyl(C₁-C₆)alkoxy, aminocarbonyl(C₁-C₆)-alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)-alkoxycarbonylamino, (C₁-C₁₂)alkylaminocarbonylamino, or (C₁-C₁₂)alkylcarbonylamino, and R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkylcarbonylamino, (C₁-C₆)-alkoxycarbonylamino, (C₁-C₆)alkylaminocarbonylamino, di(C₁-C₆)alkylaminocarbonylamino, (C₁-C₆)alkylsulfonylamino, (C₁-C₆)alkylaminosulfonylamino, or di(C₁-C₆)alkylaminosulfonyl-amino, provided that when R³ is hydroxyl, or halogen, then R² is not an alkoxy, alkylthio or amino group as provided above;

or L is —NH(C₂-C₆)alkyl substituted by phenyl(C₁-C₃)alkoxycarbonyl, phenyl, naphthyl, heteroaryl, phenyl(C₁-C₃)alkyl, naphthyl(C₁-C₃)alkyl, or heteroaryl(C₁-C₃)alkyl, wherein the phenyl, naphthyl and heteroaryl groups are optionally substituted by 1 to 3 groups selected from: fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl;

When L is a —NH(C₂-C₆)alkyl group, the carbonyl moiety of Formula I is attached to nitrogen atom of L and G is attached to (C₂-C₆)alkyl moiety of L;

In the compounds of Formula I, G is —OH, —OR⁸, —NH₂, —NHR⁸, —NR⁸R⁹, —NHC(═NH)NH₂, —NHC(═NH)NHR⁸; —C(═NH)NH₂, or —C(═NH)NHR⁸;

where R⁸ is: a) (C₁-C₁₂)alkyl, (C₄-C₁₂)cycloalkyl(C₁-C₆)alkyl, halo(C₁-C₁₂)alkyl, halo(C₄-C₁₂)cycloalkyl(C₁-C₆)alkyl, (C₂-C₁₂)alkenyl, (C₅-C₁₂)cycloalkyl(C₂-C₆)alkenyl, halo(C₂-C₁₂)alkenyl, halo(C₅-C₁₂)cycloalkyl(C₂-C₆)alkenyl, (C₂-C₁₂)alkynyl, (C₅-C₁₂)cycloalkyl(C₂-C₆)alkynyl, halo(C₂-C₁₂)alkynyl, halo(C₅-C₁₂)cycloalkyl(C₂-C₆)alkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkylsulfinyl(C₁-C₆)alkyl, halo(C₁-C₆)alkylsulfinyl(C₁-C₆)alkyl, (C₁-C₆)alkylsulfonyl(C₁-C₆)alkyl, halo(C₁-C₆)alkylsulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, amino(C₁-C₄)alkyl-carbonyl, (C₁-C₄)alkylamino(C₁-C₄)alkylcarbonyl, di((C₁-C₄)alkyl)-amino(C₁-C₄)alkylcarbonyl, cyano(C₁-C₆)alkyl, hydroxy(C₁-C₁₂)alkyl, amino(C₁-C₁₂)alkyl, (C₁-C₆)alkyl-amino(C₁-C₆)alkyl, di((C₁-C₆)alkyl)-amino C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or

b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from: 1) fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl; and

2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkylsulfonyl, and (C₁-C₃)-alkoxycarbonyl; and

R⁹ is (C₁-C₆)alkyl or halo(C₁-C₆)alkyl.

In one embodiment the present invention is directed to a pharmaceutical composition comprising a therapeutically effective amount of a compound described herein or an enantiomer, diastereomer, or salt thereof and a pharmaceutically acceptable carrier or excipient.

In one embodiment the present invention is directed to a method for treating or ameliorating an aspartic protease mediated disorder in a subject in need thereof comprising administering to said subject a therapeutically effective amount of a compound described herein or an enantiomer, diastereomer, or salt thereof.

In another embodiment the present invention is a method for treating or ameliorating a renin mediated disorder in a subject in need thereof comprising administering to said subject a therapeutically effective amount of a compound described herein or enantiomer, diastereomer, or salt thereof.

### DETAILED DESCRIPTION OF THE INVENTION

In one embodiment, the present invention is directed to compounds of Formula I:

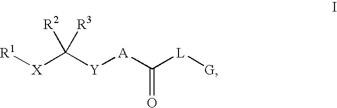

or a pharmaceutically acceptable salt thereof

wherein, R¹ is:

a) (C₁-C₁₂)alkyl, (C₃-C₇)cycloalkyl, (C₄-C₁₂)cycloalkyl(C₁-C₆)alkyl, halo(C₁-C₁₂)alkyl, halo(C₃-C₇)-cycloalkyl, halo(C₄-C₁₂)cycloalkyl(C₁-C₆)alkyl, or saturated heterocyclyl, each optionally substituted with 1 to 5 groups independently selected from the group consisting of halogen, (C₁-C₆)alkyl, halo(C₁-C₆)alkyl, and oxo; or

b) phenyl, naphthyl, heteroaryl, or bicyclic heteroaryl, each optionally substituted with 1 to 5 groups independently selected from the groups consisting of:

1) fluorine, chlorine, bromine, iodine, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkyl(C₁-C₆)alkyl, (C₂-C₆)alkenyl, (C₅-C₈)cycloalkenyl, (C₅-C₈)cycloalkyl(C₂-C₆)alkenyl, (C₂-C₈)alkynyl, (C₃-C₈)cycloalkyl(C₂-C₄)alkynyl, halo(C₁-C₈)alkyl, halo(C₃-C₆)cycloalkyl, halo(C₄-C₈)cycloalkyl(C₁-C₆)alkyl, (C₁-C₃)alkyl(C₄-C₈)cycloalkyl(C₁-C₆)alkyl, di(C₁-C₃)alkyl(C₄-C₈)cycloalkyl(C₁-C₆)alkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkyl(C₂-C₆)alkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)-cycloalkylalkoxy, (C₁-C₈)alkylthio, (C₃-C₈)cycloalkythio, (C₄-C₈)cycloalkyl(C₁-C₆)alkylthio, (C₁-C₃)alkyl(C₃-C₈)cycloalkythio, (C₁-C₃)alkyl(C₄-C₈)cycloalkyl(C₁-C₆)alkylthio, di(C₁-C₃)alkyl(C₃-C₈)-cycloalkythio, di(C₁-C₃)alkyl(C₄-C₈)cycloalkyl(C₁-C₆)alkylthio, halo(C₁-C₈)alkylthio, halo(C₃-C₈)-cycloalkythio, halo(C₄-C₈)-cycloalkyl(C₁-C₆)alkylthio, (C₁-C₈)alkylsulfinyl, (C₃-C₈)-cycloalkyl-sulfinyl, (C₄-C₈)cycloalkyl-alkylsulfinyl, (C₁-C₃)alkyl(C₃-C₈)cycloalkyl-sulfinyl, (C₁-C₃)alkyl(C₄-C₈)cycloalkyl-alkylsulfinyl, di(C₁-C₃)alkyl(C₃-C₈)cycloalkyl-sulfinyl, di(C₁-C₃)alkyl(C₄-C₈)cycloalkyl-alkylsulfinyl, halo(C₁-C₈)alkylsulfinyl, halo(C₃-C₈)cycloalkylsulfinyl, halo(C₄-C₈)cycloalkyl(C₁-C₆)alkylsulfinyl, (C₁-C₈)alkyl-sulfonyl, (C₃-C₈)cycloalkylsulfonyl, (C₄-C₈) cycloalkyl(C₁-C₆)alkylsulfonyl, (C₁-C₃)alkyl(C₃-C₈)cycloalkylsulfonyl, (C₁-C₃)alkyl(C₄-C₈)-cycloalkyl-alkylsulfonyl, di(C₁-C₃)alkyl(C₃-C₈)cycloalkylsulfonyl, di(C₁-C₃)alkyl(C₄-C₈)-cycloalkyl-alkylsulfonyl, halo(C₁-C₈)alkylsulfonyl, halo(C₃-C₈)cycloalkylsulfonyl, halo(C₄-C₈)cycloalkyl(C₁-C₆)alkyl-sulfonyl, (C₁-C₈)alkylamino, di(C₁-C₈)alkylamino, (C₁-C₆)alkoxy-(C₁-C₆)-alkoxy, halo(C₁-C₆)alkoxy(C₁-C₆)alkoxy, (C₁-C₈)alkoxycarbonyl, aminocarbonyl, (C₁-C₈)alkylamino-carbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)-cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio-(C₁-C₆)alkyl, (C₃-C₈)cycloalkythio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio-(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)-cycloalkyl-sulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈)-cycloalkyl(C₁-C₆)alkyl-sulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino-(C₁-C₆)alkyl, (C₁-C₈)alkoxycarbonyl(C₁-C₆)alkyl, (C₁-C₈)acyloxy(C₁-C₆)alkyl, aminocarbonyl-(C₁-C₆)alkyl, (C₁-C₈)alkylamino-carbonyl(C₁-C₆)alkyl, di(C₁-C₈)alkylamino-carbonyl(C₁-C₆)alkyl, (C₁-C₈)acylamino(C₁-C₆)alkyl, piperidino(C₁-C₆)alkyl, pyrrolidino(C₁-C₆)alkyl, (C₁-C₈)alkoxy-carbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl, and di(C₁-C₈)alkylaminocarboxy(C₁-C₆)alkyl; and

2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, and bicyclic heteroaryl(C₁-C₃)alkoxy, each optionally substituted with 1 to 5 groups independently selected from the group consisting of fluorine, chlorine, cyano, (C₁-C₆)alkyl, halo(C₁-C₆)alkyl, (C₁-C₆)alkoxy, halo(C₁-C₆)alkoxy, (C₁-C₆)alkylsulfonyl, (C₁-C₆)alkoxycarbonyl, and aminocarbonyl.

In the compounds of Formula I, X and Y are each independently CH₂ or a single bond.

In the compounds of Formula I, R² is a substituted or unsubstituted (C₁-C₁₂)alkyl, (C₂-C₁₂)alkenyl, (C₂-C₁₂)alkynyl, (C₁-C₁₂)alkoxy, (C₂-C₁₂)alkenyloxy, (C₁-C₁₂)alkylthio, (C₁-C₆)alkoxy(C₁-C₆)alkyl, (C₁-C₆)alkylthio-(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonyl-amino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonylamino(C₁-C₆)alkylthio, (C₃-C₄)cycloalkylcarbonyl-amino(C₁-C₆)alkyl, (C₃-C₄)-cycloalkylcarbonylamino(C₁-C₆)alkoxy, (C₃-C₄)cycloalkylcarbonyl-amino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkylsulfonylamino(C₁-C₆)alkyl, (C₁-C₆)alkyl-sulfonylamino(C₁-C₆)-alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)-alkylthio, aminocarbonyl(C₁-C₆)alkyl, aminocarbonyl(C₁-C₆)alkoxy, aminocarbonyl(C₁-C₆)-alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)-alkoxycarbonylamino, (C₁-C₁₂)alkylaminocarbonylamino, or (C₁-C₁₂)alkylcarbonylamino, wherein the substituted (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino —(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonyl-amino(C₁-C₆)alkylthio, (C₃-C₄)cycloalkylcarbonylamino(C₁-C₆)alkyl, (C₃-C₄)cycloalkyl-carbonyl-amino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonyl-amino(C₁-C₁₂)alkylthio, C₁-C₆)alkylsulfonylamino(C₁-C₆)alkyl, (C₁-C₆)alkylsulfonyl-amino(C₁-C₆)alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxy-carbonylamino(C₁-C₆)alkyl, (C₁-C₆)alkoxycarbonyl-amino(C₁-C₆)alkoxy, (C₁-C₆)-alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylamino-carbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino represented by R² is substituted by at least one of:

a) 1 to 6 halogen atoms; or

b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl, and halo(C₃-C₆)cycloalkoxy; and

wherein the thio-moiety of said unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonyl-amino(C₁-C₆)alkylthio, aminosulfonylamino(C₁-C₁₂)alkylthio, (C₁-C₆)alkyl-sulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio is optionally replaced by —S(O)— or —S(O)₂—; and

wherein the carbonyl moiety of said unsubstituted or substituted aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonyl-amino(C₁-C₆)alkoxy, (C₁-C₆)alkylcarbonylamino(C₁-C₆)alkylthio, (C₃-C₄)-cycloalkylcarbonyl-amino(C₁-C₆)alkyl, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonyl-amino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonyl-amino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino is optionally replaced by a thiocarbonyl moiety.

In the compounds of Formula I, R³ is: a) —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkylcarbonylamino, (C₁-C₆)-alkoxycarbonylamino, (C₁-C₆)alkylaminocarbonylamino, di(C₁-C₆)alkylaminocarbonylamino, (C₁-C₆)alkylsulfonylamino, (C₁-C₆)alkylaminosulfonylamino, or di(C₁-C₆)alkylaminosulfonyl-amino; or

b) phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkyl(C₁-C₆)alkylthio, halo(C₁-C₆)alkylthio, halo(C₃-C₆)cycloalkythio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkyl(C₁-C₆)alkylthio-(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈) cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl;

provided that when R³ is hydroxyl, halogen, or optionally substituted phenylamino or heteroarylamino, then R² is not an optionally substituted alkoxy, alkylthio or amino group as follows:

1) when R³ is hydroxyl, halogen, or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonyl-amino(C₁-C₁₂)alkoxy, (C₁-C₆)alkylcarbonyl-amino(C₁-C₆)alkoxy, (C₃-C₄)cycloalkylcarbonylamino-(C₁-C₆)alkoxy, aminosulfonylamino(C₁-C₁₂)alkoxy, (C₁-C₆)alkyl-sulfonylamino(C₁-C₆)alkoxy, formylamino(C₁-C₆)alkoxy, (C₁-C₆)alkoxycarbonyl-amino(C₁-C₆)alkoxy, (C₁-C₆)alkylamino-carbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl-(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkylcarbonylamino;

2) when R³ is hydroxyl, halogen, or optionally substituted phenylamino or heteroarylamino, then R² is not an unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkylsulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio, wherein the thio moiety is replaced by —S(O)— or —S(O)₂—; and

3) when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkylcarbonyl-amino(C₁-C₆)alkoxy, (C₁-C₆)alkylcarbonylamino(C₁-C₆)alkylthio, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkoxy, formylamino(C₁-C₆)alkylthio, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkoxy, aminocarboxy(C₁-C₆)alkylthio, (C₁-C₆)alkylamino-carboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino, wherein the carbonyl moiety is replaced by a thiocarbonyl moiety.

In the compounds of Formula I, A is a saturated or unsaturated 4-, 5-, 6-, or 7-membered ring which is optionally bridged by (CH₂)ₘ via bonds to two members of said ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from the group consisting of halogen, (C₁-C₆)alkyl, halo(C₁-C₆)alkyl, and oxo; where m is 1 to 3; and the carbonyl moiety and Y are attached to carbon or nitrogen atoms in ring A in a 1,2 or 1,3 or 1,4 relationship.

In the compounds of Formula I, L is a linear (C₁-C₈)alkyl chain which is optionally substituted by 1-4 groups independently selected from R⁴, R⁵, R⁶, and R⁷; wherein each R⁴, R⁵, R⁶, and R⁷ is independently selected from hydroxyl, carboxy (hydroxycarbonyl), amino, amido (aminocarbonyl-), and from the following optionally substituted groups:

1) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₂-C₃)alkenyl, (C₂-C₁₂)alkynyl, (C₃-C₈)cycloalkyl(C₂-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, (C₁-C₁₂)alkoxycarbonyl, (C₃-C₈)cycloalkyl-oxy-carbonyl, (C₁-C₆)alkylaminocarbonyl, di[(C₁-C₆)alkyl]aminocarbonyl, saturated heterocyclyl, and saturated heterocyclyl(C₁-C₃)alkyl; wherein each is optionally and independently substituted by a group selected from: halogen, cyano, hydroxyl, carboxy, amino, amido, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and

2) phenyl, naphthyl, heteroaryl, phenoxycarbonyl, naphthyloxycarbonyl, heteroaryloxycarbonyl, phenylaminocarbonyl, naphthylaminocarbonyl, heteroarylaminocarbonyl, (phenyl)((C₁-C₆)alkyl)aminocarbonyl, (naphthyl)((C₁-C₆)alkyl)aminocarbonyl, (hetero aryl)((C₁-C₆)alkyl)aminocarbonyl, phenyl(C₁-C₃)alkyl, naphthyl(C₁-C₃)alkyl, heteroaryl(C₁-C₃)alkyl, phenyl(C₁-C₃)alkoxycarbonyl, naphthyl(C₁-C₃)alkoxycarbonyl, heteroaryl(C₁-C₃)alkoxycarbonyl, phenyl(C₁-C₃)alkylaminocarbonyl, naphthyl(C₁-C₃)alkylaminocarbonyl, heteroaryl(C₁-C₃)alkylaminocarbonyl, (phenyl(C₁-C₃)alkyl)((C₁-C₆)alkyl)aminocarbonyl, (naphthyl(C₁-C₃)alkyl)((C₁-C₆)alkyl)aminocarbonyl, and (heteroaryl(C₁-C₃)alkyl))((C₁-C₆)alkyl)aminocarbonyl; each optionally substituted with 1 to 3 groups independently selected from fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkylsulfonyl, and (C₁-C₃)alkoxycarbonyl.

In the compounds of Formula I, G is —OH, —OR⁸, —NH₂, —NHR⁸, —NR⁸R⁹, —NHC(═NH)NH₂, —NHC(═NH)NHR⁸; —C(═NH)NH₂, or —C(═NH)NHR⁸;

where R⁸ is: a) (C₁-C₁₂)alkyl, (C₄-C₁₂)cycloalkyl(C₁-C₆)alkyl, halo(C₁-C₁₂)alkyl, halo(C₄-C₁₂)cycloalkyl(C₁-C₆)alkyl, (C₂-C₁₂)alkenyl, (C₅-C₁₂)cycloalkyl(C₂-C₆)alkenyl, halo(C₂-C₁₂)alkenyl, halo(C₅-C₁₂)cycloalkyl(C₂-C₆)alkenyl, (C₂-C₁₂)alkynyl, (C₅-C₁₂)cycloalkyl(C₂-C₆)alkynyl, halo(C₂-C₁₂)alkynyl, halo(C₅-C₁₂)cycloalkyl(C₂-C₆)alkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkylsulfinyl(C₁-C₆)alkyl, halo(C₁-C₆)alkylsulfinyl(C₁-C₆)alkyl, (C₁-C₆)alkylsulfonyl(C₁-C₆)alkyl, halo(C₁-C₆)alkylsulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, amino(C₁-C₄)alkyl-carbonyl, (C₁-C₄)alkylamino(C₁-C₄)alkylcarbonyl, di((C₁-C₄)alkyl)-amino(C₁-C₄)alkylcarbonyl, cyano(C₁-C₆)alkyl, hydroxy(C₁-C₁₂)alkyl, amino(C₁-C₁₂)alkyl, (C₁-C₆)alkyl-amino(C₁-C₆)alkyl, di((C₁-C₆)alkyl)-amino C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or

b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from: 1) fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl; and

2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkylsulfonyl, and (C₁-C₃)-alkoxycarbonyl; and

R⁹ is (C₁-C₆)alkyl or halo(C₁-C₆)alkyl.

In particular embodiments, the present invention is directed to compounds of Formula I, wherein X and Y are each a single bond.

In one embodiment, the present invention is directed to compounds of Formula Ia:

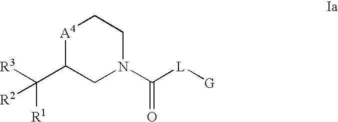

wherein R¹, R², R³, A⁴, L, and G are as defined herein, or a pharmaceutically acceptable salt thereof.

In another embodiment, the present invention is directed to compounds of Formula Ib:

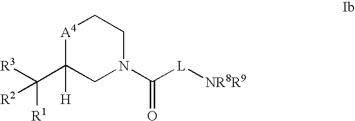

wherein R¹, R², R³, A⁴, L, R⁸, and R⁹ are as defined herein, or a pharmaceutically acceptable salt thereof.

In another embodiment, the present invention is directed to compounds of Formula Ic:

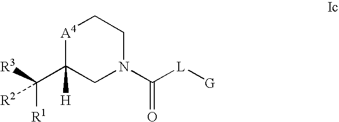

wherein R¹, R², R³, A⁴, L, and G are as defined herein, or a pharmaceutically acceptable salt thereof.

The invention also provides compounds wherein R¹ is a) (C₁-C₁₂)alkyl, (C₃-C₇)cycloalkyl, (C₄-C₁₂)cycloalkyl(C₁-C₆)alkyl, halo(C₁-C₁₂)alkyl, halo(C₃-C₇)-cycloalkyl, halo(C₄-C₁₂)cycloalkyl(C₁-C₆)alkyl, saturated heterocyclyl optionally substituted with 1 to 5 groups independently selected from: halogen, (C₁-C₆)alkyl, halo(C₁-C₆)alkyl, and oxo; or

b) phenyl, naphthyl, heteroaryl, or bicyclic heteroaryl each optionally substituted with 1 to 5 groups independently selected from:

1) fluorine, chlorine, bromine, iodine, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkyl(C₁-C₆)alkyl, (C₂-C₆)alkenyl, (C₅-C₈)cycloalkenyl, (C₅-C₈)cycloalkyl(C₂-C₆)alkenyl, (C₂-C₈)alkynyl, (C₃-C₈)cycloalkyl(C₂-C₆)alkynyl, halo(C₁-C₈)alkyl, halo(C₃-C₆)cycloalkyl, halo(C₄-C₈)cycloalkyl(C₁-C₆)alkyl, (C₁-C₃)alkyl(C₄-C₈)cycloalkyl(C₁-C₆)alkyl, di(C₁-C₃)alkyl(C₄-C₈)cycloalkyl(C₁-C₆)alkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkyl(C₂-C₆)alkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)-cycloalkylalkoxy, (C₁-C₈)alkylthio, (C₃-C₈)cycloalkythio, (C₄-C₈)cycloalkyl(C₁-C₆)alkylthio, (C₁-C₃)alkyl(C₃-C₈)cycloalkythio, (C₁-C₃)alkyl(C₄-C₈)cycloalkyl(C₁-C₆)alkylthio, di(C₁-C₃)alkyl(C₃-C₈)-cycloalkythio, di(C₁-C₃)alkyl(C₄-C₈)cycloalkylalkylthio, halo(C₁-C₈)alkylthio, halo(C₃-C₈)-cycloalkythio, halo(C₄-C₈)-cycloalkyl(C₁-C₆)alkylthio, (C₁-C₈)alkylsulfinyl, (C₃-C₈)-cycloalkyl-sulfinyl, (C₄-C₈)cycloalkyl-alkylsulfinyl, (C₁-C₃)alkyl(C₃-C₈)cycloalkyl-sulfinyl, (C₁-C₃)alkyl(C₄-C₈)cycloalkyl-alkylsulfinyl, di(C₁-C₃)alkyl(C₃-C₈)cycloalkyl-sulfinyl, di(C₁-C₃)alkyl (C₄-C₈)cycloalkyl-alkylsulfinyl, halo(C₁-C₈)alkylsulfinyl, halo(C₃-C₈)cycloalkylsulfinyl, halo(C₄-C₈)cycloalkyl(C₁-C₆)alkylsulfinyl, (C₁-C₈)alkylsulfonyl, (C₃-C₈)cycloalkylsulfonyl, (C₄-C₈)cycloalkyl(C₁-C₆)alkylsulfonyl, (C₁-C₃)alkyl(C₃-C₈)cycloalkylsulfonyl, (C₁-C₃)alkyl(C₄-C₈)-cycloalkyl-alkylsulfonyl, di(C₁-C₃)alkyl(C₃-C₈)cycloalkylsulfonyl, di(C₁-C₃)alkyl(C₄-C₈)-cycloalkyl-alkylsulfonyl, halo(C₁-C₈)alkylsulfonyl, halo(C₃-C₈)cycloalkylsulfonyl, halo(C₄-C₈)cycloalkyl(C₁-C₆)alkyl-sulfonyl, (C₁-C₈)alkylamino, di(C₁-C₈)alkylamino, (C₁-C₆)alkoxy-(C₁-C₆)-alkoxy, halo(C₁-C₆)alkoxy(C₁-C₆)alkoxy, (C₁-C₈)alkoxycarbonyl, aminocarbonyl, (C₁-C₈)alkyl-amino-carbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)-cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio-(C₁-C₆)alkyl, (C₃-C₈)cycloalkythio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio-(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)-cycloalkyl-sulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈)-cycloalkyl(C₁-C₆)alkyl-sulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino-(C₁-C₆)alkyl, (C₁-C₈)alkoxycarbonyl(C₁-C₆)alkyl, (C₁-C₈)acyloxy(C₁-C₆)alkyl, aminocarbonyl-(C₁-C₆)alkyl, (C₁-C₈)alkylamino-carbonyl(C₁-C₆)alkyl, di(C₁-C₈)alkylamino-carbonyl(C₁-C₆)alkyl, (C₁-C₈)acylamino(C₁-C₆)alkyl, piperidino(C₁-C₆)alkyl, pyrrolidino(C₁-C₆)alkyl, (C₁-C₈)alkoxy-carbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl, and di(C₁-C₈)alkylaminocarboxy(C₁-C₆)alkyl; and

2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, or bicyclic heteroaryl(C₁-C₃)alkoxy, each optionally substituted with 1 to 5 groups independently selected from: fluorine, chlorine, cyano, (C₁-C₆)alkyl, halo(C₁-C₆)alkyl, (C₁-C₆)alkoxy, halo(C₁-C₆)alkoxy, (C₁-C₆)alkylsulfonyl, (C₁-C₆)alkoxy-carbonyl, and aminocarbonyl.

In one embodiment, R¹ is phenyl, 2-thienyl, 3-thienyl, 2-pyridyl, 2-imidazolyl, 2-thiazolyl, 2-benzothienyl, 4-benzofuryl, 4-benzothienyl, 7-benzofuryl, 2,3-dihydro-7-benzofuryl, 7-benzothienyl, 1,3-benzodioxol-4-yl, 7-indazolyl, or 8-quinolinyl, each optionally substituted with 1 to 3 substituents independently selected from: fluorine, chlorine, bromine, cyano, methyl, ethyl, isopropyl, t-butyl, isobutyl, trifluoromethyl, allyl, cyclohexyl, cyclohexen-1-yl, cyclopropylethynyl, methoxy, trifluoromethoxy, neopentyloxy, methylthio, allyloxy, cyclopropylmethoxy, 2-(cyclopropyl)ethoxy, cyclopentyloxy, cyclopentylmethoxy, benzyloxy, hydroxyl, aminocarbonyl, methoxycarbonyl, phenyl, heteroaryl, phenoxy, benzyloxy, and heteroaryloxy,

wherein the phenyl, heteroaryl, phenoxy, benzyloxy, and heteroaryloxy groups are optionally substituted with 1 to 3 substituents independently selected from fluorine, chlorine, cyano, methyl, ethyl, isopropyl, trifluoromethyl, methoxy, and aminocarbonyl;

wherein the heteroaryl (and the heteroaryl moiety of the heteroaryloxy group) group is selected from 2-furanyl, 2-thienyl, 3-thienyl, 2-pyridyl, 2-imidazolyl, 2-thiazolyl, 2-benzothienyl, 4-benzofuryl, 4-benzothienyl, 7-benzofuryl, 2,3-dihydro-7-benzofuryl, 2,3-dihydro-6-benzofuryl, 7-benzothienyl, 1,3-benzodioxol-4-yl, 7-indazolyl, 8-quinolinyl, or 3-quinolinyl.

In another embodiment, R¹ is phenyl, optionally substituted with 1 to 3 substituents independently selected from halogen, cyano, hydroxyl, C₁-C₆ alkyl, haloC₁-C₆ alkyl, C₁-C₆ alkoxy, haloC₁-C₆ alkoxy, C₃-C₇ cycloalkyl, C₃-C₇ cycloalkoxy, (C₃-C₇)cycloalkyl(C₁-C₄ alkyl), (C₃-C₇)cycloalkyl(C₁-C₄ alkoxy), aminocarbonyl, phenyl, heteroaryl, phenoxy, benzyloxy, and heteroaryloxy, wherein the phenyl, heteroaryl, phenoxy, benzyloxy, and heteroaryloxy groups are optionally substituted with 1 to 3 substituents independently selected from halogen, cyano, C₁-C₄ alkyl, haloC₁-C₄ alkyl, C₁-C₄ alkoxy, and aminocarbonyl;

wherein the heteroaryl (and the heteroaryl moiety of the heteroaryloxy group) group is selected from a 5-6 membered monocyclic heteroaryl containing one or two heteroatoms selected from N, O, and S, and a 9-10 membered bicyclic heteroaryl containing 1 or 2 heteroatoms selected from N, O, and S, wherein at least one of the rings of the bicyclic group is aromatic.

In a further embodiment, R¹ is phenyl, optionally substituted with 1 to 3 substituents independently selected from: fluorine, chlorine, and methyl, or is a bi-aromatic group comprised of a phenyl substituted with another aromatic moiety selected from phenyl, phenoxy, furanyl, quinolinyl, and dihydrobenzofuranyl, wherein the bi-aromatic group is optionally substituted with 1 to 3 substituents independently selected from fluorine, chlorine, methyl, ethyl, isopropyl, trifluoromethyl, and methoxy, wherein the optional substituents are substituted on either one of or both of the phenyl moiety and the other aromatic moiety of the bi-aromatic group.

In a further embodiment, R¹ is phenyl, optionally substituted with 1 to 3 substituents independently selected from: fluorine, chlorine, and methyl, or is a bi-aromatic group comprised of a phenyl substituted with another aromatic moiety selected from phenyl, phenoxy, furanyl, quinolinyl, and dihydrobenzofuranyl, wherein the bi-aromatic group is optionally substituted with 1 to 3 substituents independently selected from fluorine, chlorine, methyl and ethyl, wherein the optional substituents are substituted on either one of or both of the phenyl moiety and the other aromatic moiety of the bi-aromatic group.

In specific embodiments, R¹ is phenyl, 3-chlorophenyl or 3-fluorophenyl. In other specific embodiments, R¹ is 6-chloro-3′-ethyl-2-biphenyl, 3′-ethyl-6-fluoro-2-biphenyl, 3′-methyl-6-fluoro-2-biphenyl, 3-fluoro-2-(3-quinolinyl)phenyl, 3-chloro-2-(2,3-dihydro-1-benzofuran-6-yl)phenyl, 2-[(2-methylphenyl)oxy]phenyl, 3-chloro-2-(5-methyl-2-furanyl)phenyl, 4′,6-difluoro-3′-methyl-2-biphenyl, 6-chloro-3′-fluoro-5′-methyl-2-biphenyl, 2′,6-difluoro-5′-methyl-2-biphenyl, or 3′,6-difluoro-5′-methyl-2-biphenyl.

In specific embodiments, R¹ is phenyl, 3-chlorophenyl, or 3-fluorophenyl. In other specific embodiments, R¹ is 6-chloro-3′-ethyl-2-biphenyl, 3′-ethyl-6-fluoro-2-biphenyl, 3′-methyl-6-fluoro-3′-methyl-2-biphenyl, 6-fluoro-3′-(1-methylethyl)-2-biphenyl, 3-fluoro-2-(3-quinolinyl)phenyl, 3-chloro-2-(3-quinolinyl)phenyl, 3-chloro-2-(2,3-dihydro-1-benzofuran-6-yl)phenyl, 2-[(2-methylphenyl)oxy]phenyl, 3-chloro-2-(5-methyl-2-furanyl)phenyl, 4′,6-difluoro-3′-methyl-2-biphenyl, 6-chloro-3′-fluoro-5′-methyl-2-biphenyl, 2′,6-difluoro-5′-methyl-2-biphenyl, or 3′,6-difluoro-5′-methyl-2-biphenyl, or 2′-(methoxy)-5′-(trifluoromethyl)-2-biphenyl.

In one embodiment, R² is substituted or unsubstituted (C₁-C₁₂)alkyl, (C₂-C₁₂)alkenyl, (C₂-C₁₂)alkynyl, (C₁-C₁₂)alkoxy, (C₂-C₁₂)alkenyloxy, (C₁-C₁₂)alkylthio, (C₁-C₆)alkoxy(C₁-C₆)alkyl, (C₁-C₆)alkylthio-(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonyl-amino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonylamino(C₁-C₆)alkylthio, (C₃-C₄)cycloalkylcarbonyl-amino(C₁-C₆)alkyl, (C₃-C₄)cycloalkylcarbonylamino(C₁-C₆)alkoxy, (C₃-C₄)cycloalkylcarbonyl-amino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkylsulfonylamino(C₁-C₆)alkyl, (C₁-C₆)alkyl-sulfonylamino(C₁-C₆)-alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)-alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)-alkoxycarbonylamino, (C₁-C₁₂)alkylaminocarbonylamino, (C₁-C₁₂)alkylcarbonylamino,

wherein the substituted (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino-(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonyl-amino(C₁-C₆)alkylthio, (C₃-C₄)cycloalkylcarbonylamino(C₁-C₆)alkyl, (C₃-C₄)cycloalkylcarbonyl-amino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonyl-amino(C₁-C₁₂)alkylthio, C₁-C₆)alkylsulfonylamino(C₁-C₆)alkyl, (C₁-C₆)alkylsulfonyl-amino(C₁-C₆)alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxy-carbonylamino(C₁-C₆)alkyl, (C₁-C₆)alkoxycarbonyl-amino(C₁-C₆)alkoxy, (C₁-C₆)-alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylamino-carbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino represented by R² is substituted by at least one of:

a) 1 to 6 halogen atoms, and

b) one substituent selected from cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl, and halo(C₃-C₆)cycloalkoxy, and

wherein the thio-moiety of said unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonyl-amino(C₁-C₆)alkylthio, aminosulfonylamino(C₁-C₁₂)alkylthio, (C₁-C₆)alkyl-sulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, and (C₁-C₆)alkylaminocarboxy(C₁-C₆)alkylthio is optionally replaced by a sulfinyl (sulfoxide, i.e., —S(O)—) or a sulfonyl (sulfone, i.e., —S(O)₂—) moiety, and

wherein the carbonyl moiety of said unsubstituted or substituted aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonyl-amino(C₁-C₆)alkoxy, (C₁-C₆)alkylcarbonylamino(C₁-C₆)alkylthio, (C₃-C₄)-cycloalkylcarbonyl-amino(C₁-C₆)alkyl, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonyl-amino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonyl-amino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino is optionally replaced by a thiocarbonyl moiety.

In another embodiment, R² is (C₁-C₅)alkoxy(C₁-C₅)alkyl, (C₁-C₅)alkylamino(C₁-C₅)alkyl, halo(C₁-C₅)alkoxy(C₁-C₅)alkyl, (C₁-C₅)alkoxy(C₁-C₅)alkoxy, hydroxy(C₁-C₈)alkoxy, halo(C₁-C₅)alkoxy(C₁-C₅)alkoxy, (C₁-C₃)alkoxy(C₁-C₃)alkoxy(C₁-C₃)alkyl, halo(C₁-C₃)-alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkylcarbonylamino(C₁-C₅)alkyl, (C₁-C₅)alkylcarbonylamino(C₁-C₅)alkoxy, halo(C₁-C₅)alkylcarbonylamino(C₁-C₅)alkyl, halo(C₁-C₅)alkylcarbonylamino(C₁-C₅)alkoxy, (C₁-C₃)alkoxy(C₁-C₅)alkylcarbonylamino(C₁-C₅)alkyl, (C₁-C₃)alkoxy(C₁-C₅)alkylcarbonylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylaminocarbonyl-amino(C₁-C₅)alkoxy, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)—alkylaminocarbonyl-(C₁-C₅)alkoxy, aminocarboxy(C₁-C₅)alkyl, aminocarboxy(C₁-C₅)alkoxy, (C₁-C₅)alkylamino-carboxy(C₁-C₅)alkyl, (C₁-C₅)alkylaminocarboxy(C₁-C₅)alkoxy, (C₁-C₈)-alkoxycarbonylamino, (C₁-C₈)alkylaminocarbonylamino, (C₁-C₈)alkylcarbonylamino, halo(C₁-C₈)alkoxycarbonylamino, halo(C₁-C₈)alkylaminocarbonylamino, or halo(C₁-C₈)-alkylcarbonylamino.

In yet another embodiment, R² is hydrogen, methyl, ethyl, propyl, butyl, hexyl, 5-pentenyl, 3,3,3-trifluoropropyl, 4,4-difluoropentyl, 3-(cyclopropyl)propyl, 4-(cyclopropyl)butyl, 3-hydroxypropyl, 4-hydroxybutyl, 4-hydroxypentyl, 4-hydroxyhexyl, 5-hydroxyhexyl, 2-hydroxyethoxy, 5-oxohexyl, 3-ethoxypropyl, 4-methoxybutyl, 4-ethoxybutyl, butoxy, hexyloxy, 2-(ethoxy)-ethoxy, 3-methoxypropoxy, 3-ethoxypropoxy, 3-propoxypropoxy, 2-cyclopropylethoxy, (2-(methoxy)ethoxy)methyl, 3-(2,2,2-trifluoroethylamino)propyl, 3-(formylamino)propyl, 3-(acetylamino)propyl, 3-(propionyl-amino)propyl, 3-(butanoylamino)propyl, 3-((2-methoxypropionyl)amino)propyl, 3-(cyclopropyl-carbonylamino)propyl, 3-(trifluoroacetylamino)propyl, 3-(methoxycarbonylamino)propyl, 3-(ethoxycarbonylamino)propyl, 2-(methoxycarbonylamino)ethoxy, 2-(ethoxycarbonylamino)-ethoxy, 3-(methylaminocarbonylamino)propyl, 3-(dimethylaminocarbonyl-amino)propyl, 3-(aminocarbonyl)propyl, 3-(methylaminocarbonyl)propyl, 3-(ethylaminocarbonyl)propyl, 2-(acetylamino)ethoxy, 2-(propionylamino)ethoxy, aminocarbonylmethoxy, methylamino-carbonylmethoxy, ethylaminocarbonylmethoxy, propylaminocarbonylmethoxy, 2-(methylaminocarbonyl)ethoxy, 2-(ethylaminocarbonyl)ethoxy, 2-(propylaminocarbonyl)ethoxy, (2-(methoxy)ethoxy)carbonylamino, methoxymethylcarbonylaminomethyl, or 3-(aminosulfonylamino)propyl, additional values for R² are 2-(methoxy)-ethoxy, 4-(methoxy)-butoxy.

In yet another embodiment, R² is hydrogen, methyl, ethyl, propyl, butyl, hexyl, 5-pentenyl, 3,3,3-trifluoropropyl, 4,4-difluoropentyl, 3-(cyclopropyl)propyl, 4-(cyclopropyl)butyl, 3-hydroxypropyl, 4-hydroxybutyl, 4-hydroxypentyl, 4-hydroxyhexyl, 5-hydroxyhexyl, 2-hydroxyethoxy, 5-oxohexyl, 3-ethoxypropyl, 4-methoxybutyl, 4-ethoxybutyl, butoxy, hexyloxy, 2-(ethoxy)-ethoxy, 3-methoxypropoxy, 3-ethoxypropoxy, 3-propoxypropoxy, 2-cyclopropylethoxy, (2-(methoxy)ethoxy)methyl, 3-(2,2,2-trifluoroethylamino)propyl, 3-(formylamino)propyl, 3-(acetylamino)propyl, 3-(propionyl-amino)propyl, 3-(butanoylamino)propyl, 3-((2-methoxypropionyl)amino)propyl, 3-(cyclopropyl-carbonylamino)propyl, 3-(trifluoroacetylamino)propyl, 3-(methoxycarbonylamino)propyl, 3-(ethoxycarbonylamino)propyl, 2-(methoxycarbonylamino)ethoxy, 2-(ethoxycarbonylamino)-ethoxy, 3-(methylaminocarbonylamino)propyl, 3-(dimethylaminocarbonyl-amino)propyl, 3-(aminocarbonyl)propyl, 3-(methylaminocarbonyl)propyl, 3-(ethylaminocarbonyl)propyl, 2-(acetylamino)ethoxy, 2-(propionylamino)ethoxy, aminocarbonylmethoxy, methylamino-carbonylmethoxy, ethylaminocarbonylmethoxy, propylaminocarbonylmethoxy, 2-(methylaminocarbonyl)ethoxy, 2-(ethylaminocarbonyl)ethoxy, 2-(propylaminocarbonyl)ethoxy, (2-(methoxy)ethoxy)carbonylamino, methoxymethylcarbonylaminomethyl, or 3-(aminosulfonylamino)propyl, additional values for R² are 2-(methoxy)-ethoxy, 4-(methoxy)-butoxy.

In another embodiment, R² is selected from (C₁-C₃)alkoxyprop-3-yloxy-, (C₁-C₃)alkoxy)but-4-yl, ((C₁-C₃)alkoxycarbonylamino)prop-3-yl and [(optionally substituted (C₁-C₃)alkyl)carbonylamino]prop-3-yl, wherein the optionally substituted (C₁-C₃)alkyl moiety is optionally substituted with one or more substituents selected from halogen and hydroxyl. In a specific embodiment, R² is 3-(methoxy)propoxy, 4-(methoxy)butyl, 3-(methoxycarbonylamino)propyl, or 3-(methylcarbonylamino)propyl. In another embodiment, R² is selected from (C₁-C₃)alkoxycarbonylamino)prop-3-yl and [optionally substituted (C₁-C₃)alkyl]carbonylamino)prop-3-yl, wherein the optionally substituted (C₁-C₃)alkyl moiety is optionally substituted with one or more substituents selected from halogen and hydroxyl. In another embodiment, R² is selected from (C₁-C₃)alkoxycarbonylamino)prop-3-yl and (C₁-C₃)alkylcarbonylamino)prop-3-yl. In a specific embodiment, R² is 3-(methoxycarbonylamino)propyl or 3-(methylcarbonylamino)propyl.

In another embodiment, R² is selected from (C₁-C₃)alkoxyprop-3-yloxy-, (C₁-C₃)alkoxy)but-4-yl, ((C₁-C₃)alkoxycarbonylamino)eth-2-yloxy, ((C₁-C₃)alkoxycarbonylamino)prop-3-yl, and [(optionally substituted (C₁-C₃)alkyl)carbonylamino]prop-3-yl, wherein the optionally substituted (C₁-C₃)alkyl moiety is optionally substituted with one or more substituents selected from halogen and hydroxyl. In a specific embodiment, R² is 3-(methoxy)propoxy, 4-(methoxy)butyl, 3-(trifluoroacetylamino)propyl, 3-(methoxycarbonylamino)propyl, 3-(methylcarbonylamino)propyl, or 2-(methoxycarbonylamino)ethoxy. In another embodiment, R² is selected from (C₁-C₃)alkoxycarbonylamino)prop-3-yl and [(optionally substituted (C₁-C₃)alkyl)carbonylamino]prop-3-yl, wherein the optionally substituted (C₁-C₃)alkyl moiety is optionally substituted with one or more substituents selected from halogen and hydroxyl. In another embodiment, R² is selected from (C₁-C₃)alkoxycarbonylamino)prop-3-yl and (C₁-C₃)alkylcarbonylamino)prop-3-yl. In a specific embodiment, R² is 3-(methoxycarbonylamino)propyl, 3-(trifluoroacetylamino)propyl, or 3-(methylcarbonylamino)propyl.

In one embodiment of this invention, R³ is 1) H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkylcarbonylamino, (C₁-C₆)-alkoxycarbonylamino, (C₁-C₆)alkylaminocarbonylamino, di(C₁-C₆)alkylaminocarbonylamino, (C₁-C₆)alkylsulfonylamino, (C₁-C₆)alkylaminosulfonylamino, or di(C₁-C₆)alkylaminosulfonyl-amino, or

2) phenylamino or heteroarylamino in which each phenylamino and heteroarylamino group is optionally substituted with 1 to 5 groups independently selected from: fluorine, chlorine, bromine, iodine, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkyl(C₁-C₆)alkylthio, halo(C₁-C₆)alkylthio, halo(C₃-C₆)cycloalkythio, halo(C₄-C₇)-cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkyl-sulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkyl(C₁-C₆)alkylthio-(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxy-carbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl;

provided that when R³ is hydroxyl, halogen or optionally substituted phenylamino or heteroarylamino, then R² is not an optionally substituted alkoxy, alkylthio or amino group as follows:

provided that when R³ is hydroxyl, halogen or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonyl-amino(C₁-C₁₂)alkoxy, (C₁-C₆)alkylcarbonyl-amino(C₁-C₆)alkoxy, (C₃-C₄)cycloalkylcarbonylamino-(C₁-C₆)alkoxy, aminosulfonylamino(C₁-C₁₂)alkoxy, (C₁-C₆)alkyl-sulfonylamino(C₁-C₆)alkoxy, formylamino(C₁-C₆)alkoxy, (C₁-C₆)alkoxycarbonyl-amino(C₁-C₆)alkoxy, (C₁-C₆)alkylamino-carbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl-(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkylcarbonylamino;

provided further that when R³ is hydroxyl, halogen, or optionally substituted phenylamino or heteroarylamino, then R² is not a unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkylsulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, amino-carbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio, wherein the thio moiety is replaced by a sulfinyl (sulfoxide, i.e., —S(O)—) or a sulfonyl (sulfone, i.e., —S(O)₂—) moiety, and provided further that when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkylcarbonyl-amino(C₁-C₆)alkoxy, (C₁-C₆)alkylcarbonylamino(C₁-C₆)alkylthio, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkoxy, formylamino(C₁-C₆)alkylthio, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkoxy, di(C₁-C₆)alkylaminocarbonyl-amino(C₁-C₆)alkoxy, (C₁-C₆)alkylaminocarbonylamino(C₁-C₆)alkylthio, di(C₁-C₆)alkylamino-carbonylamino(C₁-C₆)alkylthio, aminocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkoxy, aminocarboxy(C₁-C₆)alkylthio, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, (C₁-C₁₂)alkylcarbonylamino, wherein the carbonyl moiety is replaced by a thiocarbonyl moiety.

In another embodiment, R² is selected from (C₁-C₃)alkoxyprop-3-yloxy, (C₁-C₃)alkoxybut-4-yl, ((C₁-C₃)alkoxycarbonylamino)prop-3-yl and [(optionally substituted (C₁-C₃)alkyl)carbonylamino]prop-3-yl, wherein the optionally substituted (C₁-C₃)alkyl moiety is optionally substituted with one or more substituents selected from halogen and hydroxyl; and R³ is H, halogen, or hydroxyl, provided that when R³ is halogen or hydroxyl, R² is not (C₁-C₃)alkoxyprop-3-yloxy.

In another embodiment, R² is selected from (C₁-C₃)alkoxyprop-3-yloxy, (C₁-C₃)alkoxybut-4-yl, ((C₁-C₃)alkoxycarbonylamino)eth-2-yloxy, ((C₁-C₃)alkoxycarbonylamino)prop-3-yl, and [(optionally substituted (C₁-C₃)alkyl)carbonylamino]prop-3-yl, wherein the optionally substituted (C₁-C₃)alkyl moiety is optionally substituted with one or more substituents selected from halogen and hydroxyl; and R³ is H, halogen, or hydroxyl, provided that when R³ is halogen or hydroxyl, R² is not (C₁-C₃)alkoxyprop-3-yloxy or ((C₁-C₃)alkoxycarbonylamino)eth-2-yloxy.

In a further embodiment, R² is 3-(methoxy)propoxy, 4-(methoxy)butyl, 3-(methoxycarbonylamino)propyl, or 3-(methylcarbonylamino)propyl, and R³ is H, halogen, or hydroxyl, provided that when R³ is halogen or hydroxyl, R² is not 3-(methoxy)propoxy. In a specific embodiment, R² is 4-(methoxy)butyl, 3-(methoxycarbonylamino)propyl, or 3-(methylcarbonylamino)propyl; and R³ is OH.

In a further embodiment, R² is 3-(methoxy)propoxy, 4-(methoxy)butyl, 3-(trifluoroacetylamino)propyl, 3-(methoxycarbonylamino)propyl, 3-(methylcarbonylamino)propyl, or 2-(methoxycarbonylamino)ethoxy, and R³ is H, halogen, or hydroxyl, provided that when R³ is halogen or hydroxyl, R² is not 3-(methoxy)propoxy or 2-(methoxycarbonylamino)ethoxy. In a specific embodiment, R² is 4-(methoxy)butyl, 3-(methoxycarbonylamino)propyl, 3-(trifluoroacetylamino)propyl, or 3-(methylcarbonylamino)propyl; and R³ is OH.

In another embodiment of this invention, A is a saturated or unsaturated 5- or 6-membered ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from halogen, (C₁-C₆)alkyl, halo(C₁-C₆)alkyl, and oxo, and the carbonyl moiety and Y are attached to carbon or nitrogen atoms in ring A in a 1,3 or 1,4 relationship. In another embodiment, ring A in the moiety —Y-A-C(═O)— is represented by:

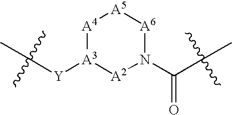

wherein A¹ is represented in the structure above by N, and 1) each of A², A⁴, A⁵, and A⁶ is CH₂; and A³ is CH; or 2) each of A², A⁴, A⁵, and A⁶ is CH; and A³ is C; or 3) A⁴ is CH₂CH₂, each of A², A⁵, and A⁶ is CH₂; and A³ is CH; or 4) A⁴ is O, each of A², A⁵, and A⁶ is CH₂; and A³ is CH; or 5) A⁴ is CH₂O, each of A², A⁵, and A⁶ is CH₂; and A³ is CH; or 6) each of A², A⁴, and A⁶ is CH₂, A⁵ is a single bond and A³ is CH, or 7) each of A², A⁴, and A⁶ is CH; A⁵ is a single bond and A³ is C.

In another embodiment of this invention, A⁴ is CH₂ or O, and A², A⁵, and A⁶ is CH₂; and A³ is CH.

In a further embodiment, L is a linear (C₁-C₆)alkyl chain which is optionally substituted by 1-4 groups independently selected from R⁴, R⁵, R⁶, and R⁷; wherein each R⁴, R⁵, R⁶, and R⁷ is independently selected from hydroxyl, carboxy (hydroxycarbonyl), amino, amido (aminocarbonyl), and from the following optionally substituted groups:

1) (C₁-C₁₂)alkyl, (C₃-C₈)cycloalkyl, (C₃-C₈)cycloalkyl(C₁-C₃)alkyl, (C₁-C₆)alkoxy, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, (C₁-C₁₂)alkoxycarbonyl, (C₃-C₈)cycloalkyl-oxy-carbonyl, (C₁-C₆)alkylaminocarbonyl, di[(C₁-C₆)alkyl]aminocarbonyl, saturated heterocyclyl, and saturated heterocyclyl(C₁-C₃)alkyl; each is optionally and independently substituted by a group selected from: halogen, cyano, hydroxyl, carboxy, amino, amido, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and

2) phenyl, naphthyl, heteroaryl, phenoxycarbonyl, naphthyloxycarbonyl, heteroaryloxycarbonyl, phenylaminocarbonyl, naphthylaminocarbonyl, heteroarylaminocarbonyl, (phenyl)((C₁-C₆)alkyl)aminocarbonyl, (naphthyl)((C₁-C₆)alkyl)aminocarbonyl, (hetero aryl)((C₁-C₆)alkyl)aminocarbonyl, phenyl(C₁-C₃)alkyl, naphthyl(C₁-C₃)alkyl, heteroaryl(C₁-C₃)alkyl, phenyl(C₁-C₃)alkoxycarbonyl, naphthyl(C₁-C₃)alkoxycarbonyl, heteroaryl(C₁-C₃)alkoxycarbonyl, phenyl(C₁-C₃)alkylaminocarbonyl, naphthyl(C₁-C₃)alkylaminocarbonyl, heteroaryl(C₁-C₃)alkylaminocarbonyl, (phenyl(C₁-C₃)alkyl)((C₁-C₆)alkyl)aminocarbonyl, (naphthyl(C₁-C₃)alkyl)((C₁-C₆)alkyl)aminocarbonyl, and (heteroaryl(C₁-C₃)alkyl)) ((C₁-C₆)alkyl)aminocarbonyl; each optionally substituted with 1 to 3 groups independently selected from: fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkylsulfonyl, and (C₁-C₃)alkoxycarbonyl.

In another embodiment, L is a linear (C₁-C₆)alkyl chain, which is optionally substituted by 1-2 groups independently selected from hydroxyl, carboxy, amino, amido, and from the following optionally substituted groups:

1) (C₁-C₄)alkyl, (C₁-C₆)alkoxy(C₁-C₆)alkyl, (C₁-C₆)alkoxycarbonyl, (C₃-C₈)cycloalkyl-(C₁-C₆)alkoxy-carbonyl, (C₅-C₆)cycloalkyl-(C₁-C₃)alkyl, (C₁-C₆)alkylaminocarbonyl, each optionally and independently substituted by a group selected from: halogen, hydroxyl, carboxy, amino, amido, (C₁-C₃)alkyl, (C₁-C₃)alkoxy, (C₃-C₆)cycloalkyl, halo(C₁-C₃)alkyl, and halo(C₁-C₃)alkoxy; and

2) phenyl, heteroaryl, phenyl(C₁-C₃)alkyl, heteroaryl(C₁-C₃)alkyl, phenyl(C₁-C₃)alkoxycarbonyl, heteroaryl(C₁-C₃)alkoxycarbonyl, phenyl(C₁-C₃)alkylaminocarbonyl, and heteroaryl(C₁-C₃)alkylaminocarbonyl; each optionally substituted with 1 to 3 groups independently selected from halogen, (C₁-C₆)alkyl, halo(C₁-C₆)alkyl, and (C₁-C₆)alkylamino. In yet another embodiment, L is a linear (C₁-C₆)alkyl chain, which is optionally substituted by 1-2 groups independently selected from hydroxyl, carboxy, amino, amido, and from the following optionally substituted groups:

1) (C₁-C₄)alkyl, (C₁-C₆)alkoxy(C₁-C₆)alkyl, (C₁-C₆)alkoxycarbonyl, (C₃-C₆)cycloalkyl-(C₁-C₆)alkoxy-carbonyl, (C₅-C₆)cycloalkyl-(C₁-C₂)alkyl, (C₁-C₆)alkylaminocarbonyl, each optionally and independently substituted by a group selected from: halogen, hydroxyl, carboxy, amino, amido, (C₁-C₃)alkyl, (C₁-C₃)alkoxy, (C₃-C₆)cycloalkyl, halo(C₁-C₃)alkyl, and halo(C₁-C₃)alkoxy; and

2) phenyl, heteroaryl, phenyl(C₁-C₃)alkyl, heteroaryl(C₁-C₃)alkyl, phenyl(C₁-C₃)alkoxycarbonyl, heteroaryl(C₁-C₃)alkoxycarbonyl, phenyl(C₁-C₃)alkylaminocarbonyl, and heteroaryl(C₁-C₃)alkylaminocarbonyl; each optionally substituted with 1 to 3 groups independently selected from halogen, (C₁-C₆)alkyl, halo(C₁-C₆)alkyl, and (C₁-C₆)alkylamino. In a further embodiment, L is a linear (C₁-C₅)alkyl chain which is unsubstituted or substituted by 1-2 substituents selected from methyl, isopropyl, hydroxy, hydroxymethyl, carboxy, methoxycarbonyl, amino, aminopropyl, amido, amidomethyl (aminocarbonylmethyl-), 4-chlorophenyl, benzyl, cyclohexylmethyl, and benzyloxycarbonyl.

In another embodiment, L is a linear (C₁-C₆)alkyl chain, which is optionally substituted by 1-2 groups independently selected from hydroxyl, carboxy, amino, amido, and from the following optionally substituted groups:

1) (C₁-C₄)alkyl, (C₁-C₆)alkoxy, (C₁-C₆)alkoxy(C₁-C₆)alkyl, (C₁-C₆)alkoxycarbonyl, (C₃-C₈)cycloalkyl-(C₁-C₆)alkoxy-carbonyl, (C₅-C₆)cycloalkyl-(C₁-C₃)alkyl, (C₁-C₆)alkylaminocarbonyl, each optionally and independently substituted by a group selected from: halogen, hydroxyl, carboxy, amino, amido, (C₁-C₃)alkyl, (C₁-C₃)alkoxy, (C₃-C₆)cycloalkyl, halo(C₁-C₃)alkyl, and halo(C₁-C₃)alkoxy; and

2) phenyl, heteroaryl, phenyl(C₁-C₃)alkyl, heteroaryl(C₁-C₃)alkyl, phenyl(C₁-C₃)alkoxycarbonyl, heteroaryl(C₁-C₃)alkoxycarbonyl, phenyl(C₁-C₃)alkylaminocarbonyl, and heteroaryl(C₁-C₃)alkylaminocarbonyl; each optionally substituted with 1 to 3 groups independently selected from halogen, (C₁-C₆)alkyl, halo(C₁-C₆)alkyl, and (C₁-C₆)alkylamino. In a further embodiment, L is a linear (C₁-C₅)alkyl chain which is unsubstituted or substituted by 1-2 substituents independently selected from methyl, isopropyl, hydroxy, hydroxymethyl, methoxy, carboxy, methoxycarbonyl, amino, aminopropyl, amido, amidomethyl (aminocarbonylmethyl-), 4-chlorophenyl, benzyl, cyclohexylmethyl, benzyloxycarbonyl, and indolylmethyl.

In specific embodiments, L is CH₂, CH₂CH₂, CH₂CH₂CH₂, CH₂CH₂CH₂CH₂, CH₂CH₂CH₂CH₂CH₂, CH(CH₃), C(CH₃)(CH₃), CH(CH(CH₃)₂), CH(CH₂OH), C(CH₃)(CH₂OH)CH₂, CH₂CH₂CH(CO₂H), CH₂CH₂CH(CO₂CH₃), CH₂CH₂CH(CH₂OH), CH₂CH(OH)CH₂, CH(OH)CH₂CH₂, CH(CH₂-phenyl), CH₂CH(4-chlorophenyl)CH₂, CH₂CH(OCH₃)CH₂, CH₂CH(CH₂-cyclohexyl), CH₂CH(CH₂-cyclohexyl)CH₂, CH₂CH(OH)CH(CH₂-phenyl), CH(CH₂-indol-3-yl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, or CH(NH₂)CH₂CH₂CH₂.

In other specific embodiments, L is CH₂, CH₂CH₂, CH₂CH₂CH₂, CH₂CH₂CH₂CH₂, CH₂CH₂CH₂CH₂CH₂, CH(CH₃), C(CH₃)(CH₃), CH(CH(CH₃)₂), CH(CH₂OH), CH₂CH₂CH(CO₂CH₃), CH₂CH₂CH(CH₂OH), CH₂CH(OH)CH₂, CH(OH)CH₂CH₂, CH(CH₂-phenyl), CH₂CH(CH₂-cyclohexyl), CH₂CH(OH)CH(CH₂-phenyl), CH(CH₂-indol-3-yl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, or CH(NH₂)CH₂CH₂CH₂.

In other specific embodiments, L is CH₂, CH₂CH₂, CH₂CH₂CH₂, CH₂CH₂CH₂CH₂, CH₂CH₂CH₂CH₂CH₂, CH(CH₃), C(CH₃)(CH₃), CH(CH(CH₃)₂), CH(CH₂OH), CH₂CH₂CH(CO₂CH₃), CH₂CH₂CH(CH₂OH), CH₂CH(OH)CH₂, CH(OH)CH₂CH₂, CH(CH₂-phenyl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, or CH(NH₂)CH₂CH₂CH₂.

In another embodiment, L is —NH(C₂-C₆)alkyl substituted by phenyl, naphthyl, heteroaryl, phenyl(C₁-C₃)alkyl, naphthyl(C₁-C₃)alkyl, or heteroaryl(C₁-C₃)alkyl, wherein the phenyl, naphthyl and heteroaryl groups are optionally substituted by 1 to 3 groups selected from: fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl;

When L is a —NH(C₂-C₆)alkyl group the carbonyl moiety depicted in Formula I is attached to nitrogen atom of L and G is attached to (C₂-C₆)alkyl moiety of L;

In a further embodiment, L is —NH(C₂-C₆)alkyl optionally substituted by hydroxyl, carboxy (hydroxycarbonyl), amino, amido (aminocarbonyl-), or (C₁-C₆)alkoxycarbonyl, when R² is selected from ((C₁-C₃)alkoxycarbonylamino)prop-3-yl and optionally substituted [((C₁-C₃)alkyl)carbonylamino]prop-3-yl, wherein the optionally substituted (C₁-C₃)alkyl moiety is optionally substituted with one or more substituents selected from halogen and hydroxyl; and R³ is H, halogen, or hydroxyl.

In yet another embodiment, L is —NH(C₂-C₆)alkyl substituted by phenyl(C₁-C₃)alkoxycarbonyl, phenyl, heteroaryl, phenyl(C₁-C₃)alkyl, or heteroaryl(C₁-C₃)alkyl, wherein the phenyl and heteroaryl groups are optionally substituted by 1 to 3 groups selected from: fluoride, chloride, bromide, cyano, nitro, amino, hydroxy, carboxy, (C₁-C₄)alkyl, (C₃-C₆)cycloalkyl, halo(C₁-C₄)alkyl, (C₁-C₄)alkoxy, (C₃-C₆)cycloalkoxy, halo(C₁-C₄)alkoxy, (C₁-C₄)alkylamino, di(C₁-C₄)alkylamino, (C₁-C₄)alkoxycarbonyl, aminocarbonyl, (C₁-C₄)alkylaminocarbonyl, di(C₁-C₄)alkylaminocarbonyl, hydroxy(C₁-C₃)alkyl, (C₁-C₄)alkoxy(C₁-C₃)alkyl, (C₁-C₄)alkylamino(C₁-C₃)alkyl, di(C₁-C₄)alkylamino(C₁-C₃)alkyl, (C₁-C₄)alkoxycarbonyl(C₁-C₃)alkyl, aminocarbonyl(C₁-C₃)alkyl, (C₁-C₄)alkylaminocarbonyl(C₁-C₃)alkyl, and di(C₁-C₄)alkylaminocarbonyl(C₁-C₃)alkyl.

In another further embodiment, L is —NH(C₂-C₆)alkyl substituted by phenyl or phenyl(C₁-C₃)alkyl, wherein said phenyl groups are optionally substituted by 1-2 groups selected from: fluoride, chloride, bromide, cyano, nitro, amino, hydroxy, (C₁-C₄)alkyl, halo(C₁-C₄)alkyl, and (C₁-C₄)alkoxy.

In specific embodiments, L is NHCH₂CH(CH₂-4-OCH₃-phenyl) or NHCH₂CH(CH₂CH₂-4-OCH₃-phenyl).

In other specific embodiments L is NHCH₂CH₂ or NHCH₂C(CH₃)₂CH₂, provided that R² is 3-(methoxycarbonylamino)propyl, and R³ is OH.

In one embodiment, G is —OH, —NH₂, —NHR⁸, —NR⁸R⁹, —NHC(═NH)NH₂, —NHC(═NH)NHR⁸; —C(═NH)NH₂, or —C(═NH)NHR⁸; wherein: R⁸ is a) (C₁-C₁₂)alkyl, (C₄-C₁₂)cycloalkyl(C₁-C₆)alkyl, halo(C₁-C₁₂)alkyl, halo(C₄-C₁₂)cycloalkyl(C₁-C₆)alkyl, (C₂-C₁₂)alkenyl, (C₅-C₁₂)cycloalkyl(C₂-C₆)alkenyl, halo(C₂-C₁₂)alkenyl, halo(C₅-C₁₂)cycloalkyl(C₂-C₆)alkenyl, (C₂-C₁₂)alkynyl, (C₅-C₁₂)cycloalkyl(C₂-C₆)alkynyl, halo(C₂-C₁₂)alkynyl, halo(C₅-C₁₂)cycloalkyl(C₂-C₆)alkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkylsulfinyl(C₁-C₆)alkyl, halo(C₁-C₆)alkylsulfinyl(C₁-C₆)alkyl, (C₁-C₆)alkylsulfonyl(C₁-C₆)alkyl, halo(C₁-C₆)alkylsulfonyl(C₁-C₆)alkyl, amino(C₁-C₄)alkyl-carbonyl, (C₁-C₄)alkyl-amino(C₁-C₄)alkylcarbonyl, di((C₁-C₄)alkyl)-amino C₁-C₄)alkylcarbonyl, aminocarbonyl(C₁-C₆)alkyl, (C₁-C₆)alkylaminocarbonyl(C₁-C₆)alkyl, di(C₁-C₆)alkylamino-carbonyl(C₁-C₆)alkyl, cyano(C₁-C₆)alkyl, hydroxy(C₁-C₁₂)alkyl, amino(C₁-C₁₂)alkyl, (C₁-C₆)alkylamino(C₁-C₆)alkyl, di((C₁-C₆)alkyl)-amino C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or b) phenyl, naphthyl, heteroaryl, phenyl(C₁-C₃)alkyl, naphthyl(C₁-C₃)alkyl, or heteroaryl(C₁-C₃)alkyl, each optionally substituted by 1 to 3 groups independently selected from: 1) fluorine, chlorine, bromine, iodine, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkyl(C₁-C₆)alkylthio, halo(C₁-C₆)alkylthio, halo(C₃-C₆)cycloalkythio, halo(C₄-C₇)cycloalkyl(C₁-C₆)(C₁-C₆)alkylthio, (C₁-C₆)alkylsulfinyl, (C₃-C₆)cycloalkylsulfinyl, (C₄-C₇)-cycloalkyl(C₁-C₆)alkylsulfinyl, halo(C₁-C₆)alkylsulfinyl, halo(C₃-C₆)cycloalkyl-sulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)-cycloalkyl(C₁-C₆)alkylsulfonyl, (C₁-C₆)alkylamino, di(C₁-C₆)alkylamino, (C₁-C₆)alkoxy-(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkyl(C₁-C₆)alkylthio-(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)-cycloalkyl(C₁-C₆)alkylsulfinyl(C₁-C₆)alkyl, (C₁-C₈)alkyl-sulfonyl(C₁-C₆)alkyl, (C₃-C₈)-cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)-alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)-alkylaminocarbonyl(C₁-C₆)alkyl(C₁-C₈)acylamino(C₁-C₆)alkyl, (C₁-C₈)alkoxy-carbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl, and di(C₁-C₈)alkylaminocarboxy(C₁-C₆)alkyl; or 2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)alkoxy, (C₁-C₃)alkylsulfonyl, and (C₁-C₃)-alkoxycarbonyl; and R⁹ is (C₁-C₆)alkyl.

In another embodiment, G is —OH, —NH₂, NHR⁸, or —NR⁸R⁹, wherein R⁸ is a) (C₁-C₄)alkyl, halo(C₁-C₄)alkyl, hydroxy(C₁-C₄)alkyl, amino(C₁-C₄)alkyl, (C₁-C₄)alkyl-amino(C₁-C₄)alkyl, di((C₁-C₄)alkyl)-amino(C₁-C₄)alkyl, amino(C₁-C₄)alkylcarbonyl, (C₁-C₄)alkyl-amino(C₁-C₄)alkylcarbonyl, di((C₁-C₄)alkyl)-amino C₁-C₄)alkylcarbonyl, (C₄-C₁₀)cycloalkyl(C₁-C₄)alkyl, (C₁-C₄)alkoxy(C₁-C₄)alkyl, aminocarbonyl(C₁-C₄)alkyl, (C₁-C₄)alkylaminocarbonyl(C₁-C₄)alkyl, di(C₁-C₄)alkylaminocarbonyl(C₁-C₄)alkyl, or amino(imino)(C₁-C₄)alkyl; or b) phenyl(C₁-C₂)alkyl optionally substituted with 1 to 3 groups independently selected from halogen, cyano, (C₁-C₃)alkyl, halo(C₁-C₃)alkyl, (C₁-C₃)alkoxy, and halo(C₁-C₃)alkoxy; and R⁹ is (C₁-C₄)alkyl.

In yet another embodiment, G is —OH, —NH₂, NHR⁸, or —NR⁸R⁹, where R⁸ is methyl, hydroxyethyl, methylaminoethyl, aminomethylcarbonyl, amino(1-ethyl)carbonyl (L-alanyl), and R⁹ is methyl. In more specific embodiments of the compounds of this invention, G is selected from —NH₂, —NH(CH₃), —NHCH₂CH₂OH, —N(CH₃)CH₂CH₂NH(CH₃), —NHC(═O)CH₂NH₂ or —NHC(═O)CH(CH₃)NH₂.

In yet another embodiment, G is —OH, —NH₂, NHR⁸, or —NR⁸R⁹, where R⁸ is methyl, hydroxyethyl, methylaminoethyl, aminomethylcarbonyl, amino(1-ethyl)carbonyl (L-alanyl), amino(imino)methyl. and R⁹ is methyl. In more specific embodiments of the compounds of this invention, G is selected from —NH₂, —NH(CH₃), —N(CH₃)₂, —NHCH₂CH₂OH, —N(CH₃)CH₂CH₂NH(CH₃), —NHC(═O)CH₂NH₂, —NHC(═O)CH(CH₃)NH₂, or —NHC(═NH)NH₂.

In a further embodiment of the compounds of Formula Ia, Ib, or Ic of this invention, R¹ is phenyl, optionally substituted with 1 to 3 substituents independently selected from: fluorine, chlorine, and methyl, or is a bi-aromatic group comprised of a phenyl substituted with another aromatic moiety selected from phenyl, phenoxy, furanyl, quinolinyl, and dihydrobenzofuranyl, wherein the bi-aromatic group is optionally substituted with 1 to 3 substituents independently selected from fluorine, chlorine, methyl, ethyl, isopropyl, trifluoromethyl, and methoxy, wherein the optional substituents are substituted on either one of or both of the phenyl moiety and the other aromatic moiety of the bi-aromatic group; R² is 3-(methoxy)propoxy, 4-(methoxy)butyl, 3-(trifluoroacetylamino)propyl, 3-(methoxycarbonylamino)propyl, 3-(methylcarbonylamino)propyl, or 2-(methoxycarbonylamino)ethoxy; R³ is H or OH; provided that when R³ is OH, R² is not 3-(methoxy)propoxy or 2-(methoxycarbonylamino)ethoxy; A⁴ is CH₂ or O; L is a linear (C₁-C₅)alkyl chain which is unsubstituted or substituted by 1-2 substituents selected from methyl, isopropyl, hydroxy, hydroxymethyl, methoxy, carboxy, methoxycarbonyl, amino, aminopropyl, amido, amidomethyl (aminocarbonylmethyl), 4-chlorophenyl, benzyl, cyclohexylmethyl, benzyloxycarbonyl, and indolylmethyl, or L is —NH(C₂-C₆)alkyl substituted by phenyl or phenyl(C₁-C₃)alkyl, wherein said phenyl groups are optionally substituted by 1 or 2 groups selected from: fluoride, chloride, bromide, cyano, nitro, amino, hydroxy, (C₁-C₄)alkyl, halo(C₁-C₄)alkyl, and (C₁-C₄)alkoxy, and G is OH, NH₂, NH(CH₃), —N(CH₃)₂, NHCH₂CH₂OH, N(CH₃)CH₂CH₂NH(CH₃), NHC(═O)CH₂NH₂, NHC(═O)CH(CH₃)NH₂, or —NHC(═NH)NH₂.

In a further embodiment of the compounds of Formula I, Ia, Ib, or Ic of this invention, R¹ is phenyl, 3-chlorophenyl, 3-fluorophenyl, 6-chloro-3′-ethyl-2-biphenyl, 3′-ethyl-6-fluoro-2-biphenyl, 6-fluoro-3′-methyl-2-biphenyl, 6-fluoro-3′-(1-methylethyl)-2-biphenyl, 3-fluoro-2-(3-quinolinyl)phenyl, 3-chloro-2-(3-quinolinyl)phenyl, 3-chloro-2-(2,3-dihydro-1-benzofuran-6-yl)phenyl, 2-[(2-methylphenyl)oxy]phenyl, 3-chloro-2-(5-methyl-2-furanyl)phenyl, 4′,6-difluoro-3′-methyl-2-biphenyl, 6-chloro-3′-fluoro-5′-methyl-2-biphenyl, 2′,6-difluoro-5′-methyl-2-biphenyl, 3′,6-difluoro-5′-methyl-2-biphenyl, or 2′-(methoxy)-5′-(trifluoromethyl)-2-biphenyl; R² is 4-(methoxy)butyl, 3-(trifluoroacetylamino)propyl, 3-(methoxycarbonylamino)propyl, or 3-(methylcarbonylamino)propyl; R³ is H or OH; and L is CH₂, CH₂CH₂, CH₂CH₂CH₂, CH₂CH₂CH₂CH₂, CH₂CH₂CH₂CH₂CH₂, CH(CH₃), C(CH₃)(CH₃), CH(CH(CH₃)₂), CH(CH₂OH), C(CH₃)(CH₂OH)CH₂, CH₂CH₂CH(CO₂H), CH₂CH₂CH(CO₂CH₃), CH₂CH₂CH(CH₂OH), CH₂CH(OH)CH₂, CH(OH)CH₂CH₂, CH(CH₂-phenyl), CH₂CH(4-chlorophenyl)CH₂, CH₂CH(OCH₃)CH₂, CH₂CH(CH₂-cyclohexyl), CH₂CH(CH₂-cyclohexyl)CH₂, CH₂CH(OH)CH(CH₂-phenyl), CH(CH₂-indol-3-yl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, CH(NH₂)CH₂CH₂CH₂, NHCH₂CH₂, NHCH₂C(CH₃)₂CH₂, NHCH₂CH(CH₂-4-OCH₃-phenyl), or NHCH₂CH(CH₂CH₂-4-OCH₃-phenyl); and G is —NH₂, NH(CH₃), —N(CH₃)₂, NHCH₂CH₂OH, N(CH₃)CH₂CH₂NH(CH₃), NHC(═O)CH₂NH₂, NHC(═O)CH(CH₃)NH₂, or —NHC(═NH)NH₂.

The present invention contemplates and includes any and all combinations of the embodiments of R¹, R², R³, X, Y, A, L and G, as defined herein.

It will be appreciated by those skilled in the art, that the compounds of this invention contain 1, 2, or more chiral centers and may exist in different enantiomeric and/or diastereomeric forms. The following compounds are recited without reference to the relative or absolute configuration of any of the chiral centers present therein, but such recitation is intended to encompass each enantiomeric and/or diastereomeric form of these compounds and all mixtures thereof, such as enantiomerically and/or diastereomerically enriched mixtures and racemic mixtures. The following are compounds of the invention:

| Cpd. | |
|---|---|
| No. | Name |
| I-1 | 1-(6-chloro-3′-ethyl-2-biphenylyl)-1-[1-(2-methylalanyl)-3-piperidinyl]-5- |
| | (methyloxy)-1-pentanol |
| I-2 | 2-({3-[1-(6-chloro-3′-ethyl-2-biphenylyl)-1-hydroxy-5-(methyloxy)pentyl]-1- |
| | piperidinyl}carbonyl)-2-methyl-1,3-propanediol |
| I-3 | 1-(6-chloro-3′-ethyl-2-biphenylyl)-1-{-1-[N-(2-hydroxyethyl)-2- |
| | methylalanyl]-3-piperidinyl}-5-(methyloxy)-1-pentanol |
| I-4 | 1-[1-(6-aminohexanoyl)-4-piperidinyl]-1-(3′-ethyl-6-fluoro-2-biphenylyl)-5- |
| | (methyloxy)-1-pentanol |
| I-5 | methyl (4-[3-fluoro-2-(3-quinolinyl)phenyl]-4-hydroxy-4-{1-[4- |
| | (methylamino)butanoyl]-3-piperidinyl}butyl)carbamate |
| I-6 | methyl (4-[3-chloro-2-(5-methyl-2-furanyl)phenyl]-4-hydroxy-4-{1-[4- |
| | (methylamino)butanoyl]-3-piperidinyl}butyl)carbamate |
| I-7 | 1-[4-(4-aminobutanoyl)-2-morpholinyl]-1-(4′,6-difluoro-3′-methyl-2- |
| | biphenylyl)-5-(methyloxy)-1-pentanol |
| I-8 | methyl [4-{1-[4-amino-3-(4-chlorophenyl)butanoyl]-3-piperidinyl}-4-(3′- |
| | ethyl-6-fluoro-2-biphenylyl)-4-hydroxybutyl]carbamate |
| I-9 | methyl (4-(6-chloro-3′-fluoro-5′-methyl-2-biphenylyl)-4-hydroxy-4-{1-[4- |
| | (methylamino)butanoyl]-3-piperidinyl}butyl)carbamate |
| I-10 | methyl (4-(3′,6-difluoro-5′-methyl-2-biphenylyl)-4-hydroxy-4-{1-[4- |
| | (methylamino)butanoyl]-3-piperidinyl}butyl)carbamate |
| I-11 | methyl (4-hydroxy-4-{1-[4-(methylamino)butanoyl]-3-piperidinyl}-4-{2-[(2- |
| | methylphenyl)oxy]phenyl}butyl)carbamate |
| I-12 | methyl [4-[1-β-alanyl-3-piperidinyl]-4-(3′-ethyl-6-fluoro-2-biphenylyl)-4- |
| | hydroxybutyl]carbamate |
| I-13 | methyl [4-[1-(4-aminobutanoyl)-3-piperidinyl]-4-(3′-ethyl-6-fluoro-2- |
| | biphenylyl)-4-hydroxybutyl]carbamate |
| I-14 | methyl [4-[1-(5-aminopentanoyl)-3-piperidinyl]-4-(3′-ethyl-6-fluoro-2- |
| | biphenylyl)-4-hydroxybutyl]carbamate |
| I-15 | methyl 2-amino-5-[3-(1-(3′-ethyl-6-fluoro-2-biphenylyl)-1-hydroxy-4- |
| | {[(methyloxy)carbonyl]amino}butyl)-1-piperidinyl]-5-oxopentanoate |
| I-16 | methyl (4-(6-chloro-3′-ethyl-2-biphenylyl)-4-hydroxy-4-{1-[4- |
| | (methylamino)butanoyl]-3-piperidinyl}butyl)carbamate |
| I-17 | methyl [4-{1-[4-(dimethylamino)butanoyl]-3-piperidinyl}-4-(3′-ethyl-6- |
| | fluoro-2-biphenylyl)-4-hydroxybutyl]carbamate |
| I-18 | methyl (4-[3-chloro-2-(2,3-dihydro-1-benzofuran-6-yl)phenyl]-4-hydroxy-4- |
| | {1-[4-(methylamino)butanoyl]-3-piperidinyl}butyl)carbamate |
| I-19 | methyl [4-(3′-ethyl-6-fluoro-2-biphenylyl)-4-hydroxy-4-(1-{N-methyl-N-[2- |
| | (methylamino)ethyl]glycyl}-3-piperidinyl)butyl]carbamate |
| I-20 | methyl [4-(3′-ethyl-6-fluoro-2-biphenylyl)-4-hydroxy-4-(1-{4-[(2- |
| | hydroxyethyl)amino]butanoyl}-3-piperidinyl)butyl]carbamate |
| I-21 | 2-amino-5-[3-(1-(3′-ethyl-6-fluoro-2-biphenylyl)-1-hydroxy-4- |
| | {[(methyloxy)carbonyl]amino}butyl)-1-piperidinyl]-5-oxopentanoic acid |
| I-22 | methyl [4-{1-[4-amino-5-hydroxypentanoyl]-3-piperidinyl}-4-(3′-ethyl-6- |
| | fluoro-2-biphenylyl)-4-hydroxybutyl]carbamate |
| I-23 | methyl [4-{1-[4-amino-5-hydroxypentanoyl]-3-piperidinyl}-4-(3′-ethyl-6- |
| | fluoro-2-biphenylyl)-4-hydroxybutyl]carbamate |
| I-24 | N-(4-(2′,6-difluoro-5′-methyl-2-biphenylyl)-4-hydroxy-4-{1-[4- |
| | (methylamino)butanoyl]-3-piperidinyl}butyl)acetamide |
| I-25 | N-(4-(6-fluoro-3′-methyl-2-biphenylyl)-4-hydroxy-4-{1-[4- |
| | (methylamino)butanoyl]-3-piperidinyl}butyl)acetamide |
| I-26 | methyl [4-[1-(4-amino-3-hydroxybutanoyl)-3-piperidinyl]-4-(3′-ethyl-6- |
| | fluoro-2-biphenylyl)-4-hydroxybutyl]carbamate |
| I-27 | methyl [4-{1-[4-amino-2-hydroxybutanoyl]-3-piperidinyl}-4-(3′-ethyl-6- |
| | fluoro-2-biphenylyl)-4-hydroxybutyl]carbamate |
| I-28 | methyl (4-(3′-ethyl-6-fluoro-2-biphenylyl)-4-hydroxy-4-{1-[4- |
| | (methylamino)butanoyl]-3-piperidinyl}butyl)carbamate |
| I-29 | methyl [4-[1-(6-aminohexanoyl)-3-piperidinyl]-4-(3′-ethyl-6-fluoro-2- |
| | biphenylyl)-4-hydroxybutyl]carbamate |
| I-30 | methyl [4-[1-L-asparaginyl-3-piperidinyl]-4-(3′-ethyl-6-fluoro-2-biphenylyl)- |
| | 4-hydroxybutyl]carbamate |
| I-31 | methyl {4-(3′-ethyl-6-fluoro-2-biphenylyl)-4-hydroxy-4-[1-L-valyl-3- |
| | piperidinyl]butyl}carbamate |
| I-32 | methyl {4-(3′-ethyl-6-fluoro-2-biphenylyl)-4-hydroxy-4-[1-L-seryl-3- |
| | piperidinyl]butyl}carbamate |
| I-33 | methyl [4-[1-(L-alanyl-L-alanyl)-3-piperidinyl]-4-(3′-ethyl-6-fluoro-2- |
| | biphenylyl)-4-hydroxybutyl]carbamate |
| I-34 | methyl {4-(3′-ethyl-6-fluoro-2-biphenylyl)-4-hydroxy-4-[1-phenylalanyl-3- |
| | piperidinyl]butyl}carbamate |
| I-35 | methyl {4-(3′-ethyl-6-fluoro-2-biphenylyl)-4-hydroxy-4-[1-D-tryptophyl-3- |
| | piperidinyl]butyl}carbamate |
| I-36 | methyl {4-(3′-ethyl-6-fluoro-2-biphenylyl)-4-[1-(glycylglycyl)-3-piperidinyl]- |
| | 4-hydroxybutyl}carbamate |
| I-37 | methyl {4-(3′-ethyl-6-fluoro-2-biphenylyl)-4-hydroxy-4-[1-(2-methylalanyl)- |
| | 3-piperidinyl]butyl}carbamate |
| I-38 | phenylmethyl 2-amino-5-[3-(1-(3′-ethyl-6-fluoro-2-biphenylyl)-1-hydroxy-4- |
| | {[(methyloxy)carbonyl]amino}butyl)-1-piperidinyl]-5-oxopentanoate |
| I-39 | phenylmethyl 2-amino-4-[3-(1-(3′-ethyl-6-fluoro-2-biphenylyl)-1-hydroxy-4- |
| | {[(methyloxy)carbonyl]amino}butyl)-1-piperidinyl]-4-oxobutanoate |
| I-40 | methyl {4-(3′-ethyl-6-fluoro-2-biphenylyl)-4-[1-L-α-glutaminyl-3- |
| | piperidinyl]-4-hydroxybutyl}carbamate |
| I-41 | methyl {4-(3′-ethyl-6-fluoro-2-biphenylyl)-4-hydroxy-4-[1-D-ornithyl-3- |
| | piperidinyl]butyl}carbamate |
| I-42 | 1-(3-(1-(3-chlorophenyl)-1-hydroxy-5-methoxypentyl) piperidin-1-yl)-4- |
| | cyclohexyl-3-((methylamino)methyl)butan-1-one |
| I-43 | 3-amino-4-cyclohexyl-1-(3-((3-methoxypropoxy)(phenyl)methyl)piperidin-1- |
| | yl)butan-1-one |
| I-44 | 4-amino-1-(3-(1-(3-fluorophenyl)-1-hydroxy-5-methoxypentyl)piperidin-1- |
| | yl)-3-hydroxy-5-phenylpentan-1-one |
| I-45 | 4-amino-3-hydroxy-1-(3-(-1-hydroxy-5-methoxy-1-(2-(o-tolyloxy)phenyl)pentyl)piperidin- |
| | 1-yl)butan-1-one |
| I-46 | 4-amino-1-(3-(1-hydroxy-5-methoxy-1-(2-(o-tolyloxy)phenyl)pentyl)piperidin- |
| | 1-yl)butan-1-one |
| I-47 | methyl (4-[3-chloro-2-(3-quinolinyl)phenyl]-4-hydroxy-4-{1-[4- |
| | (methylamino)butanoyl]-3-piperidinyl}butyl)carbamate |
| I-48 | methyl {4-hydroxy-4-{1-[4-(methylamino)butanoyl]-3-piperidinyl}-4-[2′- |
| | (methyloxy)-5′-(trifluoromethyl)-2-biphenylyl]butyl}carbamate |
| I-49 | methyl {4-(3′-ethyl-6-fluoro-2-biphenylyl)-4-[1-glycyl-3-piperidinyl]-4- |
| | hydroxybutyl}carbamate |
| I-50 | methyl [4-{1-[4-amino-3-hydroxybutanoyl]-3-piperidinyl}-4-(3′-ethyl-6- |
| | fluoro-2-biphenylyl)-4-hydroxybutyl]carbamate |
| I-51 | N-(4-(2′,6-difluoro-5′-methyl-2-biphenylyl)-4-hydroxy-4-{1-[4- |
| | (methylamino)butanoyl]-3-piperidinyl}butyl)-2,2,2-trifluoroacetamide |
| I-52 | N-[4-{1-[4-amino-3-(4-chlorophenyl)butanoyl]-3-piperidinyl}-4-(2′,6- |
| | difluoro-5′-methyl-2-biphenylyl)-4-hydroxybutyl]-2,2,2-trifluoroacetamide |
| I-53 | N-[4-{1-[4-amino-3-hydroxybutanoyl]-3-piperidinyl}-4-(2′,6-difluoro-5′- |
| | methyl-2-biphenylyl)-4-hydroxybutyl]-2,2,2-trifluoroacetamide |
| I-54 | N-(4-(3′-ethyl-6-fluoro-2-biphenylyl)-4-hydroxy-4-{1-[4- |
| | (methylamino)butanoyl]-3-piperidinyl}butyl)-2,2,2-trifluoroacetamide |
| I-55 | N-[4-{1-[4-amino-3-hydroxybutanoyl]-3-piperidinyl}-4-(3′-ethyl-6-fluoro-2- |
| | biphenylyl)-4-hydroxybutyl]-2,2,2-trifluoroacetamide |
| I-56 | methyl [4-[1-(4-{[amino(imino)methyl]amino}butanoyl)-3-piperidinyl]-4-(3′- |
| | ethyl-6-fluoro-2-biphenylyl)-4-hydroxybutyl]carbamate |
| I-57 | methyl [4-{1-[4-amino-3-(4-chlorophenyl)butanoyl]-3-piperidinyl}-4-(6- |
| | chloro-3′-ethyl-2-biphenylyl)-4-hydroxybutyl]carbamate |
| I-58 | methyl [(4-{1-[4-amino-3-hydroxybutanoyl]-3-piperidinyl}-4-(6-chloro-3′- |
| | ethyl-2-biphenylyl)-4-hydroxybutyl]carbamate |
| I-59 | methyl (4-(3′-ethyl-6-fluoro-2-biphenylyl)-4-hydroxy-4-{1-[3-hydroxy-4- |
| | (methylamino)butanoyl]-3-piperidinyl}butyl)carbamate |
| I-60 | N-(4-(3′-ethyl-6-fluoro-2-biphenylyl)-4-hydroxy-4-{1-[3-hydroxy-4- |
| | (methylamino)butanoyl]-3-piperidinyl}butyl)-2,2,2-trifluoroacetamide |
| I-61 | methyl (4-(3′-ethyl-6-fluoro-2-biphenylyl)-4-hydroxy-4-{1-[4-(methylamino)- |
| | 3-(methyloxy)butanoyl]-3-piperidinyl}butyl)carbamate |
| I-62 | methyl (4-[6-fluoro-3′-(1-methylethyl)-2-biphenylyl]-4-hydroxy-4-{1-[4- |
| | (methylamino)butanoyl]-3-piperidinyl}butyl)carbamate |
| I-63 | methyl {2-[((6-chloro-3′-ethyl-2-biphenylyl){4-[4-(methylamino)butanoyl]-2- |
| | morpholinyl}methyl)oxy]ethyl}carbamate |
| I-64 | methyl {2-[((6-chloro-3′-ethyl-2-biphenylyl){1-[4-(methylamino)butanoyl]-3- |
| | piperidinyl}methyl)oxy]ethyl}carbamate |
| I-65 | methyl {4-(3′-ethyl-6-fluoro-2-biphenylyl)-4-hydroxy-4-[1-({[2- |
| | (methylamino)ethyl]amino}carbonyl)-3-piperidinyl]butyl}carbamate |
| I-66 | methyl [4-(1-{[(2-aminoethyl)amino]carbonyl}-3-piperidinyl)-4-(3′-ethyl-6- |
| | fluoro-2-biphenylyl)-4-hydroxybutyl]carbamate |
| I-67 | methyl [4-(1-{[(3-amino-2,2-dimethylpropyl)amino]carbonyl}-3-piperidinyl)- |
| | 4-(3′-ethyl-6-fluoro-2-biphenylyl)-4-hydroxybutyl]carbamate |
| I-68 | methyl {2-[((6-chloro-3′-ethyl-2-biphenylyl){4-[({2-(methylamino)-4-[4- |
| | (methyloxy)phenyl]butyl}amino)carbonyl]-2- |
| | morpholinyl}methyl)oxy]ethyl}carbamate |
| I-69 | methyl {2-[((6-chloro-3′-ethyl-2-biphenylyl){4-[({2-(methylamino)-3-[4- |
| | (methyloxy)phenyl]propyl}amino)carbonyl]-2- |
| | morpholinyl}methyl)oxy]ethyl}carbamate |
| I-70 | methyl {2-[((6-chloro-3′-ethyl-2-biphenylyl){1-[({2-(methylamino)-4-[4- |
| | (methyloxy)phenyl]butyl}amino)carbonyl]-3- |
| | piperidinyl}methyl)oxy]ethyl}carbamate |
| I-71 | methyl {2-[((6-chloro-3′-ethyl-2-biphenylyl){1-[({2-(methylamino)-3-[4- |
| | (methyloxy)phenyl]propyl}amino)carbonyl]-3- |
| | piperidinyl}methyl)oxy]ethyl}carbamate |

or a diastereomer, enantiomer, or salt thereof.

It will also be appreciated by those skilled in the art that each enantiomer and diastereomer of the compounds of this invention will likely demonstrate a different level of effectiveness of inhibiting the action of aspartic proteases, particularly renin. It will be further appreciated that for the most active compounds, all enantiomers and/or diastereomers may demonstrate some level of activity, but that for compounds with lower activity, certain enantiomers and/or diastereomers may demonstrate such low levels of activity as to be considered inactive. It is understood that the following represent the preferred relative and absolute configuration of the compounds of the invention. It will be appreciated that each of the different enantiomeric and/or diastereomeric forms of the compounds of this invention, including the stereoisomeric forms depicted below, may be separately obtained using conventional procedures (e.g. stereospecific synthesis or resolution via chiral chromatography, crystallization, etc.). The following are compounds of the invention:

| Cpd. | | |
|---|---|---|
| No. | Structure | Name |
| I-1a | 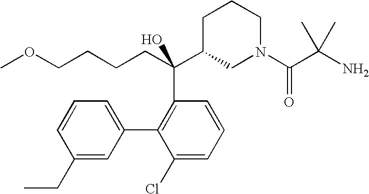 | (1S)-1-(6-chloro-3′-ethyl- 2-biphenylyl)-1-[(3R)-1-(2- methylalanyl)-3- piperidinyl]-5-(methyloxy)- 1-pentanol |
| I-2a | 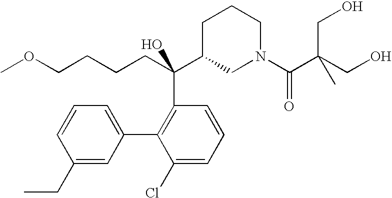 | 2-({(3R)-3-[(1S)-1-(6- chloro-3′-ethyl-2- biphenylyl)-1-hydroxy-5- (methyloxy)pentyl]-1- piperidinyl}carbonyl)-2- methyl-1,3-propanediol |
| I-3a | 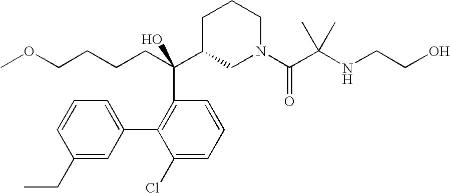 | (1S)-1-(6-chloro-3′-ethyl- 2-biphenylyl)-1-{(3R)-1- [N-(2-hydroxyethyl)-2- methylalanyl]-3- piperidinyl}-5- (methyloxy)-1-pentanol |
| I-4a | 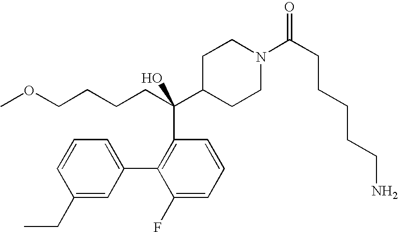 | (1S)-1-[1-(6-amino hexanoyl)-4-piperidinyl]-1- (3′-ethyl-6-fluoro-2- biphenylyl)-5-(methyloxy)- 1-pentanol |
| I-5a | 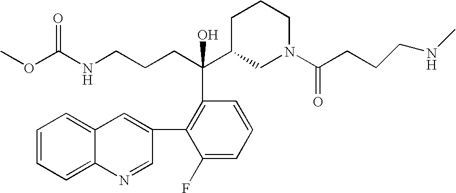 | methyl ((4S)-4-[3-fluoro-2- (3-quinolinyl)phenyl]-4- hydroxy-4-{(3R)-1-[4- (methylamino)butanoyl]-3- piperidinyl}butyl) carbamate |
| I-6a | 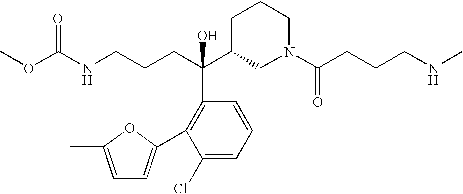 | methyl ((4S)-4-[3-chloro- 2-(5-methyl-2-furanyl) phenyl]-4-hydroxy-4- {(3R)-1-[4-(methylamino) butanoyl]-3-piperidinyl} butyl)carbamate |
| I-7a | 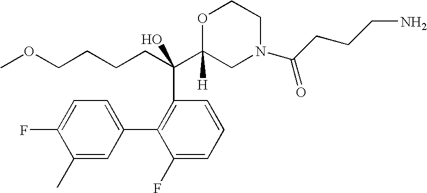 | (1R)-1-[(2R)-4-(4- aminobutanoyl)-2- morpholinyl]-1-(4′,6- difluoro-3′-methyl-2- biphenylyl)-5-(methyloxy)- 1-pentanol |
| I-8a | 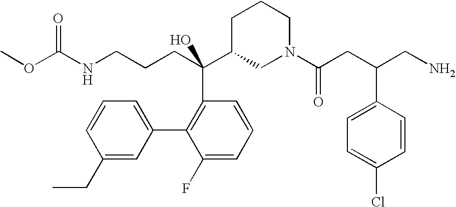 | methyl [(4S)-4-{(3R)-1-[4- amino-3-(4- chlorophenyl)butanoyl]-3- piperidinyl}-4-(3′-ethyl-6- fluoro-2-biphenylyl)-4- hydroxybutyl]carbamate |
| I-9a | 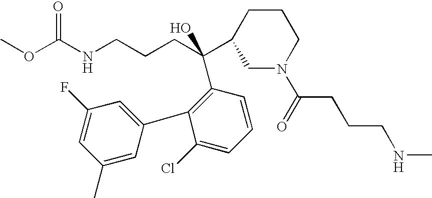 | methyl ((4S)-4-(6-chloro- 3′-fluoro-5′-methyl-2- biphenylyl)-4-hydroxy-4- {(3R)-1-[4-(methylamino) butanoyl]-3-piperidinyl} butyl)carbamate |
| I-10a | 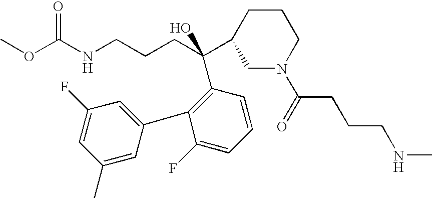 | methyl ((4S)-4-(3′,6- difluoro-5′-methyl-2- biphenylyl)-4-hydroxy-4- {(3R)-1-[4-(methylamino) butanoyl]-3-piperidinyl} butyl)carbamate |
| I-11a | 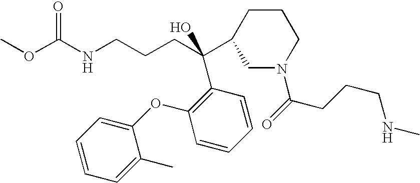 | methyl ((4S)-4-hydroxy-4- {(3R)-1-[4-(methylamino) butanoyl]-3-piperidinyl}-4- {2-[(2-methylphenyl) oxy]phenyl}butyl) carbamate |
| I-12a | 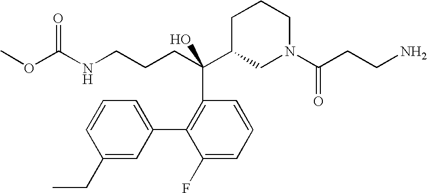 | methyl [(4S)-4-[(3R)-1-β- alanyl-3-piperidinyl]-4-(3′- ethyl-6-fluoro-2- biphenylyl)-4- hydroxybutyl]carbamate |
| I-13a | 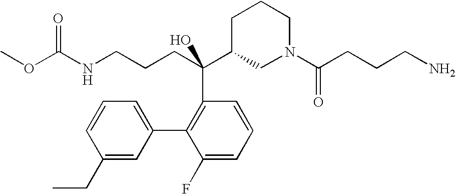 | methyl [(4S)-4-[(3R)-1-(4- aminobutanoyl)-3- piperidinyl]-4-(3′-ethyl-6- fluoro-2-biphenylyl)-4- hydroxybutyl]carbamate |
| I-14a | 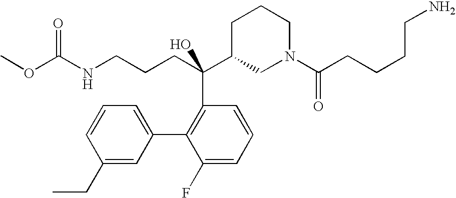 | methyl [(4S)-4-[(3R)-1-(5- aminopentanoyl)-3- piperidinyl]-4-(3′-ethyl-6- fluoro-2-biphenylyl)-4- hydroxybutyl]carbamate |
| I-15a | 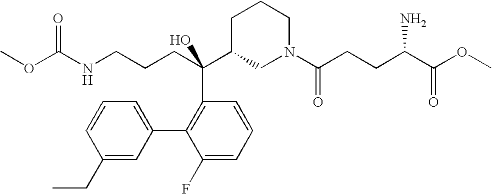 | methyl (2S)-2-amino-5- [(3R)-3-((1S)-1-(3′-ethyl-6- fluoro-2-biphenylyl)-1- hydroxy-4-{[(methyloxy) carbonyl]amino}butyl)-1- piperidinyl]-5- oxopentanoate |
| I-16a | 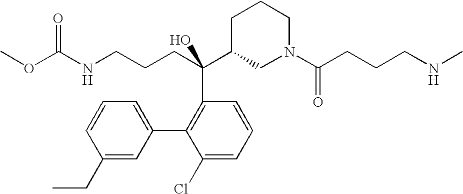 | methyl ((4S)-4-(6-chloro- 3′-ethyl-2-biphenylyl)-4- hydroxy-4-{(3R)-1-[4- (methylamino)butanoyl]-3- piperidinyl}butyl) carbamate |
| I-17a | 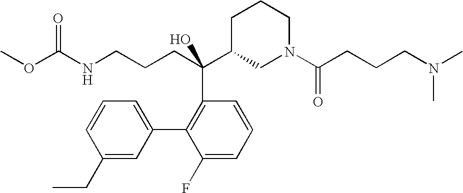 | methyl [(4S)-4-{(3R)-1-[4- (dimethylamino)butanoyl] - 3-piperidinyl}-4-(3′-ethyl- 6-fluoro-2-biphenylyl)-4- hydroxybutyl]carbamate |
| I-18a | 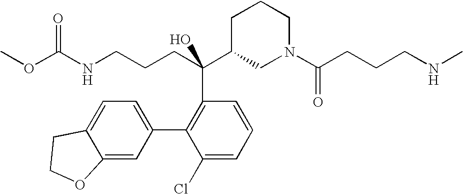 | methyl ((4S)-4-[3-chloro- 2-(2,3-dihydro-1- benzofuran-6-yl)phenyl]-4- hydroxy-4-{(3R)-1-[4- (methylamino)butanoyl]-3- piperidinyl}butyl) carbamate |
| I-19a | 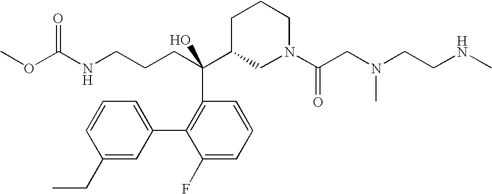 | methyl [(4S)-4-(3′-ethyl-6- fluoro-2-biphenylyl)-4- hydroxy-4-((3R)-1-{N- methyl-N-[2- (methylamino)ethyl] glycyl}-3-piperidinyl) butyl]carbamate |
| I-20a | 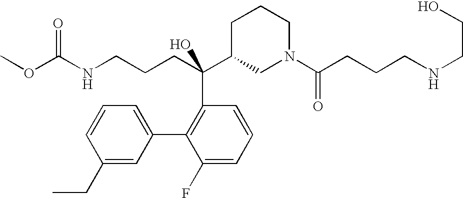 | methyl [(4S)-4-(3′-ethyl-6- fluoro-2-biphenylyl)-4- hydroxy-4-((3R)-1-{4-[(2- hydroxyethyl)amino]butanoyl}- 3-piperidinyl)butyl]carbamate |
| I-21a | 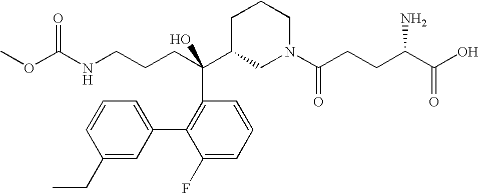 | (2S)-2-amino-5-[(3R)-3- ((1S)-1-(3′-ethyl-6-fluoro- 2-biphenylyl)-1-hydroxy- 4-{[(methyloxy) carbonyl]amino}butyl)-1- piperidinyl]-5- oxopentanoic acid |
| I-22a | 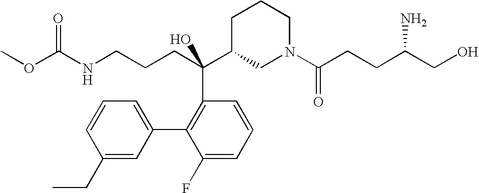 | methyl [(4S)-4-{(3R)-1- [(4S)-4-amino-5- hydroxypentanoyl]-3- piperidinyl}-4-(3′-ethyl-6- fluoro-2-biphenylyl)-4- hydroxybutyl]carbamate |
| I-23a | 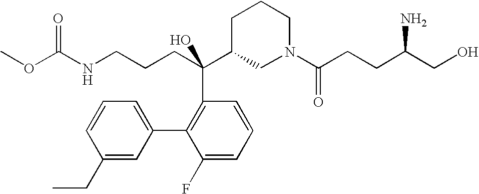 | methyl [(4S)-4-{(3R)-1- [(4R)-4-amino-5- hydroxypentanoyl]-3- piperidinyl}-4-(3′-ethyl-6- fluoro-2-biphenylyl)-4- hydroxybutyl]carbamate |
| I-24a | 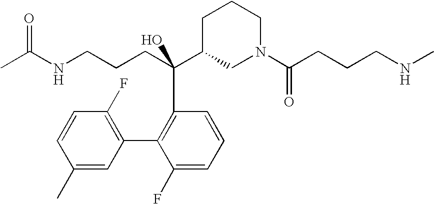 | N-((4S)-4-(2′,6-difluoro-5′- methyl-2-biphenylyl)-4- hydroxy-4-{(3R)-1-[4- (methylamino)butanoyl]-3- piperidinyl}butyl) acetamide |
| I-25a | 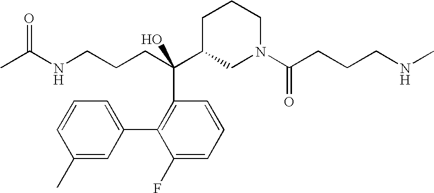 | N-((4S)-4-(6-fluoro-3′- methyl-2-biphenylyl)-4- hydroxy-4-{(3R)-1-[4- (methylamino)butanoyl]-3- piperidinyl}butyl)acetamide |
| I-26a | 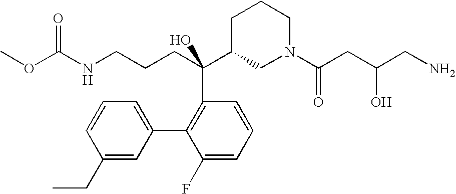 | methyl [(4S)-4-[(3R)-1-(4- amino-3- hydroxybutanoyl)-3- piperidinyl]-4-(3′-ethyl-6- fluoro-2-biphenylyl)-4- hydroxybutyl]carbamate |
| I-27a | 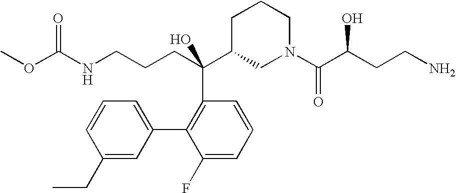 | methyl [(4S)-4-{(3R)-1- [(2S)-4-amino-2- hydroxybutanoyl]-3- piperidinyl}-4-(3′-ethyl-6- fluoro-2-biphenylyl)-4- hydroxybutyl]carbamate |
| I-28a | 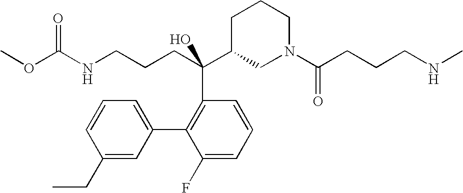 | methyl ((4S)-4-(3′-ethyl-6- fluoro-2-biphenylyl)-4- hydroxy-4-{(3R)-1-[4- (methylamino)butanoyl]-3- piperidinyl}butyl) carbamate |
| I-29a | 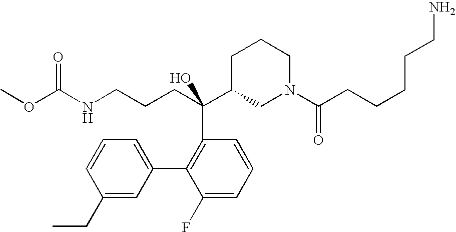 | methyl [(4S)-4-[(3R)-1-(6- aminohexanoyl)-3- piperidinyl]-4-(3′-ethyl-6- fluoro-2-biphenylyl)-4- hydroxybutyl]carbamate |
| I-30a | 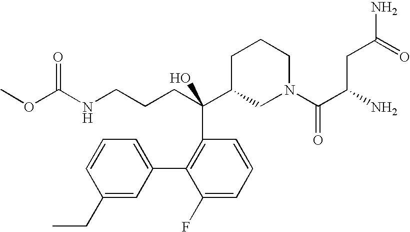 | methyl [(4S)-4-[(3R)-1-L- asparaginyl-3-piperidinyl]- 4-(3′-ethyl-6-fluoro-2- biphenylyl)-4- hydroxybutyl]carbamate |
| I-31a | 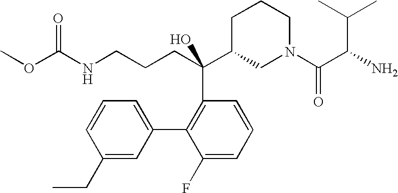 | methyl {(4S)-4-(3′-ethyl-6- fluoro-2-biphenylyl)-4- hydroxy-4-[(3R)-1-L-valyl- 3-piperidinyl]butyl} carbamate |
| I-32a | 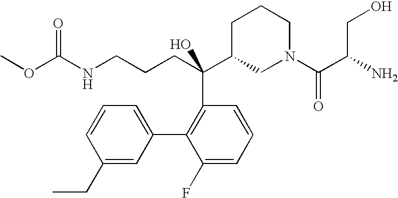 | methyl {(4S)-4-(3′-ethyl-6- fluoro-2-biphenylyl)-4- hydroxy-4-[(3R)-1-L-seryl- 3-piperidinyl]butyl} carbamate |
| I-33a | 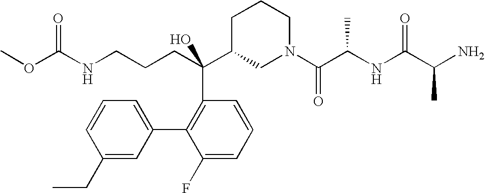 | methyl [(4S)-4-[(3R)-1-(L- alanyl-L-alanyl)-3- piperidinyl]-4-(3′-ethyl-6- fluoro-2-biphenylyl)-4- hydroxybutyl]carbamate |
| I-34a | 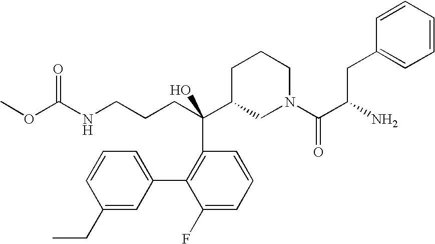 | methyl {(4S)-4-(3′-ethyl-6- fluoro-2-biphenylyl)-4- hydroxy-4-[(3R)-1-L- phenylalanyl-3-piperidinyl] butyl}carbamate |
| I-35a | 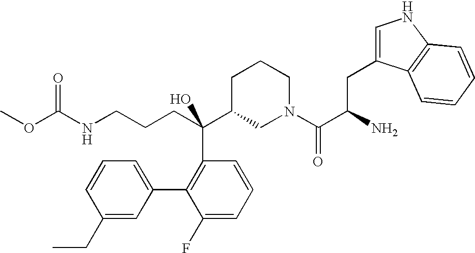 | methyl {(4S)-4-(3′-ethyl-6- fluoro-2-biphenylyl)-4- hydroxy-4-[(3R)-1-D- tryptophyl-3-piperidinyl] butyl}carbamate |
| I-36a | 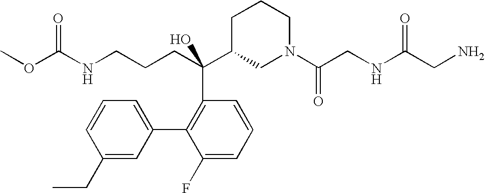 | methyl {(4S)-4-(3′-ethyl-6- fluoro-2-biphenylyl)-4- [(3R)-1-(glycylglycyl)-3- piperidinyl]-4- hydroxybutyl}carbamate |
| I-37a | 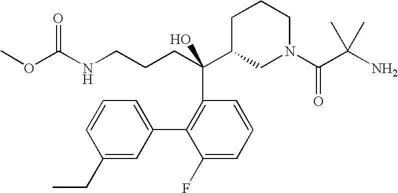 | methyl {(4S)-4-(3′-ethyl-6- fluoro-2-biphenylyl)-4- hydroxy-4-[(3R)-1-(2- methylalanyl)-3- piperidinyl]butyl} carbamate |
| I-38a | 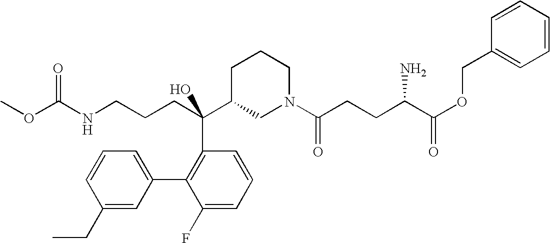 | phenylmethyl (2S)-2- amino-5-[(3R)-3-((1S)-1- (3′-ethyl-6-fluoro-2- biphenylyl)-1-hydroxy-4- {[(methyloxy)carbonyl] amino}butyl)-1- piperidinyl]-5- oxopentanoate |
| I-39a | 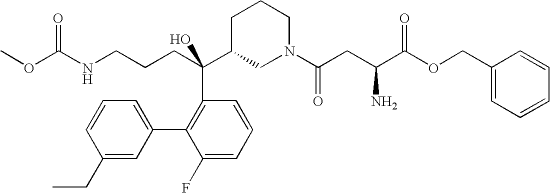 | phenylmethyl (2S)-2- amino-4-[(3R)-3-((1S)-1- (3′-ethyl-6-fluoro-2- biphenylyl)-1-hydroxy-4- {[(methyloxy)carbonyl] amino}butyl)-1- piperidinyl]-4- oxobutanoate |
| I-40a | 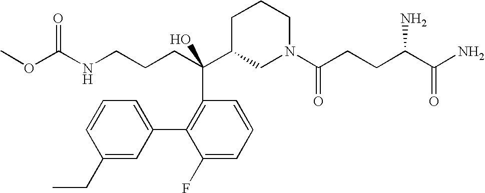 | methyl {(4S)-4-(3′-ethyl-6- fluoro-2-biphenylyl)-4- [(3R)-1-L-α-glutaminyl-3- piperidinyl]-4- hydroxybutyl}carbamate |
| I-41a | 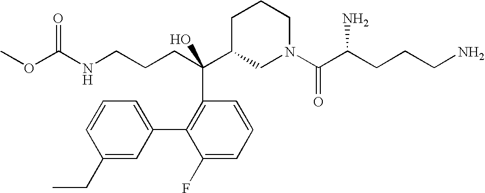 | methyl {(4)-4-(3′-ethyl-6- fluoro-2-biphenylyl)-4- hydroxy-4-[(3R)-1-D- ornithyl-3-piperidinyl] butyl}carbamate |
| I-42a | 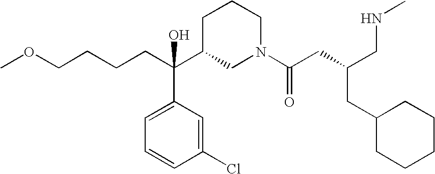 | (R)-1-((R)-3-((S)-1-(3- chlorophenyl)-1-hydroxy- 5-methoxypentyl) piperidin-1-yl)-4- cyclohexyl-3- ((methylamino)methyl) butan-1-one |
| I-43a | 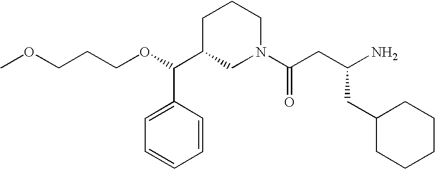 | (2R)-1-cyclohexyl-4- {(3R)-3-[(R)-{[3- (methyloxy)propyl]oxy} (phenyl)methyl]-1- piperidinyl}-4-oxo-2- butanamine |
| I-44a | 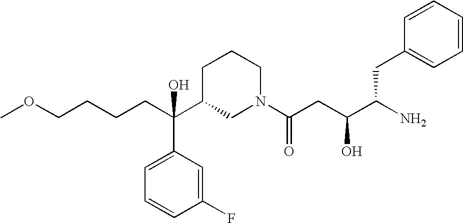 | (3S,4S)-4-amino-1-((R)-3- ((S)-1-(3-fluorophenyl)-1- hydroxy-5- methoxypentyl)piperidin- 1-yl)-3-hydroxy-5- phenylpentan-1-one |
| I-45a | 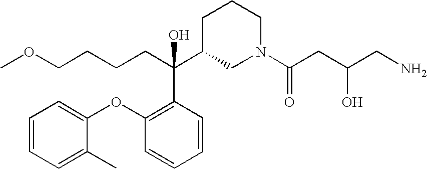 | 4-amino-3-hydroxy-1-((R)- 3-((S)-1-hydroxy-5- methoxy-1-(2-(o- tolyloxy)phenyl)pentyl) piperidin-1-yl)butan-1-one |
| I-46a | 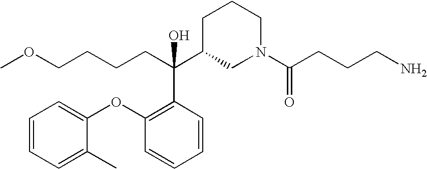 | 4-amino-1-((R)-3-((S)-1- hydroxy-5-methoxy-1-(2- (o-tolyloxy)phenyl) pentyl)piperidin-1- yl)butan-1-one |
| I-47a | 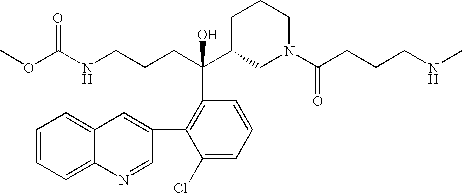 | methyl ((4S)-4-[3-chloro- 2-(3-quinolinyl)phenyl]-4- hydroxy-4-{(3R)-1-[4- (methylamino)butanoyl]-3- piperidinyl}butyl) carbamate |
| I-48a | 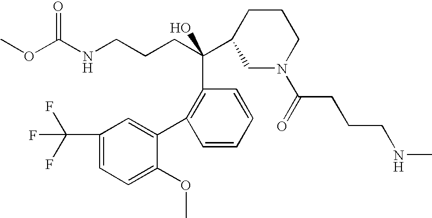 | methyl {(4S)-4-hydroxy-4- {(3R)-1-[4- (methylamino)butanoyl]-3- piperidinyl}-4-[2′- (methyloxy)-5′- (trifluoromethyl)-2- biphenylyl]butyl} carbamate |
| I-49a | 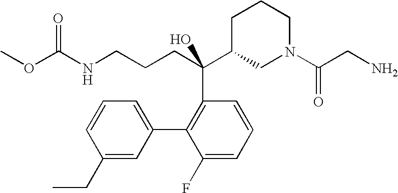 | methyl {(4S)-4-(3′-ethyl-6- fluoro-2-biphenylyl)-4- [(3R)-1-glycyl-3- piperidinyl]-4- hydroxybutyl}carbamate |
| I-50a | 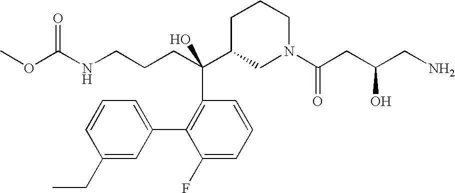 | methyl [(4S)-4-{(3R)-1- [(3S)-4-amino-3- hydroxybutanoyl]-3- piperidinyl}-4-(3′-ethyl-6- fluoro-2-biphenylyl)-4- hydroxybutyl]carbamate |
| I-51a | 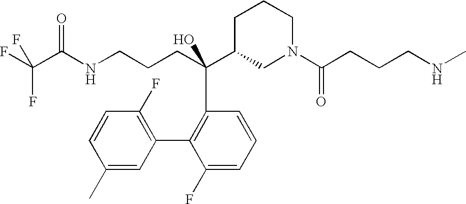 | N-((4S)-4-(2′,6-difluoro-5′- methyl-2-biphenylyl)-4- hydroxy-4-{(3R)-1-[4- (methylamino)butanoyl]-3- piperidinyl}butyl)-2,2,2- trifluoroacetamide |
| I-52a | 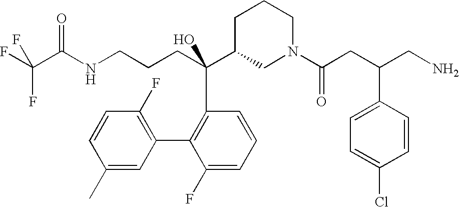 | N-[(4S)-4-{(3R)-1-[4- amino-3-(4- chlorophenyl)butanoyl]-3- piperidinyl}-4-(2′,6- difluoro-5′-methyl-2- biphenylyl)-4- hydroxybutyl]-2,2,2- trifluoroacetamide |
| I-53a | 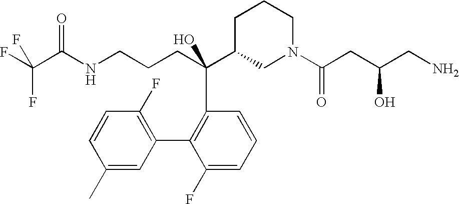 | N-[(4S)-4-{(3R)-1-[(3S)-4- amino-3- hydroxybutanoyl]-3- piperidinyl}-4-(2′,6- difluoro-5′-methyl-2- biphenylyl)-4- hydroxybutyl]-2,2,2- trifluoroacetamide |
| I-54a | 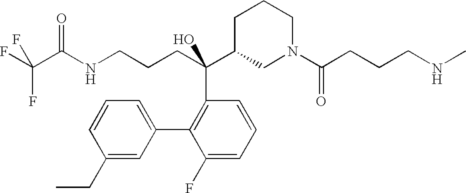 | N-((4S)-4-(3′-ethyl-6- fluoro-2-biphenylyl)-4- hydroxy-4-{(3R)-1-[4- (methylamino)butanoyl]-3- piperidinyl}butyl)-2,2,2- trifluoroacetamide |
| I-55a | 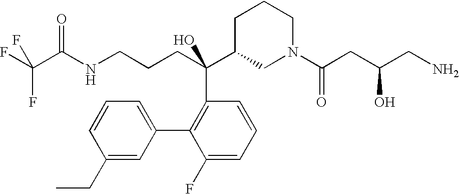 | N-[(4S)-4-{(3R)-1-[(3S)-4- amino-3- hydroxybutanoyl]-3- piperidinyl}-4-(3′-ethyl-6- fluoro-2-biphenylyl)-4- hydroxybutyl]-2,2,2- trifluoroacetamide |
| I-56a | 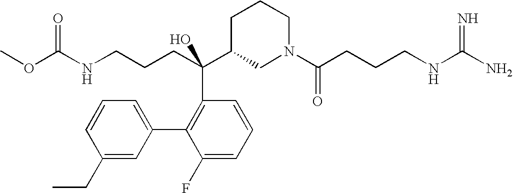 | methyl [(4S)-4-[(3R)-1-(4- {[amino(imino)methyl]amino} butanoyl)-3- piperidinyl]-4-(3′-ethyl-6- fluoro-2-biphenylyl)-4- hydroxybutyl]carbamate |
| I-57a | 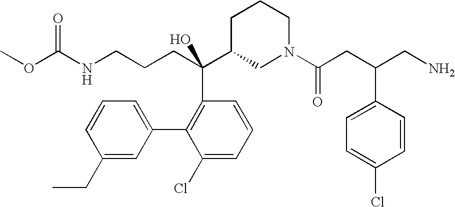 | methyl [(4S)-4-{(3R)-1-[4- amino-3-(4- chlorophenyl)butanoyl]-3- piperidinyl}-4-(6-chloro- 3′-ethyl-2-biphenylyl)-4- hydroxybutyl]carbamate |
| I-58a | 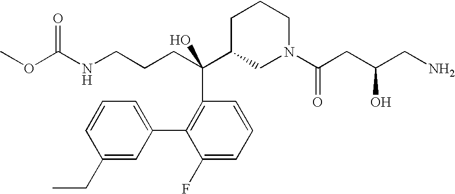 | methyl [(4S)-4-{(3R)-1- [(3S)-4-amino-3- hydroxybutanoyl]-3- piperidinyl}-4-(6-chloro- 3′-ethyl-2-biphenylyl)-4- hydroxybutyl]carbamate |
| I-59a | 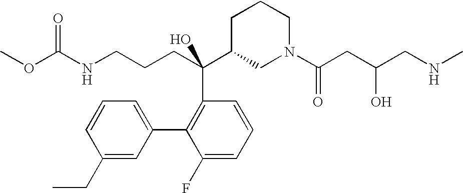 | methyl ((4S)-4-(3′-ethyl-6- fluoro-2-biphenylyl)-4- hydroxy-4-{(3R)-1-[3- hydroxy-4-(methylamino) butanoyl]-3-piperidinyl} butyl)carbamate |
| I-60a | 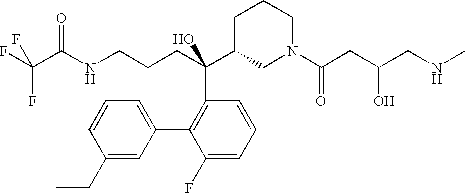 | N-((4S)-4-(3′-ethyl-6- fluoro-2-biphenylyl)-4- hydroxy-4-{(3R)-1-[3- hydroxy-4-(methylamino) butanoyl]-3-piperidinyl} butyl)-2,2,2- trifluoroacetamide |
| I-61a | 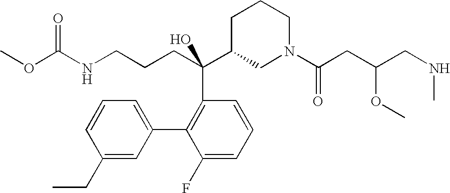 | methyl ((4S)-4-(3′-ethyl-6- fluoro-2-biphenylyl)-4- hydroxy-4-{(3R)-1-[4- (methylamino)-3- (methyloxy)butanoyl]-3- piperidinyl}butyl) carbamate |
| I-62a | 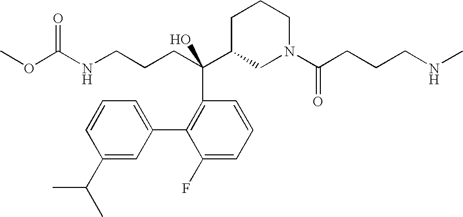 | methyl ((4S)-4-[6-fluoro- 3′-(1-methylethyl)-2- biphenylyl]-4-hydroxy-4- {(3R)-1-[4-(methylamino) butanoyl]-3-piperidinyl} butyl)carbamate |
| I-63a | 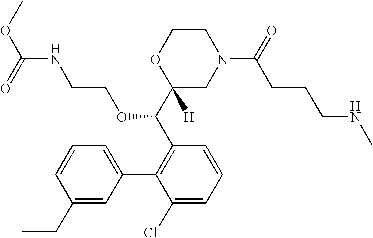 | methyl {2-[((S)-(6-chloro- 3′-ethyl-2-biphenylyl) {(2R)-4-[4-(methylamino) butanoyl]-2-morpholinyl} methyl)oxy]ethyl} carbamate |
| I-64a | 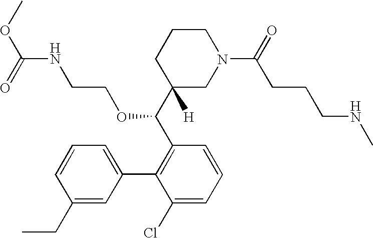 | methyl {2-[((R)-(6-chloro- 3′-ethyl-2- biphenylyl){(3R)-1-[4- (methylamino)butanoyl]-3- piperidinyl}methyl)oxy]ethyl} carbamate |
| I-65a | 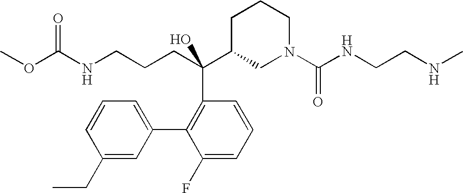 | methyl {(4S)-4-(3′-ethyl-6- fluoro-2-biphenylyl)-4- hydroxy-4-[(3R)-1-({[2- (methylamino)ethyl] amino}carbonyl)-3- piperidinyl]butyl} carbamate |
| I-66a | 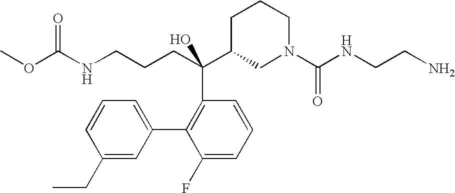 | methyl [(4S)-4-((3R)-1- {[(2-aminoethyl) amino]carbonyl}-3- piperidinyl)-4-(3′-ethyl-6- fluoro-2-biphenylyl)-4- hydroxybutyl]carbamate |
| I-67a | 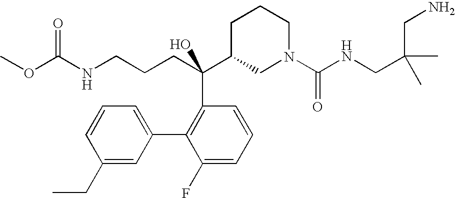 | methyl [(4S)-4-((3R)-1- {[(3-amino-2,2- dimethylpropyl)amino] carbonyl}-3-piperidinyl)-4-(3′- ethyl-6-fluoro-2- biphenylyl)-4- hydroxybutyl]carbamate |
| I-68a | 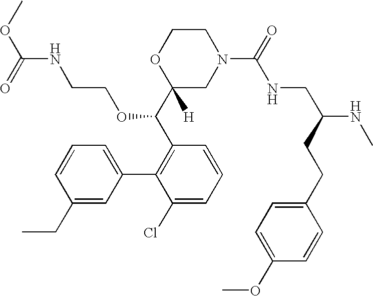 | methyl {2-[((S)-(6-chloro- 3′-ethyl-2-biphenylyl) {(2R)-4-[({(2S)-2- (methylamino)-4-[4- (methyloxy)phenyl]butyl} amino)carbonyl]-2- morpholinyl}methyl)oxy] ethyl}carbamate |
| I-69a | 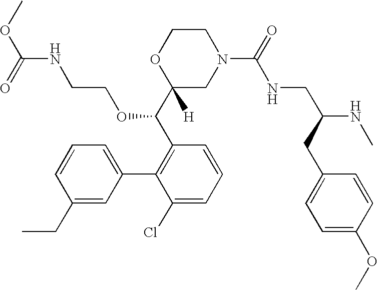 | methyl {2-[((S)-(6-chloro- 3′-ethyl-2-biphenylyl) {(2R)-4-[({(2S)-2- (methylamino)-3-[4- (methyloxy)phenyl] propyl}amino)carbonyl]-2- morpholinyl}methyl)oxy] ethyl{carbamate |
| I-70a | 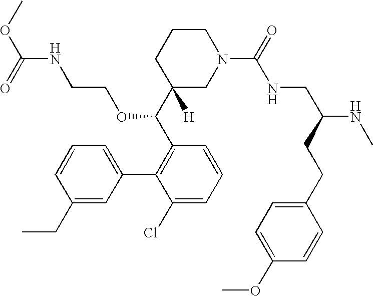 | methyl {2-[((R)-(6-chloro- 3′-ethyl-2- biphenylyl){(3R)-1- [({(2S)-2-(methylamino)- 4-[4-(methyloxy) phenyl]butyl}amino) carbonyl]-3-piperidinyl} methyl)oxy]ethyl} carbamate |
| I-71a | 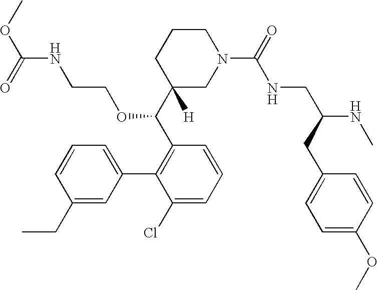 | methyl {2-[((R)-(6-chloro- 3′-ethyl-2-biphenylyl) {(3R)-1-[({(2S)-2- (methylamino)-3-[4- (methyloxy)phenyl] propyl}amino)carbonyl]-3- piperidinyl}methyl)oxy] ethyl{carbamate |

or the salts thereof.

The following Compound Nos. represent preferred compounds of this invention: I-9a, I-10a, I-15a, I-22a, I-23a, I-24a, I-25a, I-27a, I-32a, I-38a, I-47a, I-51a, I-53a, I-60a, I-63a, I-64a, I-65a, I-69a, I-70a, and I-71a, or a salt thereof. The following Compound Nos. represent the more preferred compounds of this invention: I-8a, I-13a, I-14a, I-16a, I-20a, I-26a, I-28a, I-29a, I-48a, I-50a, I-54a, I-55a, I-57a, I-58a, I-59a, I-61a, I-62a, and I-66a, or a salt thereof.

The compounds of the invention (Compound #1-71) exhibit 50% renin inhibition (as determined using the method of Biological Assay Example 2) at concentrations of from approximately 5000 nM to approximately 0.01 nM. Preferred compounds of the invention exhibit 50% inhibition at concentrations of from approximately 50 nM to approximately 0.01 nM. More preferred compounds of the invention exhibit 50% inhibition at concentrations of from approximately 5 nM to approximately 0.01 nM.

When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Cycloalkyl” means a saturated aliphatic cyclic hydrocarbon radical having the specified number of carbon atoms. Thus, (C₃-C₇)cycloalkyl means a radical having from 3-8 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

“Heterocyclyl” means a heteroaryl or a saturated heterocyclic ring group.

Saturated heterocyclic rings are 4-, 5-, 6-, and 7-membered heterocyclic rings containing 1 to 4 heteroatoms independently selected from N, O, and S, and include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic and polycyclic ring radical containing 1 to 4 heteroatoms independently selected from N, O, and S. Heteroaryl rings include furyl (furanyl), thienyl (thiophenyl), pyrrolyl, oxazolyl, thiazolyl, imidazolyl, pyrazolyl, isoxazolyl, isothiazolyl, oxadiazolyl, triazolyl, thiadiazolyl, pyridinyl, pyridinyl-N-oxide, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, 1,2,3-triazolyl, 1,2,4-triazolyl, 1,3,4-oxadiazolyl, 1,2,5-thiadiazolyl, 1,2,5-thiadiazolyl-1-oxide, 1,2,5-thiadiazolyl-1,1-dioxide, 1,3,4-thiadiazolyl, 1,2,4-triazinyl, 1,3,5-triazinyl, tetrazolyl, and pteridinyl.

Bicyclic heteroaryl rings are bicyclo[4.4.0] and bicyclo[4,3.0] fused ring systems of which at least one ring is aromatic containing 1 to 4 heteroatoms independently selected from N, O, and S, and include indole, quinoline, isoquinoline, quinazoline, benzothiophene, benzofuran, 2,3-dihydrobenzofuran, benzodioxole, benzimidazole, indazole, benzisoxazole, benzoxazole, and benzothiazole.

Bicycloalkyl rings are fused, bridged and spiro ring systems and include bicyclo[1.1.0]butane, bicyclo[1.2.0]pentane, bicyclo[2.2.0]hexane, bicyclo[3.2.0]heptane, bicyclo[3.3.0]octane, bicyclo[4.2.0]octane, bicyclo[2.2.1]heptane, bicyclo[2.2.2]octane, bicyclo[3.2.1]octane, bicyclo[3.2.2]nonane, bicyclo[3.3.1]nonane, bicyclo[3.3.2]decane, bicyclo[3.3.3]undecane, spiro[2.2]pentane, spiro[2.3]hexane, spiro[3.3]heptane, spiro[2.4]heptane, spiro[3.4]octane, and spiro[2.5]octane.

Tricycloalkyl rings are fused, bridged, and spiro ring systems and include tricyclo[3.3.1.0^{3,7}]nonane (noradamantane) and tricyclo[3.3.1.1^{3,7}]decane (adamantane).

“Alkoxy” means an alkyl radical attached through an oxygen linking atom. “(C₁-C₄)-alkoxy” includes the methoxy, ethoxy, propoxy, and butoxy.

“Aromatic” means an unsaturated cycloalkyl ring system.

“Aryl” means an aromatic monocyclic or polycyclic ring system. Aryl systems include phenyl, naphthyl (naphthalenyl), fluorenyl, indenyl, azulenyl, and anthracenyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

Certain compounds of Formula I, and the compounds of Formula Ia, Ib, and Ic exist in various stereoisomeric or tautomeric forms. The invention encompasses all enantiomeric, diastereomeric, and tautomeric forms, including mixtures thereof, of the compounds of this invention, including those not depicted structurally. For example, active compounds in the form of essentially pure enantiomers, racemic mixtures, and tautomers thereof are included within the scope of this invention.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

When a disclosed compound or its pharmaceutically acceptable salt is named or depicted by structure, it is to be understood that solvates, specifically hydrates, of the disclosed compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates”. Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The disclosed compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compounds and their pharmaceutically acceptable salts, solvates, or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

“R,” “S,” “S*,” “R*,” “E,” “Z,” “cis,” and “trans,” indicate configurations relative to the core molecule.

The point at which a group or moiety is attached to the remainder of the compound or another group or moiety can be indicated by “ [symbol] ” which represents [symbol] [symbol] or “—”.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed compound is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

For oral dosing, the renin inhibitors were formulated in 0.5% methylcellulose at dose levels of 10 and 30 mg/kg (5 mL/kg) by infant feeding tubes. For intravenous delivery, a silastic catheter was implanted into posterior vena cava via a femoral vein. The catheter was attached to the delivery pump via a tether system and a swivel joint. Test compound (dose levels of 0.1 to 10 mg/kg, formulated at 5% dextrose) was administered by continuous infusion (1.67 mL:/kg/h) or by bolus injection (3.33 mL/kg in 2 min).

Arterial blood pressures (systolic, diastolic and mean) and body temperature were recorded continuously at 500 Hz and 50 Hz, respectively, using the Dataquest™ A.R.T. (Advanced Research Technology) software. Heart rate was derived from the phasic blood pressure tracing. During the recording period, the monkeys were kept in a separate room without human presence to avoid pressure changes secondary to stress. All data were expressed as mean±SEM. Effects of the renin inhibitors on blood pressure were assessed by ANOVA, taking into account the factors dose and time compared with the vehicle group.

Beagle Dogs Non-naive Beagle dogs (2 per sex) weighing between 9 and 11 kg were used in the studies. Each animal was implanted subcutaneously with a telemetry transmitter (Data Sciences) and the blood pressure catheter was inserted into the left femoral artery. The electrocardiogram leads were also tunneled subcutaneously to the appropriate anatomical regions. The animals were housed under constant temperature and lighting conditions, were fed once daily, and were allowed free access to water. A sodium depleted state was produced by placing them on a low-sodium diet (<4 meq/day, a combination of canned Prescription Diet canine h/d, from Hill's Pet Products and dry pellets from Bio-Serv Inc., Frenchtown, N.J.) beginning 10 days before the experiment, and furosemide (3 mg/kg i.m.; Aventis Pharmaceuticals) was administered at −40 and −16 h prior to administration of test compound.

A renin inhibitor was orally administered by orogastric gavage to all overnight fasted animals at a dose level of 30 mg/kg (4 mL/kg formulated in 0.5% methylcellulose). Food was given 4 h postdose. In some experiments, the renin inhibitor was administered by bolus i.v. at increasing dose levels of 1, 3, and 6 mg/kg (2, 6, and 20 mg/mL formulated in sterile saline). Cardiovascular parameters were collected continuously at least 80 min predose and 3 h postdose, followed by every 10 min for 5 h and every 30 min for 16 h postdose. The Dataquest™ ART (version 2.2) software package from DSI (Data Sciences International) was used to collect telemetered cardiovascular data.

The efficacy of the renin inhibitors was also evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments were conducted in 6-week-old double transgenic rats (dTGRs). The model has been described in detail earlier. Briefly, the human renin construct used to generate transgenic animals made up the entire genomic human renin gene (10 exons and 9 introns), with 3.0 kB of the 5′-promoter region and 1.2 kB of 3′ additional sequences. The human angiotensinogen construct made up the entire human angiotensinogen gene (5 exons and 4 introns), with 1.3 kB of 5′-flanking and 2.4 kB of 3′-flanking sequences. The rats were purchased from RCC Ltd (Füllinsdorf, Switzerland). Radio telemetry transmitters were surgically implanted at 4 weeks of age. The telemetry system provided 24-h recordings of systolic, mean, diastolic arterial pressure (SAP, MAP, DAP, respectively) and heart rate (HR). Beginning on day 42, animals were transferred to telemetry cages. A 24 h telemetry reading was obtained. Rats were then dosed orally on the following 4 consecutive days (days 43-46). The rats were monitored continuously and allowed free access to standard 0.3%-sodium rat chow and drinking water.

The compounds of the invention are useful for ameliorating or treating disorders or diseases in which decreasing the levels of renin products is effective in treating a disease state. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; atrial fibrillation; atrial flutter; detrimental vascular remodeling; myocardial infarction and its sequalae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, such as nephropathy, vasculopathy and neuropathy; diseases of the coronary vessels; proteinuria; albumenuria; post-surgical hypertension; metabolic syndrome; obesity, restenosis following angioplasty, ocular vascular complications, for example, raised intra-ocular pressure, glaucoma, and retinopathy; abnormal vascular growth, angiogenesis-related disorders, such as neovascular age related macular degeneration; hyperaldosteronism; anxiety states; and cognitive disorders (Fisher N.D.; Hollenberg N. K. Expert Opin. Investig. Drugs. 2001, 10, 417-26).

A pharmaceutical composition of the invention may, alternatively or in addition to a compound of Formula I, Ia, Ib, or Ic, comprise a pharmaceutically acceptable salt of a compound of Formula I, Ia, Ib, or Ic, or a prodrug or pharmaceutically active metabolite of such a compound or salt and one or more pharmaceutically acceptable carriers therefor.

The compositions of the invention are aspartic protease inhibitors. Said compositions contain compounds having a mean inhibition constant (IC₅₀) against aspartic proteases of between about 5,000 nM to about 0.001 nM; preferably between about 100 nM to about 0.001 nM; and more preferably between about 10 nM to about 0.01 nM.

The compositions of the invention reduce blood pressure. Said compositions include compounds having an IC₅₀ for renin of between about 5,000 nM to about 0.001 nM; preferably between about 100 nM to about 0.001 nM; and more preferably between about 10 nM to about 0.01 nM.

The invention includes a therapeutic method for treating or ameliorating an aspartic protease mediated disorder in a subject in need thereof comprising administering to a subject in need thereof an effective amount of a compound of Formula I, Ia, Ib, or Ic, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

An embodiment of the invention includes administering a renin inhibiting compound of Formula I, Ia, Ib, or Ic, or a composition thereof in a combination therapy (see U.S. Pat. No. 5,821,232, U.S. Pat. No. 6,716,875, U.S. Pat. No. 5,663,188, or Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antihypertensives, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, or endothelin receptor antagonists.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are amlodipine, felodipine, ryosidine, isradipine, lacidipine, nicardipine, nifedipine, nigulpidine, niludipine, nimodiphine, nisoldipine, nitrendipine, and nivaldipine, and their pharmaceutically acceptable salts. Non-DHPs are flunarizine, prenylamine, diltiazem, fendiline, gallopamil, mibefradil, anipamil, tiapamil, and verampimil, and their pharmaceutically acceptable salts.

A diuretic is, for example, a thiazide derivative selected from amiloride, chlorothiazide, hydrochlorothiazide, methylchlorothiazide, and chlorothalidon.

Centrally acting antihypertensives include clonidine, guanabenz, guanfacine and methyldopa.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxsentan, and tezosentan, and their pharmaceutically acceptable salts.

An embodiment of the invention includes administering a disclosed compound or composition thereof in a combination therapy with one or more additional agents for the treatment of AIDS including reverse transcriptase inhibitors, non-nucleoside reverse transcriptase inhibitors, other HIV protease inhibitors, HIV integrase inhibitors, attachment and fusion inhibitors, antisense drugs, and immune stimulators.

Specific reverse transcriptase inhibitors are zidovudine, didanosine, zalcitabine, stavudine, lamivudine, abacavir, tenofovir, and emtricitabine.

Specific non-nucleoside reverse transcriptase inhibitors are nevirapine, delaviridine, and efavirenz.

Specific HIV protease inhibitors are saquinavir, ritonavir, indinavir, nelfinavir, amprenavir, lopinavir, atazanavir, and fosamprenavir.

Specific HIV integrase inhibitors are L-870,810 and S-1360.

A specific attachment and fusion inhibitor is enfuvirtide.

An embodiment of the invention includes administering a disclosed compound or composition thereof in a combination therapy with one or more additional agents for the treatment of Alzheimer's disease including tacrine, donepezil, rivastigmine, galantamine, and memantine.

Combination therapy includes co-administration of the compound of the invention and said other agent, sequential administration of the compound and the other agent, administration of a composition containing the compound and the other agent, or simultaneous administration of separate compositions containing the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I, Ia, Ib, or Ic, may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

### Methods of Preparation

In the discussion below R¹, R², R³, X, Y, A, A⁴, L, and G are defined as described above for compounds of Formula I, Ia, Ib, or Ic. In cases where the synthetic intermediates and final products of Formula I, Ia, Ib, or Ic described below contain potentially reactive functional groups, for example amino, hydroxyl, thiol, and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). Such protecting group manipulations are assumed in the discussion below and not described explicitly.

In the first process of the invention, a compound of Formula I, in which a nitrogen atom that is part of A is attached to the carbonyl moiety, is prepared by reaction of an amine of Formula II with an intermediate of Formula III:

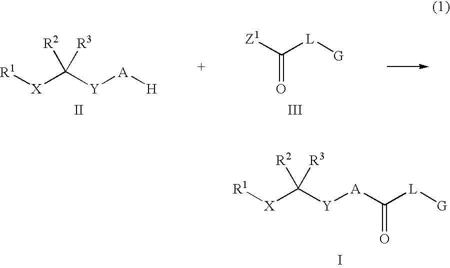

wherein Z¹ in III is a leaving group such as halide, alkanesulfonate, haloalkanesulfonate, arylsulfonate, aryloxide, heteroaryloxide, azole, azolium salt, or alkoxide.

Intermediates of Formula II wherein H is attached to a nitrogen atom that is part of A are prepared from intermediates of Formula IV:

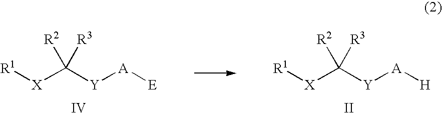

wherein E is an amine protecting group, including carbamate, amide, and sulfonamide protecting groups known in the art (T. W. Greene and P. G. M. Wuts “Protective Groups in Organic Synthesis” John Wiley & Sons, Inc., New York 1999).

Intermediates of Formula IV wherein R³═OH are prepared from ketone intermediates of formula V by addition of an organometallic reagent of formula VI, wherein M is for example Li, MgCl, MgBr, or MgI:

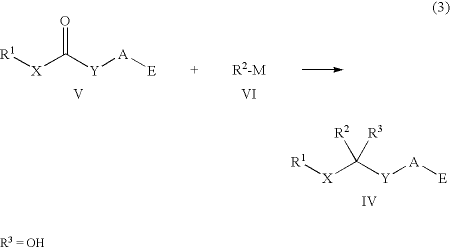

Intermediates of Formula IV wherein R³═H and R² is a group attached by an ether linkage are prepared from alcohol intermediates of formula VII by reaction with an alkylating agent under basic conditions or by reaction with an alcohol of formula R²OH under acidic conditions.

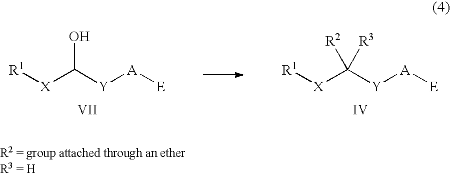

Alcohol intermediates of formula VII are prepared by reduction of ketone intermediates of formula V:

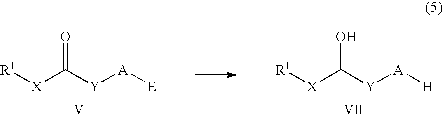

or by addition of an organometallic reagent of formula VIII, wherein M is, for example Li, MgCl, MgBr, or MgI, to an aldehyde of Formula IX:

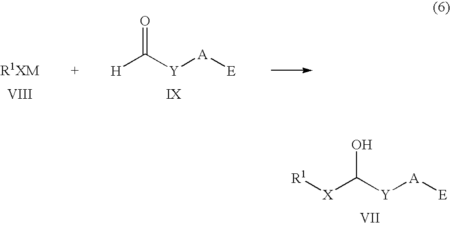

Ketone intermediates of formula V are prepared by the addition of an organometallic reagent of formula VIII to a carboxylic acid derivative of formula X wherein Z² is an alkoxide, dialkylamino group, or an N-alkoxy-N-alkylamino group:

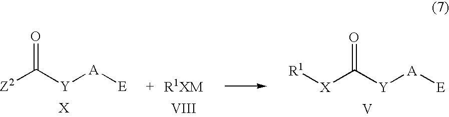

Organometallic reagents of formula VIII are prepared by known process including halogen-lithium exchange, ortho-lithiation, and treatment of halides R¹X-Hal with magnesium or lithium metal.

Intermediates of Formula III, wherein Z¹ is halide, alkanesulfonate, haloalkanesulfonate, arylsulfonate, or represents an active ester, are prepared by activation of carboxylic acids of Formula XI:

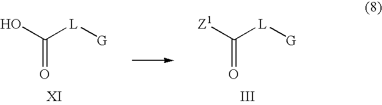

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula III are often prepared and used in situ without isolation.

Intermediates of Formula III, in which a nitrogen atom that is part of L is attached to the carbonyl moiety, are prepared by reaction of an amine of Formula XII with an intermediate of Formula XIII, wherein Z¹ is halide, aryloxy, heteroaryloxy, azole, azolium salt, alkoxy, alkylthio, or arylthio:

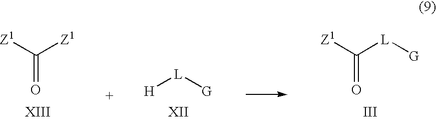

Intermediates of formula III are often prepared and used in situ without isolation.

In the discussion below R¹, R², R³, A⁴, L, and G are defined as described above for compounds of Formula Ia, Ib, or Ic. In cases where the synthetic intermediates and final products of Formula Ia, Ib, or Ic described below contain potentially reactive functional groups, for example amino, hydroxyl, thiol, and carboxylic acid groups, that may interfere with the desired reaction, it may be advantageous to employ protected forms of the intermediate. Methods for the selection, introduction, and subsequent removal of protecting groups are well known to those skilled in the art. (T. W. Greene and P. G. M. Wuts “Protective Groups in Organic Synthesis” John Wiley & Sons, Inc., New York 1999). Such protecting group manipulations are assumed in the discussion below and not described explicitly.

In the first process of the invention a compound of Formula Ia, Ib, or Ic, is prepared by reaction of an amine of Formula IIa with an intermediate of Formula III:

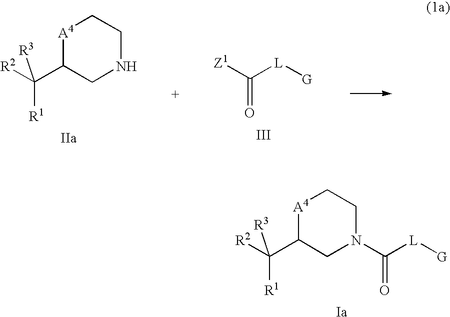

wherein Z¹ in IIa is a leaving group such as halide, alkanesulfonate, haloalkanesulfonate, arylsulfonate, aryloxide, heteroaryloxide, azole, azolium salt, or alkoxide.

Intermediates of Formula IIa are prepared from intermediates of Formula IVa:

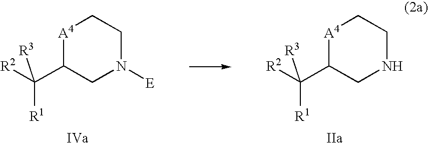

wherein E is an amine protecting group, including carbamate, amide, and sulfonamide protecting groups known in the art (T. W. Greene and P. G. M. Wuts “Protective Groups in Organic Synthesis” John Wiley & Sons, Inc., New York 1999).

Intermediates of Formula IVa wherein R³═OH are prepared from ketone intermediates of formula Va by addition of an organometallic reagent of formula VI, wherein M is for example Li, MgCl, MgBr, or MgI:

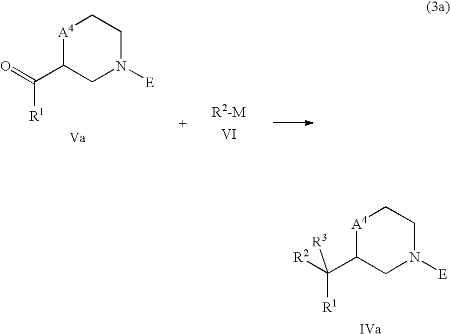

Intermediates of Formula IVa wherein R³═H and R² is a group attached by an ether linkage are prepared from alcohol intermediates of formula VIIa by reaction with an alkylating agent under basic conditions or by reaction with an alcohol of formula R²OH under acidic conditions.

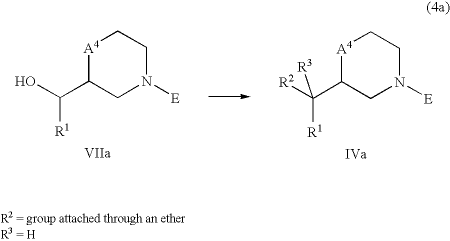

Alcohol intermediates of formula VIIa are prepared by reduction of ketone intermediates of formula Va:

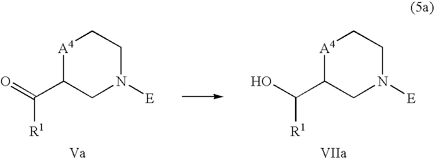

or by addition of an organometallic reagent of formula VIIIa, wherein M is, for example Li, MgCl, MgBr, or MgI, to an aldehyde of Formula IXa:

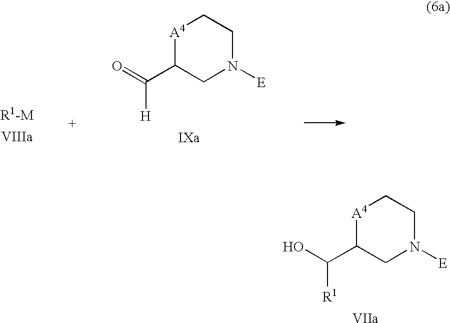

Ketone intermediates of formula Va are prepared by the addition of an organometallic reagent of formula VIIIa to a carboxylic acid derivative of formula Xa wherein Z² is an alkoxide, dialkylamino group, or an N-alkoxy-N-alkylamino group:

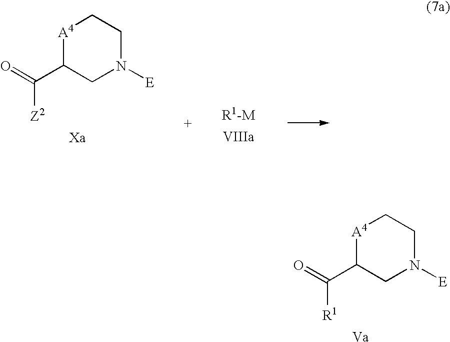

Organometallic reagents of formula VIIIa are prepared by known process including halogen-lithium exchange, ortho-lithiation, and treatment of halides R¹X-Hal with magnesium or lithium metal.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art. During the course of preparing aryl 3-piperidinyl ketones, as described in the following protocols (e.g. Preparations 4 and 5), racemization of the stereocenter adjacent to the carbonyl group can occur and was specifically observed during the preparation of (R)-tert-butyl 3-(6-chloro-3′-ethylbiphenylcarbonyl)piperidine-1-carboxylate. In this case, the racemic product was detected when the reaction mixture was allowed to stir at room temperature for prolonged times (e.g. overnight) but was not observed when the ketone forming reaction was quenched at −78° C. (by addition of aqueous ammonium chloride). When racemization does occur, the resulting stereoisomers may be resolved using conventional methods well known to those skilled in the art. Accordingly, it will be appreciated by those skilled in the art, that in the following Experimental section, any identification of a specific stereoisomer (e.g., assignment of configuration of a chiral center) in a final or intermediate product compound name or structure is to be understood to represent the intended relative or absolute configuration of that chiral center, but not necessarily the only stereoisomer obtained.

The following abbreviations have the indicated meanings

| Abbreviation | Meaning |
|---|---|
| aq | aqueous |
| Boc | tert-butoxy carbonyl or t-butoxy carbonyl |
| (Boc)₂O | di-tert-butyl dicarbonate |
| Brine | saturated aqueous NaCl |
| CH₂Cl₂ | methylene chloride |
| CH₃CN or MeCN | acetonitrile |
| Cpd | compound |
| d | day(s) |
| DBU | 1,8-diazabicyclo[5.4.0]undec-7-ene |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| EDC•HCl | 1-[3-(dimethylamino)propyl]-3-ethylcarbodiimide |
| | hydrochloride |
| equiv | equivalents |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour(s) |
| HOBt | 1-hydroxybenzotriazole |
| HATU | 2-(7-Aza-1H-benzotriazole-1-yl)-1,1,3,3- |
| | tetramethyluronium |
| | hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LAH or LiAlH₄ | lithium aluminum hydride |
| LC-MS | liquid chromatography-mass spectroscopy |
| LHMDS | lithium hexamethyldisilazane |
| Me | methyl |
| MeCN | acetonitrile |
| MeOH | methanol |
| MsCl | methanesulfonyl chloride |
| min | minute(s) |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| P₄-t-Bu | 1-tert-butyl-4,4,4-tris(dimethylamino)-2,2- |
| | bis[tris(dimethylamino)- |
| | phosphoranylidenamino]-2Λ⁵,4Λ⁵- |
| | catenadi(phosphazene) |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin- |
| | 2,5-dione |
| TFA | trifluoroacetic acid |
| THF | tetrahydrofuran |
| TMSCl | chlorotrimethylsilane or trimethylsilyl chloride |
| t_{R} | retention time |

### LC-MS Methods

Method 1 [Instrument 1]: Analytical LC-MS was conducted on an Agilent 1100 Series LC/MSD SL or VL using electrospray positive [ES+ve to give MH⁺] equipped with a Sunfire C₁₈ 5.0 μm column (3.050 mm×50 3.0 mm, i.d.), eluting with 0.05% TFA in water (solvent A) and 0.05% TFA in acetonitrile (solvent B), using the following elution gradient 10%-99% (solvent B) over 3.0 min and holding at 99% for 1.0 min at a flow rate of 1.0 ml/min.

Method 2 [Instrument 2]: Analytical LC-MS was conducted on an PE Sciex API 150 single quadrupole mass spectrometer using electrospray positive [ES+ve to give MH+] equipped with a Aquasil C18 5 μm column (1 mm×40 mm), eluting with 0.02% TFA in water (solvent A) and 0.018% TFA in acetonitrile (solvent B), using the following elution gradient 4.5%-90% (solvent B) over 3.2 min and holding at 90% for 0.4 min at a flow rate of 0.3 ml/min.

Method 3 [LC-MS (3 min)]: Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

The following procedures describe preparation of intermediates used in the synthesis of compounds of Formula I:

### Preparation 1—Weinreb Amide

### (R)-tert-butyl 3-(N-methoxy-N-methylcarbamoyl)piperidine-1-carboxylate

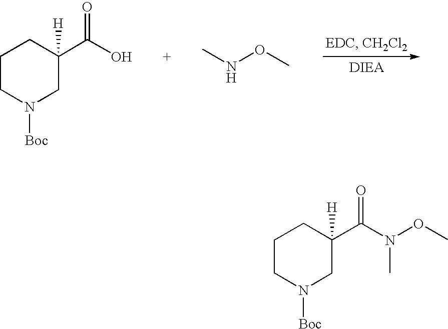

(R)-1-(tert-butoxy carbonyl)piperidine-3-carboxylic acid (25 g, 0.11 mol, 1.0 equiv), N,O-dimethylhydroxylamine hydrochloride, (10.5 g, 0.14 mol, 1.25 equiv), EDC.HCl (26.3 g, 0.14 mol, 1.25 equiv) and DIEA (48 mL, 0.28 mol, 2.5 equiv) were dissolved in CH₂Cl₂ (400 mL) and stirred overnight at rt. The reaction mixture was diluted with EtOAc, washed with 5% aq HCl (2×150 mL), satd aq NaHCO₃ (150 mL), brine (100 mL), and dried over Na₂SO₄. Concentration afforded (R)-tert-butyl 3-(N-methoxy-N-methylcarbamoyl)-piperidine-1-carboxylate (24.42 g, 82%) as a clear oil.

### Preparation 2

### Halodiphenyl Ethers from Phenoxyanilines

### 1-(O-tolyloxy)-2-iodobenzene

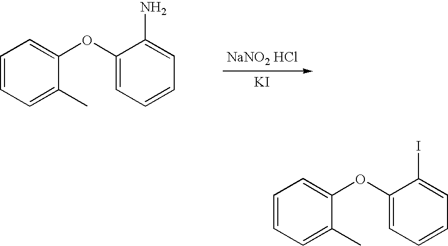

To a solution of 2-(o-tolyloxy)aniline (40 g, 0.2 mol) in 1N aq HCl (400 mL, 0.4 mol, 2 equiv) cooled to 0° C. was added dropwise a solution of NaNO₂ (18 g, 0.26 mol, 1.3 equiv) in water (520 ml). The mixture was stirred for 1 h at 0° C. and a solution of KI (83 g, 0.5 mol, 2.5 equiv) in water (500 mL) was added dropwise with vigorous stirring. After 0.5 h the mixture was warmed to 90-100° C. for 1 h, cooled to rt and washed with satd NaHSO₃ until the aqueous layer become clear. The mixture was extracted with EtOAc (3×200 mL) and the combined organic layers were washed with aq Na₂S₂O₄ and dried over Na₂SO₄. After evaporation of the solvent, the solution was passed through a short silica gel column to afford 1-(o-tolyloxy)-2-iodobenzene (40.0 g, 65%).

### Preparation 3—Biaryl Syntheses

### a) 6-Bromo-2-fluoro-3′-methylbiphenyl

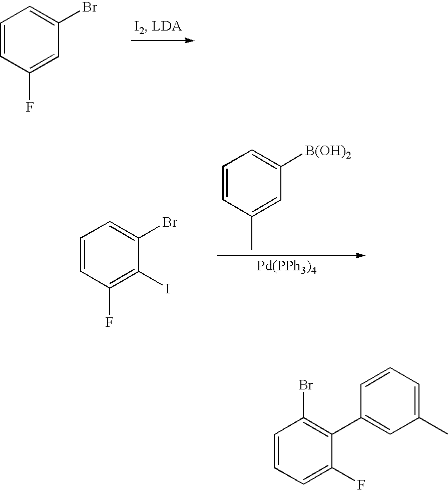

### Step 1. 1-Bromo-3-fluoro-2-iodobenzene

To a solution of diisopropylamine (76 mL, 0.4 mol) in dry THF (664 mL) and n-hexane (220 mL) was added 2.5 M n-BuLi (160 mL. 0.4 mol) dropwise at −78° C. during a period of 1 h. The mixture was stirred for 1 h at −78° C. Then a solution of 1-bromo-3-fluoro-benzene (69 g, 0.4 mol) in dry THF (300 mL) at −78° C. was added to the above mixture dropwise. After stirring for an additional 1 h at −78° C., the mixture was added a solution of iodine (101 g, 0.4 mol) in dry THF (400 mL) dropwise at −78° C. The temperature was raised from −78° C. to rt during 2 h. After stirring for 18 h at rt, the mixture was concentrated in vacuo to give crude product (120 g) which was distilled under reduced pressure to afford 1-bromo-3-fluoro-2-iodobenzene (110 g). ¹H NMR (400 MHz, DMSO) δ ppm 7.24-7.19 (t, 1H), 7.38-7.32 (m, 1H), 7.55-7.53 (d, 1H).

### Step 2. 6-Bromo-2-fluoro-3′-methylbiphenyl

Pd(Ph₃P)₄ in a 500-mL round-bottom flask under N₂ atmosphere was treated sequentially with a solution of 1-bromo-3-fluoro-2-iodo-benzene (30 g, 0.1 mol) in toluene (250 mL), a solution of 2N aq Na₂CO₃ (200 mL) and 3-methyl phenylboronic acid in ethanol (62 mL). This mixture was heated at reflux under N₂ for 12 h, then cooled to rt. The mixture was partitioned between water and EtOAc. The combined organic layers were washed with brine, dried over MgSO₄, evaporated and purified by column chromatography to give 6-bromo-2-fluoro-3′-methyl-biphenyl (12 g). ¹H NMR (400 MHz, CD₃OD) δ ppm 7.03 (m, 2H), 7.48-7.04 (m, 4H), 7.50 (d, 1H).

### b) 6-Bromo-2-chloro-3′-methyl-biphenyl

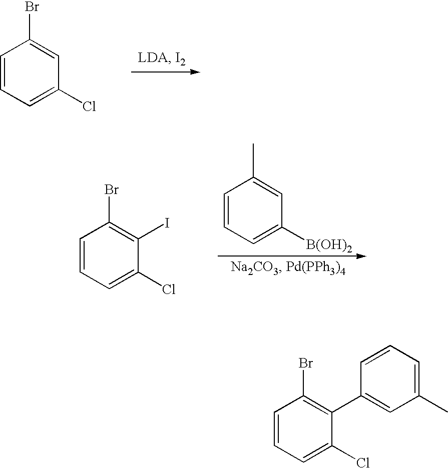

### Step 1. 1-bromo-3-chloro-2-iodobenzene

To a solution of diisopropylamine (76 mL, 0.4 mol) in anhydrous THF (664 mL) and n-hexane (220 mL) was added 2.5 M n-BuLi (160 mL, 0.4 mol) dropwise at −78° C. over 1 h. The mixture was stirred for 1 h at −78° C. and a solution of 1-bromo-3-chlorobenzene (76 g, 0.4 mol) in anhydrous THF (300 mL) was added dropwise at −78° C. After stirring for an additional 1 h at the same temperature, a solution of iodine (101 g, 0.4 mol) in anhydrous THF (400 mL) was added dropwise at −78° C. The temperature was raised from −78° C. to rt during 2 h. After stirring for 18 h at rt, the mixture was concentrated in vacuo to give the crude product (120 g) which was distilled under reduced pressure to give 1-bromo-3-fluoro-2-iodobenzene (115 g, 91%). ¹H NMR (400 MHz, CDCl₃) δ ppm 7.12-7.18 (t, 1H), 7.35-7.41 (dd, 1H), 7.49-7.54 (dd, 1H); MS (E/Z): 317 (M+H⁺)

### Step 2. 6-bromo-2-chloro-3′-methyl-biphenyl

A 500-mL round-bottom flask under N₂ atmosphere was charged sequentially with Pd(Ph₃P)₄, 1-bromo-3-fluoro-2-iodobenzene (10 g, 0.032 mol) in toluene (80 mL), 2N aqueous sodium carbonate (55 mL) and 3-methylphenylboronic acid (5.16 g, 0.032 mol) dissolved in ethanol (40 mL). This mixture was heated at reflux under N₂ for 12 h and cooled to rt. The mixture was partitioned between water and EtOAc. The combined organic layers were washed with brine, dried over MgSO₄, and concentrated. The residue was purified by column chromatography to give 6-bromo-2-chloro-3′-methyl-biphenyl (6 g, 67%). ¹H NMR (400 MHz, CD₃OD) δ ppm 6.90-7.00 (t, 2H), 7.14-7.24 (m, 2H), 7.26-7.33 (t, 1H), 7.44-7.50 (d, 1H), 7.58-7.62 (d, 1H); MS (E/Z): 281 (M+H⁺)

The following biaryls were prepared from aryl halides and the boronic acids indicated using procedures analogous to those described above:

| Biaryl | Aryl halide | Boronic acid |
|---|---|---|
| 2-bromo-6-chloro-3′- | 1-bromo-3-chloro- | 3-ethylphenylboronic acid |
| ethylbiphenyl | 2-iodobenzene | |
| 2-bromo-3′-ethyl-6- | 1-bromo-3-fluoro- | 3-ethylphenylboronic acid |
| fluorobiphenyl | 2-iodobenzene | |
| 2-bromo-6-fluoro-3′- | 1-bromo-3-fluoro- | 3-methylphenylboronic acid |
| methylbiphenyl | 2-iodobenzene | |
| 2′-bromo-2,6′-difluoro-5- | 1-bromo-3-fluoro- | 2-fluoro-5-methylphenylboronic |
| methylbiphenyl | 2-iodobenzene | acid |
| 2-bromo-3′,6-difluoro-5′- | 1-bromo-3-fluoro- | 3-fluoro-5-methylphenylboronic |
| methylbiphenyl | 2-iodobenzene | acid |
| 2-bromo-4′,6-difluoro-3′- | 1-bromo-3-fluoro- | 4-fluoro-3-methylphenylboronic acid |
| methylbiphenyl | 2-iodobenzene | |
| 2-bromo-6-chloro-3′-fluoro- | 1-bromo-3-chloro- | 3-fluoro-5-methylphenylboronic |
| 5′-methylbiphenyl | 2-iodobenzene | acid |
| 3-(2-bromo-6- | 1-bromo-3-fluoro- | quinolin-3-ylboronic acid |
| fluorophenyl)quinoline | 2-iodobenzene | |
| 2-(2-bromo-6- | 1-bromo-3-chloro- | 5-methylfuran-2-ylboronic acid |
| chlorophenyl)-5-methylfuran | 2-iodobenzene | |
| 6-(2-bromo-6- | 1-bromo-3-chloro- | 2,3-dihydrobenzofuran-6- |
| chlorophenyl)-2,3- | 2-iodobenzene | ylboronic acid |
| dihydrobenzofuran | | |
| 3-(2-bromo-6- | 1-bromo-3-chloro- | quinolin-3-ylboronic acid |
| chlorophenyl)quinoline | 2-iodobenzene | |
| 2-bromo-3′-isopropyl-6- | 1-bromo-3-fluoro- | 3-isopropylphenylboronic acid |
| fluorobiphenyl | 2-iodobenzene | |
| 2′-bromo-6′-fluoro-2- | 1-bromo-3-chloro- | 2-methoxy-5- |
| methoxy-5- | 2-iodobenzene | (trifluoromethyl)phenylboronic |
| (trifluoromethyl)biphenyl | | acid |

### Preparation 4

### Methyl {4-(6-chloro-3′-methyl-2-biphenylyl)-4-hydroxy-4-[(3R)-3-piperidinyl]butyl}carbamate

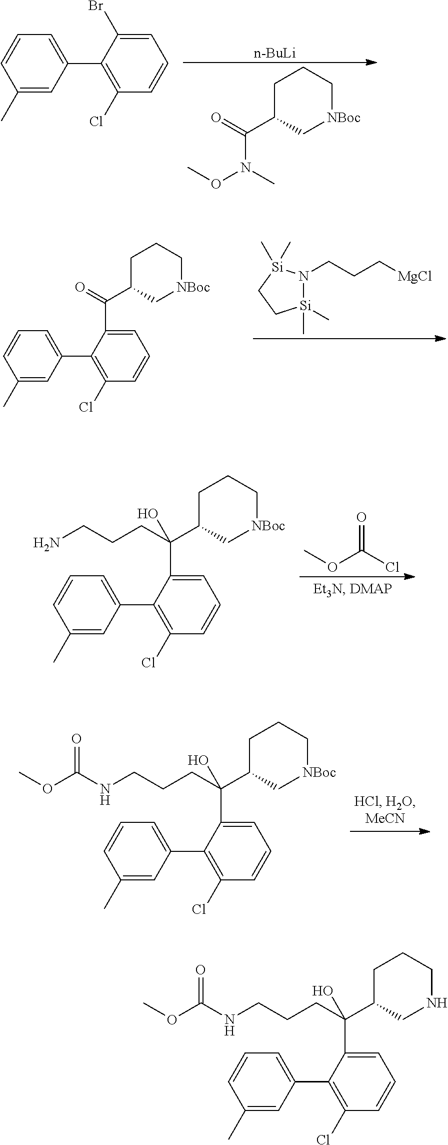

Step 1. (R)-tert-butyl 3-(6-chloro-3′-methylbiphenylcarbonyl)piperidine-1-carboxylate: To a solution of 6-bromo-2-fluoro-3′-methylbiphenyl (2 g, 7.14 mmol) in anhydrous THF (30 mL) cooled to −78° C. was added dropwise a solution of 1.6 M of n-BuLi in hexane (4.46 mL). The reaction mixture was stirred at −78° C. for 1 h and a solution of (R)-tert-butyl 3-(methoxy(methyl)carbamoyl)piperidine-1-carboxylate (1.94 g, 7.14 mmol) in anhydrous THF (20 mL) was added. The mixture was allowed to warm to rt and stirred overnight. The mixture was quenched with satd aq NH₄Cl (40 mL) and extracted with EtOAc (40 mL). The combined organic layers were dried over Na₂SO₄ and concentrated to give crude product, which was purified by flash column chromatography to afford (R)-tert-butyl 3-(6-chloro-3′-methylbiphenylcarbonyl)piperidine-1-carboxylate (1 g, 34%). ¹H NMR (400 MHz, CD₃OD) δ ppm 0.80-1.20 (m, 8H), 1.30 (s, 1H), 1.40 (s, 1H), 1.40-1.60 (m, 2H), 2.00-2.18 (s, 1H), 2.30-2.40 (s, 3H), 2.60-2.80 (m, 2H), 3.50-3.80 (m, 2H), 7.00-7.15 (s, 2H), 7.20-7.30 (d, 1H), 7.30-7.40 (t, 2H), 7.39-7.48 (t, 1H), 7.60-7.70 (d, 1H); MS (E/Z): 414 (M+H⁺)

Step 2. 1,1-dimethylethyl (3R)-3-[4-amino-1-(6-chloro-3′-methyl-2-biphenylyl)-1-hydroxybutyl]-1-piperidinecarboxylate: To a solution of (R)-tert-butyl 3-(6-chloro-3′-methylbiphenylcarbonyl)piperidine-1-carboxylate (800 mg, 1.94 mmol) in anhydrous THF (15 mL) cooled to −78° C. was added dropwise a solution of 2 M (3-(2,2,5,5-tetramethyl-1,2,5-azadisilolidin-1-yl)propyl)magnesium chloride in THF (0.968 mL, 1.94 mmol). After addition, the reaction mixture was allowed to warm slowly to rt while stirring overnight. The mixture was quenched with satd aq NH₄Cl (15 mL) and extracted with CH₂Cl₂ (3×). The combined organic layers were dried over Na₂SO₄ and concentrated to give crude-1,1-dimethylethyl (3R)-3-[4-amino-1-(6-chloro-3′-methyl-2-biphenylyl)-1-hydroxybutyl]-1-piperidinecarboxylate (900 mg), which was used in the next step without further purification.

Step 3. 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate: To a solution of 1,1-dimethylethyl (3R)-3-[4-amino-1-(6-chloro-3′-methyl-2-biphenylyl)-1-hydroxybutyl]-1-piperidinecarboxylate (800 mg, 1.69 mmol) in anhydrous CH₂Cl₂ (15 mL) were added 4-dimethylaminopyridine (1.24 g, 10.17 mmol) and Et₃N (2.35 mL, 16.95 mmol). The mixture was cooled with an ice bath and methyl chloroformate (0.65 mL, 8.47 mmol) in CH₂Cl₂ (5 mL) was added. The reaction mixture was allowed to warm slowly to rt while stirring overnight. The solvent was removed in vacuo and the residue was purified by column chromatography to afford 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (700 mg, 78%). ¹H NMR (400 MHz, CD₃OD) δ ppm 1.00-1.70 (m, 17H), 2.30-2.50 (d, 3H), 2.50-2.70 (s, 1H), 2.90-2.31 (m, 2H), 3.50-3.52 (m, 3H), 3.80-4.20 (m, 2H), 6.0-7.15 (m, 3H), 7.15-7.40 (m, 3H), 7.50-7.70 (m, 1H); MS (E/Z): 531 (M+H⁺)

Step 4. Methyl {4-(6-chloro-3′-methyl-2-biphenylyl)-4-hydroxy-4-[(3R)-3-piperidinyl]butyl}carbamate: To a solution of 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (600 mg, 1.13 mg) in CH₃CN (18 mL) was added 2N aq HCl (15 mL) and the reaction mixture was vigorously stirred overnight at rt. The solvents were removed in vacuo to give methyl {4-(6-chloro-3′-methyl-2-biphenylyl)-4-hydroxy-4-[(3R)-3-piperidinyl]butyl}carbamate as its hydrochloride salt (500 mg, 95.8%). ¹H NMR (400 MHz, CD₃OD) δ ppm 1.00-1.20 (m, 1H), 1.30-1.80 (m, 8H), 1.80-2.00 (m, 2H), 2.40-2.50 (d, 3H), 2.75-2.90 (t, 1H), 2.90-3.05 (m, 3H), 3.05-3.12 (t, 1H), 3.20-3.30 (m, 1H), 3.30-3.40 (m, 1H), 3.60-3.70 (d, 4H), 6.90-6.98 (d, 1H), 7.00-7.12 (m, 1H), 7.25-7.50 (m, 4H), 7.75-7.85 (d, 1H); MS (E/Z): 431 (M+H⁺)

The following compounds were prepared using procedures analogous to those described above:

Methyl 4-(3′-ethyl-6-fluorobiphenyl-2-yl)-4-hydroxy-4-((R)-piperidin-3-yl)butylcarbamate using 2-bromo-6-fluoro-3′-ethylbiphenyl in Step 1.

Methyl 4-hydroxy-4-((R)-piperidin-3-yl)-4-(2-(o-tolyloxy)phenyl)butylcarbamate using 1-bromo-2-(o-tolyloxy)benzene in Step 1.

N-(4-(6-fluoro-3′-methylbiphenyl-2-yl)-4-hydroxy-4-(piperidin-3-yl)butyl)acetamide using 2-bromo-6-fluoro-3′-methylbiphenyl in Step 1 and acetic anhydride in Step 3.

N-(4-(2′,6-difluoro-5′-methylbiphenyl-2-yl)-4-hydroxy-4-(piperidin-3-yl)butyl)acetamide using 2′-bromo-2,6′-difluoro-5-methylbiphenyl in Step 1 and acetic anhydride in Step 3.

Methyl 4-(3′,6-difluoro-5′-methylbiphenyl-2-yl)-4-hydroxy-4-(piperidin-3-yl)butylcarbamate using 2-bromo-3′,6-difluoro-5′-methylbiphenyl in Step 1.

Methyl 4-(6-chloro-3′-fluoro-5′-methylbiphenyl-2-yl)-4-hydroxy-4-(piperidin-3-yl)butylcarbamate using 2-bromo-6-chloro-3′-fluoro-5′-methylbiphenyl in Step 1.

Methyl 4-(3-fluoro-2-(quinolin-3-yl)phenyl)-4-hydroxy-4-(piperidin-3-yl)butylcarbamate using 3-(2-bromo-6-fluorophenyl)quinoline in Step 1.

Methyl 4-(3-chloro-2-(5-methylfuran-2-yl)phenyl)-4-hydroxy-4-(piperidin-3-yl)butylcarbamate using 2-(2-bromo-6-chlorophenyl)-5-methylfuran in Step 1.

Methyl 4-(3-chloro-2-(2,3-dihydrobenzofuran-6-yl)phenyl)-4-hydroxy-4-((R)-piperidin-3-yl)butylcarbamate using 6-(2-bromo-6-chlorophenyl)-2,3-dihydrobenzofuran in Step 1.

Methyl 4-(3-fluoro-2-(quinolin-3-yl)phenyl)-4-hydroxy-4-(piperidin-3-yl)butylcarbamate using 3-(2-bromo-6-chlorophenyl)quinoline in Step 1.

Methyl 4-(6-chloro-2′-methoxy-5′-(trifluoromethyl)biphenyl-2-yl)-4-hydroxy-4-((R)-piperidin-3-yl)butylcarbamate using 2′-bromo-6′-chloro-2-methoxy-5-(trifluoromethyl)biphenyl in Step 1.

N-4-(2′,6-difluoro-5′-methylbiphenyl-2-yl)-4-hydroxy-4-((R)-piperidin-3-yl)butyl)-2,2,2-trifluoroacetamide using 2′-bromo-2,6′-difluoro-5-methylbiphenyl in Step 1 and trifluoroacetic anhydride in Step 3.

N-(4-(3′-ethyl-6-fluorobiphenyl-2-yl)-4-hydroxy-4-((R)-piperidin-3-yl)butyl)-2,2,2-trifluoroacetamide using 2-bromo-3′-ethyl-6-fluorobiphenyl in Step 1 and trifluoroacetic anhydride in Step 3.

Methyl 4-(6-chloro-3′-ethylbiphenyl-2-yl)-4-hydroxy-4-((R)-piperidin-3-yl)butylcarbamate using 2-bromo-6-chloro-3′-ethylbiphenyl in Step 1.

Methyl 4-(6-fluoro-3′-isopropylbiphenyl-2-yl)-4-hydroxy-4-((R)-piperidin-3-yl)butylcarbamate using 2-bromo-6-fluoro-3′-isopropylbiphenyl in Step 1.

### Preparation 5

### (5-methoxy-1-(2-phenoxyphenyl)-1-((R)-piperidin-3-yl)pentan-1-ol

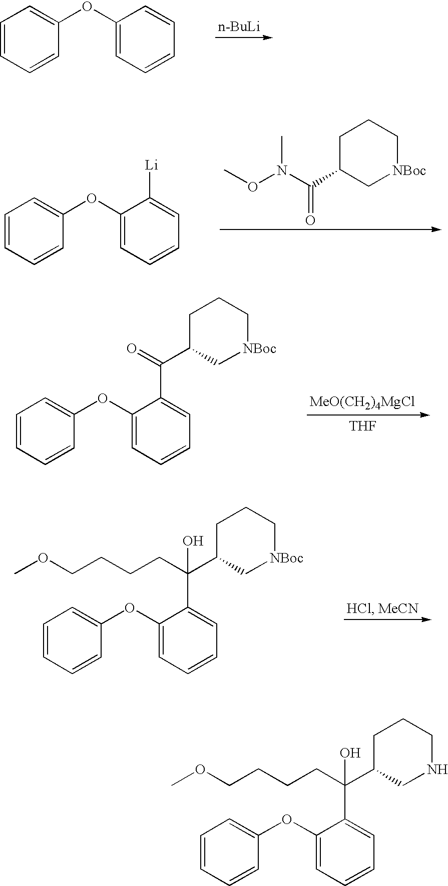

### Step 1. 2-(Phenoxy)phenyllithium

To a solution of diphenyl ether (8.60 g, 50.0 mmol) in Et₂O (75 mL) was added n-BuLi (1.6 M in hexane, 32.8 mL, 52.5 mmol). The mixture was refluxed for 48 h, and the resulting solution of 2-(phenoxy)phenyllithium was used in the next step without any further analysis.

### Step 2. (3R)-1-(tert-butoxycarbonyl)-3-(2-phenoxybenzoyl)piperidine

To a solution of (R)-tert-butyl 3-(N-methoxy-N-methylcarbamoyl)piperidine-1-carboxylate (4.40 g, 16.2 mmol) in anhydrous THF (18 mL) at −10° C., was added dropwise the solution of 2-phenoxyphenyllithium prepared in Step 1 (80 mL, 32 mmol). The mixture was then warmed to rt, and stirred until no starting material remained (˜30 min). The reaction was quenched with 1 N HCl (˜30 mL) and extracted with Et₂O (4×10 mL). The combined organic layers were washed with satd aq NaHCO₃ and brine, and dried over Na₂SO₄. The solvent was removed to give (3R)-1-(tert-butoxycarbonyl)-3-(2-phenoxybenzoyl)piperidine (7.44 g, quantitative).

### Step 3. (3R)-tert-Butyl 3-(1-hydroxy-5-methoxy-1-(2-phenoxyphenyl)pentyl)piperidine-1-carboxylate

To a solution of (3R)-1-(tert-butoxycarbonyl)-3-(2-phenoxybenzoyl)piperidine (6.17 g, 16.2 mmol) in THF (30 mL) at −10° C. was added dropwise 2.54 M 4-methoxybutylmagnesium chloride in THF (15 mL, 38 mmol). The resulting solution was warmed to rt slowly, and stirred over night. The reaction was quenched with satd NH₄Cl (10 mL) and extracted with Et₂O (4×10 mL). The combined organic layers were washed with water and brine. The solvent was removed and the residue was purified by flash chromatography to give (3R)-tert-Butyl 3-(1-hydroxy-5-methoxy-1-(2-phenoxyphenyl)pentyl)piperidine-1-carboxylate (1.97 g, 26% from (R)-tert-butyl 3-(N-methoxy-N-methylcarbamoyl)piperidine-1-carboxylate).

### Step 4. (5-methoxy-1-(2-phenoxyphenyl)-1-((R)-piperidin-3-yl)pentan-1-ol

To a solution of (R)-tert-butyl 3-((S)-1-hydroxy-5-methoxy-1-(2-phenoxyphenyl) pentyl)piperidine-1-carboxylate (1.97 g, 4.19 mmol) in MeCN (100 mL) was added 2 N aq HCl (100 mL) slowly at rt. The resulting solution was stirred at rt until no starting material remained (˜16 h), basified to pH=10 with 10 N aq NaOH, and evaporated under reduced pressure to remove MeCN. The aq layer was extracted with CH₂Cl₂ (4×10 mL). The combined organic layers were washed with brine and dried over Na₂SO₄. The solvent was removed in vacuo to afford (5-methoxy-1-(2-phenoxyphenyl)-1-((R)-piperidin-3-yl)pentan-1-ol (1.56 g, quantitative) as a free amine.

### Preparation 6

### Morpholine Synthesis

### (R)-1-(6-Fluoro-3′-methylbiphenyl-2-yl)-5-methoxy-1-((R)-morpholin-2-yl)pentan-1-ol

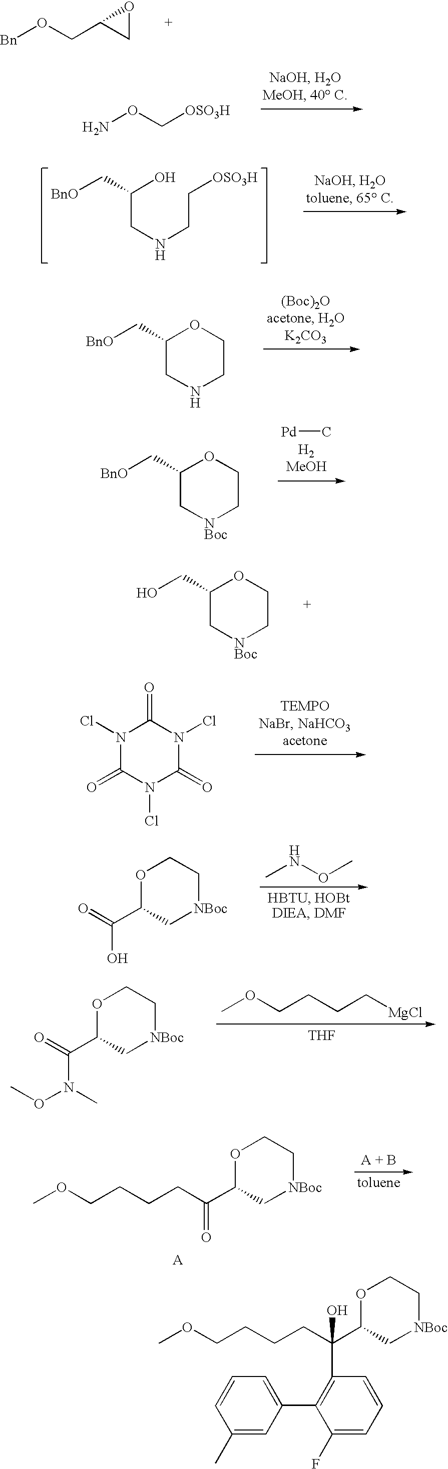

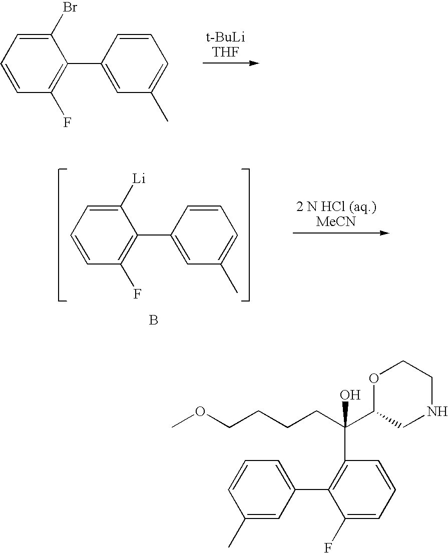

### Step 1. (R)-2-(Benzyloxymethyl)morpholine

To a stirred mixture of (R)-2-(benzyloxymethyl)oxirane (10.0 g, 60.9 mmol) and NaOH (19.49 g, 487.2 mmol) in H₂O (46 mL) and MeOH (18 mL), there was added 2-aminoethyl hydrogen sulfate (36.8 g, 255.8 mmol) in portions. After addition was complete, the reaction mixture was stirred at 40° C. for 2 h. After cooling, the mixture was treated with NaOH (15.0 g, 375.0 mmol), followed by toluene (70 mL), and stirred at 65° C. overnight. The mixture was cooled, diluted with toluene (27 mL) and H₂O (92 mL). The toluene layer was separated and the aqueous layer was extracted with CH₂Cl₂ (2×50 mL). The combined organic layers were concentrated to give crude (R)-2-(benzyloxymethyl)morpholine (˜14 g), which was used without purification. MS m/z 208 (M+H⁺).

### Step 2. (R)-tert-Butyl 2-(benzyloxymethyl)morpholine-4-carboxylate

To a solution of crude (R)-2-(benzyloxymethyl)morpholine (˜14 g) in acetone (100 mL) and H₂O (30 mL) at 0° C., there was added K₂CO₃ (25.2 g, 182.7 mmol), followed by (Boc)₂O (14.6 g, 67.0 mmol). The resulting solution was warmed to rt, and stirred until no starting material remained (˜30 min). Acetone was removed under vacuum, and the aqueous solution was extracted with CH₂Cl₂ (4×10 mL). The combined organic layers were washed with H₂O (10 mL) and the solvent was removed. The residue was purified by flash column chromatography to give (R)-tert-butyl 2-(benzyloxymethyl)morpholine-4-carboxylate (8.33 g, 44% over 2 steps). ¹H NMR (400 MHz, CDCl₃) δ ppm 7.34 (m, 5H), 4.56 (s, 2H), 3.88 (d, 2H), 3.82 (br, 1H), 3.40 (m, 1H), 3.48 (m, 3H), 2.94 (m, 1H), 2.76 (m, 1H), 1.44 (s, 9H); MS m/z 330 (M+Na⁺).

### Step 3. (R)-tert-Butyl 2-(hydroxymethyl)morpholine-4-carboxylate

To a solution of (R)-tert-butyl 2-(benzyloxymethyl)morpholine-4-carboxylate (8.33 g, 27.1 mmol) in EtOH was added Pd—C (wet, 3.6 g), and the resulting mixture was stirred at rt under a H₂ balloon overnight. After filtration, the solvent was removed under vacuum, and the residue was purified by flash column chromatography to give (R)-tert-butyl 2-(hydroxymethyl)morpholine-4-carboxylate (5.84 g, 99%) as a clear oil. ¹H NMR (400 MHz, CDCl₃) δ ppm 3.88 (d, 2H), 3.82 (br, 1H), 3.64 (d, 1H), 3.56 (m, 3H), 2.94 (m, 1H), 2.76 (m, 1H), 1.90 (br, 1H), 1.44 (s, 9H); MS m/z 218 (M+H⁺).

### Step 4. (R)-4-(tert-Butoxycarbonyl)morpholine-2-carboxylic acid

Satd aq NaHCO₃ (15 mL) was added to a solution of (R)-tert-butyl 2-(hydroxymethyl)-morpholine-4-carboxylate (1.09 g, 5.0 mmol) in acetone (50 mL), stirred and maintained at 0° C. Solid NaBr (0.1 g, 1 mmol) and TEMPO (0.015 g, 0.1 mmol) were added. Trichloroisocyanuric acid (2.32 g, 10.0 mmol) was then added slowly within 20 min at 0° C. After addition, the mixture was warmed to rt and stirred overnight. 2-Propanol (3 mL) was added, and the resulting solution was stirred at rt for 30 min, filtered through a pad of Celite®, concentrated under vacuum, and treated with satd aq Na₂CO₃ (15 mL). The aqueous solution was washed with EtOAc (5 mL), acidified with 6 N HCl, and extracted with EtOAc (5×10 mL). The combined organic layers were dried over Na₂SO₄ and the solvent was removed to give (R)-4-(tert-butoxycarbonyl)morpholine-2-carboxylic acid (1.07 g, 92%) as a white solid. ¹H NMR (400 MHz, CDCl₃) δ ppm 4.20 (br, 1H), 4.12 (d, 1H), 4.02 (d, 1H), 3.84 (m, 1H), 3.62 (m, 1H), 3.04 (m, 2H), 1.44 (s, 9H); MS m/z 232 (M+H⁺).

### Step 5. (R)-tert-Butyl 2-(methoxy(methyl)carbamoyl)morpholine-4-carboxylate

To a solution of (R)-4-(tert-butoxycarbonyl)morpholine-2-carboxylic acid (1.05 g, 4.54 mmol) in DMF (10 mL) at 0° C., was added DIEA (3.9 mL, 22.7 mmol), followed by HBTU (1.89 g, 4.99 mmol) and HOBt (0.67 g, 4.99 mmol). MeONMHMe.HCl (0.48 g, 4.92 mmol) was added and the resulting solution was warmed to rt and stirred until no starting material remained (˜2 h). The mixture was diluted with H₂O (10 mL) and extracted with EtOAc (4×10 mL). The combined organic layers were washed with 1 N aq HCl (10 mL), 1 N aq NaOH (3×10 mL), water (2×10 mL) and brine (10 mL), and dried over Na₂SO₄. The solvent was removed under vacuum to give (R)-tert-butyl 2-(methoxy(methyl)carbamoyl)morpholine-4-carboxylate (1.40 g, quant.), which was used without further purification. ¹H NMR (400 MHz, CDCl₃) δ ppm 4.36 (br, 1H), 4.08 (m, 1H), 4.00 (d, 1H), 3.84 (m, 1H), 3.76 (s, 3H), 3.58 (m, 1H), 3.20 (s, 3H), 3.04 (m, 2H), 1.44 (s, 9H); MS m/z 297 (M+Na⁺).

### Step 6. (R)-tert-Butyl 2-(5-methoxypentanoyl)morpholine-4-carboxylate

To a stirred solution of (R)-tert-butyl 2-(methoxy(methyl)carbamoyl)morpholine-4-carboxylate (1.37 g, 5.0 mmol) in THF (10 mL) at −20° C., there was added 1.47 M 4-methoxybutylmagnesium chloride in THF (10.2 mL, 15.0 mmol) dropwise to keep the temperature below −20° C. After addition, the resulting solution was warmed to rt and quenched with 1 N aq HCl (10 mL). The organic layer was separated, and the aqueous layer was extracted with ether (3×5 mL). Combined organic layers were washed with satd aq NaHCO₃ (10 mL) and brine (5 mL) and dried over Na₂SO₄. Removal of the solvent under vacuum gave (R)-tert-butyl 2-(5-methoxypentanoyl)morpholine-4-carboxylate (1.41 g, 93%), which was used without purification. MS m/s 324 (M+Na⁺).

### Step 7. (R)-tert-Butyl 2-((R)-1-(6-fluoro-3′-methylbiphenyl-2-yl)-1-hydroxy-5-methoxypentyl)-morpholine-4-carboxylate

To a solution of 2-bromo-6-fluoro-3′-methylbiphenyl (1.90 g, 7.17 mmol) in ether (8 mL) at −78° C., there was added t-BuLi in pentane (1.70 M, 8.43 mL, 14.33 mmol) dropwise to keep the temperature below −70° C. The resulting solution was stirred at −78° C.

To a solution of (R)-tert-butyl 2-(5-methoxypentanoyl)morpholine-4-carboxylate (0.68 g, 2.26 mmol) in toluene (8 mL) at −20° C. there was added the above lithium reagent dropwise to keep the solution temperature below −20° C. After addition, the resulting mixture was warmed to rt slowly, and quenched with saturated NH₄Cl (8 mL). The organic layer was separated, and aqueous layer was extracted with ether (3×5 mL). Combined organic layers were washed with water (10 mL), concentrated, and the residue was purified by flash column chromatography to give (R)-tert-butyl 2-((R)-1-(6-fluoro-3′-methylbiphenyl-2-yl)-1-hydroxy-5-methoxypentyl)-morpholine-4-carboxylate (0.48 g, 44%) as a foam. ¹H NMR (400 MHz, CDCl₃) δ ppm 7.40 (m, 1H), 7.32 (m, 2H), 7.20 (d, 1H), 7.04 (m, 3H), 3.84 (m, 1H), 3.78 (m, 2H), 3.40-3.24 (ms, 7H), 2.82 (s, 3H), 1.70-1.20 (m, 5H), 1.44 (s, 9H), 0.94 (m, 1H); MS m/z 510 (M+Na⁺).

### Step 8. (R)-1-(6-Fluoro-3′-methylbiphenyl-2-yl)-5-methoxy-1-((R)-morpholin-2-yl)-pentan-1-ol

To a solution of (R)-tert-butyl 2-((R)-1-(6-fluoro-3′-methylbiphenyl-2-yl)-1-hydroxy-5-methoxypentyl)morpholine-4-carboxylate (0.46 g, 0.96 mmol) in acetonitrile (50 mL) was added 2 N aq HCl (50 mL). The resulting solution was stirred at rt overnight and basified with 10 N aq NaOH to pH 10. Acetonitrile was removed under vacuum, and the aqueous residue was extracted with CH₂Cl₂ (4×5 mL). The combined organic layers were washed with brine (5 mL), dried over Na₂SO₄, and concentrated to give (R)-1-(6-fluoro-3′-methylbiphenyl-2-yl)-5-methoxy-1-((R)-morpholin-2-yl)pentan-1-ol (0.38, quant.). MS m/z 388 (M+H⁺).

The following compound was prepared using procedures analogous to those described above:

(R)-1-(4′,6-difluoro-3′-methylbiphenyl-2-yl)-5-methoxy-1-((R)-morpholin-2-yl)pentan-1-ol using 2-bromo-4′,6-difluoro-3′-methylbiphenyl in Step 7.

### Preparation 7

### 1-(3′-ethyl-6-fluorobiphenyl-2-yl)-5-methoxy-1-(piperidin-4-yl)pentan-1-ol

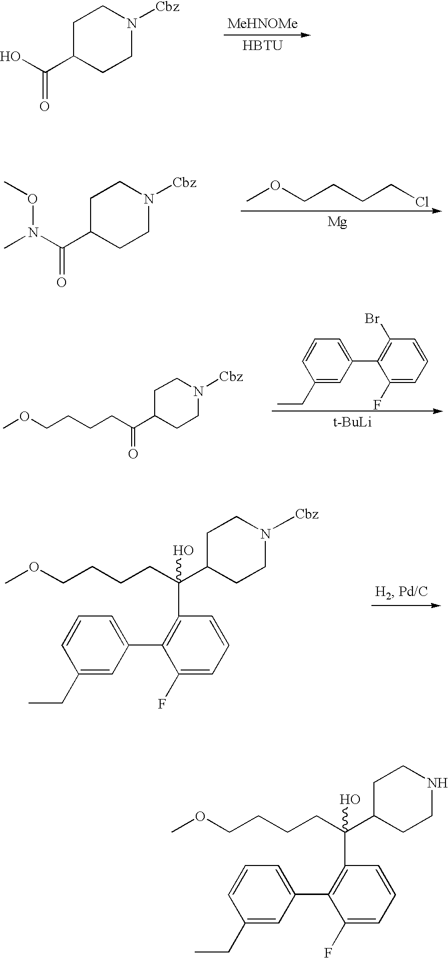

### Step 1. Benzyl 4-(methoxy(methyl)carbamoyl)piperidine-1-carboxylate

A solution of 1-(benzyloxycarbonyl)piperidine-4-carboxylic acid (2.1 g, 8.0 mmol) in 20 ml, of DMF at 0° C. was treated with N,O-dimethylhydroxylamine hydrochloride (0.84 g, 8.6 mmol), i-Pr₂NEt (7 mL, 40.0 mmol), HBTU (3.3 g, 8.8 mmol), and HOBt (1.2 g, 8.8 mmol) and the mixture was stirred and warmed to 25° C. After 16 h, H₂O (50 mL) was added and the mixture was extracted with EtOAc (3×50 mL). The combined organic extracts were washed (1N HCl, 1N NaOH, H₂O, brine), dried (Na₂SO₄), and concentrated to provide benzyl 4-(methoxy(methyl)carbamoyl)piperidine-1-carboxylate as a yellow oil (2.1 g, 89%).

### Step 2. Benzyl 4-(5-methoxypentanoyl)piperidine-1-carboxylate

A solution of benzyl 4-(methoxy(methyl)carbamoyl)piperidine-1-carboxylate (0.7 g, 2.3 mmol) in 4 mL of THF at −20° C. was treated with a solution of 4-(methyloxy)butyl magnesium chloride (7 mL of 1.28 M in THF, 9.0 mmol) and the mixture was stirred and warmed to 25° C. over 2 hours before being quenched with the addition of aqueous 1N HCl and extracted with Et₂O. The combined organic extracts were dried (Na₂SO₄), concentrated, and subjected to flash chromatography to provide benzyl 4-(5-methoxypentanoyl)piperidine-1-carboxylate as a colorless oil (0.67 g, 88%). MS (m/z) 334.2 (M+H⁺).

### Step 3. benzyl 4-(1-(3′-ethyl-6-fluorobiphenyl-2-yl)-1-hydroxy-5-methoxypentyl)piperidine-1-carboxylate

A solution of 2-bromo-3′-ethyl-6-fluorobiphenyl (0.5 mg, 1.8 mmol) in 2 mL of Et₂O at −78° C. was treated with t-BuLi (2.1 mL of 1.7 M in pentane, 3.6 mmol). After 5 minutes, a solution of benzyl 4-(5-methoxypentanoyl)piperidine-1-carboxylate (0.3 g, 0.9 mmol) in 2 mL of THF was added and the mixture was stirred for 1 h before being quenched with the addition of saturated aqueous NH₄Cl and extracted with Et₂O. The combined organic extracts were dried (Na₂SO₄), concentrated, and subjected to flash chromatography to provide benzyl 4-(1-(3′-ethyl-6-fluorobiphenyl-2-yl)-1-hydroxy-5-methoxypentyl)piperidine-1-carboxylate as a colorless oil (0.15 g, 31%). MS (m/z) 556.2 (M+Na⁺).

### Step 4. 1-(3′-ethyl-6-fluorobiphenyl-2-yl)-5-methoxy-1-(piperidin-4-yl)pentan-1-ol

A solution of benzyl 4-(1-(3′-ethyl-6-fluorobiphenyl-2-yl)-1-hydroxy-5-methoxypentyl)piperidine-1-carboxylate (70 mg, 0.13 mmol) in 2 mL of MeOH at 25° C. was treated with 10% Pd/C (20 mg) and stirred under an atmosphere of hydrogen. After 2 h, the mixture was filtered and concentrated to provide 1-(3′-ethyl-6-fluorobiphenyl-2-yl)-5-methoxy-1-(piperidin-4-yl)pentan-1-ol as a colorless oil (53 mg, quantitative). MS (m/z) 400.3 (M+H⁺).

### Preparation 8

### Piperidines from Weinreb Amides and Bromobiaryls

### 1-(2′-chloro-2-biphenylyl)-5-(methyloxy)-1-[(3R)-3-piperidinyl]-1-pentanol

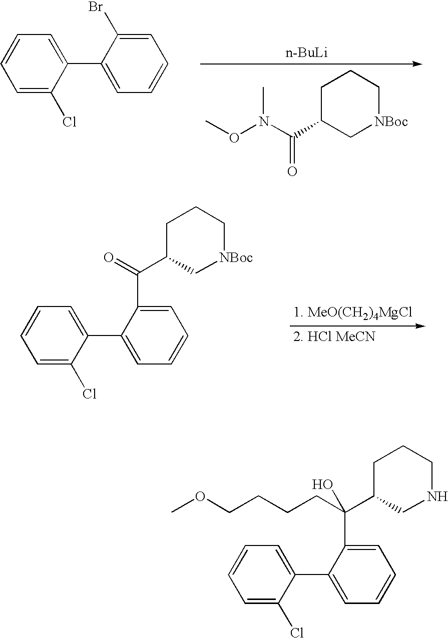

Step 1. (3R)-1-(tert-butoxycarbonyl)-3-((2-(2-chlorophenyl))benzoyl)piperidine To a solution of 2′-bromo-2-chloro-biphenyl (5.34 g, 20 mmol) in anhydrous THF (50 mL) cooled to −78° C. was added dropwise a solution of 1.6 M n-BuLi in hexane (12.5 mL, 20 mmol). The reaction mixture was stirred at −78° C. for 1 h and a solution of (R)-tert-butyl 3-(N-methoxy-N-methylcarbamoyl)-piperidine-1-carboxylate (5.44 g, 20 mmol) in anhydrous THF (50 mL) was added. The mixture was allowed to warm to rt and stirred overnight. The mixture was quenched with satd aq NH₄Cl (100 mL) and extracted with EtOAc (3×75 mL). The combined organic layers were dried over Na₂SO₄ and concentrated to give the crude product, which was purified by flash column chromatography to afford (3R)-1-(tert-butoxycarbonyl)-3-((2-(2-chlorophenyl))benzoyl)piperidine (4.43 g, 55%).

Step 2. 1,1-dimethylethyl (3R)-3-[1-(2′-chloro-2-biphenylyl)-1-hydroxy-5-(methyloxy)pentyl]-1-piperidinecarboxylate: A 250 mL three-necked flask was charged with magnesium turning (2.88 g, 0.12 mol) and a small crystal of iodine. The flask was evacuated and refilled with N₂. A solution of 1-chloro-4-methoxybutane (15 g, 0.12 mol) in THF (60 ml) was added dropwise to the above mixture. After heating under reflux for 2 h most of magnesium had been consumed and the Grignard solution was cooled to rt. A 250 mL three-necked flask was charged with (3R)-1-(tert-butoxycarbonyl)-3-((2-(2-chlorophenyl))benzoyl)piperidine (4.43 g, 11 mmol) and THF (50 mL), evacuated and refilled with N₂. The mixture was cooled in a dry ice-acetone bath and the Grignard reagent was added dropwise. The mixture was allowed to warm slowly to rt and stirred overnight. The mixture was quenched with satd aq NH₄Cl (100 mL) and extracted with EtOAc. The combined organic layers were dried over Na₂SO₄ and concentrated to give the crude product which was purified by flash column chromatography to afford pure 1,1-dimethylethyl (3R)-3-[1-(2′-chloro-2-biphenylyl)-1-hydroxy-5-(methyloxy)pentyl]-1-piperidinecarboxylate (2.5 g, 47%).

Step 3. 1-(2′-chloro-2-biphenylyl)-5-(methyloxy)-1-[(3R)-3-piperidinyl]-1-pentanol: The Boc protecting group was removed using the protocol described in Preparation 5 Step 4.

The following compound was prepared using procedures analogous to those described above:

1-(6-chloro-3′-ethylbiphenyl-2-yl)-5-methoxy-1-((R)-piperidin-3-yl)pentan-1-ol using 2-bromo-6-chloro-3′-ethylbiphenyl in Step 1.

### Preparation 9

### 4-[{[(1,1-dimethylethyl)oxy]carbonyl}(methyl)amino]butanoic acid

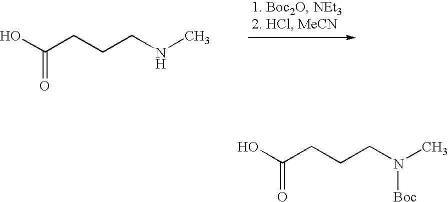

A solution of 4-(methylamino)butyric acid hydrochloride (2.0 g, 13.0 mmol) in 25 mL of THF at 25° C. was treated with Boc₂O (4.3 g, 19.5 mmol) and Et₃N (7.3 mL, 52.0 mmol), and the mixture was stirred overnight before being quenched with the addition of 40 mL of aqueous 1N NaOH. The mixture was washed with CH₂Cl₂ (2×100 mL), cooled to 0° C., acidified (pH 5-6) with the addition of aqueous 3.5 N HCl, and extracted with CH₂Cl₂ (2×75 mL). The combined organic extracts were dried (MgSO₄) and concentrated under reduced pressure to give 4-[{[(1,1-dimethylethyl)oxy]carbonyl}(methyl)amino]butanoic acid as a white solid.

The following compound was prepared using procedures analogous to those described above:

4-(tert-butoxycarbonylamino)-3-(4-chlorophenyl)butanoic acid using 4-amino-3-(4-chlorophenyl)butanoic acid instead of 4-(methylamino)butyric acid hydrochloride.

### Preparation 10

### Sodium 4-[{[(1,1-dimethylethyl)oxy]carbonyl}(methyl)amino]-3-hydroxybutanoate

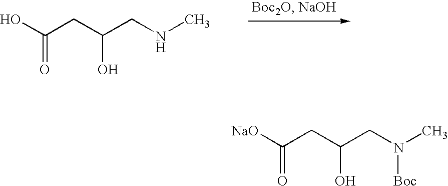

A solution of 4-amino-3-hydroxybutyric acid (1.0 g, 8.4 mmol) in 5 mL of dioxane at 25° C. was treated with 5 mL of aqueous 1N NaOH and Boc₂O (2.75 g, 12.6 mmol) and the mixture was stirred overnight before being washed with CH₂Cl₂ (2×15 mL) and concentrated under reduced pressure. The resulting mixture containing sodium 4-[{[(1,1-dimethylethyl)oxy]carbonyl}(methyl)amino]-3-hydroxybutanoate was used directly in the subsequent reaction without purification.

The following compounds were prepared using procedures analogous to those described above:

Sodium (2S)-4-[{[(1,1-dimethylethyl)oxy]carbonyl}(methyl)amino]-2-hydroxybutanoate using (2S)-2-hydroxy-4-(methylamino)butanoic acid instead of 4-amino-3-hydroxybutyric acid.

Sodium (3S)-4-({[(1,1-dimethylethyl)oxy]carbonyl}amino)-3-hydroxybutanoate using (3S)-4-({[(1,1-dimethylethyl)oxy]carbonyl}amino)-3-hydroxybutanoic acid instead of 4-amino-3-hydroxybutyric acid.

### Preparation 11

### Lithium 3-hydroxy-4-{methyl[(2-nitrophenyl)sulfonyl]amino}butanoate

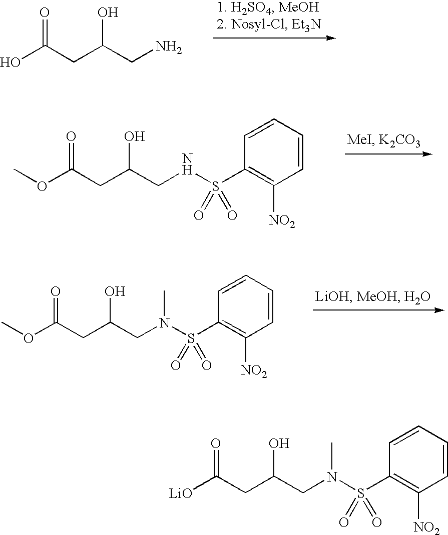

### Step 1. Methyl 4-amino-3-hydroxybutanoate

A solution of 4-amino-3-hydroxybutanoic acid (10.0 g, 83.94 mmol) in 40 mL of MeOH at 25° C. was treated with concentrated H₂SO₄ (3 mL) and the mixture was stirred and heated at 65° C. overnight before being cooled to 0° C. and basified by the addition of solid KHCO₃. The suspension was filtered thru Celite® and concentrated to give a gum, which was dissolved in 80 mL of acetonitrile and slowly treated with 21 ml, of 4N HCl in dioxane solution. The resulting solution was concentrated under reduced pressure to give methyl 4-amino-3-hydroxybutanoate as an oil.

### Step 2. Methyl 3-hydroxy-4-{[(2-nitrophenyl)sulfonyl]amino}butanoate

A solution of methyl 4-amino-3-hydroxybutanoate (4.0 g, 23.65 mmol) in 35 mL of CH₂Cl₂ at 0° C. was treated with Et₃N (9.9 mL, 70.95 mmol) and a solution of 2-nitrosulfonyl chloride in 10 mL of CH₂Cl₂, and the mixture was stirred at 0° C. for 30 min before being quenched with the addition of saturated aqueous KHCO₃ (25 mL). The organic layer was separated, dried (MgSO₄), concentrated under reduced pressure, and subjected to flash chromatography to give methyl 3-hydroxy-4-{[(2-nitrophenyl)sulfonyl]amino}butanoate as a light brown oil (3.07 g, 41%). ESI-MS (m/z): 341.0 (M+Na⁺).

### Step 3. Methyl 3-hydroxy-4-{methyl[(2-nitrophenyl)sulfonyl]amino}butanoate

A solution of methyl 3-hydroxy-4-{[(2-nitrophenyl)sulfonyl]amino}butanoate (0.5 g, 1.57 mmol) in 5 mL of DMF at 25° C. was treated with iodomethane (0.2 mL, 3.14 mmol) and K₂CO₃ (0.65 g, 3.14 mmol) and the mixture was stirred overnight before being quenched with the addition of 15 mL of water and extracted with CH₂Cl₂ (2×20 mL). The combined organic extracts were washed with brine, dried (MgSO₄), concentrated under reduced pressure, and subjected to flash chromatography to give methyl 3-hydroxy-4-{methyl[(2-nitrophenyl)sulfonyl]amino}butanoate as a gum (0.38 g, 73%). ESI-MS (m/z): 333.1 (M+H⁺).

### Step 4. Lithium 3-hydroxy-4-{methyl[(2-nitrophenyl)sulfonyl]amino}butanoate

A solution of methyl 3-hydroxy-4-{methyl[(2-nitrophenyl)sulfonyl]amino}butanoate (0.10 g, 0.30 mmol) in 1.6 mL of MeOH and 0.4 mL of water at 25° C. was treated with LiOH (0.008 g, 0.33 mmol) and the mixture was stirred for 1 h before being concentrated under reduced pressure to give lithium 3-hydroxy-4-{methyl[(2-nitrophenyl)sulfonyl]amino}butanoate as a brown solid which was used without purification. ESI-MS (m/z): 341.0 (M+Na⁺).

### Preparation 12

### Lithium 4-{methyl[(2-nitrophenyl)sulfonyl]amino}-3-(methyloxy)butanoate

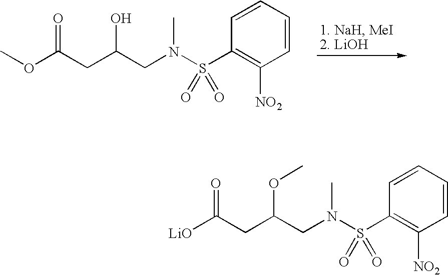

### Step 1. Methyl 4-{methyl[(2-nitrophenyl)sulfonyl]amino}-3-(methyloxy)butanoate

A solution of methyl 3-hydroxy-4-{methyl[(2-nitrophenyl)sulfonyl]amino}butanoate (0.12 g, 0.36 mmol) in 1.2 mL of DMF at 0° C. was treated with iodomethane (0.067 mL, 1.08 mmol) and NaH (0.017 g, 0.72 mmol) and the mixture was stirred for 15 minutes before being filtered through Celite® and subjected to reverse phase HPLC to give methyl 4-{methyl[(2-nitrophenyl)sulfonyl]amino}-3-(methyloxy)butanoate as a brown gum (0.056 g, 45%). ESI-MS (m/z): 347.1 (M+H⁺).

### Step 2. Lithium 4-{methyl[(2-nitrophenyl)sulfonyl]amino}-3-(methyloxy)butanoate

Lithium 4-{methyl[(2-nitrophenyl)sulfonyl]amino}-3-(methyloxy)butanoate was prepared from methyl 4-{methyl[(2-nitrophenyl)sulfonyl]amino}-3-(methyloxy)butanoate using the procedure described in Preparation 11 Step 4. ESI-MS (m/z): 355.0 (M+Na⁺).

### Preparation 13

### methyl 1,2,3-oxathiazolidine-3-carboxylate 2,2-dioxide

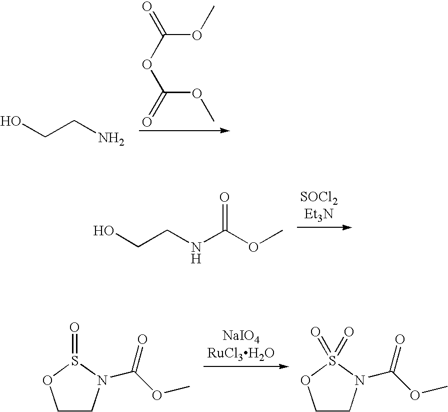

### Step 1. Methyl (2-hydroxyethyl)carbamate

To a stirred solution of 2-aminoethanol (6.11 g, 100 mmol) in dry dichloromethane (120 mL) at room temperature was added dropwise a solution of dimethyl dicarbonate (14.1 g, 105 mmol) in 20 mL. The resulting mixture was stirred for 5 hours before the solvent was removed in vacuo to afford methyl (2-hydroxyethyl)carbamate (12.1 g) as a colorless oil. ¹H NMR (400 MHz, CDCl₃) δ ppm 2.21 (broad, 1H), 3.37 (q, 2H), 3.70-3.73 [s (3H)+q (2H)], 5.20 (broad, 1H).

### Step 2. Methyl 1,2,3-oxathiazolidine-3-carboxylate 2-oxide

To a stirred suspension of methyl (2-hydroxyethyl)carbamate (100 mmol, 12.1 g) in dry dichloromethane (700 mL) at −78° C. was added triethylamine (30.4 g, 42 ml, 300 mmol) followed by thionyl chloride (17.9 g, 11 mL, 150 mmol). The resulting yellow suspension was stirred at −78° C. for 3 hours before it was quenched with methanol (3.2 g, 4 ml, 100 mmol) and warmed to room temperature. The reaction mixture was filtered and the filtrate was concentrated to remove all the dichloromethane before being re-dissolved in 900 ml of Et₂O, filtered and concentrated again. The resulting crude was purified by passing through a 15 cm silica plug eluted with 30% ethyl acetate in hexane to afford methyl 1,2,3-oxathiazolidine-3-carboxylate 2-oxide (7.905 g, 48%) as a yellowish oil. ¹H NMR (400 MHz, CDCl₃) δ ppm 3.64 (m, 1H), 3.88 (s, 3H), 3.97 (m, 1H), 4.77 (m, 1H), 5.03 (m, 1H).

### Step 3. Methyl 1,2,3-oxathiazolidine-3-carboxylate 2,2-dioxide

To a stirred solution of methyl 1,2,3-oxathiazolidine-3-carboxylate 2-oxide (7.905 g, 47.9 mmol) in acetonitrile (45 mL) at 0° C. was added RuCl₃.H₂O (54 mg, 0.24 mmol) followed by NaIO₄ (15.4 g, 71.8 mmol) and water (45 mL). The resulting mixture was allowed to warm to room temperature and stir for two hours before it was filtered. The filtrate was concentrated in vacuo and then redistributed in 800 mL MTBE and filtered again. The resulting solution was washed with water (50 mL), brine (2×100 mL), dried over Na₂SO₄, filtered and concentrated in vacuo to provide methyl 1,2,3-oxathiazolidine-3-carboxylate 2,2-dioxide (6.5 g, 75%) as an off-white solid. ¹H NMR (400 MHz, CDCl₃) δ ppm 3.95 (s, 3H), 4.14 (t, 2H), 4.69 (t, 2H).

### Preparation 14

### methyl [2-({(S)-(6-chloro-3′-ethyl-2-biphenylyl)[(2R)-2-morpholinyl]methyl}oxy)ethyl]carbamate

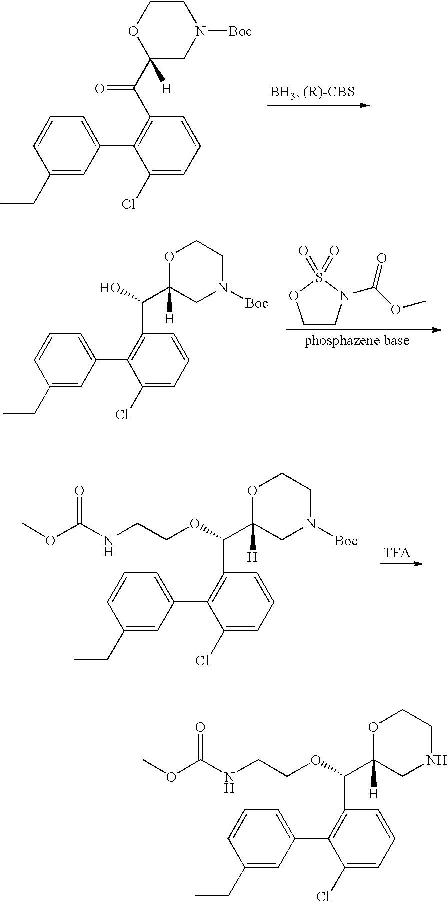

### Step 1. (1,1-dimethylethyl (2R)-2-[(S)-(6-chloro-3′-ethyl-2-biphenylyl)(hydroxy)methyl]-4-morpholinecarboxylate

To a stirred solution of 1,1-dimethylethyl (2R)-2-[(6-chloro-3′-ethyl-2-biphenylyl)carbonyl]-4-morpholinecarboxylate (2.87 g, 6.7 mmol) in TBME (75 mL) under argon at room temperature was added drop-wise borane-methyl sulfide complex (2M in toluene, 4.5 mL, 9 mmol) and (R)-2-methyl-CBS-oxazaborolidine (1 M in toluene, 0.7 mL, 0.7 mmol). The resulting solution was heated at 40° C. for 4 hours at which time TLC analysis showed complete consumption of the ketone. The reaction was quenched with 2 mL of water added slowly and then partitioned between 700 mL Et₂O and 150 mL brine. The organic layer was washed with brine (1×50 mL), dried over Na₂SO₄, filtered, and concentrated under reduced pressure. The crude was purified by flash chromatography to provide (1,1-dimethylethyl (2R)-2-[(S)-(6-chloro-3′-ethyl-2-biphenylyl)(hydroxy)methyl]-4-morpholinecarboxylate (1.392 g, 48%, the less polar diastereomer).

### Step 2. 1,1-dimethylethyl (2R)-2-{(S)-(6-chloro-3′-ethyl-2-biphenylyl)[(2-{[(methyloxy)carbonyl]amino}ethyl)oxy]methyl}-4-morpholinecarboxylate

To a stirred solution of (1,1-dimethylethyl (2R)-2-[(S)-(6-chloro-3′-ethyl-2-biphenylyl)(hydroxy)methyl]-4-morpholinecarboxylate (0.432 g, 1 mmol) in 6 mL of dry DMF at room temperature was added phosphazene base P₄-t-Bu (1.0M in n-hexane, 2 ml, 2 mmol). The resulting mixture was stirred for 10 minutes under argon before a solution of methyl 1,2,3-oxathiazolidine-3-carboxylate 2,2-dioxide (0.362 g, 2 mmol) in 2 mL dry DMF was added. The resulting solution was stirred at room temperature overnight (18 hours). The crude reaction mixture was purified via reverse phase HPLC to provide 1,1-dimethylethyl (2R)-2-{(S)-(6-chloro-3′-ethyl-2-biphenylyl)[(2-{[(methyloxy)carbonyl]amino}ethyl)oxy]methyl}-4-morpholinecarboxylate as colorless oil. MS (E/Z): 533.4 (M+H⁺).

### Step 3. Methyl [2-({(S)-(6-chloro-3′-ethyl-2-biphenylyl)[(2R)-2-morpholinyl]methyl}oxy)ethyl]carbamate

To a stirred solution of 1,1-dimethylethyl (2R)-2-{(S)-(6-chloro-3′-ethyl-2-biphenylyl)[(2-{[(methyloxy)carbonyl]amino}ethyl)oxy]methyl}-4-morpholinecarboxylate in DCM (4 mL) at room temperature was added TFA (4 mL). The resulting mixture was stirred at room temperature for 1.5 h. The crude was concentrated under reduced pressure and then partitioned between 450 mL DCM and 50 mL saturated Na₂CO₃ solution. The organic layer was washed with brine, dried over Na₂SO₄, filtered and concentrated to provide methyl [2-({(S)-(6-chloro-3′-ethyl-2-biphenylyl)[(2R)-2-morpholinyl]methyl}oxy)ethyl]carbamate (407 mg, 68% for steps 2 and 3). MS (E/Z): 433.0 (M+H⁺).

### Preparation 15

### Methyl [2-({(R)-(6-chloro-3′-ethyl-2-biphenylyl)[(3R)-3-piperidinyl]methyl}oxy)ethyl]carbamate

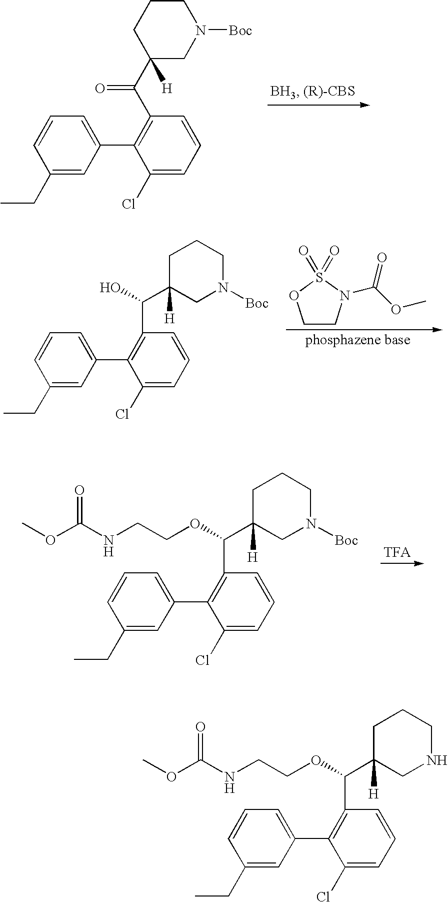

### Step 1. 1,1-dimethylethyl (3R)-3-[(R)-(6-chloro-3′-ethyl-2-biphenylyl)(hydroxy)methyl]-1-piperidinecarboxylate

To a stirred solution of 1,1-dimethylethyl (3R)-3-[(6-chloro-3′-ethyl-2-biphenylyl)carbonyl]-1-piperidinecarboxylate (1.6 g, 3.74 mmol) in TBME (60 mL) under argon at room temperature was added drop-wise simultaneously borane-methyl sulfide complex (2M in toluene, 2.5 ml, 5 mmol) and (R)-2-methyl-CBS-oxazaborolidine (1 M in toluene, 0.4 ml, 0.4 mmol). The resulting solution was heated at 40° C. for 3 h at which time TLC showed that the reaction was complete. The reaction was quenched with 1 mL water added slowly and then partitioned between 600 mL Et₂O and 50 mL water. The organic layer was washed with brine (1×50 mL), dried over Na₂SO₄, filtered, and concentrated under reduced pressure. The crude was purified by flash chromatography to provide 1,1-dimethylethyl (3R)-3-[(R)-(6-chloro-3′-ethyl-2-biphenylyl)(hydroxy)methyl]-1-piperidinecarboxylate (1.2 g, 74.6%, less polar diastereomer). MS (E/Z): 430.4 (M+H⁺)

### Step 2. 1,1-dimethylethyl (3R)-3-{(R)-(6-chloro-3′-ethyl-2-biphenylyl)[(2-{[(methyloxy)carbonyl]amino}ethyl)oxy]methyl}-1-piperidinecarboxylate

To a stirred solution of 1,1-dimethylethyl (3R)-3-[(R)-(6-chloro-3′-ethyl-2-biphenylyl)(hydroxy)methyl]-1-piperidinecarboxylate (0.354 g, 0.82 mmol) in 8 mL of dry DMF at room temperature was added phosphazene base P₄-t-Bu (1.0M in n-hexane, 1.64 mL, 1.64 mmol). The resulting mixture was stirred for 10 minutes under argon before a solution of methyl 1,2,3-oxathiazolidine-3-carboxylate 2,2-dioxide (0.298 g, 1.64 mmol) in 2 ml dry DMF was added. The resulting solution was stirred at room temperature overnight (18 hours) before it was quenched with 10 mL saturated NH₄Cl solution. The product was extracted with 300 mL EtOAc and the organic layer was washed with saturated NH₄Cl solution (3×50 mL), HCl (2M, 3×50 mL), brine, and dried over Na₂SO₄, filtered and concentrated in vacuo to provide 1,1-dimethylethyl (3R)-3-{(R)-(6-chloro-3′-ethyl-2-biphenylyl)[(2-{[(methyloxy)carbonyl]amino}ethyl)oxy]methyl}-1-piperidinecarboxylate (452 mg), which was directly used in the next step without further purification. MS (E/Z): 531.4 (M+H⁺)

### Step 3. Methyl [2-({(R)-(6-chloro-3′-ethyl-2-biphenylyl)[(3R)-3-piperidinyl]methyl}oxy)ethyl]carbamate

To a solution of 1,1-dimethylethyl (3R)-3-{(R)-(6-chloro-3′-ethyl-2-biphenylyl)[(2-{[(methyloxy)carbonyl]amino}ethyl)oxy]methyl}-1-piperidinecarboxylate (0.452 g, 0.82 mmol) in DCM (14 mL) at room temperature was added TFA (4 mL). The resulting solution was stirred for 1.5 h. At this time the solvent was removed under reduced pressure and the crude material partitioned between 200 mL DCM and 50 mL 5% Na₂CO₃ solution. The organic layer was washed with water (50 mL), brine, dried over Na₂SO₄, filtered, and concentrated under reduced pressure to provide methyl [2-({(R)-(6-chloro-3′-ethyl-2-biphenylyl)[(3R)-3-piperidinyl]methyl}oxy)ethyl]carbamate (0.35 g, 99%). MS (E/Z): 431.5 (M+H⁺).

### Preparation 16

### 1,1-dimethylethyl((1S)-2-amino-1-{[4-(methyloxy)phenyl]methyl}ethyl)methylcarbamate

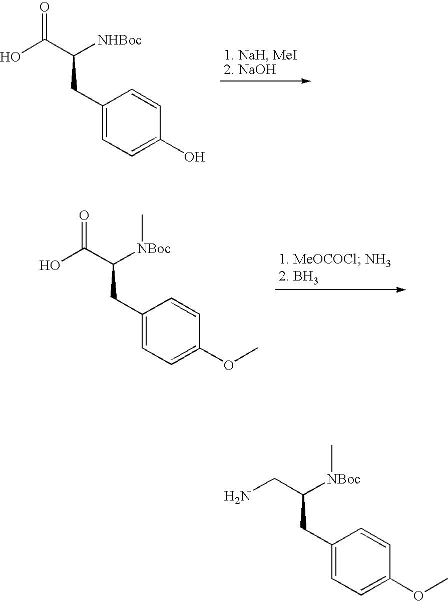

### Step 1. Methyl N-{[(1,1-dimethylethyl)oxy]carbonyl}-N,O-dimethyl-L-tyrosinate

A solution of N-{[(1,1-dimethylethyl)oxy]carbonyl}-L-tyrosine (1.34 g, 5.0 mmol) in DMF at 25° C. was treated with NaH (0.82 g of 60% in oil, 20.0 mmol) and the mixture was stirred for 20 minutes before being treated with iodomethane (1.3 mL, 20.0 mmol). The mixture was stirred overnight before being quenched with the addition of saturated aqueous NH₄Cl and extracted with EtOAc. The organic extract was washed with saturated aqueous NH₄Cl, dried (Na₂SO₄), concentrated under reduced pressure, and subjected to flash chromatography to give methyl N-{[(1,1-dimethylethyl)oxy]carbonyl}-N,O-dimethyl-L-tyrosinate as a brown oil (1.53 g, 94%). ESI-MS (m/z): 324.6 (M+H⁺).

### Step 2. N-{[(1,1-dimethylethyl)oxy]carbonyl}-N,O-dimethyl-L-tyrosine

A solution of methyl N-{[(1,1-dimethylethyl)oxy]carbonyl}-N,O-dimethyl-L-tyrosinate (1.50 g, 4.6 mmol) in 44 mL of THF/MeOH/H₂O (4:1:1) at 25° C. was treated with a solution of aqueous NaOH (6 mL of 2.5 N, 15 mmol) and the mixture was stirred for 6 hours before being concentrated under reduced pressure. The residue was diluted with water (60 mL), acidified by addition of aqueous 3N HCl, and extracted with EtOAc (3×50 mL). The organic extracts were concentrated under reduced pressure to give N-{[(1,1-dimethylethyl)oxy]carbonyl}-N,O-dimethyl-L-tyrosine as an oily solid (1.36 g, 91%). ESI-MS (m/z): 310.0 (M+H⁺).

### Step 3. N-{[(1,1-dimethylethyl)oxy]carbonyl}-N,O-dimethyl-L-tyrosinamide

A solution of N-{[(1,1-dimethylethyl)oxy]carbonyl}-N,O-dimethyl-L-tyrosine (1.36 g, 4.4 mmol) in THF (30 mL) at 25° C. was treated with ethyl chloroformate (0.94 ml, 8.8 mmol) and Et₃N (1.2 mL, 8.8 mmol) and the mixture was stirred for 20 minutes before being treated with a solution NH₄OH (30% in water). The resulting mixture was stirred for 30 minutes before being treated with brine (50 mL) and extracted with EtOAc (400 mL). The organic extract was washed with brine, dried (Na₂SO₄), and concentrated under reduced pressure to give N-{[(1,1-dimethylethyl)oxy]carbonyl}-N,O-dimethyl-L-tyrosinamide as a white solid (0.4 g, 29%). ESI-MS (m/z): 308.9 (M+H⁺).

### Step 4. 1,1-dimethylethyl((1S)-2-amino-1-{[4-(methyloxy)phenyl]methyl}ethyl)methylcarbamate

A solution of N-{[(1,1-dimethylethyl)oxy]carbonyl}-N,O-dimethyl-L-tyrosinamide (400 mg, 1.3 mmol) in THF (20 mL) at 25° C. was treated with a solution of borane-methyl sulfide (2.6 mL of 2M, 5.2 mmol) and the mixture was stirred overnight before being quenched with the addition of aqueous KHSO₄ (1 mL) and brine (50 mL) and extracted with EtOAc. The organic extract was washed with brine, dried (Na₂SO₄), concentrated under reduced pressure, and subjected to SCX purification (NH₃/MeOH) to give 1,1-dimethylethyl((1S)-2-amino-1-{[4-(methyloxy)phenyl]methyl}ethyl)methylcarbamate as a solid (70 mg, 18%). ESI-MS (m/z): 295.1 (M+H⁺).

The following compound was prepared using procedures analogous to those described above:

1,1-dimethylethyl {(1S)-1-(aminomethyl)-3-[4-(methyloxy)phenyl]propyl}methylcarbamate by using (2S)-2-({[(1,1-dimethylethyl)oxy]carbonyl}amino)-4-(4-hydroxyphenyl)butanoic acid instead of N-{[(1,1-dimethylethyl)oxy]carbonyl}-L-tyrosine in Step 1.

The following procedures describe preparation of compounds of Formula I.

### Example 1

### methyl (4-(3′-ethyl-6-fluoro-2-biphenylyl)-4-hydroxy-4-{(3R)-1-[4-(methylamino)butanoyl]-3-piperidinyl}butyl)carbamate (#28)

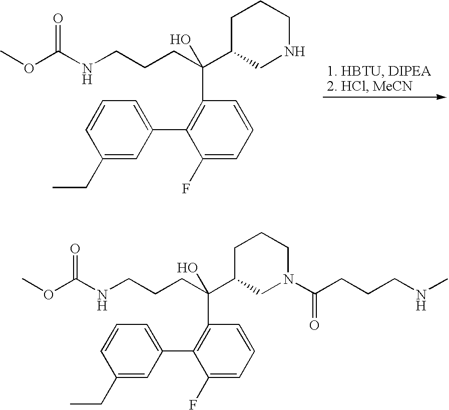

A solution of methyl [4-(3′-ethyl-6-fluoro-2-biphenylyl)-4-hydroxy-4-(3-piperidinyl)butyl]carbamate (0.04 g, 0.093 mmol) in 1 mL of MeCN at 25° C. was treated with 4-[{[(1,1-dimethylethyl)oxy]carbonyl}(methyl)amino]butanoic acid (0.024 mg, 0.11 mmol), HBTU (0.042 g, 0.011 mmol) and diisopropylethylamine (0.05 mL, 0.28 mmol) and stirred for 3 h before being filtered and subjected to reverse phase HPLC. The resulting solid was dissolved in 1 mL of MeCN, treated with 1 mL of aqueous 2N HCl, and the mixture was stirred at 25° C. overnight before being concentrated under reduced pressure to provide methyl (4-(3′-ethyl-6-fluoro-2-biphenylyl)-4-hydroxy-4-{(3R)-1-[4-(methylamino)butanoyl]-3-piperidinyl}butyl)carbamate as a white solid.

The following compounds were prepared following procedures analogous to those described above using the appropriate amine intermediate and the indicated acid:

| | Product | Acid used in Step 1 |
|---|---|---|
| #1 | 1-(6-chloro-3′-ethyl-2-biphenylyl)-1- | N-{[(1,1-dimethylethyl)oxy]carbonyl}- |
| | [(3R)-1-(2-methylalanyl)-3-piperidinyl]-5- | 2- |
| | (methyloxy)-1-pentanol | methylalanine |
| #2 | 2-({(3R)-3-[1-(6-chloro-3′-ethyl-2- | 3-hydroxy-2- |
| | biphenylyl)-1-hydroxy-5- | (hydroxymethyl)-2- |
| | (methyloxy)pentyl]-1- | methylpropanoic acid |
| | piperidinyl}carbonyl)-2-methyl-1,3- | |
| | propanediol | |
| #4 | 1-[1-(6-aminohexanoyl)-4-piperidinyl]-1- | 6-({[(1,1-dimethylethyl)oxy]carbonyl}amino)hexanoic |
| | (3′-ethyl-6-fluoro-2-biphenylyl)-5- | acid |
| | (methyloxy)-1-pentanol | |
| #5 | methyl (4-[3-fluoro-2-(3- | 4-[{[(1,1-dimethylethyl)oxy]carbonyl}(methyl)amino]butanoic |
| | quinolinyl)phenyl]-4-hydroxy-4-{(3R)-1- | acid |
| | [4-(methylamino)butanoyl]-3- | |
| | piperidinyl}butyl)carbamate | |
| #6 | methyl (4-[3-chloro-2-(5-methyl-2- | 4-[{[(1,1-dimethylethyl)oxy]carbonyl}(methyl)amino]butanoic |
| | furanyl)phenyl]-4-hydroxy-4-{(3R)-1-[4- | acid |
| | (methylamino)butanoyl]-3- | |
| | piperidinyl}butyl)carbamate | |
| #7 | (1R)-1-[(2R)-4-(4-aminobutanoyl)-2- | 4-({[(1,1-dimethylethyl)oxy]carbonyl}amino)butanoic |
| | morpholinyl]-1-(4′,6-difluoro-3′-methyl-2- | acid |
| | biphenylyl)-5-(methyloxy)-1-pentanol | |
| #8 | methyl [4-{(3R)-1-[4-amino-3-(4- | 4-(tert- |
| | chlorophenyl)butanoyl]-3-piperidinyl}-4- | butoxycarbonylamino)-3-(4- |
| | (3′-ethyl-6-fluoro-2-biphenylyl)-4- | chlorophenyl)butanoic acid |
| | hydroxybutyl]carbamate | |
| #9 | methyl (4-(6-chloro-3′-fluoro-5′-methyl-2- | 4-[{[(1,1-dimethylethyl)oxy]carbonyl}(methyl)amino]butanoic |
| | biphenylyl)-4-hydroxy-4-{(3R)-1-[4- | acid |
| | (methylamino)butanoyl]-3- | |
| | piperidinyl}butyl)carbamate | |
| #10 | methyl (4-(3′,6-difluoro-5′-methyl-2- | 4-[{[(1,1-dimethylethyl)oxy]carbonyl}(methyl)amino]butanoic |
| | biphenylyl)-4-hydroxy-4-{(3R)-1-[4- | acid |
| | (methylamino)butanoyl]-3- | |
| | piperidinyl}butyl)carbamate | |
| #11 | methyl (4-hydroxy-4-{(3R)-1-[4- | 4-[{[(1,1-dimethylethyl)oxy]carbonyl}(methyl)amino]butanoic |
| | (methylamino)butanoyl]-3-piperidinyl}-4- | acid |
| | {2-[(2-methylphenyl)oxy]phenyl}butyl)- | |
| | carbamate | |
| #12 | methyl [4-[(3R)-1-β-alanyl-3-piperidinyl]- | N-{[(1,1-dimethylethyl)oxy]carbonyl}- |
| | 4-(3′-ethyl-6-fluoro-2-biphenylyl)-4- | β-alanine |
| | hydroxybutyl]carbamate | |
| #13 | methyl [4-[(3R)-1(4-aminobutanoyl)-3- | 4-({[(1,1-dimethylethyl)oxy]carbonyl}amino)butanoic |
| | piperidinyl]-4-(3′-ethyl-6-fluoro-2- | acid |
| | biphenylyl)-4-hydroxybutyl]carbamate | |
| #14 | methyl [4-[(3R)-1-(5-aminopentanoyl)-3- | 5-({[(1,1-dimethylethyl)oxy]carbonyl}amino)pentanoic |
| | piperidinyl]-4-(3′-ethyl-6-fluoro-2- | acid |
| | biphenylyl)-4-hydroxybutyl]carbamate | |
| #15 | methyl (2S)-2-amino-5-[(3R)-3-(1-(3′- | (4S)-4-({[(1,1- |
| | ethyl-6-fluoro-2-biphenylyl)-1-hydroxy-4- | dimethylethyl)oxy]carbonyl}amino)- |
| | {[(methyloxy)carbonyl]amino}butyl)-1- | 5-(methyloxy)-5- |
| | piperidinyl]-5-oxopentanoate | oxopentanoic acid |
| #16 | methyl ((4S)-4-(6-chloro-3′-ethyl-2- | 4-[{[(1,1-dimethylethyl)oxy]carbonyl}(methyl)amino]butanoic |
| | biphenylyl)-4-hydroxy-4-{(3R)-1-[4- | acid |
| | (methylamino)butanoyl]-3- | |
| | piperidinyl}butyl)carbamate | |
| #17 | methyl [4-{(3R)-1-[4- | 4-(dimethylamino)butanoic |
| | (dimethylamino)butanoyl]-3-piperidinyl}- | acid |
| | 4-(3′-ethyl-6-fluoro-2-biphenylyl)-4- | |
| | hydroxybutyl]carbamate | |
| #18 | methyl (4-[3-chloro-2-(2,3-dihydro-1- | 4-[{[(1,1-dimethylethyl)oxy]carbonyl}(methyl)amino]butanoic |
| | benzofuran-6-yl)phenyl]-4-hydroxy-4- | acid |
| | {(3R)-1-[4-(methylamino)butanoyl]-3- | |
| | piperidinyl}butyl)carbamate | |
| #19 | methyl [4-(3′-ethyl-6-fluoro-2- | N-{3-[{[(1,1- |
| | biphenylyl)-4-hydroxy-4-((3R)-1-{N- | dimethylethyl)oxy]carbonyl}(methyl)amino]propyl}- |
| | methyl-N-[2-(methylamino)ethyl]glycyl}- | N- |
| | 3-piperidinyl)butyl]carbamate | methylglycine |
| #22 | methyl [4-{(3R)-1-[(4S)-4-amino-5- | (4S)-4-({[(1,1- |
| | hydroxypentanoyl]-3-piperidinyl}-4-(3′- | dimethylethyl)oxy]carbonyl}amino)- |
| | ethyl-6-fluoro-2-biphenylyl)-4- | 5-hydroxypentanoic |
| | hydroxybutyl]carbamate | acid |
| #23 | methyl [4-{(3R)-1-[(4R)-4-amino-5- | (4R)-4-({[(1,1- |
| | hydroxypentanoyl]-3-piperidinyl}-4-(3′- | dimethylethyl)oxy]carbonyl}amino)- |
| | ethyl-6-fluoro-2-biphenylyl)-4- | 5- |
| | hydroxybutyl]carbamate | hydroxypentanoic acid |
| #24 | N-(4-(2′,6-difluoro-5′-methyl-2- | 4-[{[(1,1-dimethylethyl)oxy]carbonyl}(methyl)amino]butanoic |
| | biphenylyl)-4-hydroxy-4-{(3R)-1-[4- | acid |
| | (methylamino)butanoyl]-3- | |
| | piperidinyl}butyl)acetamide | |
| #25 | N-(4-(6-fluoro-3′-methyl-2-biphenylyl)-4- | 4-[{[(1,1-dimethylethyl)oxy]carbonyl}(methyl)amino]butanoic |
| | hydroxy-4-{(3R)-1-[4- | acid |
| | (methylamino)butanoyl]-3- | |
| | piperidinyl}butyl)acetamide | |
| #26 | methyl [4-[(3R)-1-(4-amino-3- | 4-({[(1,1-dimethylethyl)oxy]carbonyl}amino)- |
| | hydroxybutanoyl)-3-piperidinyl]-4-(3′- | 3- |
| | ethyl-6-fluoro-2-biphenylyl)-4- | hydroxybutanoic acid |
| | hydroxybutyl]carbamate | |
| #27 | methyl [4-{(3R)-1-[(2S)-4-amino-2- | (2S)-4-({[(1,1- |
| | hydroxybutanoyl]-3-piperidinyl}-4-(3′- | dimethylethyl)oxy]carbonyl}amino)- |
| | ethyl-6-fluoro-2-biphenylyl)-4- | 2-hydroxybutanoic |
| | hydroxybutyl]carbamate | acid |
| #28 | methyl ((4S)-4-(3′-ethyl-6-fluoro-2- | 4-[{[(1,1-dimethylethyl)oxy]carbonyl}(methyl)amino]butanoic |
| | biphenylyl)-4-hydroxy-4-{(3R)-1-[4- | acid |
| | (methylamino)butanoyl]-3- | |
| | piperidinyl}butyl)carbamate | |
| #29 | methyl [4-[(3R)-1-(6-aminohexanoyl)-3- | 6-({[(1,1-dimethylethyl)oxy]carbonyl}amino)hexanoic |
| | piperidinyl]-4-(3′-ethyl-6-fluoro-2- | acid |
| | biphenylyl)-4-hydroxybutyl]carbamate | |
| #30 | methyl [4-[(3R)-1-L-asparaginyl-3- | N²-{[(1,1-dimethylethyl)oxy]carbonyl}- |
| | piperidinyl]-4-(3′-ethyl-6-fluoro-2- | L-asparagine |
| | biphenylyl)-4-hydroxybutyl]carbamate | |
| #31 | methyl {4-(3′-ethyl-6-fluoro-2- | N-{[(1,1-dimethylethyl)oxy]carbonyl}- |
| | biphenylyl)-4-hydroxy-4-[(3R)-1-L-valyl- | L-valine |
| | 3-piperidinyl]butyl}carbamate | |
| #32 | methyl {4-(3′-ethyl-6-fluoro-2- | N-{[(1,1-dimethylethyl)oxy]carbonyl}- |
| | biphenylyl)-4-hydroxy-4-[(3R)-1-L-seryl- | L-serine |
| | 3-piperidinyl]butyl}carbamate | |
| #33 | methyl [4-[(3R)-1-(L-alanyl-L-alanyl)-3- | N-{[(1,1-dimethylethyl)oxy]carbonyl}- |
| | piperidinyl]-4-(3′-ethyl-6-fluoro-2- | L-alanyl-L- |
| | biphenylyl)-4-hydroxybutyl]carbamate | alanine |
| #34 | methyl {4-(3′-ethyl-6-fluoro-2- | N-{[(1,1-dimethylethyl)oxy]carbonyl}- |
| | biphenylyl)-4-hydroxy-4-[(3R)-1-L- | L-phenylalanine |
| | phenylalanyl-3- | |
| | piperidinyl]butyl}carbamate | |
| #35 | methyl {4-(3′-ethyl-6-fluoro-2- | N-{[(1,1-dimethylethyl)oxy]carbonyl}- |
| | biphenylyl)-4-hydroxy-4-[(3R)-1-D- | L-tryptophan |
| | tryptophyl-3-piperidinyl]butyl}carbamate | |
| #36 | methyl {4-(3′-ethyl-6-fluoro-2- | N-{[(1,1-dimethylethyl)oxy]carbonyl}glycylglycine |
| | biphenylyl)-4-[(3R)-1-(glycylglycyl)-3- | |
| | piperidinyl]-4-hydroxybutyl}carbamate | |
| #37 | methyl {4-(3′-ethyl-6-fluoro-2- | N-{[(1,1-dimethylethyl)oxy]carbonyl}- |
| | biphenylyl)-4-hydroxy-4-[(3R)-1-(2- | 2-methylalanine |
| | methylalanyl)-3- | |
| | piperidinyl]butyl}carbamate | |
| #38 | phenylmethyl (2S)-2-amino-5-[(3R)-3-(1- | (4S)-4-({[(1,1- |
| | (3′-ethyl-6-fluoro-2-biphenylyl)-1- | dimethylethyl)oxy]carbonyl}amino)- |
| | hydroxy-4- | 5-oxo-5- |
| | {[(methyloxy)carbonyl]amino}butyl)-1- | [(phenylmethyl)oxy]pentanoic |
| | piperidinyl]-5-oxopentanoate | acid |
| #39 | phenylmethyl (2S)-2-amino-4-[(3R)-3-(1- | (3S)-3-({[(1,1- |
| | (3′-ethyl-6-fluoro-2-biphenylyl)-1- | dimethylethyl)oxy]carbonyl}amino)- |
| | hydroxy-4- | 4-oxo-4- |
| | {[(methyloxy)carbonyl]amino}butyl)-1- | [(phenylmethyl)oxy]butanoic |
| | piperidinyl]-4-oxobutanoate | acid |
| #40 | methyl {4-(3′-ethyl-6-fluoro-2- | N²-{[(1,1-dimethylethyl)oxy]carbonyl}- |
| | biphenylyl)-4-[(3R)-1-L-α-glutaminyl-3- | L-α-glutamine |
| | piperidinyl]-4-hydroxybutyl}carbamate | |
| #41 | methyl {4-(3′-ethyl-6-fluoro-2- | N⁵-{[(1,1-dimethylethyl)oxy]carbonyl}- |
| | biphenylyl)-4-hydroxy-4-[(3R)-1-D- | D-ornithine |
| | ornithyl-3-piperidinyl]butyl}carbamate | |
| #47 | methyl (4-[3-chloro-2-(3- | 4-(tert- |
| | quinolinyl)phenyl]-4-hydroxy-4-{(3R)-1- | butoxycarbonyl(methyl)amino)butanoic |
| | [4-(methylamino)butanoyl]-3- | acid |
| | piperidinyl}butyl)carbamate | |
| #48 | methyl {4-hydroxy-4-{(3R)-1-[4- | 4-(tert- |
| | (methylamino)butanoyl]-3-piperidinyl}-4- | butoxycarbonyl(methyl)amino)butanoic |
| | [2′-(methyloxy)-5′-(trifluoromethyl)-2- | acid |
| | biphenylyl]butyl}carbamate | |
| #49 | methyl {4-(3′-ethyl-6-fluoro-2- | 2-(tert- |
| | biphenylyl)-4-[(3R)-1-glycyl-3- | butoxycarbonylamino)acetic |
| | piperidinyl]-4-hydroxybutyl}carbamate | acid |
| #50 | methyl [4-{(3R)-1-[(3S)-4-amino-3- | (S)-4-(tert- |
| | hydroxybutanoyl]-3-piperidinyl}-4-(3′- | butoxycarbonylamino)-3- |
| | ethyl-6-fluoro-2-biphenylyl)-4- | hydroxybutanoic acid |
| | hydroxybutyl]carbamate | |
| #51 | N-(4-(2′,6-difluoro-5′-methyl-2- | 4-(tert- |
| | biphenylyl)-4-hydroxy-4-{(3R)-1-[4- | butoxycarbonyl(methyl)amino)butanoic |
| | (methylamino)butanoyl]-3- | acid |
| | piperidinyl}butyl)-2,2,2- | |
| | trifluoroacetamide | |
| #52 | N-[(4S)-4-{(3R)-1-[4-amino-3-(4- | 4-(tert- |
| | chlorophenyl)butanoyl]-3-piperidinyl}-4- | butoxycarbonylamino)-3-(4- |
| | (2′,6-difluoro-5′-methyl-2-biphenylyl)-4- | chlorophenyl)butanoic acid |
| | hydroxybutyl]-2,2,2-trifluoroacetamide | |
| #53 | N-[4-{(3R)-1-[(3S)-4-amino-3- | (S)-4-(tert- |
| | hydroxybutanoyl]-3-piperidinyl}-4-(2′,6- | butoxycarbonylamino)-3- |
| | difluoro-5′-methyl-2-biphenylyl)-4- | hydroxybutanoic acid |
| | hydroxybutyl]-2,2,2-trifluoroacetamide | |
| #54 | N-(4-(3′-ethyl-6-fluoro-2-biphenylyl)-4- | 4-(tert- |
| | hydroxy-4-{(3R)-1-[4- | butoxycarbonyl(methyl)amino)butanoic |
| | (methylamino)butanoyl]-3- | acid |
| | piperidinyl}butyl)-2,2,2- | |
| | trifluoroacetamide | |
| #55 | N-[4-{(3R)-1-[(3S)-4-amino-3- | (S)-4-(tert- |
| | hydroxybutanoyl]-3-piperidinyl}-4-(3′- | butoxycarbonylamino)-3- |
| | ethyl-6-fluoro-2-biphenylyl)-4- | hydroxybutanoic acid |
| | hydroxybutyl]-2,2,2-trifluoroacetamide | |
| #56 | methyl [4-[(3R)-1-(4- | 4-guanidinobutanoic acid |
| | {[amino(imino)methyl]amino}butanoyl)- | |
| | 3-piperidinyl]-4-(3′-ethyl-6-fluoro-2- | |
| | biphenylyl)-4-hydroxybutyl]carbamate | |
| #57 | methyl [4-{(3R)-1-[4-amino-3-(4- | 4-(tert- |
| | chlorophenyl)butanoyl]-3-piperidinyl}-4- | butoxycarbonylamino)-3-(4- |
| | (6-chloro-3′-ethyl-2-biphenylyl)-4- | chlorophenyl)butanoic acid |
| | hydroxybutyl]carbamate | |
| #58 | methyl [4-{(3R)-1-[(3S)-4-amino-3- | (S)-4-(tert- |
| | hydroxybutanoyl]-3-piperidinyl}-4-(6- | butoxycarbonylamino)-3- |
| | chloro-3′-ethyl-2-biphenylyl)-4- | hydroxybutanoic acid |
| | hydroxybutyl]carbamate | |
| #62 | methyl (4-[6-fluoro-3′-(1-methylethyl)-2- | 4-(tert- |
| | biphenylyl]-4-hydroxy-4-{(3R)-1-[4- | butoxycarbonyl(methyl)amino)butanoic |
| | (methylamino)butanoyl]-3- | acid |
| | piperidinyl}butyl)carbamate | |
| #63 | methyl {2-[((S)-(6-chloro-3′-ethyl-2- | 4-(tert- |
| | biphenylyl){(2R)-4-[4- | butoxycarbonyl(methyl)amino)butanoic |
| | (methylamino)butanoyl]-2- | acid |
| | morpholinyl}methyl)oxy]ethyl}carbamate | |
| #64 | methyl {2-[((R)-(6-chloro-3′-ethyl-2- | 4-(tert- |
| | biphenylyl){(3R)-1-[4- | butoxycarbonyl(methyl)amino)butanoic |
| | (methylamino)butanoyl]-3- | acid |
| | piperidinyl}methyl)oxy]ethyl}carbamate | |

### Example 2

### methyl [4-(3′-ethyl-6-fluoro-2-biphenylyl)-4-hydroxy-4-((3R)-1-{-4-[(2-hydroxyethyl)amino]butanoyl}-3-piperidinyl)butyl]carbamate (#20)

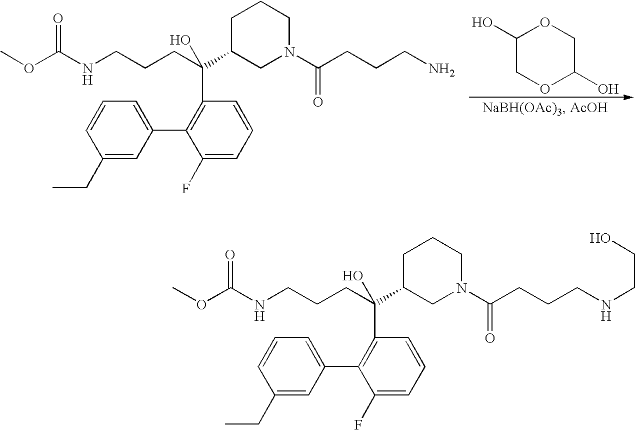

A solution of methyl [4-[(3R)-1-(4-aminobutanoyl)-3-piperidinyl]-4-(3′-ethyl-6-fluoro-2-biphenylyl)-4-hydroxybutyl]carbamate (0.027 g, 0.05 mmol) in 2 mL of CH₂Cl₂ at 25° C. was treated with acetic acid (0.003 mL, 0.05 mmol), glycolaldehyde dimer (3 mg, 0.025 mmol), and sodium triacetoxyborohydride (20 mg, 0.10 mmol) and the mixture was stirred overnight before being quenched with the addition of 0.5 mL of aqueous 2N NaOH and extracted with 10% MeOH/CH₂Cl₂ (3×2 mL). The combined organic extract was dried (MgSO₄), concentrated under reduced pressure, and subjected to reverse phase HPLC to provide methyl [4-(3′-ethyl-6-fluoro-2-biphenylyl)-4-hydroxy-4-((3R)-1-{4-[(2-hydroxyethyl)amino]butanoyl}-3-piperidinyl)butyl]carbamate.

The following compound was prepared following procedures analogous to those described above:
#3 1-(6-chloro-3′-ethyl-2-biphenylyl)-1-{(3R)-1-[N-(2-hydroxyethyl)-2-methylalanyl]-3-piperidinyl}-5-(methyloxy)-1-pentanol

### Example 3

### (2S)-2-amino-5-[(3R)-3-(1-(3′-ethyl-6-fluoro-2-biphenylyl)-1-hydroxy-4-{[(methyloxy)carbonyl]amino}butyl)-1-piperidinyl]-5-oxopentanoic acid (#21)

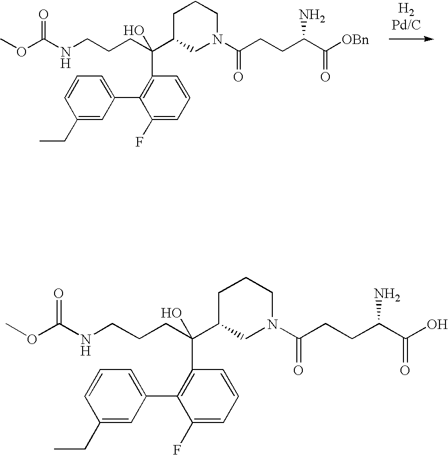

A solution of phenylmethyl (2S)-2-amino-5-[(3R)-3-(1-(3′-ethyl-6-fluoro-2-biphenylyl)-1-hydroxy-4-{[(methyloxy)carbonyl]amino}butyl)-1-piperidinyl]-5-oxopentanoate (0.078 g, 0.12 mmol) in EtOH at 25° C. was treated with 0.05 g of 10% Pd/C and stirred under an atmosphere of hydrogen overnight. The mixture was filtered through Celite®, concentrated under reduced pressure, and subjected to reverse phase HPLC to provide (2S)-2-amino-5-[(3R)-3-(1-(3′-ethyl-6-fluoro-2-biphenylyl)-1-hydroxy-4-{[(methyloxy)carbonyl]amino}butyl)-1-piperidinyl]-5-oxopentanoic acid as a white solid.

### Example 4

### (3R)-1-((3R)-3-(1-(3-chlorophenyl)-1-hydroxy-5-methoxypentyl)piperidin-1-yl)-4-cyclohexyl-3-((methylamino)methyl)butan-1-one (#42)

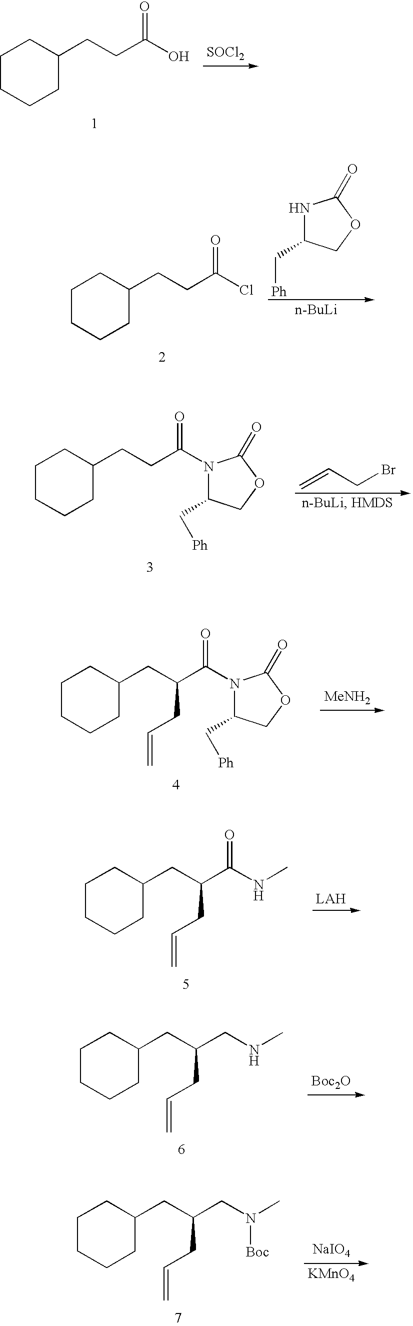

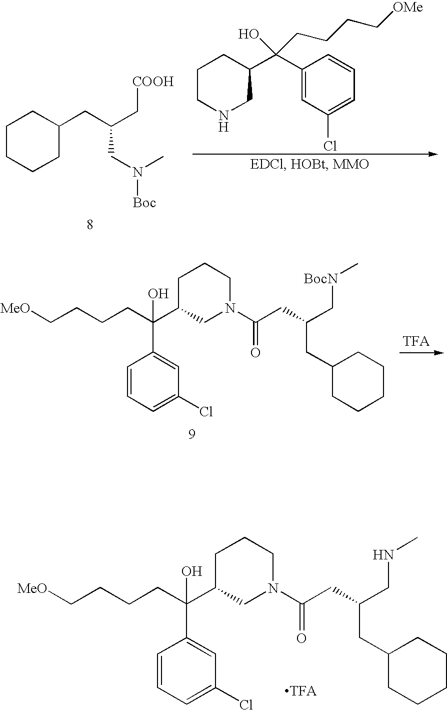

### Step 1. 3-cyclohexylpropanoyl chloride

To a solution of 3-cyclohexyl-propionic acid (15.6 g, 100 mmol) in anhydrous dichloromethane (200 mL) was added dropwise thionyl chloride (23.8 g, 200 mmol) at 0° C., and the resulting mixture was heated at reflux overnight. Volatiles were evaporated in vacuo to give 3-cyclohexyl-propionyl chloride (17.8 g, 100%), which was used to the next step without further purification.

### Step 2. (S)-4-benzyl-3-(3-cyclohexylpropanoyl)oxazolidin-2-one

To a solution of (4S)-4-benzyl-oxazolidin-2-one (17.7 g, 100 mol) in anhydrous THF (150 mL) was added dropwise n-BuLi (2.5 M/L, 40 mL, 100 mol) at −78° C. under N₂. After stirring for additional 2 hours at −78° C., a solution of 3-cyclohexyl-propionyl chloride (17.8 g, 100 mmol) obtained from above step in THF (50 mL) was added dropwise at −78° C. for about 30 min. After the addition, the reaction was stirred at −78° C. for 2 hours before allowed to warm to room temperature, then stirred at room temperature overnight. The reaction was quenched by the addition of 10% aqueous NH₄Cl solution (50 mL), the organic layer was separated and the aqueous phase extracted with ethyl acetate (3×50 mL). The combined organic layers were washed with brine (100 mL), dried, evaporated, then purified by column chromatography to give (S)-4-benzyl-3-(3-cyclohexyl-propionyl)-oxazolidin-2-one as a pale solid (25 g, 79% in two steps). ¹H NMR (CDCl₃, 400 MHz) δ ppm 7.36-7.20 (m, 5H), 4.69 (m, 1H), 4.20 (m, 2H), 3.37-3.28 (dd, J=13.2 Hz, 3.2 Hz, 1H), 2.95 (m, 2H), 2.79-2.73 (dd, J=13.2 Hz, 9.6 Hz, 1H), 1.72-1.51 (m, 13H). MS (m/z): 316 (M+H)⁺.

### Step 3. (S)-4-benzyl-3-((S)-2-(cyclohexylmethyl)pent-4-enoyl)oxazolidin-2-one

To a stirred solution of HMDS (1.61 g, 10 mmol) in anhydrous THF (20 mL) was added dropwise n-BuLi (2.5 M/L, 4 mL, 10 mmol) at −78° C. under N₂ during 30 min, after stirring for an additional 30 min, a solution of 4-benzyl-3-(3-cyclohexyl-propionyl)-oxazolidin-2-one (3.15 g, 10 mmol) in THF (10 mL) was added dropwise to the reaction mixture, then stirred for an additional 2 hours at −78° C. Then a solution of allyl bromide (1.21 g, 10 mmol) in THF (10 mL) was added dropwise, and the temperature was raised from −78° C. to 0° C. during 2 h. After stirring for 18 h at room temperature, the reaction was quenched by addition of 10% aqueous NH₄Cl solution (20 mL). The aqueous layer was extracted with ethyl acetate (3×25 mL), and the combined organic layers were washed with brine, dried with MgSO₄, and evaporated, then the residue was purified by flash chromatography on silica gel to give (S)-4-benzyl-3-((S)-2-(cyclohexylmethyl)pent-4-enoyl)oxazolidin-2-one (1.8 g, 51%). ¹H NMR (CDCl₃, 400 MHz) δ ppm 7.36-7.20 (m, 5H), 5.86 (m, 1H), 5.10-5.03 (m, 2H), 4.68 (m, 1H), 4.17 (m, 2H), 4.05 (m, 1H), 3.32-3.29 (dd, J=13.6 Hz, 3.2 Hz, 1H), 2.67-2.61 (dd, J=13.2 Hz, 10.0 Hz, 1H), 2.46-2.28 (m, 2H), 1.72-0.80 (m, 13H). MS (m/z): 356 (M+H)⁺.

### Step 4. (S)-2-(cyclohexylmethyl)-N-methylpent-4-enamide

A solution of (S)-4-benzyl-3-((S)-2-(cyclohexylmethyl)pent-4-enoyl)oxazolidin-2-one (1.8 g, 5.1 mmol) in methylamine ethanol solution (20 mL) was refluxed overnight. Evaporated, purified by column chromatography on silica gel to give (S)-2-(cyclohexylmethyl)-N-methylpent-4-enamide (450 mg, 42%). ¹H NMR (CDCl₃, 400 MHz) δ ppm 5.75 (m, 1H), 5.43 (br, 1H), 5.07-4.97 (m, 2H), 2.80 (d, J=4.8 Hz, 3H), 2.33-2.10 (m, 3H), 1.73-0.78 (m, 13H). MS (m/z): 210 (M+H)⁺.

### Step 5. (S)-2-(cyclohexylmethyl)-N-methylpent-4-en-1-amine

To a suspension solution of LiAlH₄ (123 mg, 3.23 mol) in anhydrous THF (10 mL) was added dropwise a solution of (S)-2-(cyclohexylmethyl)-N-methylpent-4-enamide (450 g, 2.15 mol) in anhydrous THF (500 mL) at 0° C. under N₂ during 5 min, then the reaction mixture was refluxed for 6 hours. After cooling to 0° C., water (0.13 mL) was added dropwise, followed by 10% aqueous NaOH solution (0.13 mL). The precipitated was filtered off and the filtrate was evaporated, then the residue was partitioned between ethyl acetate (20 mL) and water (20 mL), and the aqueous layer was extracted with ethyl acetate (3×20 mL). The combined organic layers were washed with brine, and dried over Na₂SO₄, filtered and evaporated to give (S)-2-(cyclohexylmethyl)-N-methylpent-4-en-1-amine (380 mg, 91%). ¹H NMR (CDCl₃, 400 MHz) δ ppm 5.81 (m, 1H), 2.48 (m, 2H), 2.43 (s, 3H), 2.1 (t, J=6.4 Hz, 2H), 1.77-0.84 (m, 16H). MS (m/z): 196 (M+H)⁺.

### Step 6. (S)-tert-butyl 2-(cyclohexylmethyl)pent-4-enyl(methyl)carbamate

A mixture of solution of (S)-2-(cyclohexylmethyl)-N-methylpent-4-en-1-amine (380 mg, 1.95 mmol) and Et₃N (220 mg, 2.2 mmol) in methanol (10 mL) was added dropwise a solution of Boc₂O (480 mg, 2.2 mmol) in methanol (5 mL) at 0° C., then the resulting mixture was stirred at room temperature for 2 hours. Volatiles were evaporated and the residue was purified by column chromatography on silica gel to give (S)-tert-butyl 2-(cyclohexylmethyl)pent-4-enyl(methyl)carbamate (500 mg, 87%). ¹H NMR (CDCl₃, 400 MHz) δ ppm 5.75 (m, 1H), 5.05-4.95 (m, 2H), 3.10 (m, 2H), 2.80 (m, 3H), 2.00 (t, J=6.4 Hz, 2H), 1.86 (m, 1H), 1.75 (m, 4H), 1.45 (s, 9H), 1.40-0.75 (m, 9H). MS (m/z): 296 (M+H)⁺.

### Step 7. (R)-4-(tert-butoxycarbonyl(methyl)amino)-3-(cyclohexylmethyl)butanoic acid

To a solution of (S)-tert-butyl 2-(cyclohexylmethyl)pent-4-enyl(methyl)carbamate (295 mg, 1.0 mol) in acetone (10 mL) was added a solution of KMnO₄ (88 mg, 0.56 mmol) and NaIO₄ (759 mg, 3.54 mmol) in water (10 mL) at room temperature and the mixture was stirred for 4 h at room temperature. The precipitate was removed by filtration and the acetone was removed under reduced pressure. The resulting mixture was basified to pH=13 by addition of 1M aqueous sodium hydroxide and then washed with ether (10 mL×3). The aqueous phase was acidified to pH=1 by addition of aqueous 1N HCl and then extracted with CH₂Cl₂ (20 mL×3). The organic layers were combined, washed with brine (20 mL), dried over Na₂SO₄ and then concentrated in vacuo to give (R)-4-(tert-butoxycarbonyl(methyl)amino)-3-(cyclohexylmethyl)butanoic acid (240 mg, 77%). ¹H NMR (CDCl₃, 400 MHz) δ ppm 3.12 (m, 1H), 2.85 (s, 3H), 2.25 (m, 2H), 1.70 (m, 5H), 1.48 (s, 9H), 1.27-0.80 (m, 10H). MS (m/z): 314 (M+H)⁺.

### Step 8. tert-butyl (2R)-4-((3R)-3-(1-(3-chlorophenyl)-1-hydroxy-5-methoxypentyl)piperidin-1-yl)-2-(cyclohexylmethyl)-4-oxobutyl(methyl)carbamate

A mixture of (R)-4-(tert-butoxycarbonyl(methyl)amino)-3-(cyclohexylmethyl)butanoic acid (120 mg, 0.38 mmol), EDCI (110 mg, 0.57 mmol), HOBT (77 mg, 0.57 mmol), NMM (115 mg, 1.14 mmol) and 1-(3-chlorophenyl)-5-methoxy-1-((3R)-piperidin-3-yl)pentan-1-ol (119 mg, 0.38 mmol) in anhydrous dichloromethane (10 mL) was stirred at room temperature overnight. Washed by water (10 mL), dried, evaporated, then purified by preparative TLC to give tert-butyl (2R)-4-((3R)-3-(1-(3-chlorophenyl)-1-hydroxy-5-methoxypentyl)piperidin-1-yl)-2-(cyclohexylmethyl)-4-oxobutyl(methyl)carbamate (125 mg, 54%). MS (m/z): 607 (M+H)⁺.

### Step 9. (3R)-1-((3R)-3-(1-(3-chlorophenyl)-1-hydroxy-5-methoxypentyl)piperidin-1-yl)-4-cyclohexyl-3-((methylamino)methyl)butan-1-one

To a solution of tert-butyl (2R)-4-((3R)-3-(1-(3-chlorophenyl)-1-hydroxy-5-methoxypentyl)piperidin-1-yl)-2-(cyclohexylmethyl)-4-oxobutyl(methyl)carbamate (65 mg, 0.11 mol) in dichloromethane (3 mL) at 0° C. was added TFA (1 mL) and the resulting mixture was stirred at room temperature for 30 min. Volatiles were evaporated, and the residue was purified by preparative HPLC to give the title compound (25 mg, 38%). ¹H NMR (MeOD, 400 MHz) δ ppm 7.46-7.21 (m, 1H), 4.50-3.80 (m, 2H), 3.36-3.31 (m, 5H), 3.00-2.30 (m, 10H), 1.93 (m, 2H), 1.70-0.95 (m, 23H). MS (m/z): 507 (M+H)⁺.

### Example 5

### 4-amino-1-((3R)-3-(1-hydroxy-5-methoxy-1-(2-(o-tolyloxy)phenyl)pentyl)piperidin-1-yl)butan-1-one (#46)

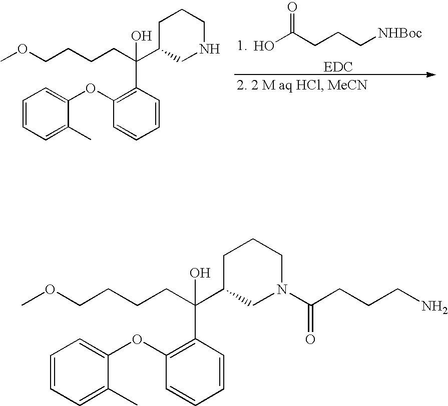

The title compound was prepared by application of procedures analogous to those described in Step 8 of Example 4 using 5-methoxy-1-((3R)-piperidin-3-yl)-1-(2-(o-tolyloxy)phenyl)pentan-1-ol and 4-(tert-butoxycarbonylamino)butanoic acid, followed by removal of the Boc group using a 1:1 2 M aq HCl/MeCN at rt overnight. The title compound was purified by preparative HPLC. LC-MS (3 min) t_{R}=1.45 min, m/z=469; ¹H NMR (CD₃OD) δ ppm 0.88-1.60 (m), 1.64 (m), 1.88 (m), 2.22 (s), 2.24-2.66 (m), 2.92 (m), 3.06 (dd), 3.26 (s), 3.28 (m), 3.82 (d), 4.14 and 4.44 (d), 6.58 (d), 6.74 (d), 7.04 (m), 7.16 (m), 7.26 (m), 7.64 (d).

### Example 6

### (3R)-3-amino-4-cyclohexyl-1-(3-((3-methoxypropoxy)(phenyl)methyl)piperidin-1-yl)butan-1-one (#43)

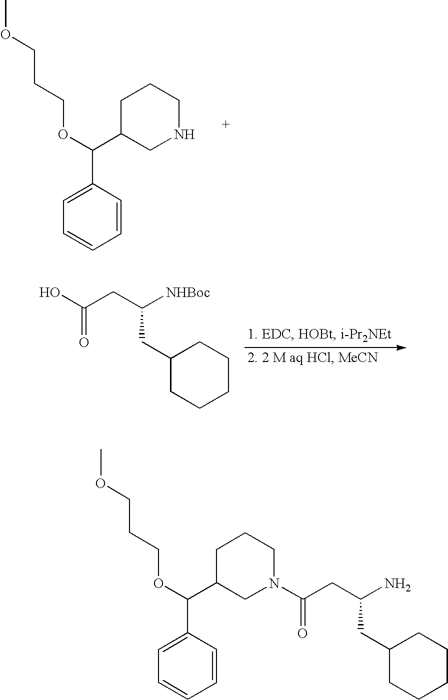

The title compound was prepared by application of procedures analogous to those described in Step 8 of Example 4 using 3-((3-methoxypropoxy)(phenyl)methyl)piperidine and (R)-3-(tert-butoxycarbonylamino)-4-cyclohexylbutanoic acid, followed by removal of the Boc group using a 1:1 2 M aq HCl/MeCN at rt overnight. The title compound was purified by preparative HPLC. LC-MS (3 min) tR=1.56 min, m/z=431.

### Example 7

### 4-amino-3-hydroxy-1-((3R)-3-(1-hydroxy-5-methoxy-1-(2-(o-tolyloxy)phenyl)pentyl)piperidin-1-yl)butan-1-one (#45)

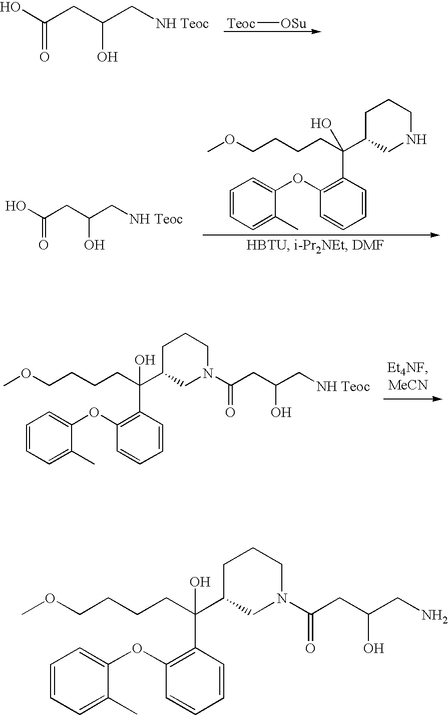

LC-MS (3 min) t_{R}=1.41 min, m/z=485; ¹H NMR (CD₃OD) δ ppm 0.86-1.60 (m), 1.66 (m), 1.92 (m), 2.22 (s), 2.24-2.98 (m), 3.08 (m), 3.26 (s), 3.28 (m), 3.84 and 4.44 (d), 4.18 (m), 6.56 (dd), 6.76 (m), 7.04 (m), 7.16 (m), 7.36 (dd), 7.64 (d).

### Example 8

### (3S,4S)-4-amino-1-((3R)-3-(1-(3-fluorophenyl)-1-hydroxy-5-methoxypentyl)piperidin-1-yl)-3-hydroxy-5-phenylpentan-1-one (#44)

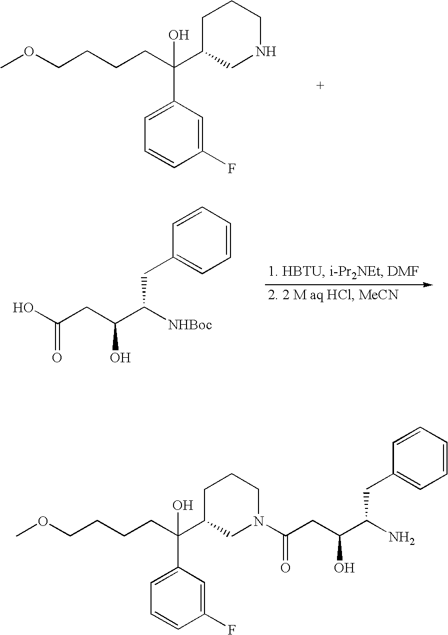

LC-MS (3 min) t_{R}=1.34 min, m/z=487; ¹H NMR (CD₃OD) δ ppm 0.88-1.18 (m), 1.20-1.42 (m), 1.42-1.80 (m), 1.92 (m), 2.24 (m), 2.54 (m), 2.60-2.82 (m), 2.96 (dd), 3.06 (dd), 3.26 (s), 3.32 (m), 3.52 (m), 3.78 and 4.10 (d), 4.04 (m), 4.42 and 4.84 (d), 6.96 (m), 7.28 (m), 7.30 (m)

### Example 9

### methyl (4-(3′-ethyl-6-fluoro-2-biphenylyl)-4-hydroxy-4-{(3R)-1-[3-hydroxy-4-(methylamino)butanoyl]-3-piperidinyl}butyl)carbamate (#59)

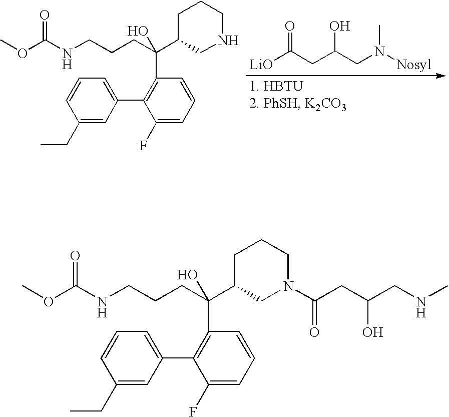

### Step 1. Methyl {4-(3′-ethyl-6-fluoro-2-biphenylyl)-4-hydroxy-4-[(3R)-1-(3-hydroxy-4-{methyl[(2-nitrophenyl)sulfonyl]amino}butanoyl)-3-piperidinyl]butyl}carbamate

A solution of methyl {4-(3′-ethyl-6-fluoro-2-biphenylyl)-4-hydroxy-4-[(3R)-3-piperidinyl]butyl}carbamate (95 mg, 0.20 mmol) in 1 mL of DMF at 25° C. was treated with lithium 3-hydroxy-4-{methyl[(2-nitrophenyl)sulfonyl]amino}butanoate (60 mg, 0.19 mmol), DIPEA (0.1 mL, 0.6 mmol), and HBTU (91 mg, 0.24 mmol), and the mixture was stirred for 1 h before being filtered through Celite® and subjected to reverse phase HPLC to give methyl {4-(3′-ethyl-6-fluoro-2-biphenylyl)-4-hydroxy-4-[(3R)-1-(3-hydroxy-4-{methyl[(2-nitrophenyl)sulfonyl]amino}butanoyl)-3-piperidinyl]butyl}carbamate as a solid (82 mg, 57%). ESI-MS (m/z): 729.2 (M+H⁺).

### Step 2. Methyl (4-(3′-ethyl-6-fluoro-2-biphenylyl)-4-hydroxy-4-{(3R)-1-[3-hydroxy-4-(methylamino)butanoyl]-3-piperidinyl}butyl)carbamate

A solution of methyl {4-(3′-ethyl-6-fluoro-2-biphenylyl)-4-hydroxy-4-[(3R)-1-(3-hydroxy-4-{methyl[(2-nitrophenyl)sulfonyl]amino}butanoyl)-3-piperidinyl]butyl}carbamate (0.082 g, 0.011 mmol) in 2 mL of DMF at 25° C. was treated with K₂CO₃ (0.047 g, 0.034 mmol) and thiophenol (0.038 mL, 0.034 mmol), and the mixture was stirred for 2 h before being filtered through Celite® and subjected to reverse phase HPLC to give methyl {4-(3′-ethyl-6-fluoro-2-biphenylyl)-4-hydroxy-4-[(3R)-1-(3-hydroxy-4-{methyl[(2-nitrophenyl)sulfonyl]amino}butanoyl)-3-piperidinyl]butyl}carbamate as a solid (0.038 g, 63%). ESI-MS (m/z): 544.3 (M+H⁺).

The following compounds were prepared using procedures analogous to those described above:
#60 N-(4-(3′-ethyl-6-fluorobiphenyl-2-yl)-4-hydroxy-4-((3R)-1-(3-hydroxy-4-(methylamino)butanoyl)piperidin-3-yl)butyl)-2,2,2-trifluoroacetamide using N-(4-(3′-ethyl-6-fluorobiphenyl-2-yl)-4-hydroxy-4-((R)-piperidin-3-yl)butyl)-2,2,2-trifluoroacetamide instead of methyl {4-(3′-ethyl-6-fluoro-2-biphenylyl)-4-hydroxy-4-[(3R)-3-piperidinyl]butyl}carbamate in Step 1.
#61 methyl 4-(3′-ethyl-6-fluorobiphenyl-2-yl)-4-hydroxy-4-((3R)-1-(3-methoxy-4-(methylamino)butanoyl)piperidin-3-yl)butylcarbamate using lithium 4-{methyl[(2-nitrophenyl)sulfonyl]amino}-3-(methyloxy)butanoate instead of lithium 3-hydroxy-4-{methyl[(2-nitrophenyl)sulfonyl]amino}butanoate in Step 1.

### Example 10

### methyl [4-((3R)-1-{[(2-aminoethyl)amino]carbonyl}-3-piperidinyl)-4-(3′-ethyl-6-fluoro-2-biphenylyl)-4-hydroxybutyl]carbamate (#66)

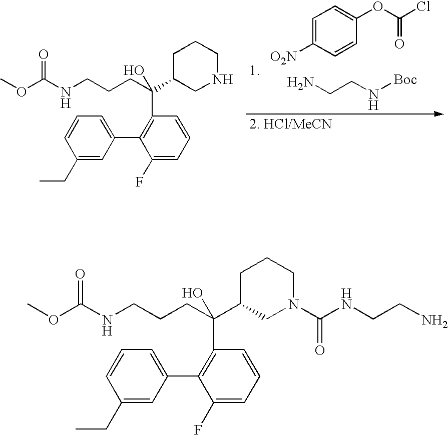

A solution of 4-nitrophenyl chloridocarbonate (64 mg, 0.32 mmol) in CH₂Cl₂ (2 mL) at 25° C. was treated with Et₃N (0.11 mL, 0.75 mmol) and 1,1-dimethylethyl (2-aminoethyl)carbamate (51 mg, 0.32 mmol) and the mixture was stirred for 0.5 h before methyl {4-(3′-ethyl-6-fluoro-2-biphenylyl)-4-hydroxy-4-[(3R)-3-piperidinyl]butyl}carbamate (80 mg g, 0.15 mmol) was added. The mixture was stirred for an additional 3 h before being concentrated under reduced pressure and subjected to reverse phase HPLC. The purified residue was treated with 2 mL of 1:1 2N HCl (aq): MeCN and the mixture was stirred at 25° C. overnight before being concentrated under reduced pressure to give a solid. The solid was dissolved in water and lyophilized to give methyl [4-((3R)-1-{[(2-aminoethyl)amino]carbonyl}-3-piperidinyl)-4-(3′-ethyl-6-fluoro-2-biphenylyl)-4-hydroxybutyl]carbamate as a solid (16.9 mg, 27%): ESI-MS (m/z): 515.3 (M+H⁺).

The following compounds were prepared following procedures analogous to those described above by using the appropriate piperidine intermediate and the indicated amine in place of 1,1-dimethylethyl (2-aminoethyl)carbamate:
#65 methyl {4-(3′-ethyl-6-fluoro-2-biphenylyl)-4-hydroxy-4-[(3R)-1-({[2-(methylamino)ethyl]amino}carbonyl)-3-piperidinyl]butyl}carbamate using 1,1-dimethylethyl (2-amino ethyl)methylcarbamate.
#67 methyl [4-((3R)-1-{[(3-amino-2,2-dimethylpropyl)amino]carbonyl}-3-piperidinyl)-4-(3′-ethyl-6-fluoro-2-biphenylyl)-4-hydroxybutyl]carbamate using 1,1-dimethylethyl (3-amino-2,2-dimethylpropyl)methylcarbamate.
#68 methyl {2-[((S)-(6-chloro-3′-ethyl-2-biphenylyl) {(2R)-4-[({(2S)-2-(methylamino)-4-[4-(methyloxy)phenyl]butyl}amino)carbonyl]-2-morpholinyl}methyl)oxy]ethyl}carbamate using 1,1-dimethylethyl {(1S)-1-(aminomethyl)-3-[4-(methyloxy)phenyl]propyl}methylcarbamate.
#69 methyl {2-[((S)-(6-chloro-3′-ethyl-2-biphenylyl){(2R)-4-[({(2S)-2-(methylamino)-3-[4-(methyloxy)phenyl]propyl}amino)carbonyl]-2-morpholinyl}methyl)oxy]ethyl}carbamate using 1,1-dimethylethyl((1S)-2-amino-1-{[4-(methyloxy)phenyl]methyl}ethyl)methylcarbamate.
#70 methyl {2-[((R)-(6-chloro-3′-ethyl-2-biphenylyl){(3R)-1-[({(2S)-2-(methylamino)-4-[4-(methyloxy)phenyl]butyl}amino)carbonyl]-3-piperidinyl}methyl)oxy]ethyl}carbamate using 1,1-dimethylethyl {(1S)-1-(aminomethyl)-3-[4-(methyloxy)phenyl]propyl}methylcarbamate.
#71 methyl {2-[((R)-(6-chloro-3′-ethyl-2-biphenylyl){(3R)-1-[({(2S)-2-(methylamino)-3-[4-(methyloxy)phenyl]propyl}amino)carbonyl]-3-piperidinyl}methyl)oxy]ethyl}carbamate using 1,1-dimethylethyl((1S)-2-amino-1-{[4-(methyloxy)phenyl]methyl}ethyl)methylcarbamate.

The following are compounds of the invention:

| | | Synthetic | | | |
|---|---|---|---|---|---|
| | | Method | LC_MS | t_{R} | Mass |
| Cpd. No. | Name | Example No. | Method | (min) | observed |
| 1 | 1-(6-chloro-3′-ethyl-2-biphenylyl)-1-[(3R)-1-(2- | 1 | 1 | 2.55 | 501.2 |
| | methylalanyl)-3-piperidinyl]-5-(methyloxy)-1-pentanol | | | | |
| 2 | 2-({(3R)-3-[1-(6-chloro-3′-ethyl-2-biphenylyl)-1- | 1 | 1 | 2.77 | 532.2 |
| | hydroxy-5-(methyloxy)pentyl]-1-piperidinyl}carbonyl)- | | | | |
| | 2-methyl-1,3-propanediol | | | | |
| 3 | 1-(6-chloro-3′-ethyl-2-biphenylyl)-1-{(3R)-1-[N-(2- | 2 | 1 | 2.56 | 545.3 |
| | hydroxyethyl)-2-methylalanyl]-3-piperidinyl}-5- | | | | |
| | (methyloxy)-1-pentanol | | | | |
| 4 | 1-[1-(6-aminohexanoyl)-4-piperidinyl]-1-(3′-ethyl-6- | 1 | 1 | 2.48 | 513.3 |
| | fluoro-2-biphenylyl)-5-(methyloxy)-1-pentanol | | | | |
| 5 | methyl (4-[3-fluoro-2-(3-quinolinyl)phenyl]-4-hydroxy- | 1 | 1 | 2.33 | 551.3 |
| | 4-{(3R)-1-[4-(methylamino)butanoyl]-3- | | | | |
| | piperidinyl}butyl)carbamate | | | | |
| 6 | methyl (4-[3-chloro-2-(5-methyl-2-furanyl)phenyl]-4- | 1 | 1 | 2.34 | 520.3 |
| | hydroxy-4-{(3R)-1-[4-(methylamino)butanoyl]-3- | | | | |
| | piperidinyl}butyl)carbamate | | | | |
| 7 | (1R)-1-[(2R)-4-(4-aminobutanoyl)-2-morpholinyl]-1- | 1 | 1 | 2.46 | 491.2 |
| | (4′,6-difluoro-3′-methyl-2-biphenylyl)-5-(methyloxy)-1- | | | | |
| | pentanol | | | | |
| 8 | methyl [4-{(3R)-1-[4-amino-3-(4- | 1 | 1 | 2.45 | 624.3 |
| | chlorophenyl)butanoyl]-3-piperidinyl}-4-(3′-ethyl-6- | | | | |
| | fluoro-2-biphenylyl)-4-hydroxybutyl]carbamate | | | | |
| 9 | methyl (4-(6-chloro-3′-fluoro-5′-methyl-2-biphenylyl)- | 1 | 1 | 2.30 | 548.3 |
| | 4-hydroxy-4-{(3R)-1-[4-(methylamino)butanoyl]-3- | | | | |
| | piperidinyl}butyl)carbamate | | | | |
| 10 | methyl (4-(3′,6-difluoro-5′-methyl-2-biphenylyl)-4- | 1 | 1 | 2.23 | 532.3 |
| | hydroxy-4-{(3R)-1-[4-(methylamino)butanoyl]-3- | | | | |
| | piperidinyl}butyl)carbamate | | | | |
| 11 | methyl (4-hydroxy-4-{(3R)-1-[4- | 1 | 1 | 2.42 | 513.3 |
| | (methylamino)butanoyl]-3-piperidinyl}-4-{2-[(2- | | | | |
| | methylphenyl)oxy]phenyl}butyl)carbamate | | | | |
| 12 | methyl [4-[(3R)-1-$$-alanyl-3-piperidinyl]-4-(3′-ethyl-6- | 1 | 1 | 2.31 | 500.3 |
| | fluoro-2-biphenylyl)-4-hydroxybutyl]carbamate | | | | |
| 13 | methyl [4-[(3R)-1-(4-aminobutanoyl)-3-piperidinyl]-4- | 1 | 1 | 2.31 | 514.3 |
| | (3′-ethyl-6-fluoro-2-biphenylyl)-4- | | | | |
| | hydroxybutyl]carbamate | | | | |
| 14 | methyl [4-[(3R)-1-(5-aminopentanoyl)-3-piperidinyl]-4- | 1 | 1 | 2.31 | 528.3 |
| | (3′-ethyl-6-fluoro-2-biphenylyl)-4- | | | | |
| | hydroxybutyl]carbamate | | | | |
| 15 | methyl (2S)-2-amino-5-[(3R)-3-(1-(3′-ethyl-6-fluoro-2- | 1 | 1 | 2.46 | 572.3 |
| | biphenylyl)-1-hydroxy-4- | | | | |
| | {[(methyloxy)carbonyl]amino}butyl)-1-piperidinyl]-5- | | | | |
| | oxopentanoate | | | | |
| 16 | methyl ((4S)-4-(6-chloro-3′-ethyl-2-biphenylyl)-4- | 1 | 1 | 2.46 | 544.3 |
| | hydroxy-4-{(3R)-1-[4-(methylamino)butanoyl]-3- | | | | |
| | piperidinyl}butyl)carbamate | | | | |
| 17 | methyl [4-{(3R)-1-[4-(dimethylamino)butanoyl]-3- | 1 | 1 | 2.47 | 542.3 |
| | piperidinyl}-4-(3′-ethyl-6-fluoro-2-biphenylyl)-4- | | | | |
| | hydroxybutyl]carbamate | | | | |
| 18 | methyl (4-[3-chloro-2-(2,3-dihydro-1-benzofuran-6- | 1 | 1 | 1.64 | 558.2 |
| | yl)phenyl]-4-hydroxy-4-{(3R)-1-[4- | | | | |
| | (methylamino)butanoyl]-3-piperidinyl}butyl)carbamate | | | | |
| 19 | methyl [4-(3′-ethyl-6-fluoro-2-biphenylyl)-4-hydroxy-4- | 1 | 1 | 2.40 | 557.3 |
| | ((3R)-1-{N-methyl-N-[2-(methylamino)ethyl]glycyl}-3- | | | | |
| | piperidinyl)butyl]carbamate | | | | |
| 20 | methyl [4-(3′-ethyl-6-fluoro-2-biphenylyl)-4-hydroxy-4- | 2 | 1 | 2.43 | 558.3 |
| | ((3R)-1-{4-[(2-hydroxyethyl)amino]butanoyl}-3- | | | | |
| | piperidinyl)butyl]carbamate | | | | |
| 21 | (2S)-2-amino-5-[(3R)-3-(1-(3′-ethyl-6-fluoro-2- | 3 | 1 | 2.37 | 558.2 |
| | biphenylyl)-1-hydroxy-4- | | | | |
| | {[(methyloxy)carbonyl]amino}butyl)-1-piperidinyl]-5- | | | | |
| | oxopentanoic acid | | | | |
| 22 | methyl [4-{(3R)-1-[(4S)-4-amino-5-hydroxypentanoyl]- | 1 | 1 | 2.38 | 544.3 |
| | 3-piperidinyl}-4-(3′-ethyl-6-fluoro-2-biphenylyl)-4- | | | | |
| | hydroxybutyl]carbamate | | | | |
| 23 | methyl [4-{(3R)-1-[(4R)-4-amino-5-hydroxypentanoyl]- | 1 | 1 | 2.38 | 544.3 |
| | 3-piperidinyl}-4-(3′-ethyl-6-fluoro-2-biphenylyl)-4- | | | | |
| | hydroxybutyl]carbamate | | | | |
| 24 | N-(4-(2′,6-difluoro-5′-methyl-2-biphenylyl)-4-hydroxy- | 1 | 1 | 2.39 | 516.3 |
| | 4-{(3R)-1-[4-(methylamino)butanoyl]-3- | | | | |
| | piperidinyl}butyl)acetamide | | | | |
| 25 | N-(4-(6-fluoro-3′-methyl-2-biphenylyl)-4-hydroxy-4- | 1 | 1 | 2.40 | 498.3 |
| | {(3R)-1-[4-(methylamino)butanoyl]-3- | | | | |
| | piperidinyl}butyl)acetamide | | | | |
| 26 | methyl [4-[(3R)-1-(4-amino-3-hydroxybutanoyl)-3- | 1 | 1 | 2.35 | 530.2 |
| | piperidinyl]-4-(3′-ethyl-6-fluoro-2-biphenylyl)-4- | | | | |
| | hydroxybutyl]carbamate | | | | |
| 27 | methyl [4-{(3R)-1-[(2S)-4-amino-2-hydroxybutanoyl]- | 1 | 1 | 2.39 | 530.3 |
| | 3-piperidinyl}-4-(3′-ethyl-6-fluoro-2-biphenylyl)-4- | | | | |
| | hydroxybutyl]carbamate | | | | |
| 28 | methyl ((4S)-4-(3′-ethyl-6-fluoro-2-biphenylyl)-4- | 1 | 1 | 2.41 | 528.3 |
| | hydroxy-4-{(3R)-1-[4-(methylamino)butanoyl]-3- | | | | |
| | piperidinyl}butyl)carbamate | | | | |
| 29 | methyl [4-[(3R)-1-(6-aminohexanoyl)-3-piperidinyl]-4- | 1 | 1 | 2.32 | 542.3 |
| | (3′-ethyl-6-fluoro-2-biphenylyl)-4- | | | | |
| | hydroxybutyl]carbamate | | | | |
| 30 | methyl [4-[(3R)-1-L-asparaginyl-3-piperidinyl]-4-(3′- | 1 | 1 | 2.24 | 543.3 |
| | ethyl-6-fluoro-2-biphenylyl)-4-hydroxybutyl]carbamate | | | | |
| 31 | methyl {4-(3′-ethyl-6-fluoro-2-biphenylyl)-4-hydroxy-4- | 1 | 1 | 2.39 | 528.3 |
| | [(3R)-1-L-valyl-3-piperidinyl]butyl}carbamate | | | | |
| 32 | methyl {4-(3′-ethyl-6-fluoro-2-biphenylyl)-4-hydroxy-4- | 1 | 1 | 2.26 | 516.2 |
| | [(3R)-1-L-seryl-3-piperidinyl]butyl}carbamate | | | | |
| 33 | methyl [4-[(3R)-1-(L-alanyl-L-alanyl)-3-piperidinyl]-4- | 1 | 1 | 2.30 | 571.3 |
| | (3′-ethyl-6-fluoro-2-biphenylyl)-4- | | | | |
| | hydroxybutyl]carbamate | | | | |
| 34 | methyl {4-(3′-ethyl-6-fluoro-2-biphenylyl)-4-hydroxy-4- | 1 | 1 | 2.45 | 576.3 |
| | [(3R)-1-L-phenylalanyl-3-piperidinyl]butyl}carbamate | | | | |
| 35 | methyl {4-(3′-ethyl-6-fluoro-2-biphenylyl)-4-hydroxy-4- | 1 | 1 | 2.52 | 615.3 |
| | [(3R)-1-D-tryptophyl-3-piperidinyl]butyl}carbamate | | | | |
| 36 | methyl {4-(3′-ethyl-6-fluoro-2-biphenylyl)-4-[(3R)-1- | 1 | 1 | 2.26 | 543.2 |
| | (glycylglycyl)-3-piperidinyl]-4-hydroxybutyl}carbamate | | | | |
| 37 | methyl {4-(3′-ethyl-6-fluoro-2-biphenylyl)-4-hydroxy-4- | 1 | 1 | 2.30 | 514.3 |
| | [(3R)-1-(2-methylalanyl)-3-piperidinyl]butyl}carbamate | | | | |
| 38 | phenylmethyl (2S)-2-amino-5-[(3R)-3-(1-(3′-ethyl-6- | 1 | 1 | 2.53 | 648.3 |
| | fluoro-2-biphenylyl)-1-hydroxy-4- | | | | |
| | {[(methyloxy)carbonyl]amino}butyl)-1-piperidinyl]-5- | | | | |
| | oxopentanoate | | | | |
| 39 | phenylmethyl (2S)-2-amino-4-[(3R)-3-(1-(3′-ethyl-6- | 1 | 1 | 2.48 | 634.3 |
| | fluoro-2-biphenylyl)-1-hydroxy-4- | | | | |
| | {[(methyloxy)carbonyl]amino}butyl)-1-piperidinyl]-4- | | | | |
| | oxobutanoate | | | | |
| 40 | methyl {4-(3′-ethyl-6-fluoro-2-biphenylyl)-4-[(3R)-1-L- | 1 | 1 | 2.40 | 557.3 |
| | α-glutaminyl-3-piperidinyl]-4-hydroxybutyl}carbamate | | | | |
| 41 | methyl {4-(3′-ethyl-6-fluoro-2-biphenylyl)-4-hydroxy-4- | 1 | 1 | 2.39 | 543.3 |
| | [(3R)-1-D-ornithyl-3-piperidinyl]butyl}carbamate | | | | |
| 42 | (R)-1-((R)-3-((S)-1-(3-chlorophenyl)-1-hydroxy-5- | 4 | 3 | | 507 |
| | methoxypentyl) piperidin-1-yl)-4-cyclohexyl-3- | | | | |
| | ((methylamino)methyl)butan-1-one | | | | |
| 43 | (3R)-3-amino-4-cyclohexyl-1-(3-((3- | 6 | 3 | 1.56 | 431 |
| | methoxypropoxy)(phenyl)methyl)piperidin-1-yl)butan- | | | | |
| | 1-one | | | | |
| 44 | (3S,4S)-4-amino-1-((R)-3-((S)-1-(3-fluorophenyl)-1- | 8 | 3 | 1.34 | 487 |
| | hydroxy-5-methoxypentyl)piperidin-1-yl)-3-hydroxy-5- | | | | |
| | phenylpentan-1-one | | | | |
| 45 | 4-amino-3-hydroxy-1-((R)-3-((S)-1-hydroxy-5- | 7 | 3 | 1.41 | 485 |
| | methoxy-1-(2-(o-tolyloxy)phenyl)pentyl)piperidin-1- | | | | |
| | yl)butan-1-one | | | | |
| 46 | 4-amino-1-((R)-3-((S)-1-hydroxy-5-methoxy-1-(2-(o- | 5 | 3 | 1.45 | 469 |
| | tolyloxy)phenyl)pentyl)piperidin-1-yl)butan-1-one | | | | |
| 47 | methyl (4-[3-chloro-2-(3-quinolinyl)phenyl]-4-hydroxy- | 1 | 1 | 2.37 | 567.2 |
| | 4-{(3R)-1-[4-(methylamino)butanoyl]-3- | | | | |
| | piperidinyl}butyl)carbamate | | | | |
| 48 | methyl {4-hydroxy-4-{(3R)-1-[4- | 1 | 1 | 2.34 | 614.2 |
| | (methylamino)butanoyl]-3-piperidinyl}-4-[2′- | | | | |
| | (methyloxy)-5′-(trifluoromethyl)-2- | | | | |
| | biphenylyl]butyl}carbamate | | | | |
| 49 | methyl {4-(3′-ethyl-6-fluoro-2-biphenylyl)-4-[(3R)-1- | 1 | 1 | 2.31 | 486.3 |
| | glycyl-3-piperidinyl]-4-hydroxybutyl}carbamate | | | | |
| 50 | methyl [4-{(3R)-1-[(3S)-4-amino-3-hydroxybutanoyl]- | 1 | 1 | 2.43 | 530.2 |
| | 3-piperidinyl}-4-(3′-ethyl-6-fluoro-2-biphenylyl)-4- | | | | |
| | hydroxybutyl]carbamate | | | | |
| 51 | N-(4-(2′,6-difluoro-5′-methyl-2-biphenylyl)-4-hydroxy- | 1 | 1 | 2.5 | 570.2 |
| | 4-{(3R)-1-[4-(methylamino)butanoyl]-3- | | | | |
| | piperidinyl}butyl)-2,2,2-trifluoroacetamide | | | | |
| 52 | N-[(4S)-4-{(3R)-1-[4-amino-3-(4- | 1 | 1 | 2.56 | 666.2 |
| | chlorophenyl)butanoyl]-3-piperidinyl}-4-(2′,6-difluoro- | | | | |
| | 5′-methyl-2-biphenylyl)-4-hydroxybutyl]-2,2,2- | | | | |
| | trifluoroacetamide | | | | |
| 53 | N-[4-{(3R)-1-[(3S)-4-amino-3-hydroxybutanoyl]-3- | 1 | 1 | 2.46 | 572.2 |
| | piperidinyl}-4-(2′,6-difluoro-5′-methyl-2-biphenylyl)-4- | | | | |
| | hydroxybutyl]-2,2,2-trifluoroacetamide | | | | |
| 54 | N-(4-(3′-ethyl-6-fluoro-2-biphenylyl)-4-hydroxy-4- | 1 | 1 | 2.47 | 566.2 |
| | {(3R)-1-[4-(methylamino)butanoyl]-3- | | | | |
| | piperidinyl}butyl)-2,2,2-trifluoroacetamide | | | | |
| 55 | N-[4-{(3R)-1-[(3S)-4-amino-3-hydroxybutanoyl]-3- | 1 | 1 | 2.45 | 568.2 |
| | piperidinyl}-4-(3′-ethyl-6-fluoro-2-biphenylyl)-4- | | | | |
| | hydroxybutyl]-2,2,2-trifluoroacetamide | | | | |
| 56 | methyl [4-[(3R)-1-(4- | 1 | 1 | 2.46 | 556.3 |
| | {[amino(imino)methyl]amino}butanoyl)-3-piperidinyl]- | | | | |
| | 4-(3′-ethyl-6-fluoro-2-biphenylyl)-4- | | | | |
| | hydroxybutyl]carbamate | | | | |
| 57 | methyl [4-{(3R)-1-[4-amino-3-(4- | 1 | 1 | 2.5 | 640.2 |
| | chlorophenyl)butanoyl]-3-piperidinyl}-4-(6-chloro-3′- | | | | |
| | ethyl-2-biphenylyl)-4-hydroxybutyl]carbamate | | | | |
| 58 | methyl [4-{(3R)-1-[(3S)-4-amino-3-hydroxybutanoyl]- | 1 | 1 | 2.41 | 546.2 |
| | 3-piperidinyl}-4-(6-chloro-3′-ethyl-2-biphenylyl)-4- | | | | |
| | hydroxybutyl]carbamate | | | | |
| 59 | methyl (4-(3′-ethyl-6-fluoro-2-biphenylyl)-4-hydroxy-4- | 9 | 1 | 2.44 | 544.3 |
| | {(3R)-1-[3-hydroxy-4-(methylamino)butanoyl]-3- | | | | |
| | piperidinyl}butyl)carbamate | | | | |
| 60 | N-(4-(3′-ethyl-6-fluorobiphenyl-2-yl)-4-hydroxy-4- | 9 | 1 | 2.49 | 582.2 |
| | ((3R)-1-(3-hydroxy-4- | | | | |
| | (methylamino)butanoyl)piperidin-3-yl)butyl)-2,2,2- | | | | |
| | trifluoroacetamide | | | | |
| 61 | methyl 4-(3′-ethyl-6-fluorobiphenyl-2-yl)-4-hydroxy-4- | 9 | 1 | 2.46 | 558.3 |
| | ((3R)-1-(3-methoxy-4- | | | | |
| | (methylamino)butanoyl)piperidin-3-yl)butylcarbamate | | | | |
| 62 | methyl (4-[6-fluoro-3′-(1-methylethyl)-2-biphenylyl]-4- | 1 | 1 | 2.5 | 542.3 |
| | hydroxy-4-{(3R)-1-[4-(methylamino)butanoyl]-3- | | | | |
| | piperidinyl}butyl)carbamate | | | | |
| 63 | methyl {2-[((S)-(6-chloro-3′-ethyl-2-biphenylyl){(2R)-4- | 1 | 2 | 1.54 | 532.4 |
| | [4-(methylamino)butanoyl]-2- | | | | |
| | morpholinyl}methyl)oxy]ethyl}carbamate | | | | |
| 64 | methyl {2-[((R)-(6-chloro-3′-ethyl-2-biphenylyl){(3R)-1- | 1 | 2 | 1.65 | 530.5 |
| | [4-(methylamino)butanoyl]-3- | | | | |
| | piperidinyl}methyl)oxy]ethyl}carbamate | | | | |
| 65 | methyl {4-(3′-ethyl-6-fluoro-2-biphenylyl)-4-hydroxy-4- | 10 | 1 | 2.46 | 529.3 |
| | [(3R)-1-({[2-(methylamino)ethyl]amino}carbonyl)-3- | | | | |
| | piperidinyl]butyl}carbamate | | | | |
| 66 | methyl [4-((3R)-1-{[(2-aminoethyl)amino]carbonyl}-3- | 10 | 1 | 2.42 | 515.3 |
| | piperidinyl)-4-(3′-ethyl-6-fluoro-2-biphenylyl)-4- | | | | |
| | hydroxybutyl]carbamate | | | | |
| 67 | methyl [4-((3R)-1-{[(3-amino-2,2- | 10 | 1 | 2.50 | 557.3 |
| | dimethylpropyl)amino]carbonyl}-3-piperidinyl)-4-(3′- | | | | |
| | ethyl-6-fluoro-2-biphenylyl)-4-hydroxybutyl]carbamate | | | | |
| 68 | methyl {2-[((S)-(6-chloro-3′-ethyl-2-biphenylyl){(2R)-4- | 10 | 2 | 1.51 | 667.2 |
| | [({(2S)-2-(methylamino)-4-[4- | | | | |
| | (methyloxy)phenyl]butyl}amino)carbonyl]-2- | | | | |
| | morpholinyl}methyl)oxy]ethyl}carbamate | | | | |
| 69 | methyl {2-[((S)-(6-chloro-3′-ethyl-2-biphenylyl){(2R)-4- | 10 | 2 | 1.48 | 653.2 |
| | [({(2S)-2-(methylamino)-3-[4- | | | | |
| | (methyloxy)phenyl]propyl}amino)carbonyl]-2- | | | | |
| | morpholinyl}methyl)oxy]ethyl}carbamate | | | | |
| 70 | methyl {2-[((R)-(6-chloro-3′-ethyl-2-biphenylyl){(3R)-1- | 10 | 2 | 1.64 | 665.1 |
| | [({(2S)-2-(methylamino)-4-[4- | | | | |
| | (methyloxy)phenyl]butyl}amino)carbonyl]-3- | | | | |
| | piperidinyl}methyl)oxy]ethyl}carbamate | | | | |
| 71 | methyl {2-[((R)-(6-chloro-3′-ethyl-2-biphenylyl){(3R)-1- | 10 | 2 | 1.57 | 651.3 |
| | [({(2S)-2-(methylamino)-3-[4- | | | | |
| | (methyloxy)phenyl]propyl}amino)carbonyl]-3- | | | | |
| | piperidinyl}methyl)oxy]ethyl}carbamate | | | | |

### Biological Assay Example 1

### In Vitro Activity Studies

### IC₅₀ Values for Renin Inhibition

The action of renin inhibitors was demonstrated experimentally by means of an in vitro test which measures the increase in fluorescence of an internally quenched peptide substrate. The sequence of this peptide corresponds to the sequence of human angiotensinogen. The following test protocol was used:

All reactions were carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) was added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer was added, and the solution was mixed by pipetting. The increase in fluorescence at 495 nm (excitation at 340 nm) was measured for 60-360 minutes at room temperature using a Perkin-Elmer Fusion microplate reader. The slope of a linear portion of the plot of fluorescence increase as a function of time was then determined, and the rate was used for calculating percent inhibition in relation to uninhibited control. The percent inhibition values were plotted as a function of inhibitor concentration, and the IC₅₀ was determined from a fit of this data to a four parameter equation. The IC₅₀ was defined as the concentration of a particular inhibitor that reduces the formation of product by 50% relative to a control sample containing no inhibitor. The compounds exemplified herein exhibit inhibiting activity with an IC₅₀ of between about 5,000 nM to about 0.001 nM. The compounds identified as I-5a, I-8a, I-13a, I-14a, I-15a, I-16a, I-19a, I-20a, I-22a, I-23a, I-24a, I-25a, I-26a, I-27a, I-28a, I-28b, I-29a, I-32a, I-34a, I-36a, and I-38a each exhibit an in vitro IC₅₀ of between about 100 nM and about 0.01 nM.

In the in vitro systems the compounds of the invention exhibit inhibiting activities at minimum concentrations of from approximately 5×10⁻⁵ M to approximately 10⁻¹² M. Preferred compounds of the invention exhibit inhibiting activities at minimum concentrations of from approximately 10⁻⁷ M to approximately 10⁻¹² M. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

### Biological Assay Example 2

### In Vitro Activity Studies

### IC₅₀ Values for Renin Inhibition

The potency of renin inhibitors was measured using an in vitro renin assay. In this assay, renin-catalyzed proteolysis of a fluorescently labeled peptide converts the peptide from a weakly fluorescent to a strongly fluorescent molecule. The following test protocol was used. Substrate solution (5 μA; 2 μM Arg-Glu-Lys(5-Fam)-Ile-His-Pro-Phe-His-Leu-Val-Ile-His-Thr-Lys(5,6 Tamra)-Arg-CONH₂ in 50 mM Hepes, 125 mM NaCl, 0.1% CHAPS, pH 7.4) then trypsin-activated recombinant human renin (Scott, Martin J. et. al. Protein Expression and Purification 2007, 52(1), 104-116; 5 μL; 600 pM renin in 50 mM Hepes, 125 mM NaCl, 0.1% CHAPS, pH 7.4) were added sequentially to a black Greiner low volume 384-well plate (cat. #784076) pre-stamped with a 100 nl DMSO solution of compound at the desired concentration. The assay plates were incubated at room temperature for 2 hours with a cover plate then quenched by the addition of a stop solution (2 μL; 5 μM of Bachem C-3195 in 50 mM Hepes, 125 mM NaCl, 0.1% CHAPS, pH 7.4, 10% DMSO). The assay plates were read on an LJL Acquest using a 485 nm excitation filter, a 530 nm emission filter, and a 505 nm dichroic filter. Compounds were initially prepared in neat DMSO at a concentration of 10 mM. For inhibition curves, compounds were diluted using a three fold serial dilution and tested at 11 concentrations (e.g. 50 μM-0.8 nM or 25 μM-0.42 nM or 2.5 μM to 42 pM). Curves were analyzed using ActivityBase and XLfit, and results were expressed as pIC₅₀ values. In the in vitro systems the compounds of the invention exhibit inhibiting activities at minimum concentrations of from approximately 5×10⁻⁵ M to approximately 10⁻¹² M. Preferred compounds of the invention exhibit inhibiting activities at minimum concentrations of from approximately 10⁻⁷ M to approximately 10⁻¹² M.

### Biological Assay Example 3

### Inhibition in Human Plasma

The action of renin inhibitors in vitro in human plasma can also be demonstrated experimentally by the decrease in plasma renin activity (PRA) levels observed in the presence of the compounds. Incubations mixtures contain in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/mL sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 pM of recombinant human renin is added to achieve PRA of 3-4 ng/ml/hr. The cleavage of endogenous angiotensinogen in plasma is carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH are then used for deriving percent of inhibition for each concentration of inhibitors and fitting dose-response data into a four parametric model from which IC₅₀ values, defined as concentrations of inhibitors at which 50% inhibition occurs, are determined.

### Biological Assay Example 4

The efficacy of the renin inhibitors can also be evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434). Experiments are conducted in 6-week-old double transgenic rats (dTGRs). The model has been described in detail earlier. Briefly, the human renin construct used to generate transgenic animals made up the entire genomic human renin gene (10 exons and 9 introns), with 3.0 kB of the 5′-promoter region and 1.2 kB of 3′ additional sequences. The human angiotensinogen construct made up the entire human angiotensinogen gene (5 exons and 4 introns), with 1.3 kB of 5′-flanking and 2.4 kB of 3′-flanking sequences are used to generate rats producing human angiotensinogen (hAogen). The hRen and hAogen rats are rederived using embryo transfer from breeding pairs obtained under license from Ascencion Gmbh (Germany). The hAogen and hRen are then crossed to produce the double transgenic dTGR) off-spring. The dTGr rats are maintained on irradiated rodent chow (5VO2, Purina Mills Inc) and normal water. Radio telemetry transmitters (TA11PAC40, Data Sciences International) are surgically implanted at 5-6 weeks of age. The telemetry system provides 24-h recordings of systolic, mean, diastolic arterial pressure (SAP, MAP, DAP, respectively) and heart rate (HR). Prior to dosing, baseline hemodynamic measures are obtained for 24 hours. Rats are then dosed orally with vehicle or drug and monitored up to 48 hours post-dose.

While this invention has been particularly shown and described with references to preferred embodiments thereof, it will be understood by those skilled in the art that various changes in form and details may be made therein without departing from the scope of the invention encompassed by the appended claims.

1. A compound represented by Formula I: 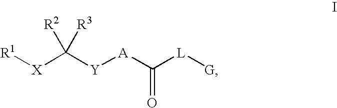
or a pharmaceutically acceptable salt thereof, wherein:
R¹ is:
a) (C₁-C₁₂)alkyl, (C₃-C₇)cycloalkyl, (C₄-C₁₂)cycloalkyl(C₁-C₆)alkyl, halo(C₁-C₁₂)alkyl, halo(C₃-C₇)-cycloalkyl, halo(C₄-C₁₂)cycloalkyl(C₁-C₆)alkyl, or saturated heterocyclyl, each optionally substituted with 1 to 5 groups independently selected from the group consisting of halogen, (C₁-C₆)alkyl, halo(C₁-C₆)alkyl, and oxo; or
b) phenyl, naphthyl, heteroaryl, or bicyclic heteroaryl, each optionally substituted with 1 to 5 groups independently selected from the groups consisting of:
1) fluorine, chlorine, bromine, iodine, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkyl(C₁-C₆)alkyl, (C₂-C₆)alkenyl, (C₅-C₈)cycloalkenyl, (C₅-C₈)cycloalkyl(C₂-C₆)alkenyl, (C₂-C₈)alkynyl, (C₃-C₈)cycloalkyl(C₂-C₄)alkynyl, halo(C₁-C₈)alkyl, halo(C₃-C₆)cycloalkyl, halo(C₄-C₈)cycloalkyl(C₁-C₆)alkyl, (C₁-C₃)alkyl(C₄-C₈)cycloalkyl(C₁-C₆)alkyl, di(C₁-C₃)alkyl(C₄-C₈)cycloalkyl(C₁-C₆)alkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkyl(C₂-C₆)alkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)-cycloalkylalkoxy, (C₁-C₈)alkylthio, (C₃-C₈)cycloalkythio, (C₄-C₈)cycloalkyl(C₁-C₆)alkylthio, (C₁-C₃)alkyl(C₃-C₈)cycloalkythio, (C₁-C₃)alkyl(C₄-C₈)cycloalkyl(C₁-C₆)alkylthio, di(C₁-C₃)alkyl(C₃-C₈)-cycloalkythio, di(C₁-C₃)alkyl(C₄-C₈)cycloalkyl(C₁-C₆)alkylthio, halo(C₁-C₈)alkylthio, halo(C₃-C₈)-cycloalkythio, halo(C₄-C₈)-cycloalkyl(C₁-C₆)alkylthio, (C₁-C₈)alkylsulfinyl, (C₃-C₈)-cycloalkyl-sulfinyl, (C₄-C₈)cycloalkyl-alkylsulfinyl, (C₁-C₃)alkyl(C₃-C₈)cycloalkyl-sulfinyl, (C₁-C₃)alkyl(C₄-C₈)cycloalkyl-alkylsulfinyl, di(C₁-C₃)alkyl(C₃-C₈)cycloalkyl-sulfinyl, di(C₁-C₃)alkyl(C₄-C₈)cycloalkyl-alkylsulfinyl, halo(C₁-C₈)alkylsulfinyl, halo(C₃-C₈)cycloalkylsulfinyl, halo(C₄-C₈)cycloalkyl(C₁-C₆)alkylsulfinyl, (C₁-C₈)alkyl-sulfonyl, (C₃-C₈)cycloalkylsulfonyl, (C₄-C₈)cycloalkyl(C₁-C₆)alkylsulfonyl, (C₁-C₃)alkyl(C₃-C₈)cycloalkylsulfonyl, (C₁-C₃)alkyl(C₄-C₈)-cycloalkyl-alkylsulfonyl, di(C₁-C₃)alkyl(C₃-C₈)cycloalkylsulfonyl, di(C₁-C₃)alkyl(C₄-C₈)-cycloalkyl-alkylsulfonyl, halo(C₁-C₈)alkylsulfonyl, halo(C₃-C₈)cycloalkylsulfonyl, halo(C₄-C₈)cycloalkyl(C₁-C₆)alkyl-sulfonyl, (C₁-C₈)alkylamino, di(C₁-C₈)alkylamino, (C₁-C₆)alkoxy-(C₁-C₆)-alkoxy, halo(C₁-C₆)alkoxy(C₁-C₆)alkoxy, (C₁-C₈)alkoxycarbonyl, aminocarbonyl, (C₁-C₈)alkyl-amino-carbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)-cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio-(C₁-C₆)alkyl, (C₃-C₈)cycloalkythio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio-(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)-cycloalkyl-sulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈)-cycloalkyl(C₁-C₆)alkyl-sulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino-(C₁-C₆)alkyl, (C₁-C₈)alkoxycarbonyl(C₁-C₆)alkyl, (C₁-C₈)acyloxy(C₁-C₆)alkyl, aminocarbonyl-(C₁-C₆)alkyl, (C₁-C₈)alkylaminocarbonyl(C₁-C₆)alkyl, di(C₁-C₈)alkylamino-carbonyl(C₁-C₆)alkyl, (C₁-C₈)acylamino(C₁-C₆)alkyl, piperidino(C₁-C₆)alkyl, pyrrolidino(C₁-C₆)alkyl, (C₁-C₈)alkoxy-carbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl; and
2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, and bicyclic heteroaryl(C₁-C₃)alkoxy, each optionally substituted with 1 to 5 groups independently selected from the group consisting of fluorine, chlorine, cyano, (C₁-C₆)alkyl, halo(C₁-C₆)alkyl, (C₁-C₆)alkoxy, halo(C₁-C₆)alkoxy, (C₁-C₆)alkylsulfonyl, (C₁-C₆)alkoxy-carbonyl, and aminocarbonyl;
X and Y are each independently C₂ or a single bond;
R² is a substituted or unsubstituted (C₁-C₂)alkyl, (C₂-C₁₂)alkenyl, (C₂-C₂)alkynyl, (C₁-C₁₂)alkoxy, (C₂-C₁₂)alkenyloxy, (C₁-C₂)alkylthio, (C₁-C₆)alkoxy(C₁-C₆)alkyl, (C₁-C₆)alkylthio-(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonylamino(C₁-C₆)alkylthio, (C₃-C₄)cycloalkylcarbonyl-amino(C₁-C₆)alkyl, (C₃-C₄)-cycloalkylcarbonylamino(C₁-C₆)alkoxy, (C₃-C₄)cycloalkylcarbonyl-amino(C₁-C₆)alkylthio, aminosulfonylamino(C₁-C₆)alkyl, aminosulfonylamino(C₁-C₁₂)alkoxy, aminosulfonylamino(C₁-C₁₂)alkylthio, (C₁-C₆)alkylsulfonylamino(C₁-C₆)alkyl, (C₁-C₆)alkylsulfonylamino(C₁-C₆)-alkoxy, (C₁-C₆)alkylsulfonylamino(C₁-C₆)alkylthio, formylamino(C₁-C₆)alkyl, form ylamino(C₁-C₆)alkoxy, formylamino(C₁-C₆)alkylthio, (C₁-C₆)alkoxycarbonylamino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)-alkylthio, aminocarbonyl(C₁-C₆)alkyl, aminocarbonyl(C₁-C₆)alkoxy, aminocarbonyl(C₁-C₆)-alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)-alkoxycarbonylamino, (C₁-C₁₂)alkylaminocarbonylamino, or (C₁-C₁₂)alkylcarbonylamino, wherein the substituted C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino-(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonyl-amino(C₁-C₆)alkylthio, (C₃-C₄)cycloalkylcarbonylamino(C₁-C₆)alkyl, (C₃-C₄)cycloalkyl-carbonyl-amino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, C₁-C₆)alkylsulfonylamino(C₁-C₆)alkyl, (C₁-C₆)alkylsulfonyl-amino(C₁-C₆)alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxy-carbonylamino(C₁-C₆)alkyl, (C₁-C₆)alkoxycarbonyl-amino(C₁-C₆)alkoxy, (C₁-C₆)-alkoxycarbonyl-amino(C₁-C₆)alkylthio, (C₁-C₆)alkylaminocarbonylamino(C₁-C₆)alkyl, di(C₁-C₆)alkylaminocarbonylamino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylamino-carbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino represented by R² is substituted by at least one of:
a) 1 to 6 halogen atoms; or
b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl, and halo(C₃-C₆)cycloalkoxy; and
wherein the thio-moiety of said unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonyl-amino(C₁-C₆)alkylthio, aminosulfonylamino(C₁-C₁₂)alkylthio, (C₁-C₆)alkyl-sulfonylamino(C₁-C₆)alkylthio, formylamino(C₁-C₆)alkylthio, (C₁-C₆)alkoxycarbonyl-amino(C₁-C₆)alkylthio, C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio is optionally replaced by —S(O)— or —S(O)₂—; and
wherein the carbonyl moiety of said unsubstituted or substituted aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonyl-amino(C₁-C₆)alkoxy, (C₁-C₆)alkylcarbonylamino(C₁-C₆)alkylthio, (C₃-C₄)-cycloalkylcarbonyl-amino(C₁-C₆)alkyl, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonyl-amino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonyl-amino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylamino-carboxyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino is optionally replaced by a thiocarbonyl moiety;
R³ is: a) —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkylcarbonylamino, (C₁-C₆)-alkoxycarbonylamino, (C₁-C₆)alkylamino-carbonylamino, di(C₁-C₆)alkylaminocarbonylamino, (C₁-C₆)alkylsulfonylamino, (C₁-C₆)alkylaminosulfonylamino, or di(C₁-C₆)alkylaminosulfonyl-amino; or
b) phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkyl(C₁-C₆)alkylthio, halo(C₁-C₆)alkylthio, halo(C₃-C₆)cycloalkythio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkyl-sulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cyclo-alkylalkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkyl(C₁-C₆)alkylthio-(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)-cycloalkylsulfinyl (C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl, and di(C₁-C₈)alkylaminocarboxy(C₁-C₆)alkyl;
provided that when R³ is hydroxyl, halogen, or optionally substituted phenylamino or heteroarylamino, then R² is not an optionally substituted alkoxy alkylthio or amino group as follows:
1) when R³ is hydroxyl, halogen, or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonyl-amino(C₁-C₁₂)alkoxy, (C₁-C₆)alkylcarbonyl-amino(C₁-C₆)alkoxy, (C₃-C₄)cycloalkylcarbonylamino-(C₁-C₆)alkoxy, aminosulfonylamino(C₁-C₁₂)alkoxy, (C₁-C₆)alkyl-sulfonylamino(C₁-C₆)alkoxy, formylamino(C₁-C₆)alkoxy, (C₁-C₆)alkoxycarbonyl-amino(C₁-C₆)alkoxy, (C₁-C₆)alkylaminocarbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl-(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkylcarbonylamino;
2) when R³ is hydroxyl, halogen, or optionally substituted phenylamino or heteroarylamino, then R² is not an unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkylsulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio, wherein the thin moiety is replaced by —S(O)— or —S(O)₂—; and
3) when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkylcarbonyl-amino(C₁-C₆)alkoxy, (C₁-C₆)alkylcarbonylamino(C₁-C₆)alkylthio, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkoxy, formylamino(C₁-C₆)alkylthio, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, ant inocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkoxy, aminocarboxy(C₁-C₆)alkylthio, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino, wherein the carbonyl moiety is replaced by a thiocarbonyl moiety;
A is a saturated or unsaturated 4-, 5-, 6-, or 7-membered ring which is optionally bridged by (CH₂)ₘ via bonds to two members of said ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from the group consisting of halogen, (C₁-C₆)alkyl, halo(C₁-C₆)alkyl, and oxo; where m is 1 to 3;
the carbonyl moiety and Y are attached to carbon or nitrogen atoms in ring A in a 1,2 or 1,3 or 1,4 relationship;
L is a linear (C₁-C₆)alkyl chain which is optionally substituted by 1-4 groups independently selected from R⁴, R⁵, R⁶, and R⁷; wherein each R⁴, R⁵, R⁶, and R⁷ is independently selected from hydroxyl, carboxy (hydroxycarbonyl), amino, amido (aminocarbonyl), and from the following optionally substituted groups:
1) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁C₂-C₃)alkenyl, (C₂-C₁₂)alkynyl, (C₃-C₈)cycloalkyl(C₁C₂-C₃)alkynyl, (C₁-C₆)alkoxy, (C₃-C₆)cycloalkoxy, halo(C₁-C₆)alkoxy, halo(C₃-C₆)cycloalkoxy, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, (C₁-C₁₂)alkoxycarbonyl, (C₃-C₈)cycloalkyl-oxy-carbonyl, (C₁-C₆)alkylaminocarbonyl, di[(C₁-C₆)alkyl]aminocarbonyl, saturated heterocyclyl, and saturated heterocyclyl(C₁-C₃)alkyl; each is optionally and independently substituted by a group selected from: halogen, cyano, hydroxyl, carboxy, amino, amido, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and
2) phenyl, naphthyl, heteroaryl, phenoxycarbonyl, naphthyloxycarbonyl, heteroaryloxycarbonyl, phenylaminocarbonyl, naphthylaminocarbonyl, heteroarylaminocarbonyl, (phenyl)((C₁-C₆)alkyl)aminocarbonyl, (naphthyl)((C₁-C₆)alkyl)aminocarbonyl, (heteroaryl)((C₁-C₆)alkyl)aminocarbonyl, phenyl(C₁-C₃)alkyl, naphthyl(C₁-C₃)alkyl, heteroaryl(C₁-C₃)alkyl, phenyl(C₁-C₃)alkoxycarbonyl, naphthyl(C₁-C₃)alkoxycarbonyl, heteroaryl(C₁-C₃)alkoxycarbonyl, phenyl(C₁-C₃)alkylaminocarbonyl, naphthyl(C₁-C₃)alkylaminocarbonyl, heteroaryl(C₁-C₃)alkylaminocarbonyl, (phenyl(C₁-C₃)alkyl)((C₁-C₆)alkyl)aminocarbonyl, (naphthyl(C₁-C₃)alkyl)((C₁-C₆)alkyl)aminocarbonyl, and (heteroaryl(C₁-C₃)alkyl))((C₁-C₆)alkyl)aminocarbonyl: each optionally substituted with 1 to 3 groups independently selected from fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₁)alkoxy, halo(C₁-C₃)-alkoxy, (C₁-C₃)alkylsulfonyl, and (C₁-C₃)alkoxycarbonyl;
or L is —NH(C₂-C₆)alkyl optionally substituted by hydroxyl, carboxy (hydroxycarbonyl), amino, amido (aminocarbonyl-), or (C₁-C₆)alkoxycarbonyl, when R² is aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkyl carbonylamino(C₁-C₆)alkoxy, (C₁-C₆)alkylcarbonylamino(C₁-C₆)alkylthio, (C₃-C₄)cycloalkylcarbonyl-amino(C₁-C₆)alkyl, (C₃-C₄)-cycloalkylcarbonylamino(C₁-C₆)alkoxy, (C₃-C₄)cycloalkylcarbonyl-amino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkylsulfonylamino(C₁-C₆)alkyl, (C₁-C₆)alkyl-sulfonylamino(C₁-C₆)-alkoxy, (C₁-C₆)alkylsulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)-alkylthio, aminocarbonyl(C₁-C₆)alkyl, aminocarbonyl(C₁-C₆)alkoxy, aminocarbonyl(C₁-C₆)-alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)-alkoxycarbonylamino, (C₁-C₁₂)alkylaminocarbonylamino, or (C₁-C₁₂)alkylcarbonylamino, and R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkylcarbonylamino, (C₁-C₆)-alkoxycarbonylamino, (C₁-C₆)alkylamino-carbonylamino, di(C₁-C₆)alkylaminocarbonylamino, (C₁-C₆)alkylsulfonylamino, (C₁-C₆)alkylaminosulfonylamino, or di(C₁-C₆)alkylaminosulfonyl-amino, provided that when R³ is hydroxyl, or halogen, then R² is not an alkoxy, alkylthio or amino group as provided above;
or L is —NH(C₂-C₆)alkyl substituted by phenyl(C₁-C₃)alkoxycarbonyl, phenyl, naphthyl, heteroaryl, phenyl(C₁-C₃)alkyl, naphthyl(C₁-C₃)alkyl, or heteroaryl(C₁-C₃)alkyl, wherein the aromatic and heteroaromatic groups are optionally substituted by 1 to 3 groups selected from: fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₃-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acyl amino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl;
G is —OH, —OR⁸, —NH₂, —NHR⁸, —NR⁸R⁹, —NHC(═NH)NH₂, —NHC(═NH)NHR⁸; —C(═NH)NH₂, or —C(═NH)NHR⁸;
where R⁸ is: a) (C₁-C₁₂)alkyl, (C₄-C₁₂)cycloalkyl(C₁-C₆)alkyl, halo(C₁-C₁₂)alkyl, halo(C₄-C₁₂)cycloalkyl(C₁-C₆)alkyl, (C₂-C₁₂)alkenyl, (C₅-C₁₂)cycloalkyl(C₂-C₆)alkenyl, halo(C₂-C₁₂)alkenyl, halo(C₅-C₁₂)cycloalkyl(C₂-C₆)alkenyl, (C₂-C₁₂)alkynyl, (C₅-C₁₂)cycloalkyl(C₂-C₆)alkynyl, halo(C₂-C₁₂)alkynyl, halo(C₅-C₁₂)cycloalkyl(C₂-C₆)alkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkylsulfinyl(C₁-C₆)alkyl, halo(C₁-C₆)alkylsulfinyl(C₁-C₆)alkyl, (C₁-C₆)alkylsulfonyl(C₁-C₆)alkyl, halo(C₁-C₆)alkylsulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, amino(C₁-C₄)alkyl-carbonyl, (C₁-C₄)alkyl-amino(C₁-C₄)alkylcarbonyl, di((C₁-C₄)alkyl)-amino(C₁-C₄)alkylcarbonyl, cyano(C₁-C₆)alkyl, hydroxy(C₁-C₁₂)alkyl, amino(C₁-C₁₂)alkyl, (C₁-C₆)alkylamino(C₁-C₆)alkyl, di((C₁-C₆)alkyl)-aminocarboxy(C₁-C₆)alkyl, (C₁-C₆)alkoxycarbonyl(C₁-C₆)alkyl, saturated heterocyclyl, or saturated heterocyclyl(C₁-C₆)alkyl; or
b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from: 1) fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₃-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkyl(C₁-C₆)alkylthio(C₁-C₆)alkyl, (C₁-C₈)alkylsulfinyl(C₁-C₆)alkyl(C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acyl amino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl; and
2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkylsulfonyl, and (C₁-C₃)-alkoxycarbonyl; and
R⁹ is (C₁-C₆)alkyl or halo(C₁-C₆)alkyl.

2. A compound represented by Formula I: 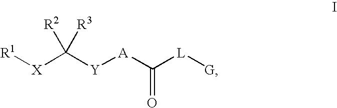
or a pharmaceutically acceptable salt thereof, wherein:
R¹ is:
a) (C₁-C₁₂)alkyl, (C₃-C₇)cycloalkyl, (C₄-C₁₂)cycloalkyl(C₁-C₆)alkyl, halo(C₁-C₁₂)alkyl, halo(C₃-C₇)-cycloalkyl, halo(C₄-C₁₂)cycloalkyl(C₁-C₆)alkyl, or saturated heterocyclyl, each optionally substituted with 1 to 5 groups independently selected from the group consisting of halogen, (C₁-C₆)alkyl, halo(C₁-C₆)alkyl, and oxo; or
b) phenyl, naphthyl, heteroaryl, or bicyclic heteroaryl, each optionally substituted with 1 to 5 groups independently selected from the groups consisting of:
1) fluorine, chlorine, bromine, iodine, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkyl(C₁-C₆)alkyl, (C₂-C₆)alkenyl, (C₅-C₈)cycloalkenyl, (C₅-C₈)cycloalkyl(C₂-C₆)alkenyl, (C₂-C₈)alkynyl, (C₃-C₈)cycloalkyl(C₂-C₄)alkynyl, halo(C₁-C₈)alkyl, halo(C₃-C₆)cycloalkyl, halo(C₄-C₈)cycloalkyl(C₁-C₆)alkyl, (C₁-C₃)alkyl(C₄-C₈)cycloalkyl(C₁-C₆)alkyl, di(C₁-C₃)alkyl(C₄-C₅)cycloalkyl(C₁-C₆)alkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkyl(C₂-C₆)alkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)-cycloalkylalkoxy, (C₁-C₈)alkylthio, (C₃-C₈)cycloalkythio, (C₄-C₈)cycloalkyl(C₁-C₆)alkylthio, (C₁-C₃)alkyl(C₃-C₈)cycloalkythio, (C₁-C₃)alkyl(C₄-C₈)cycloalkyl(C₁-C₆)alkylthio, di(C₁-C₃)alkyl(C₃-C₈)-cycloalkythio, di(C₁-C₃)alkyl(C₄-C₈)cycloalkyl(C₁-C₆)alkylthio, halo(C₁-C₈)alkylthio, halo(C₃-C₈)-cycloalkythio, halo(C₄-C₈)-cycloalkyl(C₁-C₆)alkylthio, (C₁-C₈)alkylsulfinyl, (C₃-C₈)-cycloalkyl-sulfinyl, (C₄-C₈)cycloalkyl-alkylsulfinyl, (C₁-C₃)alkyl(C₃-C₈)cycloalkyl-sulfinyl, (C₁-C₃)alkyl(C₄-C₈)cycloalkyl-alkylsulfinyl, di(C₁-C₃)alkyl(C₃-C₈)cycloalkyl-sulfinyl, di(C₁-C₃)alkyl(C₄-C₈)cycloalkyl-alkylsulfinyl, halo(C₁-C₈)alkylsulfinyl, halo(C₃-C₈)cycloalkylsulfinyl, halo(C₄-C₈)cycloalkyl(C₁-C₆)alkylsulfinyl, (C₁-C₈)alkyl-sulfonyl, (C₃-C₈)cycloalkylsulfonyl, (C₄-C₈) cycloalkyl(C₁-C₆)alkylsulfonyl, (C₁-C₃)alkyl(C₃-C₈)cycloalkylsulfonyl, (C₁-C₃)alkyl(C₄-C₈)-cycloalkyl-alkylsulfonyl, di(C₁-C₃)alkyl(C₃-C₈)cycloalkylsulfonyl, di(C₁-C₃)alkyl(C₄-C₈)-cycloalkyl-alkylsulfonyl, halo(C₁-C₈)alkylsulfonyl, halo(C₃-C₈)cycloalkylsulfonyl, halo(C₄-C₈)cycloalkyl(C₁-C₆)alkyl-sulfonyl, (C₁-C₈)alkylamino, di(C₁-C₈)alkylamino, (C₁-C₆)alkoxy-(C₁-C₆)-alkoxy, halo(C₁-C₆)alkoxy(C₁-C₆)alkoxy, (C₁-C₈)alkoxycarbonyl, aminocarbonyl, (C₁-C₈)alkyl-amino-carbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)-cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio-(C₁-C₆)alkyl, (C₃-C₈)cycloalkythio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio-(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)-cycloalkyl-sulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈)-cycloalkyl(C₁-C₆)alkyl-sulfonyl(C₁-C₆)alkyl, halo(C₁-C₈)alkylsulfonyl(C₁-C₆)alkyl, halo(C₃-C₈)cycloalkyl sulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino-(C₁-C₆)alkyl, (C₁-C₈)alkoxycarbonyl(C₁-C₆)alkyl, (C₁-C₈)acyloxy(C₁-C₆)alkyl, aminocarbonyl-(C₁-C₆)alkyl, (C₁-C₈)alkylaminocarbonyl(C₁-C₆)alkyl, di(C₁-C₈)alkylamino-carbonyl(C₁-C₆)alkyl, (C₁-C₈)acyl amino(C₁-C₆)alkyl, piperidino(C₁-C₆)alkyl, pyrrolidino(C₁-C₆)alkyl, (C₁-C₈)alkoxy-carbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl; and
2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, and bicyclic heteroaryl(C₁-C₃)alkoxy, each optionally substituted with 1 to 5 groups independently selected from the group consisting of fluorine, chlorine, cyano, (C₁-C₆)alkyl, halo(C₁-C₆)alkyl, (C₁-C₆)alkoxy, halo(C₁-C₆)alkoxy, (C₁-C₆)alkylsulfonyl, (C₁-C₆)alkoxy-carbonyl, and aminocarbonyl;
X and Y are each independently CH₂ or a single bond;
R² is a substituted or unsubstituted (C₁-C₁₂)alkyl, (C₂-C₁₂)alkenyl, (C₂-C₁₂)alkynyl, (C₁-C₁₂)alkoxy, (C₂-C₁₂)alkenyloxy, (C₁-C₁₂)alkylthio, (C₁-C₆)alkoxy(C₁-C₆)alkyl, (C₁-C₆)alkylthio-(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonylamino(C₁-C₆)alkoxy, (C₁-C₆)alkylcarbonylamino(C₁-C₆)alkylthio, (C₃-C₄)cycloalkylcarbonyl-amino(C₁-C₆)alkyl, (C₃-C₄)-cycloalkylcarbonylamino(C₁-C₆)alkoxy, (C₃-C₄)cycloalkylcarbonyl-amino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkylsulfonylamino(C₁-C₆)alkyl, (C₁-C₆)alkyl-sulfonylamino(C₁-C₆)-alkoxy, (C₁-C₆)alkyl sulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)-alkylthio, aminocarbonyl(C₁-C₆)alkyl, aminocarbonyl(C₁-C₆)alkoxy, aminocarbonyl(C₁-C₆)-alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)-alkoxycarbonylamino, (C₁-C₁₂)alkylaminocarbonylamino, or (C₁-C₁₂)alkylcarbonylamino, wherein the substituted (C₁-C₁₂)alkyl, (C₂-C₁₂)alkenyl, (C₂-C₁₂)alkynyl, (C₁-C₁₂)alkoxy, (C₁-C₁₇)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino-(C₁-C₁₂)alkyl, aminocarbonylamino(C₁-C₁₂)alkoxy, aminocarbonylamino(C₁-C₁₂)alkylthio, (C₁-C₆)-alkyl carbonylamino(C₁-C₆)alkyl, (C₁-C₆)alkylcarbonylamino(C₁-C₆)alkoxy, (C₁-C₆)alkylcarbonyl-amino(C₁-C₆)alkylthio, (C₃-C₄)cycloalkylcarbonylamino(C₁-C₆)alkyl, (C₃-C₄)cycloalkyl-carbonyl-amino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, C₁-C₆)alkylsulfonylamino(C₁-C₆)alkyl, (C₁-C₆)alkylsulfonyl-amino(C₁-C₆)alkoxy, (C₁-C₆)alkylsulfonylamino(C₁-C₆)alkylthio, formylamino(C₁-C₆)alkyl, formylamino(C₁-C₆)alkoxy, formyl amino(C₁-C₆)alkylthio, (C₁-C₆)alkoxy-carbonylamino(C₁-C₆)alkyl, (C₁-C₆)alkoxycarbonyl-amino(C₁-C₆)alkoxy, (C₁-C₆)-alkoxycarbonyl-amino(C₁-C₆)alkylthio, (C₁-C₆)alkylaminocarbonylamino(C₁-C₆)alkyl, di(C₁-C₆)alkylaminocarbonylamino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylamino-carbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminoearbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino represented by R² is substituted by at least one of:
a) 1 to 6 halogen atoms; or
b) one substituent selected from the group consisting of cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl, and halo(C₃-C₆)cycloalkoxy; and
wherein the thio-moiety of said unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkyl carbonyl-amino(C₁-C₆)alkylthio, aminosulfonylamino(C₁-C₁₂)alkylthio, (C₁-C₆)alkyl-sulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio is optionally replaced by —S(O)— or —S(O)₂—; and
wherein the carbonyl moiety of said unsubstituted or substituted aminocarbonylamino(C₁-C₁₂)alkyl, aminocarbonylamino(C₁-C₁₂)alkoxy, aminocarbonylamino(C₁-C₁₂)alkylthio, (C₁-C₆)-alkylcarbonylamino(C₁-C₆)alkyl, (C₁-C₆)alkylcarbonyl-amino(C₁-C₆)alkoxy, (C₁-C₆)alkylcarbonylamino(C₁-C₆)alkylthio, (C₃-C₄)-cycloalkylcarbonylamino(C₁-C₆)alkyl, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkyl, formylamino(C₁-C₆)alkoxy, formyl amino(C₁-C₆)alkylthio, (C₁-C₆)alkoxycarbonyl-amino(C₁-C₆)alkyl, (C₁-C₆)alkoxycarbonyl-amino(C₁-C₆)alkoxy, (C₃-C₆)alkoxycarbonyl-amino(C₁-C₆)alkylthio, (C₁-C₆)alkylaminocarbonylamino(C₁-C₆)alkyl, di(C₁-C₆)alkylaminocarbonyl-amino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonyl-amino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylamino-carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino is optionally replaced by a thiocarbonyl moiety;
R³ is: a) —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkylcarbonylamino, (C₁-C₆)-alkoxycarbonylamino, (C₁-C₆)alkylamino-carbonylamino, di(C₁-C₆)alkylaminocarbonylamino, (C₁-C₆)alkylsulfonylamino, (C₁-C₆)alkylaminosulfonylamino, or di(C₁-C₆)alkylaminosulfonyl-amino; or
b) phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkyl(C₁-C₆)alkylthio, halo(C₁-C₆)alkylthio, halo(C₃-C₆)cycloalkythio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkyl-sulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cyclo-alkylalkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio-(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkyl(C₁-C₆)alkylthio(C₁-C₆)alkyl, (C₁-C₈)alkylsulfinyl(C₁-C₆)alkyl, (C₃-C₈)-cycloalkylsulfinyl(C₁-C₆)alkyl, (C₄-C₈)cycloalkyl-alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkyl-sulfonyl(C₁-C₆)alkyl, (C₃-C₈)cycloalkylsulfonyl(C₁-C₆)alkyl, (C₄-C₈) cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkyl-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, C₈)alkylaminocarbonyl(C₁-C₆)alkyl(C₁-C₈)acylamino(C₁-C₆)alkyl, C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl, and di(C₁-C₈)alkylaminocarboxy(C₁-C₆)alkyl;
provided that when R³ is hydroxyl, halogen, or optionally substituted phenylamino or heteroarylamino, then R² is not an optionally substituted alkoxy alkylthio or amino group as follows:
1) when R³ is hydroxyl, halogen, or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonyl-amino(C₁-C₁₂)alkoxy, (C₁-C₆)alkylcarbonyl-amino(C₁-C₆)alkoxy, (C₃-C₄)cycloalkylcarbonylamino-(C₁-C₆)alkoxy, aminosulfonylamino(C₁-C₁₂)alkoxy, (C₁-C₆)alkyl-sulfonylamino(C₁-C₆)alkoxy, formylamino(C₁-C₆)alkoxy, (C₁-C₆)alkoxycarbonyl-amino(C₁-C₆)alkoxy, (C₁-C₆)alkylaminocarbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl-(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkylcarbonylamino;
2) when R³ is hydroxyl, halogen, or optionally substituted phenylamino or heteroarylamino, then R² is not an unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkylcarbonylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkylsulfonylamino(C₁-C₆)alkylthio, formylamino(C₁-C₆)alkylthio, (C₁-C₆)alkoxycarbonyl-amino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl-(C₁-C₆)alkylthio, aminocarboxy(C₁-C₆)alkylthio, or (C₁-C₆)alkylaminocarboxy(C₁-C₆)alkylthio, wherein the thio moiety is replaced by —S(O)— or —S(O)₂—; and
3) when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkylcarbonyl-amino(C₁-C₆)alkoxy, (C₁-C₆)alkylcarbonylamino(C₁-C₆)alkylthio, (C₃-C₄)cycloalkylcarbonylamino(C₁-C₆)alkoxy, (C₃-C₄)cycloalkyl-carbonylamino(C₁-C₆)alkylthio, formylamino(C₁-C₆)alkoxy, formylamino(C₁-C₆)alkylthio, (C₁-C₆)alkoxycarbonyl-amino(C₁-C₆)alkoxy, (C₁-C₆)alkoxycarbonyl-amino(C₁-C₆)alkylthio, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl-(C₁-C₆)alkylthio, aminocarboxy(C₁-C₆)alkoxy, aminocarboxy(C₁-C₆)alkylthio, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkylcarbonylamino, wherein the carbonyl moiety is replaced by a thiocarbonyl moiety;
A is a saturated or unsaturated 4-, 5-, 6-, or 7-membered ring which is optionally bridged by (CH₂)ₘ via bonds to two members of said ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from the group consisting of halogen, (C₁-C₆)alkyl, halo(C₁-C₆)alkyl, and oxo; where m is 1 to 3;
the carbonyl moiety and Y are attached to carbon or nitrogen atoms in ring A in a 1,2 or 1,3 or 1,4 relationship;
L is a linear (C₁-C₆)alkyl chain which is optionally substituted by 1-4 groups independently selected from R⁴, R⁵, R⁶, and R⁷; wherein each R⁴, R⁵, R⁶, and R⁷ is independently selected from hydroxyl, carboxy (hydroxycarbonyl), amino, amido (aminocarbonyl), and from the following optionally substituted groups:
1) (C₁-C₁₂)allyl, (C₃-C₈)cycloalkyl, (C₃-C₈)cycloalkyl(C₁-C₃)alkyl, (C₂-C₇₂)alkenyl, (C₅-C₈)cycloalkyl(C₂-C₃)alkenyl, (C₂-C₁₂)alkynyl, (C₃-C₈)cycloalkyl(C₂-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, (C₁-C₁₂)alkoxycarbonyl(C₃-C₈)cycloalkyl-oxy-carbonyl, (C₁-C₆)alkylaminocarbonyl, di[(C₁-C₆)alkyl]aminocarbonyl, saturated heterocyclyl, and saturated heterocyclyl(C₁-C₃)alkyl: each is optionally and independently substituted by a group selected from: halogen, cyano, hydroxyl, carboxy, amino, amido, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and
2) phenyl, naphthyl, heteroaryl, phenoxycarbonyl, naphthyloxycarbonyl, heteroaryloxycarbonyl, phenylaminocarbonyl, naphthylaminocarbonyl, heteroaryl aminocarbonyl, (phenyl)((C₁-C₆)alkyl)aminocarbonyl, (naphthyl)((C₁-C₆)alkyl)aminocarbonyl, (heteroaryl)((C₁-C₆)alkyl)aminocarbonyl, phenyl(C₁-C₃)alkyl, naphthyl(C₁-C₃)alkyl, heteroaryl(C₁-C₃)alkyl, phenyl(C₁-C₃)alkoxycarbonyl, naphthyl(C₁-C₃)alkoxycarbonyl, heteroaryl(C₁-C₃)alkoxycarbonyl, phenyl(C₁-C₃)alkylaminocarbonyl, naphthyl(C₁-C₃)alkylaminocarbonyl, heteroaryl(C₁-C₃)alkylaminocarbonyl, (phenyl(C₁-C₃)alkyl)((C₁-C₆)alkyl)aminocarbonyl, (naphthyl(C₁-C₃)alkyl)((C₁-C₆)alkyl)aminocarbonyl, and (heteroaryl(C₁-C₃)alkyl))((C₁-C₆)alkyl)aminocarbonyl; each optionally substituted with 1 to 3 groups independently selected from fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsufonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkylsulfonyl, and (C₁-C₃)alkoxycarbonyl;
G is —OH, —OR⁸, —NH₂, —NHR⁸, —NR⁸R⁹, —NHC(═NH)NHR⁸; —C(═NH)NH₂, or —C(═NH)NHR⁸;
where R⁸ is: a) (C₁-C₁₂)alkyl, (C₄-C₁₂)cycloalkyl(C₁-C₆)alkyl, halo(C₁-C₁₂)alkyl, halo(C₄-C₁₂)cycloalkyl(C₁-C₆)alkyl, (C₂-C₁₂)alkenyl, (C₅-C₁₂)cycloalkyl(C₂-C₆)alkenyl, halo(C₂-C₁₂)alkenyl, halo(C₅-C₁₂)cycloalkyl(C₂-C₆)alkenyl, (C₂-C₁₂)alkynyl, (C₅-C₁₂)cycloalkyl(C₂-C₆)alkynyl, halo(C₂-C₁₂)alkynyl, halo(C₅-C₁₂)cycloalkyl(C₂-C₆)alkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkylsulfinyl(C₁-C₆)alkyl, halo(C₁-C₆)alkylsulfinyl(C₁-C₆)alkyl, (C₁-C₆)alkylsulfonyl(C₁-C₆)alkyl, halo(C₁-C₆)alkylsulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, amino(C₁-C₄)alkyl-carbonyl, (C₁-C₄)alkyl-amino(C₁-C₄)alkylcarbonyl, di((C₁-C₄)alkyl)-amino(C₁-C₄)alkylcarbonyl, cyano(C₁-C₆)alkyl, hydroxy(C₁-C₁₂)alkyl, amino(C₁-C₁₂)alkyl, (C₁-C₆)alkylamino(C₁-C₆)alkyl, di((C₁-C₆)alkyl)-amino C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or
b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from: 1) fluoride, chloride, bromide, iodide, cyano, nitro, amino, hydroxy, carboxy, (C₁-C₆)alkyl, (C₃-C₆)cycloalkyl, (C₄-C₇)cycloalkyl(C₁-C₆)alkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkyl(C₁-C₆)alkyl, C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkyl(C₁-C₆)alkylthio, halo(C₁-C₆)alkylthio, halo(C₃-C₆)cycloalkylthio, halo(C₄-C₇)cycloalkyl(C₁-C₆)alkylthio, (C₁-C₆)alkylsulfinyl, (C₃-C₆)cycloalkylsulfinyl, (C₄-C₇)cycloalkyl(C₁-C₆)alkylsulfinyl, halo(C₁-C₆)alkylsulfinyl, halo(C₃-C₆)cycloalkylsulfinyl, halo(C₄-C₇)cycloalkyl(C₁-C₆)alkylsulfinyl, (C₁-C₆)alkylsulfonyl, (C₃-C₆)cycloalkylsulfonyl, (C₄-C₇)cycloalkyl(C₁-C₆)alkylsulfonyl, halo(C₁-C₆)alkylsulfonyl, halo(C₃-C₆)cycloalkylsulfonyl, halo(C₄-C₇)cycloalkyl(C₁-C₆)alkylsulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₆)alkylthio(C₁-C₆)alkyl, (C₃-C₈)cycloalkylthio(C₁-C₆)alkyl, (C₄-C₈)cycloalkyl(C₁-C₆)alkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkyl(C₁-C₆)alkylthio(C₁-C₆)alkyl, (C₁-C₈)alkylsulfinyl(C₁-C₆)alkyl, (C₃-C₈)cycloalkylsulfinyl(C₁-C₆)alkyl, (C₄-C₈)cycloalkyl(C₁-C₆)alkylsulfinyl(C₁-C₆)alkyl, halo(C₁-C₈)alkylsulfinyl(C₁-C₆)alkyl, halo(C₃-C₈)cycloalkylsulfinyl(C₁-C₆)alkyl, halo(C₄-C₈)cycloalkyl(C₁-C₆)alkylsulfinyl(C₁-C₆)alkyl, (C₁-C₈)alkylsulfonyl(C₁-C₆)alkyl, (C₃-C₈)cycloalkylsulfonyl(C₁-C₆)alkyl, (C₄-C₈)cycloalkyl(C₁-C₆)alkylsulfonyl(C₁-C₆)alkyl, halo(C₁-C₈)alkylsulfonyl(C₁-C₆)alkyl, halo(C₃-C₈)cycloalkylsulfonyl(C₁-C₆)alkyl, halo(C₄-C₈)cycloalkyl(C₁-C₆)alkylsulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl; and
2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkylsulfonyl, and (C₁-C₃)-alkoxycarbonyl; and
R⁹ is (C₁-C₆)alkyl or halo(C₁-C₆)alkyl.

3. The compound according to claim 1, wherein A is a saturated or unsaturated 5- or 6-membered ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from halogen, (C₁-C₆)alkyl, halo(C₁-C₆)alkyl, and oxo, and the carbonyl moiety and Y are attached to carbon or nitrogen atoms in ring A in a 1,3 or 1,4 relationship.

4. The compound according to claim 1, wherein the moiety —Y-A-C(═O)— is represented by: 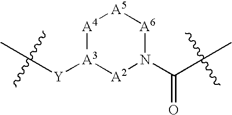
wherein A⁴ is CH₂ or O, and A², A⁵ and A⁶ are CH₂ and A³ is CH.

5. The compound according to claim 4, represented by Formula Ia: 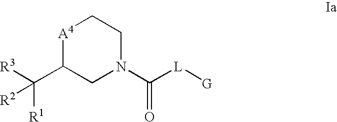
or a pharmaceutically acceptable salt thereof.

6. The compound according to claim 4, represented by Formula Ic: 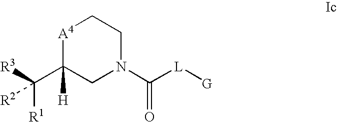
or a pharmaceutically acceptable salt thereof.

7. The compound according to claim 1, wherein R¹ is phenyl, optionally substituted with 1 to 3 substituents independently selected from halogen, cyano, hydroxyl, C₁-C₆ alkyl, haloC₁-C₆ alkyl, C₁-C₆ alkoxy, haloC₁-C₆ alkoxy, C₃-C₇ cycloalkyl, C₃-C₇ cycloalkoxy, (C₃-C₇)cycloalkyl(C₁-C₄ alkyl), (C₃-C₇)cycloalkyl(C₁-C₄ alkoxy), aminocarbonyl, phenyl, heteroaryl, phenoxy, benzyloxy, and heteroaryloxy, wherein the phenyl, heteroaryl, phenoxy, benzyloxy, and heteroaryloxy groups are optionally substituted with 1 to 3 substituents independently selected from halogen, cyano, C₁-C₄ alkyl, haloC₁-C₄ alkyl, C₁-C₄ alkoxy, and aminocarbonyl;
wherein the heteroaryl (and the heteroaryl moiety of the heteroaryloxy group) group is selected from a 5-6 membered monocyclic heteroaryl containing one or two heteroatoms selected from N, O, and S, and a 9-10 membered bicyclic heteroaryl containing 1 or 2 heteroatoms selected from N, O, and S, wherein at least one of the rings of the bicyclic group is aromatic.

8. The compound according to claim 1, wherein R¹ is phenyl, optionally substituted with 1 to 3 substituents independently selected from: fluorine, chlorine, and methyl, or is a bi-aromatic group comprised of a phenyl substituted with another aromatic moiety selected from phenyl, phenoxy, furanyl, quinolinyl, and dihydrobenzofuranyl, wherein the bi-aromatic group is optionally substituted with 1 to 3 substituents independently selected from fluorine, chlorine, methyl, ethyl, isopropyl, trifluoromethyl, and methoxy wherein the optional substituents are substituted on either one of or both of the phenyl moiety and the other aromatic moiety of the bi-aromatic group.

9. The compound according to claim 1, wherein R¹ is phenyl, 3-chlorophenyl, 3-fluorophenyl, 6-chloro-3′-ethyl-2-biphenyl, 3′-ethyl-6-fluoro-2-biphenyl, 6-fluoro-3′-methyl-2-biphenyl, 6-fluoro-3′-(1-methylethyl)-2-biphenyl, 3-fluoro-2-(3-quinolinyl)phenyl, 3-chloro-2-(3-quinolinyl)phenyl, 3-chloro-2-(2,3-dihydro-1-benzofuran-6-yl)phenyl, 2-[(2-methylphenyl)oxy]phenyl, 3-chloro-2-(5-methyl-2-furanyl)phenyl, 4′,6-difluoro-3′-methyl-2-biphenyl, 6-chloro-3′-fluoro-5′-methyl-2-biphenyl, 2′,6-difluoro-5′-methyl-2-biphenyl, 3′,6-difluoro-5′-methyl-2-biphenyl, or 2′-(methoxy)-5′-(trifluoromethyl)-2-biphenyl.

10. The compound according to claim 1, wherein R² is (C₁-C₅)alkoxy(C₁-C₅)alkyl, (C₁-C₅)alkylamino(C₁-C₅)alkyl, halo(C₁-C₅)alkoxy(C₁-C₅)alkyl, (C₁-C₅)alkoxy(C₁-C₅)alkoxy, hydroxy(C₁-C₈)alkoxy, halo(C₁-C₅)alkoxy(C₁-C₅)alkoxy, (C₁-C₃)alkoxy(C₁-C₃)alkoxy(C₁-C₃)alkyl, halo(C₁-C₃)-alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkylcarbonylamino(C₁-C₅)alkyl, (C₁-C₅)alkylcarbonylamino(C₁-C₅)alkoxy, halo(C₁-C₅)alkylcarbonylamino(C₁-C₅)alkyl, halo(C₁-C₅)alkylcarbonylamino(C₁-C₅)alkoxy, (C₁-C₃)alkoxy(C₁-C₅)alkylcarbonylamino(C₁-C₅)alkyl, (C₁-C₃)alkoxy(C₁-C₅)alkylcarbonylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylaminocarbonylamino(C₁-C₅)alkoxy, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)-alkylaminocarbonyl-(C₁-C₅)alkoxy, aminocarboxy(C₁-C₅)alkyl, aminocarboxy(C₁-C₅)alkoxy, (C₁-C₅)alkylamino-carboxy(C₁-C₅)alkyl, (C₁-C₅)alkylaminocarboxy(C₁-C₅)alkoxy, alkoxycarbonylamino, (C₁-C₈)alkylaminocarbonylamino, (C₁-C₈)alkyl carbonylamino, halo(C₁-C₈)alkoxycarbonylamino, halo(C₁-C₈)alkylaminocarbonylamino, or halo(C₁-C₈)-alkylcarbonylamino.

11. The compound according to claim 1, wherein R² is hydrogen, methyl, ethyl, propyl, butyl, hexyl, 5-pentenyl, 3,3,3-trifluoropropyl, 4,4-difluoropentyl, 3-(cyclopropyl)propyl, 4-(cyclopropyl)butyl, 3-hydroxypropyl, 4-hydroxybutyl, 4-hydroxypentyl, 4-hydroxyhexyl, 5-hydroxyhexyl, 2-hydroxyethoxy, 5-oxohexyl, 3-ethoxypropyl, 4-methoxybutyl, 4-ethoxybutyl, butoxy, hexyloxy, 2-(ethoxy)-ethoxy, 3-methoxypropoxy, 3-ethoxypropoxy, 3-propoxypropoxy, 2-cyclopropylethoxy, (2-(methoxy)ethoxy)methyl, 3-(2,2,2-trifluoroethylamino)propyl, 3-(formylamino)propyl, 3-(acetylamino)propyl, 3-(propionyl-amino)propyl, 3-(butanoylamino)propyl, 3-((2-methoxypropionyl)amino)propyl, 3-(cyclopropyl-carbonylamino)propyl, 3-(trifluoroacetylamino)propyl, 3-(methoxycarbonylamino)propyl, 3-(ethoxycarbonylamino)propyl, 2-(methoxycarbonylamino)ethoxy, 2-(ethoxycarbonylamino)-ethoxy, 3-(methylaminocarbonylamino)propyl, 3-(dimethylaminocarbonyl-amino)propyl, 3-(aminocarbonyl)propyl, 3-(methylaminocarbonyl)propyl, 3-(ethylamino-carbonyl)propyl, 2-(acetylamino)ethoxy, 2-(propionylamino)ethoxy, aminocarbonylmethoxy, methylamino-carbonylmethoxy, ethylaminocarbonylmethoxy, propylaminocarbonylmethoxy, 2-(methylaminocarbonyl)ethoxy, 2-(ethylaminocarbonyl)ethoxy, 2-(propylaminocarbonyl)ethoxy, (2-(methoxy)ethoxy)carbonylamino, methoxymethylcarbonylaminomethyl, 3-(aminosulfonylamino)propyl, 2-(methoxy)-ethoxy, or 4-(methoxy)-butoxy.

12. The compound according to claim 1, wherein R² is selected from (C₁-C₃)alkoxyprop-3-yloxy, (C₁-C₃)alkoxybut-4-yl, ((C₁-C₃)alkoxycarbonylamino)eth-2-yloxy, ((C₁-C₃)alkoxycarbonylamino)prop-3-yl, and [(optionally substituted (C₃-C₃)alkyl)carbonylamino]prop-3-yl, wherein the optionally substituted (C₁-C₃)alkyl moiety is optionally substituted with one or more substituents selected from halogen and hydroxyl; and R³ is H, halogen, or hydroxyl, provided that when R³ is halogen or hydroxyl. R² is not (C₁-C₃)alkoxyprop-3-yloxy or ((C₁-C₃)alkoxycarbonylamino)eth-2-yloxy.

13. The compound according to claim 1, wherein R² is 3-(methoxy)propoxy, 4-(methoxy)butyl, 3-(trifluoroacetylamino)propyl, 3-(methoxycarbonylamino)propyl, 3-(methylcarbonylamino)propyl, or 2-(methoxycarbonylamino)ethoxy, and R³ is 1-1, halogen, or hydroxyl, provided that when R³ is halogen or hydroxyl, R² is not 3-(methoxy)propoxy or 2-(methoxycarbonylamino)ethoxy.

14. The compound according to claim 1, wherein R² is 4-(methoxy)butyl, 3-(methoxycarbonylamino)propyl, 3-(trifluoroacetylamino)propyl, or 3-(methylcarbonylamino)propyl; and R³ is OH.

15. The compound according to claim 1, wherein L is a linear (C₁-C₆)alkyl chain, which is optionally substituted by 1-2 groups independently selected from hydroxyl, carboxy, amino, amido, and from the following optionally substituted groups:
1) (C₁-C₄)alkyl, C₁-C₆ alkoxy, (C₁-C₆)alkoxy(C₁-C₆)alkyl, (C₁-C₆)alkoxycarbonyl, (C₃-C₈)cycloalkyl-(C₁-C₆)alkoxy-carbonyl, (C₅-C₆)cycloalkyl-(C₁-C₃)alkyl, (C₁-C₆)alkylaminocarbonyl, each optionally and independently substituted by a group selected from: halogen, hydroxyl, carboxy, amino, amido, (C₁-C₃)alkyl, (C₁-C₃)alkoxy, (C₃-C₆)cycloalkyl, halo(C₁-C₃)alkyl, and halo(C₁-C₃)alkoxy; and
2) phenyl, heteroaryl, phenyl(C₁-C₃)alkyl, heteroaryl(C₁-C₃)alkyl, phenyl(C₁-C₃)alkoxycarbonyl, heteroaryl(C₁-C₃)alkoxycarbonyl, phenyl(C₁-C₃)alkylaminocarbonyl, and heteroaryl(C₁-C₃)alkylaminocarbonyl; each optionally substituted with 1 to 3 groups independently selected from halogen, (C₁-C₆)alkyl, halo(C₁-C₆)alkyl, and (C₁-C₆)alkylamino.

16. The compound according to claim 1 wherein L is a linear (C₁-C₅)alkyl chain which is unsubstituted or substituted by 1-2 substituents independently selected from methyl, isopropyl, hydroxy, hydroxymethyl, methoxy, carboxy, methoxycarbonyl, amino, aminopropyl, amido, amidomethyl, 4-chlorophenyl, benzyl, cyclohexylmethyl, benzyloxycarbonyl, and indolylmethyl.

17. The compound according to claim 1, wherein L is CH₂, CH₂CH₂, CH₂CH₂CH₂, CH₂CH₂CH₂CH₂, CH₂CH₂CH₂CH₂CH₂, CH(CH₃), C(CH₃)(CH₃), CH(CH(CH₃)₂), CH(CH₂OH), C(CH₃)(CH₂OH)CH₂, CH₂CH₂CH(CO₂H), CH₂CH₂CH(CO₂CH₃), CH₂CH₂CH(CH₂OH), CH₂CH(OH)CH₂, CH(OH)CH₂CH₂, CH(CH₂-phenyl), CH₂CH(4-chlorophenyl)CH₂, CH₂CH(OCH₃)CH₂, CH₂CH(CH₂-cyclohexyl), CH₂CH(CH₂-cyclohexyl)CH₂, CH₂CH(OH)CH(CH₂-phenyl), CH(CH₂-indol-3-yl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, or CH(NH₂)CH₂CH₂CH₂.

18. The compound according to claim 1, wherein L is —NH(C₂-C₆)alkyl optionally substituted by hydroxyl, carboxy, amino, amido, or (C₁-C₆)alkoxycarbonyl, when R² is selected from ((C₁-C₃)alkoxycarbonylamino)prop-3-yl and optionally substituted [((C₁-C₃)alkyl)carbonylamino]prop-3-yl, wherein the optionally substituted (C₁-C₃)alkyl moiety is optionally substituted with one or more substituents selected from halogen and hydroxyl; and R³ is H, halogen, or hydroxyl.
or L is —NH(C₂-C₆)alkyl substituted by phenyl(C₁-C₃)alkoxycarbonyl, phenyl, heteroaryl, phenyl(C₁-C₃)alkyl, or heteroaryl(C₁-C₃)alkyl, wherein the phenyl and heteroaromatic groups are optionally substituted by 1 to 3 groups selected from: fluoride, chloride, bromide, cyano, nitro, amino, hydroxy, carboxy, (C₁-C₄)alkyl, (C₃-C₆)cycloalkyl, halo(C₁-C₄)alkyl, (C₁-C₄)alkoxy, (C₃-C₆)cycloalkoxy, halo(C₁-C₄)alkoxy, (C₁-C₄)alkylamino, di(C₃-C₄)alkylamino, (C₁-C₄)alkoxycarbonyl, aminocarbonyl, (C₁-C₄)alkylaminocarbonyl, di(C₁-C₄)alkylaminocarbonyl, hydroxy(C₁-C₃)alkyl, (C₁-C₄)alkoxy(C₁-C₃)alkyl, (C₁-C₄)alkylamino(C₁-C₃)alkyl, di(C₁-C₄)alkylamino(C₁-C₃)alkyl, (C₁-C₄)alkoxycarbonyl(C₁-C₃)alkyl, aminocarbonyl(C₁-C₃)alkyl, (C₁-C₄)alkylaminocarbonyl(C₁-C₃)alkyl, and di(C₁-C₄)alkylaminocarbonyl(C₁-C₃)alkyl.

19. The compound according to claim 1, wherein L is —NH(C₂-C₆)alkyl substituted by phenyl or phenyl(C₁-C₃)alkyl, wherein said phenyl groups are optionally substituted by 1-2 groups selected from: fluoride, chloride, bromide, cyano, nitro, amino, hydroxy, (C₁-C₄)alkyl, halo(C₁-C₄)alkyl, and (C₁-C₄)alkoxy.

20. The compound according to claim 1, wherein L is NHCH₂CH(CH₂-4-OCH₃-phenyl), or NHCH₂CH(CH₂CH₂-4-OCH₃-phenyl,
or L is NHCH₂CH₂ or NHCH₂C(CH₃)₂CH₂, provided that R² is 3-(methoxycarbonylamino)propyl, and R³ is OH.

21. The compound according to claim 1 wherein G is —OH, —NH₂, NHR⁸, or —NR⁸R⁹, wherein R⁸ is a) (C₁-C₄)alkyl, halo(C₁-C₄)alkyl, hydroxy(C₁-C₄)alkyl, amino(C₁-C₄)alkyl, (C₁-C₄)alkyl-amino(C₁-C₄)alkyl, di((C₁-C₄)alkyl)-amino C₁-C₄)alkyl, amino(C₁-C₄)alkyl-carbonyl, (C₁-C₄)alkyl-amino(C₁-C₄)alkylcarbonyl, di((C₁-C₄)alkyl)-amino(C₁-C₄)alkylcarbonyl, (C₄-C₁₀)cycloalkyl(C₁-C₄)alkyl, (C₁-C₄)alkoxy(C₁-C₄)alkyl, aminocarbonyl(C₁-C₄)alkyl, (C₁-C₄)alkylaminocarbonyl(C₁-C₄)alkyl, di(C₁-C₄)alkylaminocarbonyl(C₁-C₄)alkyl, or amino(imino)(C₁-C₄)alkyl; or b) phenyl(C₁-C₂)alkyl optionally substituted with 1 to 3 groups independently selected from halogen, cyano, (C₁-C₃)alkyl, halo(C₁-C₃)alkyl, (C₁-C₃)alkoxy, and halo(C₁-C₃)alkoxy; and R⁹ is (C₁-C₄)alkyl.

22. The compound according to claim 1, wherein G is —OH, —NH₂, NHR⁸, or —NR⁸R⁹, where R⁸ is methyl, hydroxyethyl, methylaminoethyl, aminomethylcarbonyl, amino(1-ethyl)carbonyl (L-alanyl), or amino(imino)methyl; and R⁹ is methyl.

23. The compound according claim 1, wherein G is selected from —NH₂, —NH(CH₃), —N(CH₃)₂, —NHCH₂CH₂OH, —N(CH₃)CH₂CH₂NH(CH₃), —NHC(═O)CH₂NH₂, —NHC(═O)CH(CH₃)NH₂, or —NHC(═NH)NH₂.

24. The compound according to claim 4, wherein R¹ is phenyl, optionally substituted with 1 to 3 substituents independently selected from: fluorine, chlorine, and methyl, or is a bi-aromatic group comprised of a phenyl substituted with another aromatic moiety selected from phenyl, phenoxy, furanyl, quinolinyl, and dihydrobenzofuranyl, wherein the bi-aromatic group is optionally substituted with 1 to 3 substituents independently selected from fluorine, chlorine, methyl, ethyl, isopropyl, trifluoromethyl, and methoxy, wherein the optional substituents are substituted on either one of or both of the phenyl moiety and the other aromatic moiety of the bi-aromatic group; R² is 3-(methoxy)propoxy, 4-(methoxy)butyl, 3-(trifluoroacetylamino)propyl, 3-(methoxycarbonylamino)propyl, 3-(methylcarbonylamino)propyl, or 2-(methoxycarbonylamino)ethoxy; R³ is H or OH; provided that when R³ is OH, R² is not 3-(methoxy)propoxy or 2-(methoxycarbonylamino)ethoxy; A⁴ is CH₂ or O; L is a linear (C₁-C₅)alkyl chain which is unsubstituted or substituted by 1-2 substituents selected from methyl, isopropyl, hydroxy, hydroxymethyl, methoxy, carboxy, methoxycarbonyl, amino, aminopropyl, amide, amidomethyl (aminocarbonylmethyl), 4-chlorophenyl, benzyl, cyclohexylmethyl, benzyloxycarbonyl, and indolylmethyl, or L is —NH(C₂-C₆)alkyl substituted by phenyl or phenyl(C₁-C₃)alkyl, wherein said phenyl groups are optionally substituted by 1 or 2 groups selected from: fluoride, chloride, bromide, cyano, nitro, amino, hydroxy, (C₁-C₄)alkyl, halo(C₁-C₄)alkyl, and (C₁-C₄)alkoxy; and G is OH, NH₂, NH(CH₃), —N(CH₃)₂, NHCH₂CH₂OH, N(CH₃)CH₂CH₂NH(CH₃), NHC(═O)CH₂NH₂, NHC(═O)CH(CH₃)NH₂, or —NHC(═NH)NH₂.

25. The compound according to claim 1, wherein R¹ is phenyl, 3-chlorophenyl, 3-fluorophenyl, 6-chloro-3′-ethyl-2-biphenyl, 3′-ethyl-6-fluoro-2-biphenyl, 6-fluoro-3′-methyl-2-biphenyl, 6-fluoro-3′-(1-methylethyl)-2-biphenyl, 3-fluoro-2-(3-quinolinyl)phenyl, 3-chloro-2-(3-quinolinyl)phenyl, 3-chloro-2-(2,3-dihydro-1-benzofuran-6-yl)phenyl, 2-[(2-methylphenyl)oxy]phenyl, 3-chloro-2-(5-methyl-2-furanyl)phenyl, 4′,6-difluoro-3′-methyl-2-biphenyl, 6-chloro-3′-fluoro-5′-methyl-2-biphenyl, 2′,6-difluoro-5′-methyl-2-biphenyl, 3′,6-difluoro-5′-methyl-2-biphenyl, or 2′-(methoxy)-5′-(trifluoromethyl)-2-biphenyl; R² is 4-(methoxy)butyl, 3-(trifluoroacetylamino)propyl, 3-(methoxycarbonylamino)propyl, or 3-(methylcarbonylamino)propyl; R³ is H or OH; and L is CH₂, CH₂CH₂, CH₂CH₂CH₂, CH₂CH₂CH₂CH₂, CH₂CH₂CH₂CH₂CH₂, CH(CH₃), C(CH₃)(CH₃). CH(CH(CH₃)₂), CH(CH₂OH), C(CH₃)(CH₂OH)CH₂, CH₂CH₂CH(CO₂H), CH₂CH₂CH(CO₂CH₃), CH₂CH₂CH(CH₂OH), CH₂CH(OH)CH₂, CH(OH)CH₂CH₂, CH(CH₂-phenyl), CH₂CH(4-chlorophenyl)CH₂, CH₂CH(OCH₃)CH₂, CH₂CH(CH₂-cyclohexyl), CH₂CH(CH₂-cyclohexyl)CH₂, CH₂CH(OH)CH(CH₂-phenyl), CH(CH₂-indol-3-yl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, CH(NH₂)CH₂CH₂CH₂, NHCH₂CH₂ NHCH₂C(CH₃)₂CH₂, NHCH₂CH(CH₂-4-OCH₃-phenyl), or NHCH₂CH(CH₂CH₂-4-OCH₃-phenyl); and G is —NH₂, NH(CH₃), —N(CH₃)₂, NHCH₂CH₂OH, N(CH₃)CH₂CH₂NH(CH₃), NHC(═O)CH₂NH₂, NHC(═O)CH(CH₃)NH₂, or —NHC(═NF)NH₂.

26. The compound according to claim 2, wherein R¹ is phenyl, optionally substituted with 1 to 3 substituents independently selected from halogen, cyano, hydroxyl, C₁-C₆ alkyl, haloC₁-C₆ alkyl, C₁-C₆ alkoxy, haloC₁-C₆ alkoxy, C₃-C₇ cycloalkyl, C₃-C₇ cycloalkoxy, (C₃-C₇)cycloalkyl(C₁-C₄ alkyl), (C₃-C₇)cycloalkyl(C₁-C₄ alkoxy), aminocarbonyl, phenyl, heteroaryl, phenoxy, benzyloxy, and heteroaryloxy, wherein the phenyl, heteroaryl, phenoxy, benzyloxy and heteroaryloxy groups are optionally substituted with 1 to 3 substituents independently selected from halogen, cyano, C₁-C₄ alkyl, haloC₁-C₄ alkyl, and aminocarbonyl;
wherein the heteroaryl (and the heteroaryl moiety of the heteroaryloxy group) group is selected from a 5-6 membered monocyclic heteroaryl containing one or two heteroatoms selected from N, O and S, and a 9-10 membered bicyclic heteroaryl containing 1 or 2 heteroatoms selected from N, O and S, wherein at least one of the rings of the bicyclic group is aromatic.

27. The compound according to claim 2, wherein R¹ is phenyl, 2-thienyl, 3-thienyl, 2-pyridyl, 2-imidazolyl, 2-thiazolyl, 2-benzothienyl, 4-benzofuryl, 4-benzothienyl, 7-benzofuryl, 2,3-dihydro-7-benzofuryl, 7-benzothienyl, 1,3-benzodioxol-4-yl, 7-indazolyl, or 8-quinolinyl, each optionally substituted with 1 to 3 substituents independently selected from: fluorine, chlorine, bromine, cyano, methyl, ethyl, isopropyl, t-butyl, isobutyl, trifluoromethyl, allyl, cyclohexyl, cyclohexen-1-yl, cyclopropylethynyl, methoxy, trifluoromethoxy, neopentyloxy, methylthio, allyloxy, cyclopropylmethoxy, 2-(cyclopropyl)ethoxy, cyclopentyloxy, cyclopentylmethoxy, benzyloxy, hydroxyl, aminocarbonyl, methoxycarbonyl, phenyl, heteroaryl, phenoxy, benzyloxy, and heteroaryloxy, wherein the phenyl, heteroaryl, phenoxy, benzyloxy and heteroaryloxy groups are optionally substituted with 1 to 3 substituents independently selected from fluorine, chlorine, cyano, methyl, ethyl, trifluoromethyl, and aminocarbonyl;
wherein the heteroaryl (and the heteroaryl moiety of the heteroaryloxy group) group is selected from 2-furanyl, 2-thienyl, 3-thienyl, 2-pyridyl, 2-imidazolyl, 2-thiazolyl, 2-benzothienyl, 4-benzofuryl, 4-benzothienyl, 7-benzofuryl, 2,3-dihydro-7-benzofuryl, 2,3-dihydro-6-benzofuryl, 7-benzothienyl, 1,3-benzodioxol-4-yl, 7-indazolyl, 8-quinolinyl or 3-quinolinyl.

28. The compound according to claim 2, wherein R¹ is phenyl, optionally substituted with 1 to 3 substituents independently selected from: fluorine, chlorine, and methyl, or is a hi-aromatic group comprised of a phenyl substituted with another aromatic moiety selected from phenyl, phenoxy, furanyl, quinolinyl, and dihydrobenzofuranyl, wherein the hi-aromatic group is optionally substituted with 1 to 3 substituents independently selected from fluorine, chlorine, methyl and ethyl, wherein the optional substituents are substituted on either one of or both of the phenyl moiety and the other aromatic moiety of the bi-aromatic group.

29. The compound according to claim 2, wherein R¹ is 3-chlorophenyl, 3-fluorophenyl, 6-chloro-3′-ethyl-2-biphenyl, 3′-ethyl-6-fluoro-2-biphenyl, 3′-methyl-6-fluoro-2-biphenyl, 3-fluoro-2-(3-quinolinyl)phenyl, 3-chloro-2-(2,3-dihydro-1-benzofuran-6-yl)phenyl, 2-[(2-methylphenyl)oxy]phenyl, 3-chloro-2-(5-methyl-2-furanyl)phenyl, 4′,6-difluoro-3′-methyl-2-biphenyl, 6-chloro-3′-fluoro-5′-methyl-2-biphenyl, 2′,6-difluoro-5′-methyl-2-biphenyl, or 3′,6-difluoro-5′-methyl-2-biphenyl.

30. The compound according to claim 2, wherein R² is (C₁-C₅)alkoxy(C₁-C₅)alkyl, (C₁-C₅)alkylamino(C₁-C₅)alkyl, fluoro(C₁-C₅)alkoxy(C₁-C₅)alkyl, (C₁-C₅)alkoxy(C₁-C₅)alkoxy, hydroxy(C₁-C₈)alkoxy, fluoro(C₁-C₅)alkoxy(C₁-C₅)alkoxy, (C₁-C₃)alkoxy(C₁-C₃)alkoxy(C₁-C₃)alkyl, fluoro(C₁-C₃)-alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkylcarbonylamino(C₁-C₅)alkyl, (C₁-C₅)alkylcarbonylamino(C₁-C₅)alkoxy, fluoro(C₁-C₅) alkylcarbonylamino(C₁-C₅)alkyl, fluoro(C₁-C₅) alkylcarbonylamino(C₁-C₅)alkoxy, (C₁-C₃)alkoxy(C₁-C₅) alkylcarbonylamino(C₁-C₅)alkyl, (C₁-C₃)alkoxy(C₁-C₅) alkylcarbonylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylaminocarbonyl-amino(C₁-C₅)alkoxy, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)-alkylaminocarbonyl-(C₁-C₅)alkoxy, aminocarboxy(C₁-C₅)alkyl, aminocarboxy(C₁-C₅)alkoxy, (C₁-C₅)alkylamino-carboxy(C₁-C₅)alkyl, (C₁-C₅)alkylaminocarboxy(C₁-C₅)alkoxy, (C₁-C₈)-alkoxycarbonylamino, (C₁-C₈)alkylaminocarbonylamino, (C₁-C₈)alkylcarbonylamino, fluoro(C₁-C₈)alkoxycarbonylamino, fluoro(C₁-C₈)alkylaminocarbonylamino, or fluoro(C₁-C₈)-alkylcarbonylamino.

31. The compound according to claim 2, wherein R² is hydrogen, methyl, ethyl, propyl, butyl, hexyl, 5-pentenyl, 3,3,3-trifluoropropyl, 4,4-difluoropentyl, 3-(cyclopropyl)propyl, 4-(cyclopropyl)butyl, 3-hydroxypropyl, 4-hydroxybutyl, 4-hydroxypentyl, 4-hydroxyhexyl, 5-hydroxyhexyl, 2-hydroxyethoxy, 5-oxohexyl, 3-ethoxypropyl, 4-methoxybutyl, 4-ethoxybutyl, butoxy, hexyloxy, 2-(ethoxy)-ethoxy, 3-methoxypropoxy, 3-ethoxypropoxy, 3-propoxypropoxy, 2-cyclopropylethoxy, (2-(methoxy)ethoxy)methyl, 3-(2,2,2-trifluoroethylamino)propyl, 3-(formylamino)propyl, 3-(acetylamino)propyl, 3-(propionyl-amino)propyl, 3-(butanoylamino)propyl, 3-((2-methoxypropionyl)amino)propyl, 3-(cyclopropyl-carbonylamino)propyl, 3-(trifluoroacetylamino)propyl, 3-(methoxycarbonylamino)propyl, 3-(ethoxycarbonylamino)propyl, 2-(methoxycarbonylamino)ethoxy, 2-(ethoxycarbonylamino)-ethoxy, 3-(methylaminocarbonylamino)propyl, 3-(dimethylaminocarbonyl-amino)propyl, 3-(aminocarbonyl)propyl, 3-(methylaminocarbonyl)propyl, 3-(ethylamino-carbonyl)propyl, 2-(acetylamino)ethoxy, 2-(propionylamino)ethoxy, aminocarbonylmethoxy, methylamino-carbonylmethoxy, ethylaminocarbonylmethoxy, propylaminocarbonylmethoxy, 2-(methylaminocarbonyl)ethoxy, 2-(ethylaminocarbonyl)ethoxy, 2-(propylaminocarbonyl)ethoxy, (2-(methoxy)ethoxy)carbonylamino, methoxymethylcarbonylaminomethyl, 3-(aminosulfonylamino)propyl, 2-(methoxy)-ethoxy, or 4-(methoxy)-butoxy.

32. The compound according to claim 2, wherein R² is selected from (C₁-C₃)alkoxyprop-3-yloxy, (C₁-C₃)alkoxybut-4-yl, ((C₁-C₃)alkoxycarbonylamino)prop-3-yl and [(optionally substituted (C₁-C₃)alkyl)carbonylamino]prop-3-yl, wherein the optionally substituted (C₁-C₃)alkyl moiety is optionally substituted with one or more substituents selected from halogen and hydroxyl; and R³ is H, halogen, or hydroxyl, provided that when R³ is halogen or hydroxyl, R² is not (C₁-C₃)alkoxyprop-3-yloxy.

33. The compound according to claim 2, wherein R² is 3-(methoxy)propoxy, 4-(methoxy)butyl, 3-(methoxycarbonylamino)propyl, or 3-(methylcarbonylamino)propyl, and R³ is H, halogen, or hydroxyl, provided that when R³ is halogen or hydroxyl, R² is not 3-(methoxy)propoxy.

34. The compound according to claim 2, wherein R² is 4-(methoxy)butyl, 3-(methoxycarbonylamino)propyl, or 3-(methylcarbonylamino)propyl; and R³ is OH.

35. The compound according to claim 2, wherein L is a linear (C₁-C₆)alkyl chain, which is optionally substituted by 1-2 groups independently selected from hydroxyl, carboxy, amino, amido, and from the following optionally substituted groups:
1) (C₁-C₄)alkyl, (C₁-C₆)alkoxy(C₁-C₆)alkyl, (C₁-C₆)alkoxycarbonyl, (C₃-C₈)cycloalkyl-(C₁-C₆)alkoxy-carbonyl, (C₅-C₆)cycloalkyl-(C₁-C₃)alkyl, (C₁-C₆)alkylaminocarbonyl, each optionally and independently substituted by a group selected from: halogen, hydroxyl, carboxy, amino, amido, (C₁-C₃)alkyl, (C₁-C₃)alkoxy, (C₃-C₆)cycloalkyl, halo(C₁-C₃)alkyl, and halo(C₁-C₃)alkoxy; and
2) phenyl, heteroaryl, phenyl(C₁-C₃)alkyl, heteroaryl(C₁-C₃)alkyl, phenyl(C₁-C₃)alkoxycarbonyl, heteroaryl(C₁-C₃)alkoxycarbonyl, phenyl(C₁-C₃)alkylaminocarbonyl, and heteroaryl(C₁-C₃)alkylaminocarbonyl; each optionally substituted with 1 to 3 groups independently selected from halogen, (C₁-C₆)alkyl, halo(C₁-C₆)alkyl, and (C₁-C₆)alkylamino.

36. The compound according to claim 1, wherein L is a linear (C₁-C₆)alkyl chain, which is optionally substituted by 1-2 groups independently selected from hydroxyl, carboxy, amino, amido, and from the following optionally substituted groups:
1) (C₁-C₄)alkyl, (C₁-C₆)alkoxy(C₁-C₆)alkyl, (C₁-C₆)alkoxycarbonyl, (C₃-C₆)cycloalkyl-(C₁-C₆)alkoxy-carbonyl, (C₅-C₆)cycloalkyl-(C₁-C₂)alkyl, (C₁-C₆)alkylaminocarbonyl, each optionally and independently substituted by a group selected from: halogen, hydroxyl, carboxy, amino, amido, (C₁-C₃)alkyl, (C₁-C₃)alkoxy, (C₃-C₆)cycloalkyl, halo(C₁-C₃)alkyl, and halo(C₁-C₃)alkoxy; and
2) phenyl, heteroaryl, phenyl(C₁-C₃)alkyl, heteroaryl(C₁-C₃)alkyl, phenyl(C₁-C₃)alkoxycarbonyl, heteroaryl(C₁-C₃)alkoxycarbonyl, phenyl(C₁-C₃)alkylaminocarbonyl, and heteroaryl(C₁-C₃)alkylaminocarbonyl; each optionally substituted with 1 to 3 groups independently selected from halogen, (C₁-C₆)alkyl, halo(C₁-C₆)alkyl, and (C₁-C₆)alkylamino.

37. The compound according to claim 1, wherein L is a linear (C₁-C₅)alkyl chain which is unsubstituted or substituted by 1-2 substituents selected from methyl, isopropyl, hydroxy, hydroxymethyl, carboxy, methoxycarbonyl, amino, aminopropyl, amido, am idomethyl, 4-chlorophenyl, benzyl, cyclohexylmethyl, and benzyloxycarbonyl.

38. The compound according to claim 1, wherein L is CH₂, CH₂CH₂, CH₂CH₂CH₂, CH₂CH₂CH₂CH₂, CH₂CH₂CH₂CH₂CH₂, CH(CH₃), C(CH₃)(CH₃), CH(CH(CH₃)₂), CH(CH₂OH), CH₂CH₂CH(CO₂CH₃), CH₂CH₂CH(CH₂OH), CH₂CH(OH)CH₂, CH(OH)CH₂CH₂, CH(CH₂-phenyl), CH₂CH(CH₂-cyclohexyl), CH₂CH(OH)CH(CH₂-phenyl), CH(CH₂-indol-3-yl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, or CH(NH₂)CH₂CH₂CH₂.

39. The compound according to claim 1, wherein G is —OH, —NH₂, NHR⁸, or —NR⁸R⁹, wherein R⁸ is a) (C₁-C₄)alkyl, halo(C₁-C₄)alkyl, hydroxy(C₁-C₄)alkyl, amino(C₁-C₄)alkyl, (C₁-C₄)alkyl-amino(C₁-C₄)alkyl, di((C₁-C₄)alkyl)-amino C₁-C₄)alkyl, amino(C₁-C₄)alkyl-carbonyl, (C₁-C₄)alkyl-amino(C₁-C₄)alkylcarbonyl, di((C₁-C₄)alkyl)-amino C₁-C₄)alkylcarbonyl, (C₄-C₁₀)cycloalkyl(C₁-C₄)alkyl, (C₁-C₄)alkoxy(C₁-C₄)alkyl, aminocarbonyl(C₁-C₄)alkyl, (C₁-C₄)alkylaminocarbonyl(C₁-C₄)alkyl, or di(C₁-C₄)alkyl-aminocarbonyl(C₁-C₄)alkyl; or b) phenyl(C₁-C₂)alkyl optionally substituted with 1 to 3 groups independently selected from halogen, cyano, (C₁-C₃)alkyl, halo(C₁-C₃)alkyl, (C₁-C₃)alkoxy, and halo(C₁-C₃)alkoxy; and R⁹ is (C₁-C₄)alkyl.

40. The compound according to claim 1, wherein G is —OH, —NH₂, NHR⁸, or —NR⁸R⁹, where R⁸ is methyl, hydroxyethyl, methylaminoethyl, aminomethylcarbonyl, amino(1-ethyl)carbonyl (L-alanyl), and R⁹ is methyl.

41. The compound according to claim 1, wherein G is selected from —NH₂, —NH(CH₃), —NHCH₂CH₂OH, —N(CH₃)CH₂CH₂NH(CH₃), —NHC(═O)CH₂NH₂ or —NHC(═O)CH(CH₃)NH₂.

42. The compound according to claim 4, wherein R¹ is phenyl, optionally substituted with 1 to 3 substituents independently selected from: fluorine, chlorine, and methyl, or is a bi-aromatic group comprised of a phenyl substituted with another aromatic moiety selected from phenyl, phenoxy, furanyl, quinolinyl, and dihydrobenzofuranyl, wherein the bi-aromatic group is optionally substituted with 1 to 3 substituents independently selected from fluorine, chlorine, methyl and ethyl, wherein the optional substituents are substituted on either one of or both of the phenyl moiety and the other aromatic moiety of the bi-aromatic group; R² is 3-(methoxy)propoxy, 4-(methoxy)butyl, 3-(methoxycarbonylamino)propyl, or 3-(methylcarbonylamino)propyl; R³ is H or OH; provided that when le is OH, R² is not 3-(methoxy)propoxy; A⁴ is CH₂ or O; L is a linear (C₁-C₅)alkyl chain which is unsubstituted or substituted by 1-2 substituents selected from methyl, isopropyl, hydroxy, hydroxymethyl, carboxy, methoxycarbonyl, amino, aminopropyl, amido, amidomethyl (aminocarbonylmethyl), 4-chlorophenyl, benzyl, cyclohexylmethyl, and benzyloxycarbonyl, and G is OH, NH₂, NH(CH₃), NHCH₂CH₂OH, N(CH₃)CH₂CH₂NH(CH₃), NHC(═O)CH₂NH₂ or NHC(═O)CH(CH₃)NH₂.

43. The compound according to claim 2, wherein R¹ is 6-chloro-3′-ethyl-2-biphenyl, 3′-ethyl-6-fluoro-2-biphenyl, 3′-methyl-6-fluoro-2-biphenyl, 3-fluoro-2-(3-quinolinyl)phenyl, 3-chloro-2-(2,3-dihydro-1-benzofuran-6-yl)phenyl, 2-[(2-methylphenyl)oxy]phenyl, 3-chloro-2-(5-methyl-2-furanyl)phenyl, 4′,6-difluoro-3′-methyl-2-biphenyl, 6-chloro-3′-fluoro-5′-methyl-2-biphenyl, 2′,6-difluoro-5′-methyl-2-biphenyl, or 3′,6-difluoro-5′-methyl-2-biphenyl; R² is 4-(methoxy)butyl, 3-(methoxycarbonylamino)propyl, or 3-(methylcarbonylamino)propyl; R³ is OH; and L is CH₂, CH₂CH₂, CH₂CH₂CH₂, CH₂CH₂CH₂CH₂, CH₂CH₂CH₂CH₂CH₂, CH(CH₃), C(CH₃)(CH₃), CH(CH(CH₃)₂), CH(CH₂OH), CH₂CH₂CH(CO₂CH₃), CH₂CH₂CH(CH₂OH), CH₂CH(OH)CH₂, CH(OH)CH₂CH₂, CH(CH₂-phenyl), CH(CH₂CONH₂), CH₂CH₂CH(CO₂CH₂-phenyl), CH₂CH(CO₂CH₂-phenyl), CH₂CH₂CH(CO₂NH₂), CHCH₂CH₂CH₂NH₂, or CH(NH₂)CH₂CH₂CH₂ and G is —NH₂, NH(CH₃), NHCH₂CH₂OH, N(CH₃)CH₂CH₂NH(CH₃), NHC(═O)CH₂NH₂ or NHC(═O)CH(CH₃)NH₂.

44. A compound selected from the group
1-(6-chloro-3′-ethyl-2-biphenylyl)-1-[1-(2-methylalanyl)-3-piperidinyl]-5-(methyloxy)-1-pentanol,
2-({3-[1-(6-chloro-3′-ethyl-2-biphenylyl)-1-hydroxy-5-(methyloxy)pentyl]-1-piperidinyl}carbonyl)-2-methyl-1,3-propanediol,
1-(6-chloro-3′-ethyl-2-biphenylyl)-1-{-1-[N-(2-hydroxyethyl)-2-methylalanyl]-3-piperidinyl}-5-(methyloxy)-1-pentanol,
1-[1-(6-aminohexanoyl)-4-piperidinyl]-1-(3′-ethyl-6-fluoro-2-biphenylyl)-5-(methyloxy)-1-pentanol,
methyl (4-[3-fluoro-2-(3-quinolinyl)phenyl]-4-hydroxy-4-{1-[4-(methylamino)butanoyl]-3-piperidinyl}butyl)carbamate,
methyl (4-[3-chloro-2-(5-methyl-2-furanyl)phenyl]-4-hydroxy-4-{1-[4-(methylamino)butanoyl]-3-piperidinyl}butyl)carbamate,
1-[4-(4-aminobutanoyl)-2-morpholinyl]-1-(4′,6-difluoro-3′-methyl-2-biphenylyl)-5-(methyloxy)-1-pentanol,
methyl [4-{1-[4-amino-3-(4-chlorophenyl)butanoyl]-3-piperidinyl}-4-(3′-ethyl-6-fluoro-2-biphenylyl)-4-hydroxybutyl]carbamate,
methyl (4-(6-chloro-3′-fluoro-5′-methyl-2-biphenylyl)-4-hydroxy-4-{1-[4-(methylamino)butanoyl]-3-piperidinyl}butyl)carbamate,
methyl (4-(3′,6-difluoro-5′-methyl-2-biphenylyl)-4-hydroxy-4-{1-[4-(methylamino)butanoyl]-3-piperidinyl}butyl)carbamate,
methyl (4-hydroxy-4-{1-[4-(methylamino)butanoyl]-3-piperidinyl}-4-{2-[(2-methylphenyl)oxy]phenyl}butyl)carbamate,
methyl [4-[1-β-alanyl-3-piperidinyl]-4-(3′-ethyl-6-fluoro-2-biphenylyl)-4-hydroxybutyl]carbamate,
methyl [4-[1-(4-aminobutanoyl)-3-piperidinyl]-4-(3′-ethyl-6-fluoro-2-biphenylyl)-4-hydroxybutyl]carbamate,
methyl [4-[1-(5-aminopentanoyl)-3-piperidinyl]-4-(3′-ethyl-6-fluoro-2-biphenylyl)-4-hydroxybutyl]carbamate,
methyl 2-amino-5-[3-(1-(3′-ethyl-6-fluoro-2-biphenylyl)-1-hydroxy-4-{[(methyloxy)carbonyl]amino}butyl)-1-piperidinyl]-5-oxopentanoate,
methyl (4-(6-chloro-3′-ethyl-2-biphenylyl)-4-hydroxy-4-{1-[4-(methylamino)butanoyl]-3-piperidinyl}butyl)carbamate,
methyl [4-{1-[4-(dimethylamino)butanoyl]-3-piperidinyl}-4-(3′-ethyl-6-fluoro-2-biphenylyl)-4-hydroxybutyl]carbamate,
methyl (4-[3-chloro-2-(2,3-dihydro-1-benzofuran-6-yl)phenyl]-4-hydroxy-4-{1-[4-(methylamino)butanoyl]-3-piperidinyl}butyl)carbamate,
methyl [4-(3′-ethyl-6-fluoro-2-biphenylyl)-4-hydroxy-4-(1-{N-methyl-N-[2-(methylamino)ethyl]glycyl}-3-piperidinyl)butyl]carbamate,
methyl [4-(3′-ethyl-6-fluoro-2-biphenylyl)-4-hydroxy-4-(1-{-4-[(2-hydroxyethyl)amino]butanoyl}-3-piperidinyl)butyl]carbamate,
2-amino-5-[3-(1-(3′-ethyl-6-fluoro-2-biphenylyl)-1-hydroxy-4-{[(methyloxy)carbonyl]amino}butyl)-1-piperidinyl]-5-oxopentanoic acid,
methyl [4-{1-[4-amino-5-hydroxypentanoyl]-3-piperidinyl}-4-(3′-ethyl-6-fluoro-2-biphenylyl)-4-hydroxybutyl]carbamate,
methyl [4-{1-[4-amino-5-hydroxypentanoyl]-3-piperidinyl}-4-(3′-ethyl-6-fluoro-2-biphenylyl)-4-hydroxybutyl]carbamate,
N-(4-(2′,6-difluoro-5′-methyl-2-biphenylyl)-4-hydroxy-4-{1-[4-(methylamino)butanoyl]-3-piperidinyl}butyl)acetamide,
N-(4-(6-fluoro-3′-methyl-2-biphenylyl)-4-hydroxy-4-{1-[4-(methylamino)butanoyl]-3-piperidinyl}butyl)acetamide,
methyl [4-[1-(4-amino-3-hydroxybutanoyl)-3-piperidinyl]-4-(3′-ethyl-6-fluoro-2-biphenylyl)-4-hydroxybutyl]carbamate,
methyl [4-{1-[4-amino-2-hydroxybutanoyl]-3-piperidnyl}-4-(3′-ethyl-6-fluoro-2-biphenylyl)-4-hydroxybutyl]carbamate,
methyl (4-(3′-ethyl-6-fluoro-2-biphenylyl)-4-hydroxy-4-{1-[4-(methylamino)butanoyl]-3-piperidinyl}butyl)carbamate,
methyl [4-[1-(6-aminohexanoyl)-3-piperidinyl]-4-(3′-ethyl-6-fluoro-2-biphenylyl)-4-hydroxybutyl]carbamate,
methyl [4-[1-L-asparaginyl-3-piperidinyl]-4-(3′-ethyl-6-fluoro-2-biphenylyl)-4-hydroxybutyl]carbamate,
methyl {4-(3′-ethyl-6-fluoro-2-biphenylyl)-4-hydroxy-4-[1-L-valyl-3-piperidinyl]butyl}carbamate,
methyl {4-(3′-ethyl-6-fluoro-2-biphenylyl)-4-hydroxy-4-[1-L-seryl-3-piperidinyl]butyl}carbamate,
methyl [4-[1-(L-alanyl-L-alanyl)-3-piperidinyl]-4-(3′-ethyl-6-fluoro-2-biphenylyl)-4-hydroxybutyl]carbamate,
methyl {4-(3′-ethyl-6-fluoro-2-biphenylyl)-4-hydroxy-4-[1-L-phenylalanyl-3-piperidinyl]butyl}carbamate,
methyl {4-(3′-ethyl-6-fluoro-2-biphenylyl)-4-hydroxy-4-[1-D-tryptophyl-3-piperidinyl]butyl}carbamate,
methyl {4-(3′-ethyl-6-fluoro-2-biphenylyl)-4-[1-(glycylglycyl)-3-piperidinyl]-4-hydroxybutyl}carbamate,
methyl {4-(3′-ethyl-6-fluoro-2-biphenylyl)-4-hydroxy-4-[1-(2-methylalanyl)-3-piperidinyl]butyl}carbamate,
phenylmethyl 2-amino-5-[3-(1-(3′-ethyl-6-fluoro-2-biphenylyl)-1-hydroxy-4-{[(methyloxy)carbonyl]amino}butyl)-1-piperidinyl]-5-oxopentanoate,
phenylmethyl 2-amino-4-[3-(1-(3′-ethyl-6-fluoro-2-biphenylyl)-1-hydroxy-4-{[(methyloxy)carbonyl]amino}butyl)-1-piperidinyl]-4-oxobutanoate,
methyl {4-(3′-ethyl-6-fluoro-2-biphenylyl)-4-[1-L-α-glutaminyl-3-piperidinyl]-4-hydroxybutyl}carbamate,
methyl {4-(3′-ethyl-6-fluoro-2-biphenylyl)-4-hydroxy-4-[1-D-ornithyl-3-piperidinyl]butyl}carbamate,
1-(3-(1-(3-chlorophenyl)-1-hydroxy-5-methoxypentyl)piperidin-1-yl)-4-cyclohexyl-3-((methylamino)methyl)butan-1-one,
3-amino-4-cyclohexyl-1-(3-((3-methoxypropoxy)(phenyl)methyl)piperidin-1-yl)butan-1-one,
4-amino-1-(3-(1-(3-fluorophenyl)-1-hydroxy-5-methoxypentyl)piperidin-1-yl)-3-hydroxy-5-phenylpentan-1-one,
4-amino-3-hydroxy-1-(3-(1-hydroxy-5-methoxy-1-(2-(o-tolyloxy)phenyl)pentyl)piperidin-1-yl)butan-1-one,
4-amino-1-(3-(1-hydroxy-5-methoxy-1-(2-(o-tolyloxy)phenyl)pentyl)piperidin-1-yl)butan-1-one, and
a pharmaceutically acceptable salt thereof.

45. A compound selected from the group
methyl (4-[3-chloro-2-(3-quinolinyl)phenyl]-4-hydroxy-4-{1-[4-(methylamino)butanoyl]-3-piperidinyl}butyl)carbamate,
methyl {4-hydroxy-4-{1-[4-(methylamino)butanoyl]-3-piperidinyl}-4-[2′-(methyloxy)-5′-(trifluoromethyl)-2-biphenylyl]butyl}carbamate,
methyl {4-(3′-ethyl-6-fluoro-2-biphenylyl)-4-[1-glycyl-3-piperidinyl]-4-hydroxybutyl}carbamate,
methyl [4-{1-[4-amino-3-hydroxybutanoyl]-3-piperidinyl}-4-(3′-ethyl-6-fluoro-2-biphenylyl)-4-hydroxybutyl]carbamate,
N-(4-(2′,6-difluoro-5′-methyl-2-biphenylyl)-4-hydroxy-4-{1-[4-(methylamino)butanoyl]-3-piperidinyl}butyl)-2,2,2-trifluoroacetamide,
N [4-{1-[4-amino-3-(4-chlorophenyl)butanoyl]-3-piperidinyl}-4-(2′,6-difluoro-5′-methyl-2-biphenylyl)-4-hydroxybutyl]-2,2,2-trifluoroacetamide,
N-[4-{1-[4-amino-3-hydroxybutanoyl]-3-piperidinyl}-4-(2′,6-difluoro-5′-methyl-2-biphenylyl)-4-hydroxybutyl]-2,2,2-trifluoroacetamide,
N-(4-(3′-ethyl-6-fluoro-2-biphenylyl)-4-hydroxy-4-{1-[4-(methylamino)butanoyl]-3-piperidinyl}butyl)-2,2,2-trifluoroacetamide,
N-[4-{1-[4-amino-3-hydroxybutanoyl]-3-piperidinyl}-4-(3′-ethyl-6-fluoro-2-biphenylyl)-4-hydroxybutyl]-2,2,2-trifluoroacetamide,
methyl [4-[1-(4-{[amino(imino)methyl]amino}butanoyl)-3-piperidinyl]-4-(3′-ethyl-6-fluoro-2-biphertylyl)-4-hydroxybutyl]carbamate,
methyl [4-{1-[4-amino-3-(4-chlorophenyl)butanoyl]-3-piperidinyl}-4-(6-chloro-3′-ethyl-2-biphenylyl)-4-hydroxybutyl]carbamate,
methyl [(4-{1-[4-amino-3-hydroxybutanoyl]-3-piperidinyl}-4-(6-chloro-3′-ethyl-2-biphenylyl)-4-hydroxybutyl]carbamate,
methyl (4-(3′-ethyl-6-fluoro-2-biphenylyl)-4-hydroxy-4-{1-[3-hydroxy-4-(methyl amino)butanoyl]-3-piperidinyl}butyl)carbamate,
N-(4-(3′-ethyl-6-fluoro-2-biphenylyl)-4-hydroxy-4-{1-[3-hydroxy-4-(methylamino)butanoyl]-3-piperidinyl}butyl)-2,2,2-trifluoroacetamide,
methyl (4-(3′-ethyl-6-fluoro-2-biphenylyl)-4-hydroxy-4-{1-[4-(methylamino)-3-(methyloxy)butanoyl]-3-piperidinyl}butyl)carbamate,
methyl (4-[6-fluoro-3′-(1-methylethyl)-2-biphenylyl]-4-hydroxy-4-{1-[4-(methylamino)butanoyl]-3-piperidinyl}butyl)carbamate,
methyl {2-[((6-chloro-3′-ethyl-2-biphenylyl){4-[4-(methylamino)butanoyl]-2-morpholinyl}methyl)oxy]ethyl}carbamate,
methyl {2-[((6-chloro-3′-ethyl-2-biphenylyl){1-[4-(methylamino)butanoyl]-3-piperidinyl}methyl)oxy]ethyl}carbamate,
methyl {4-(3′-ethyl-6-fluoro-2-biphertylyl)-4-hydroxy-4-[1-({[2-(methylamino)ethyl]amino}carbonyl)-3-piperidinyl]butyl}carbamate,
methyl [4-(1-{[(2-aminoethyl)amino]carbonyl}-3-piperidinyl)-4-(3′-ethyl-6-fluoro-2-biphenylyl)-4-hydroxybutyl]carbamate,
methyl [4-(1-{[(3-amino-2,2-dimethylpropyl)amino]carbonyl}-3-piperidinyl)-4-(3′-ethyl-6-fluoro-2-biphenylyl)-4-hydroxybutyl]carbamate,
methyl {2-[((6-chloro-3′-ethyl-2-biphenylyl){1-[({2-(methylamino)-4-[4-(methyloxy)phenyl]butyl}amino)carbonyl]-2-morpholinyl}methyl)oxy]ethyl}carbamate,
methyl {2-[((6-chloro-3′-ethyl-2-biphenylyl){-4-[({2-(methylamino)-3-[4-(methyloxy)phenyl]propyl}amino)carbonyl]-2-morpholinyl}methyl)oxy]ethyl}carbamate,
methyl {2-[((6-chloro-3′-ethyl-2-biphenylyl){1-[({2-(methylamino)-4-[4-(methyloxy)phenyl]butyl}amino)carbonyl]-3-piperidinyl}methyl)oxy]ethyl}carbamate,
methyl {2-[((6-chloro-3′-ethyl-2-biphenylyl){1-[({2-(methylamino)-3-[4-(methyloxy)phenyl]propyl}amino)carbonyl]-3-piperidinyl}methyl)oxy]ethyl}carbamate, and
a pharmaceutically acceptable salt thereof.

46. A pharmaceutical composition comprising the compound according to claim 1, or a pharmaceutically acceptable salt thereof, and a pharmaceutically acceptable carrier or excipient.

47. The pharmaceutical composition according to claim 46, further comprising at least one additional agent selected from the group consisting of an α-blocker, a β-blocker, a calcium channel blocker, a diuretic, an angiotensin converting enzyme inhibitor, a dual angiotensin converting enzyme-neutral endopeptidase inhibitor, an angiotensin-receptor blocker, an aldosterone synthase inhibitor, an aldosterone-receptor antagonist, and an endothelin receptor antagonist.

48-53. (canceled)

54. A pharmaceutical composition comprising the compound of claim 44, or a pharmaceutically acceptable salt thereof, and a pharmaceutically acceptable carrier or excipient.

55. A pharmaceutical composition comprising the compound of claim 45, or a pharmaceutically acceptable salt thereof, and a pharmaceutically acceptable carrier or excipient.
